SEQUENCE LISTING (278 sequences)

SEQ ID NO: 1 - protein, 29 aa, synthetic construct
AALEALAEALEALAEALEALAEAAAAGGC

SEQ ID NO: 2 - protein, 30 aa, synthetic construct
AALAEALAEALAEALAEALAEALAAAAGGC

SEQ ID NO: 3 - protein, 15 aa, synthetic construct
ALEALAEALEALAEA

SEQ ID NO: 4 - protein, 22 aa, synthetic construct
GLFEAIEGFIENGWEGMIWDYG

SEQ ID NO: 5 - protein, 23 aa, synthetic construct
GLFGAIAGFIENGWEGMIDGWYG

SEQ ID NO: 6 - protein, 48 aa, synthetic construct
GLFEAIEGFIENGWEGMIDGWYGCGLFEAIEGFIENGWEGMIDGWYGC

SEQ ID NO: 7 - protein, 44 aa, synthetic construct
GLFEAIEGFIENGWEGMIDGGCGLFEAIEGFIENGWEGMIDGGC

SEQ ID NO: 8 - protein, 35 aa, synthetic construct
GLFGALAEALAEALAEHLAEALAEALEALAAGGSC

SEQ ID NO: 9 - protein, 34 aa, synthetic construct
GLFEAIEGFIENGWEGLAEALAEALEALAAGGSC

SEQ ID NO: 10 - protein, 41 aa, synthetic construct
  SITE 17: note = Nle
  SITE 38: note = Nle
GLFEAIEGFIENGWEGXIDGKGLFEAIEGFIENGWEGXIDG

SEQ ID NO: 11 - protein, 19 aa, synthetic construct
LFEALLELLESLWELLLEA

SEQ ID NO: 12 - protein, 20 aa, synthetic construct
GLFKALLKLLKSLWKLLLKA

SEQ ID NO: 13 - protein, 20 aa, synthetic construct
GLFRALLRLLRSLWRLLLRA

SEQ ID NO: 14 - protein, 30 aa, synthetic construct
WEAKLAKALAKALAKHLAKALAKALKACEA

SEQ ID NO: 15 - protein, 22 aa, synthetic construct
GLFFEAIAEFIEGGWEGLIEGC

SEQ ID NO: 16 - protein, 26 aa, synthetic construct
GIGAVLKVLTTGLPALISWIKRKRQQ

SEQ ID NO: 17 - protein, 8 aa, synthetic construct
HHHHHWYG

SEQ ID NO: 18 - protein, 10 aa, synthetic construct
CHKKKKKKHC

SEQ ID NO: 19 - protein, 16 aa, synthetic construct
RQIKIWFQNRRMKWKK

SEQ ID NO: 20 - protein, 14 aa, synthetic construct
GRKKRRQRRRPPQC

SEQ ID NO: 21 - protein, 27 aa, synthetic construct
GALFLGWLGAAGSTMGAWSQPKKKRKV

SEQ ID NO: 22 - protein, 18 aa, synthetic construct
LLIILRRRIRKQAHAHSK

SEQ ID NO: 23 - protein, 26 aa, synthetic construct
GWTLNSAGYLLKINLKALAALAKKIL

SEQ ID NO: 24 - protein, 18 aa, synthetic construct
KLALKLALKALKAALKLA

SEQ ID NO: 25 - protein, 9 aa, synthetic construct
RRRRRRRRR

SEQ ID NO: 26 - protein, 10 aa, synthetic construct
KFFKFFKFFK

SEQ ID NO: 27 - protein, 37 aa, synthetic construct
LLGDFFRKSKEKIGKEFKRIVQRIKDFLRNLVPRTES

SEQ ID NO: 28 - protein, 31 aa, synthetic construct
SWLSKTAKKLENSAKKRISEGIAIAIQGGPR

SEQ ID NO: 29 - protein, 30 aa, synthetic construct
ACYCRIPACIAGERRYGTCIYQGRLWAFCC

SEQ ID NO: 30 - protein, 36 aa, synthetic construct
DHYNCVSSGGQCLYSACPIFTKIQGTCYRGKAKCCK

SEQ ID NO: 31 - protein, 42 aa, synthetic construct
RRRPRPPYLPRPRPPPFFPPRLPPRIPPGFPPRFPPRFPGKR

SEQ ID NO: 32 - protein, 13 aa, synthetic construct
ILPWKWPWWPWRR

SEQ ID NO: 33 - protein, 16 aa, synthetic construct
AAVALLPAVLLALLAP

SEQ ID NO: 34 - protein, 11 aa, synthetic construct
AALLPVLLAAP

SEQ ID NO: 35 - protein, 12 aa, synthetic construct
RKCRIVVIRVCR

SEQ ID NO: 36 - protein, 11 aa, synthetic construct
FRGDLAFDPNK

SEQ ID NO: 37 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,10,13): mod_base = OTHER; note = 2 prime-O-methyladenosine-3 prime-phosphate
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(12,19): mod_base = OTHER; note = 2 prime-O-methylguanosine-3 prime-phosphate
  modified_base order(9,15,17): mod_base = OTHER; note = 2 prime-O-methylcytidine-3 prime-phosphate
  modified_base order(1^2,2^3,21^22,22^23): mod_base = OTHER; note = Phosphorothioate linkage
ttatagagcaagaacactgtttt

SEQ ID NO: 38 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(10,14): mod_base = OTHER; note = 2 prime-deoxy-2 prime-fluoroadenosine-3 prime-phosphate
  modified_base order(6,7): mod_base = OTHER; note = 2 prime-deoxy-2 prime-fluoroguanosine-3 prime-phosphate
  modified_base order(12,19): mod_base = OTHER; note = 2 prime-O-methylcytidine-3 prime-phosphate
  modified_base order(9,15,17): mod_base = OTHER; note = 2 prime-O-methylcytidine-3 prime-phosphate
  modified_base order(1^2,2^3,21^22,22^23): mod_base = OTHER; note = Phosphorothioate linkage
  modified_base 16: mod_base = OTHER; note = 2 prime-deoxy-2-aminoadenosine-3 prime-phosphate (deoxy-2,6-diaminopurine)
ttatagagcaagaacnctgtttt

SEQ ID NO: 39 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-deoxy-2 prime-fluorouridine-3 prime-phosphate
  modified_base order(3,5,7,11,12): mod_base = OTHER; note = 2 prime-O-methyladenosine-3 prime-phosphate
  modified_base order(10,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 14: mod_base = OTHER; note = 2 prime-deoxy-2-aminoadenosine-3 prime-phosphate (deoxy-2,6-diaminopurine)
  modified_base order(1^2,2^3,21^22,22^23): mod_base = OTHER; note = Phosphorothioate linkage
ttatagagcaagancactgtttt

SEQ ID NO: 40 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base order(1^2,2^3,21^22,22^23): mod_base = OTHER; note = Phosphorothioate linkage
ttatagagcnagaacactgtttt

SEQ ID NO: 41 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base 14: mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base order(10,16): mod_base = OTHER; note = 2 prime-deoxy-2-aminoadenosine-3 prime-phosphate (deoxy-2,6-diaminopurine)
  modified_base order(1^2,2^3,21^22,22^23): mod_base = OTHER; note = Phosphorothioate linkage
ttatagagcnagaacnctgtttt

SEQ ID NO: 42 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base order(10,14,17): mod_base = OTHER; note = 2 prime-deoxy-2-aminoadenosine-3 prime-phosphate (deoxy-2,6-diaminopurine)
  modified_base order(1^2,2^3,21^22,22^23): mod_base = OTHER; note = Phosphorothioate linkage
ttatagagcnagancnctgtttt

SEQ ID NO: 43 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(3,5,7,11,13): mod_base = OTHER; note = 2 prime-O-methyladenosine-3 prime-phosphate
  modified_base order(10,14): mod_base = OTHER; note = 2 prime-deoxy-2 prime-fluoroadenosine-3 prime-phosphate
  modified_base order(9,15,17): mod_base = OTHER; note = 2 prime-O-methylcytidine-3 prime-phosphate
  modified_base 16: mod_base = OTHER; note = 2 prime-deoxy-2 prime-fluoro-2-aminoadenosine-3 prime-phosphate
  modified_base order(1^2,2^3,21^22,22^23): mod_base = OTHER; note = Phosphorothioate linkage
ttatagagcaagaacnctgtttt

SEQ ID NO: 44 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(10,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 14: mod_base = OTHER; note = 2 prime-deoxy-2 prime-fluoro-2-aminoadenosine-3 prime-phosphate (2 prime-F-2,6-diaminopurine)
  modified_base order(1^2,2^3,21^22,22^23): mod_base = OTHER; note = Phosphorothioate linkage
ttatagagcaagancactgtttt

SEQ ID NO: 45 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 10: mod_base = OTHER; note = 2 prime-deoxy-2 prime-fluoro-2-aminoadenosine-3 prime-phosphate (2 prime-F-2,6-diaminopurine)
  modified_base order(1^2,2^3,21^22,22^23): mod_base = OTHER; note = Phosphorothioate linkage
ttatagagcnagaacactgtttt

SEQ ID NO: 46 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base 14: mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 10: mod_base = OTHER; note = 2 prime-deoxy-2 prime-fluoro-2-aminoadenosine-3 prime-phosphate (2 prime-F-2,6-diaminopurine)
  modified_base order(1^2,2^3,21^22,22^23): mod_base = OTHER; note = Phosphorothioate linkage
ttatagagcnagaacnctgtttt

SEQ ID NO: 47 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(3,4,7,11,13): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(10,14,16): mod_base = OTHER; note = 2 prime-deoxy-2 prime-fluoro-2-aminoadenosine-3 prime-phosphate (2 prime-F-2,6-diaminopurine)
  modified_base order(1^2,2^3,21^22,22^23): mod_base = OTHER; note = Phosphorothioate linkage
ttatagagcnagancnctgtttt

SEQ ID NO: 48 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-O-methyluridine-3 prime-phosphate
  modified_base order(3,5,7,11,13): mod_base = OTHER; note = 2 prime-O-methyladenosine-3 prime-phosphate
  modified_base order(10,14): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(12,19): mod_base = OTHER; note = 2 prime-O-methylguanosine-3 prime-phosphate
  modified_base 16: mod_base = OTHER; note = 2-amino-2 prime-O,4 prime-C-methylene adenosine-3 prime-phosphate (LNA-2,6-diaminopurine)
  modified_base order(9,15,17): mod_base = OTHER; note = 2 prime-O-methylcytidine-3 prime-phosphate
  modified_base order(1^2,2^3,21^22,22^23): mod_base = OTHER; note = Phosphorothioate linkage
ttatagagcaagaacnctgtttt

SEQ ID NO: 49 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(10,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,16): mod_base = OTHER; note = 2 prime-O-methylcytidine-3 prime-phosphate
  modified_base 14: mod_base = OTHER; note = 2-amino-2 prime-O,4 prime-C-methylene adenosine-3 prime-phosphate (LNA-2,6-diaminopurine)
  modified_base order(1^2,2^3,21^22,22^23): mod_base = OTHER; note = Phosphorothioate linkage
ttatagagcaagancactgtttt

SEQ ID NO: 50 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-deoxy-2 prime-fluorouridine-3 prime-phosphate
  modified_base order(3,4,7,11,13): mod_base = OTHER; note = 2 prime-O-methyladenosine-3 prime-phosphate
  modified_base order(14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 10: mod_base = OTHER; note = 2-amino-2 prime-O,4 prime-C-methylene adenosine-3 prime-phosphate (LNA-2,6-diaminopurine)
  modified_base order(1^2,2^3,21^22,22^23): mod_base = OTHER; note = Phosphorothioate linkage
ttatagagcnagaacactgtttt

SEQ ID NO: 51 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base 14: mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base order(10,16): mod_base = OTHER; note = 2-amino-2 prime-O,4 prime-C-methylene adenosine-3 prime-phosphate (LNA-2,6-diaminopurine)
  modified_base order(1^2,2^3,21^22,22^23): mod_base = OTHER; note = Phosphorothioate linkage
ttatagagcnagaacnctgtttt

SEQ ID NO: 52 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2 prime-O-methyladenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(10,14,16): mod_base = OTHER; note = 2-amino-2 prime-O,4 prime-C-methylene adenosine-3 prime-phosphate (LNA-2,6-diaminopurine)
  modified_base order(1^2,2^3,21^22,22^23): mod_base = OTHER; note = Phosphorothioate linkage
ttatagagcnagancnctgtttt

SEQ ID NO: 53 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,11): mod_base = OTHER; note = 2 prime-O-methyladenosine-3 prime-phosphate
  modified_base order(10,14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 13: mod_base = OTHER; note = 2-aminoadenosine-3 prime-phosphate (ribo-2,6-diaminopurine)
  modified_base order(1^2,2^3,21^22,22^23): mod_base = OTHER; note = Phosphorothioate linkage
ttatagagcaagnacactgtttt

SEQ ID NO: 54 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(3,5,7,13): mod_base = OTHER; note = 2 prime-O-methyladenosine-3 prime-phosphate
  modified_base order(14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base 11: mod_base = OTHER; note = 2-aminoadenosine-3 prime-phosphate (ribo-2,6-diaminopurine)
  modified_base order(9,15,17): mod_base = OTHER; note = 2 prime-O-methylcytidine-3 prime-phosphate
  modified_base order(1^2,2^3,21^22,22^23): mod_base = OTHER; note = Phosphorothioate linkage
ttatagagcangaacactgtttt

SEQ ID NO: 55 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,19): mod_base = OTHER; note = 2 prime-deoxy-2 prime-fluorouridine-3 prime-phosphate
  modified_base order(3,5,11,13): mod_base = OTHER; note = 2 prime-O-methyladenosine-3 prime-phosphate
  modified_base order(10,14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 7: mod_base = OTHER; note = 2-aminoadenosine-3 prime-phosphate (ribo-2,6-diaminopurine)
  modified_base order(1^2,2^3,21^22,22^23): mod_base = OTHER; note = Phosphorothioate linkage
ttatagngcaagaacactgtttt

SEQ ID NO: 56 - RNA, 23 nt, synthetic construct
  source 1..23: mol_type = other RNA
  modified_base order(1,21..23): mod_base = OTHER; note = 2 prime-O-methyluridine-3 prime-phosphate
  modified_base order(2,4,18,20): mod_base = OTHER; note = 2 prime-deoxy-2 prime-fluorouridine-3 prime-phosphate
  modified_base order(1,7,11,13): mod_base = OTHER; note = 2 prime-O-methyladenosine-3 prime-phosphate
  modified_base order(10,14,16): mod_base = OTHER; note = 2 prime-deoxy-2 prime-fluoroadenosine-3 prime-phosphate
  modified_base order(12,19): mod_base = OTHER; note = 2 prime-O-methylguanosine-3 prime-phosphate
  modified_base order(6,8): mod_base = OTHER; note = 2 prime-deoxy-2 prime-fluoroguanosine-3 prime-phosphate
  modified_base order(9,15,17): mod_base = OTHER; note = 2 prime-O-methylcytidine-3 prime-phosphate
  modified_base 5: mod_base = OTHER; note = 2-aminoadenosine-3 prime-phosphate (ribo-2,6-diaminopurine)
  modified_base order(1^2,2^3,21^22,22^23): mod_base = OTHER; note = Phosphorothioate linkage
ttatngagcaagaacactgtttt

SEQ ID NO: 57 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,7): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(5,7,11,13): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 3: mod_base = OTHER; note = 2-aminoadenosine-3 prime-phosphate (ribo-2,6-diaminopurine)
  modified_base order(1^2,2^3,21^22,22^23): mod_base = OTHER; note = Phosphorothioate linkage
ttntagagcaagaacactgtttt

SEQ ID NO: 58 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(3,5,7,11,13): mod_base = OTHER; note = 2-aminoadenosine-3 prime-phosphate (ribo-2,6-diaminopurine)
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base order(1^2,2^3,21^22,22^23): mod_base = OTHER; note = Phosphorothioate linkage
ttntngngcangnacactgtttt

SEQ ID NO: 59 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(3,5,7,11): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 13: mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttatagagcaagnacactgtttt

SEQ ID NO: 60 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(3,5,7,13): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base order(1^2,2^3,21^22,22^23): mod_base = OTHER; note = Phosphorothioate linkage
  modified_base 11: mod_base = OTHER; note = 2 prime-O-methyl-2-aminoadenosine-3 prime-phosphate (2 primeOMe-2,6-diaminopurine)
ttatagagcangaacactgtttt

SEQ ID NO: 61 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,19): mod_base = OTHER; note = 2 prime-deoxy-2 prime-fluorouridine-3 prime-phosphate
  modified_base order(3,5,11,13): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 7: mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttatagngcaagaacactgtttt

SEQ ID NO: 62 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(3,7,11,13): mod_base = OTHER; note = 2 prime-O-methyladenosine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 5: mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttatngagcaagaacactgtttt

SEQ ID NO: 63 - DNA, 23 bp, synthetic construct
  source 1..23: mol_type = other DNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(5,7,11,13): mod_base = OTHER; note = 2 prime-O-methyladenosine-3 prime-phosphate
  modified_base order(9,15,16): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 3: mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttntagagcaagaacactgtttt

SEQ ID NO: 64 - RNA, 23 nt, synthetic construct
  source 1..23: mol_type = other RNA
  modified_base order(10,14,16): mod_base = OTHER; note = 2 prime-deoxy-2 prime-fluoroadenosine-3 prime-phosphate
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base order(3,5,7,11,13): mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttntngngcangnacactgtttt

SEQ ID NO: 65 - RNA, 23 nt, synthetic construct
  source 1..23: mol_type = other RNA
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base order(10,14,16): mod_base = OTHER; note = 2 prime-deoxy-2 prime-fluoro-2-aminoadenosine-3 prime-phosphate (2 prime-F-2,6-diaminopurine)
  modified_base 7: mod_base = OTHER; note = 2-amino-2 prime-O,4 prime-C-methylene adenosine-3 prime-phosphate (LNA-2,6-diaminopurine)
  modified_base order(3,5,11): mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttntngngcnngnncnctgtttt

SEQ ID NO: 66 - RNA, 23 nt, synthetic construct
  source 1..23: mol_type = other RNA
  modified_base order(6,8): mod_base = OTHER; note = 2 prime-deoxy-2 prime-fluoroguanosine-3 prime-phosphate
  modified_base order(2,4,18,20): mod_base = OTHER; note = 2 prime-deoxy-2 prime-fluorouridine-3 prime-phosphate
  modified_base order(9,15,17): mod_base = OTHER; note = 2 prime-O-methylcytidine-3 prime-phosphate
  modified_base order(12,19): mod_base = OTHER; note = 2 prime-O-methylguanosine-3 prime-phosphate
  modified_base order(1,21..23): mod_base = OTHER; note = 2 prime-O-methyluridine-3 prime-phosphate
  modified_base 3: mod_base = OTHER; note = 2-aminoadenosine-3 prime-phosphate (ribo-2,6-diaminopurine)
  modified_base order(14,16): mod_base = OTHER; note = 2 prime-deoxy-2 prime-fluoro-2-aminoadenosine-3 prime-phosphate (2 prime-F-2,6-diaminopurine)
  modified_base 7: mod_base = OTHER; note = 2-amino-2 prime-O,4 prime-C-methylene adenosine-3 prime-phosphate (LNA-2,6-diaminopurine)
  modified_base order(5,11,13): mod_base = OTHER; note = 2 prime-O-methyl-2-aminoadenosine-3 prime-phosphate (2 primeOMe-2,6-diaminopurine)
  modified_base order(1^2,2^3,21^22,22^23): mod_base = OTHER; note = Phosphorothioate linkage
ttntngngcnngnncnctgtttt

SEQ ID NO: 67 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base order(1^2,2^3): mod_base = OTHER; note = Phosphorothioate linkage
aacagtgttcttgctctataa

SEQ ID NO: 68 - RNA, 21 nt, synthetic construct
  source 1..21: mol_type = other RNA
  modified_base 1: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 21: mod_base = OTHER; note = 2 prime-deoxy-2-aminoadenosine-3 prime-phosphate (deoxy-2,6-diaminopurine)
  modified_base order(1^2,2^3): mod_base = OTHER; note = Phosphorothioate linkage
aacagtgttcttgctctatan

SEQ ID NO: 69 - RNA, 21 nt, synthetic construct
  source 1..21: mol_type = other RNA
  modified_base 21: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(6,8,12): mod_base = OTHER; note = 2 prime-O-methyladenosine-3 prime-phosphate
  modified_base order(14,17): mod_base = OTHER; note = 2 prime-O-methylcytidine-3 prime-phosphate
  modified_base 1: mod_base = OTHER; note = 2 prime-deoxy-2-aminoadenosine-3 prime-phosphate (deoxy-2,6-diaminopurine)
nacagtgttcttgctctataa

SEQ ID NO: 70 - RNA, 21 nt, synthetic construct
  source 1..21: mol_type = other RNA
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,19): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base order(1,21): mod_base = OTHER; note = 2 prime-deoxy-2-aminoadenosine-3 prime-phosphate (deoxy-2,6-diaminopurine)
  modified_base order(1^2,2^3): mod_base = OTHER; note = Phosphorothioate linkage
nacagtgttcttgctctatan

SEQ ID NO: 71 - RNA, 21 nt, synthetic construct
  source 1..21: mol_type = other RNA
  modified_base 1: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 21: mod_base = OTHER; note = 2 prime-deoxy-2 prime-fluoro-2-aminoadenosine-3 prime-phosphate (2 prime-F-2,6-diaminopurine)
  modified_base order(1^2,2^3): mod_base = OTHER; note = Phosphorothioate linkage
aacagtgttcttgctctatan

SEQ ID NO: 72 - RNA, 21 nt, synthetic construct
  source 1..21: mol_type = other RNA
  modified_base 21: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 1: mod_base = OTHER; note = 2 prime-deoxy-2 prime-fluoro-2-aminoadenosine-3 prime-phosphate (2 prime-F-2,6-diaminopurine)
  modified_base order(1^2,2^3): mod_base = OTHER; note = Phosphorothioate linkage
nacagtgttcttgctctataa

SEQ ID NO: 73 - RNA, 21 nt, synthetic construct
  source 1..21: mol_type = other RNA
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base order(1,21): mod_base = OTHER; note = 2 prime-deoxy-2 prime-fluoro-2-aminoadenosine-3 prime-phosphate (2 prime-F-2,6-diaminopurine)
  modified_base order(1^2,2^3): mod_base = OTHER; note = Phosphorothioate linkage
nacagtgttcttgctctatan

SEQ ID NO: 74 - RNA, 21 nt, synthetic construct
  source 1..21: mol_type = other RNA
  modified_base 1: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 21: mod_base = OTHER; note = 2-amino-2 prime-O,4 prime-C-methylene adenosine-3 prime-phosphate (LNA-2,6-diaminopurine)
  modified_base order(1^2,2^3): mod_base = OTHER; note = Phosphorothioate linkage
aacagtgttcttgctctatan

SEQ ID NO: 75 - RNA, 21 nt, synthetic construct
  source 1..21: mol_type = other RNA
  modified_base 21: mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 1: mod_base = OTHER; note = 2-amino-2 prime-O,4 prime-C-methylene adenosine-3 prime-phosphate (LNA-2,6-diaminopurine)
  modified_base order(1^2,2^3): mod_base = OTHER; note = Phosphorothioate linkage
nacagtgttcttgctctataa

SEQ ID NO: 76 - RNA, 21 nt, synthetic construct
  source 1..21: mol_type = other RNA
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base order(1,21): mod_base = OTHER; note = 2-amino-2 prime-O,4 prime-C-methylene adenosine-3 prime-phosphate (LNA-2,6-diaminopurine)
  modified_base order(1^2,2^3): mod_base = OTHER; note = Phosphorothioate linkage
nacagtgttcttgctctatan

SEQ ID NO: 77 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 20: mod_base = OTHER; note = 2 prime-O-methyl-2-aminoadenosine-3 prime-phosphate (2 primeOMe-2,6-diaminopurine)
  modified_base order(1^2,2^3): mod_base = OTHER; note = Phosphorothioate linkage
aacagtgttcttgctctatna

SEQ ID NO: 78 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,20): mod_base = OTHER; note = 2 prime-O-methyladenosine-3 prime-phosphate
  modified_base order(14,15): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 18: mod_base = OTHER; note = 2 prime-O-methyl-2-aminoadenosine-3 prime-phosphate (2 primeOMe-2,6-diaminopuri
  modified_base order(1^2,2^3): mod_base = OTHER; note = Phosphorothioate linkage
aacagtgttcttgctctntaa

SEQ ID NO: 79 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(3,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 2: mod_base = OTHER; note = 2 prime-O-methyl-2-aminoadenosine-3 prime-phosphate (2 primeOMe-2,6-diaminopurine)
  modified_base order(1^2,2^3): mod_base = OTHER; note = Phosphorothioate linkage
aacngtgttcttgctctataa

SEQ ID NO: 80 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 2: mod_base = OTHER; note = 2 prime-O-methyl-2-aminoadenosine-3 prime-phosphate (2 primeOMe-2,6-diaminopurine)
  modified_base order(1^2,2^3): mod_base = OTHER; note = Phosphorothioate linkage
ancagtgttcttgctctataa

SEQ ID NO: 81 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(6,8,12): mod_base = OTHER; note = Vinylphosphonate2 prime-O-methyluridine-3 prime-phosphate
  modified_base order(2,4,18,20): mod_base = OTHER; note = 2 prime-O-methyl-2-aminoadenosine-3 prime-phosphate (2 primeOMe-2,6-diaminopurine)
  modified_base order(1^2,2^3): mod_base = OTHER; note = Phosphorothioate linkage
ancngtgttcttgctctntna

SEQ ID NO: 82 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,18): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 20: mod_base = OTHER; note = 2-aminoadenosine-3 prime-phosphate (ribo-2,6-diaminopurine)
  modified_base order(1^2,2^3): mod_base = OTHER; note = Phosphorothioate linkage
aacagtgttcttgctctatna

SEQ ID NO: 83 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,4,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 18: mod_base = OTHER; note = 2-aminoadenosine-3 prime-phosphate (ribo-2,6-diaminopurine)
  modified_base order(1^2,2^3): mod_base = OTHER; note = Phosphorothioate linkage
aacagtgttcttgctctntaa

SEQ ID NO: 84 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(2,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 4: mod_base = OTHER; note = 2-aminoadenosine-3 prime-phosphate (ribo-2,6-diaminopurine)
  modified_base order(1^2,2^3): mod_base = OTHER; note = Phosphorothioate linkage
aacngtgttcttgctctataa

SEQ ID NO: 85 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluorocytidine-3 prime-phosphate
  modified_base order(9,11,15,17,19): mod_base = OTHER; note = 2 prime-deoxy-2 prime-fluorouridine-3 prime-phosphate
  modified_base order(4,18,20): mod_base = OTHER; note = 2 prime-O-methyladenosine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 2: mod_base = OTHER; note = 2-aminoadenosine-3 prime-phosphate (ribo-2,6-diaminopurine)
  modified_base order(1^2,2^3): mod_base = OTHER; note = Phosphorothioate linkage
ancagtgttcttgctctataa

SEQ ID NO: 86 - RNA, 21 nt, synthetic construct
  source 1..21: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base order(2,4,18,20): mod_base = OTHER; note = 2-aminoadenosine-3 prime-phosphate (ribo-2,6-diaminopurine)
  modified_base order(1^2,2^3): mod_base = OTHER; note = Phosphorothioate linkage
ancngtgttcttgctctntna

SEQ ID NO: 87 - RNA, 21 nt, synthetic construct
  source 1..21: mol_type = other RNA
  modified_base order(3,10): mod_base = OTHER; note = 2 prime-deoxy-2 prime-fluorocytidine-3 prime-phosphate
  modified_base order(5,7,13): mod_base = OTHER; note = 2 prime-deoxy-2 prime-fluoroguanosine-3 prime-phosphate
  modified_base order(9,11,15,17,19): mod_base = OTHER; note = 2 prime-deoxy-2 prime-fluorouridine-3 prime-phosphate
  modified_base order(14,16): mod_base = OTHER; note = 2 prime-O-methylcytidine-3 prime-phosphate
  modified_base order(6,8,12): mod_base = OTHER; note = 2 prime-O-methyluridine-3 prime-phosphate
  modified_base 1: mod_base = OTHER; note = 2-amino-2 prime-O,4 prime-C-methylene adenosine-3 prime-phosphate (LNA-2,6-diaminopurine)
  modified_base order(2,4,18,20): mod_base = OTHER; note = 2 prime-O-methyl-2-aminoadenosine-3 prime-phosphate (2 primeOMe-2,6-diaminopurine)
  modified_base 21: mod_base = OTHER; note = 2 prime-deoxy-2-aminoadenosine-3 prime-phosphate (deoxy-2,6-diaminopurine)
  modified_base order(1^2,2^3): mod_base = OTHER; note = Phosphorothioate linkage
nncngtgttcttgctctntnn

SEQ ID NO: 88 - RNA, 16 nt, synthetic construct
  source 1..16: mol_type = other RNA
  modified_base 9: mod_base = OTHER; note = 2 prime-deoxy-2 prime-fluoroadenosine-3 prime-phosphate
  modified_base 12: mod_base = OTHER; note = 2 prime-deoxy-2 prime-fluorocytidine-3 prime-phosphate
  modified_base 10: mod_base = OTHER; note = 2 prime-deoxy-2 prime-fluoroguanosine-3 prime-phosphate
  modified_base order(3,8,15,16): mod_base = OTHER; note = 2 prime-O-methyladenosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base order(1^2,2^3,3^4,4^5,5^6,6^7,7^8,8^9,9^10,10^11,11^12,12^13,13^14,14^15): mod_base = OTHER; note = Phosphorothioate linkage
tcatctcaagtcttaa

SEQ ID NO: 89 - RNA, 16 nt, synthetic construct
  source 1..16: mol_type = other RNA
  modified_base 12: mod_base = OTHER; note = 2 prime-deoxy-2 prime-fluorocytidine-3 prime-phosphate
  modified_base 10: mod_base = OTHER; note = 2 prime-deoxy-2 prime-fluoroguanosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base 16: mod_base = OTHER; note = 2 prime-deoxy-2-aminoadenosine-3 prime-phosphate (deoxy-2,6-diaminopurine)
  modified_base order(3,8,9,15): mod_base = OTHER; note = 2-amino-2 prime-O,4 prime-C-methylene adenosine-3 prime-phosphate (LNA-2,6-diaminopurine)
  modified_base order(1^2,2^3,3^4,4^5,5^6,6^7,7^8,8^9,9^10,10^11,11^12,12^13,13^14,14^15): mod_base = OTHER; note = Phosphorothioate linkage
tcntctcnngtcttnn

SEQ ID NO: 90 - RNA, 16 nt, synthetic construct
  source 1..16: mol_type = other RNA
  modified_base 12: mod_base = OTHER; note = 2 prime-deoxy-2 prime-fluorocytidine-3 prime-phosphate
  modified_base 10: mod_base = OTHER; note = 2 prime-deoxy-2 prime-fluoroguanosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base 16: mod_base = OTHER; note = 2 prime-deoxy-2-aminoadenosine-3 prime-phosphate (deoxy-2,6-diaminopurine)
  modified_base 9: mod_base = OTHER; note = 2-amino-2 prime-O,4 prime-C-methylene adenosine-3 prime-phosphate (LNA-2,6-diaminopurine)
  modified_base order(3,8,15): mod_base = OTHER; note = 2 prime-O-methyl-2-aminoadenosine-3 prime-phosphate (2 primeOMe-2,6-diaminopurine)
  modified_base order(1^2,2^3,3^4,4^5,5^6,6^7,7^8,8^9,9^10,10^11,11^12,12^13,13^14,14^15): mod_base = OTHER; note = Phosphorothioate linkage
tcntctcnngtcttnn

SEQ ID NO: 91 - RNA, 23 nt, synthetic construct
  source 1..23: mol_type = other RNA
  modified_base order(1,3..5,7,16,18,23): mod_base = OTHER; note = 2 prime-O-methyladenosine-3 prime-phosphate
  modified_base order(2,6,8,9,15,17,19,21,22): mod_base = OTHER; note = 2 prime-O-methylcytidine-3 prime-phosphate
  modified_base 13: mod_base = OTHER; note = 2 prime-O-methylguanosine-3 prime-phosphate
  modified_base order(11,12,14,20): mod_base = OTHER; note = 2 prime-O-methyluridine-3 prime-phosphate
  modified_base order(1^2,2^3,21^22,22^23): mod_base = OTHER; note = Phosphorothioate linkage
acaaacaccattgtcacactcca

SEQ ID NO: 92 - RNA, 23 nt, synthetic construct
  source 1..23: mol_type = other RNA
  modified_base order(2,6,8,9,15,17,19,21,22): mod_base = OTHER; note = 2 prime-O-methylcytidine-3 prime-phosphate
  modified_base 13: mod_base = OTHER; note = 2 prime-O-methylguanosine-3 prime-phosphate
  modified_base order(11,12,14,20): mod_base = OTHER; note = 2 prime-O-methyluridine-3 prime-phosphate
  modified_base order(1,3..5,7,10,18,23): mod_base = OTHER; note = 2-amino-2 prime-O,4 prime-C-methylene adenosine-3 prime-phosphate (LNA-2,6-diaminopurine)
  modified_base 16: mod_base = OTHER; note = 2 prime-O-methyl-2-aminoadenosine-3 prime-phosphate (2 primeOMe-2,6-diaminopurine)
  modified_base order(1^2,2^3,20^21,21^22,22^23): mod_base = OTHER; note = Phosphorothioate linkage
ncnnncnccnttgtcncnctccn

SEQ ID NO: 93 - RNA, 44 nt, synthetic construct
  source 1..44: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,39,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42..44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
  modified_base order(31,35,37): mod_base = OTHER; note = 2 prime-deoxy-2 prime-fluoro-2-aminoadenosine-3 prime-phosphate (2 prime-F-2,6-diaminopurine)
aacagtgttcttgctctataattatagagcaagaacactgtttt

SEQ ID NO: 94 - RNA, 44 nt, synthetic construct
  source 1..44: mol_type = other RNA
  modified_base order(1,21,31,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(5,7,13,27,29,33): mod_base = OTHER; note = 2 prime-deoxy-2 prime-fluoroguanosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base 37: mod_base = OTHER; note = 2 prime-deoxy-2-aminoadenosine-3 prime-phosphate (deoxy-2,6-diaminopurine)
  modified_base order(1^2,2^3,22^23,23^24,42^43,43^44): mod_base = OTHER; note = Phosphorothioate linkage
aacagtgttcttgctctataattatagagcaagaacnctgtttt

SEQ ID NO: 95 - RNA, 44 nt, synthetic construct
  source 1..44: mol_type = other RNA
  modified_base order(1,21,31,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(5,7,13,27,29,33): mod_base = OTHER; note = 2 prime-deoxy-2 prime-fluoroguanosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42..44): mod_base = OTHER; note = 2 prime-O-methyluridine-3 prime-phosphate
  modified_base 35: mod_base = OTHER; note = 2 prime-deoxy-2-aminoadenosine-3 prime-phosphate (deoxy-2,6-diaminopurine)
  modified_base order(1^2,2^3,22^23,23^24,42^43,43^44): mod_base = OTHER; note = Phosphorothioate linkage
aacagtgttcttgctctataattatagagcaagancactgtttt

SEQ ID NO: 96 - RNA, 44 nt, synthetic construct
  source 1..44: mol_type = other RNA
  modified_base order(1,21,35,37): mod_base = OTHER; note = 2 prime-deoxy-2 prime-fluoroadenosine-3 prime-phosphate
  modified_base 31: mod_base = OTHER; note = 2 prime-deoxy-2-aminoadenosine-3 prime-phosphate (deoxy-2,6-diaminopurine)
  modified_base order(3,10): mod_base = OTHER; note = 2 prime-deoxy-2 prime-fluorocytidine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(5,7,13,27,29,33): mod_base = OTHER; note = 2 prime-deoxy-2 prime-fluoroguanosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatagagcnagaacactgtttt

SEQ ID NO: 97 - RNA, 44 nt, synthetic construct
  source 1..44: mol_type = other RNA
  modified_base order(1,21,35,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluoroadenosine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(31,37): mod_base = OTHER; note = 2 prime-deoxy-2-aminoadenosine-3 prime-phosphate (deoxy-2,6-diaminopurine)
  modified_base order(1^2,2^3,22^23,23^24,42^43,43^44): mod_base = OTHER; note = Phosphorothioate linkage
aacagtgttcttgctctataattatagagcnagaacnctgtttt

SEQ ID NO: 98 - RNA, 44 nt, synthetic construct
  source 1..44: mol_type = other RNA
  modified_base order(31,35,37): mod_base = OTHER; note = 2 prime-deoxy-2-aminoadenosine-3 prime-phosphate (deoxy-2,6-diaminopurine)
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatagagcnagancnctgtttt

SEQ ID NO: 99 - RNA, 44 nt, synthetic construct; identical to SEQ ID NO: 94
  source 1..44: mol_type = other RNA
  modified_base 37: mod_base = OTHER; note = 2 prime-deoxy-2 prime-fluoro-2-aminoadenosine-3 prime-phosphate (2 prime-F-2,6-diaminopurine)
  modified_base order(1,21,31,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphat
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 100 - RNA, 44 nt, synthetic construct; identical to SEQ ID NO: 95
  source 1..44: mol_type = other RNA
  modified_base 35: mod_base = OTHER; note = 2 prime-deoxy-2 prime-fluoro-2-aminoadenosine-3 prime-phosphate (2 prime-F-2,6-diaminopurine)
  modified_base order(1,21,31,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 101 - RNA, 44 nt, synthetic construct; identical to SEQ ID NO: 96
  source 1..44: mol_type = other RNA
  modified_base 31: mod_base = OTHER; note = 2 prime-deoxy-2 prime-fluoro-2-aminoadenosine-3 prime-phosphate (2 prime-F-2,6-diaminopurine)
  modified_base order(1,21,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 102 - RNA, 44 nt, synthetic construct; identical to SEQ ID NO: 97
  source 1..44: mol_type = other RNA
  modified_base order(31,37): mod_base = OTHER; note = 2 prime-deoxy-2 prime-fluoro-2-aminoadenosine-3 prime-phosphate (2 prime-F-2,6-diaminopurine)
  modified_base order(1,21,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 103 - RNA, 44 nt, synthetic construct; identical to SEQ ID NO: 98
  source 1..44: mol_type = other RNA
  modified_base order(31,35,37): mod_base = OTHER; note = 2 prime-deoxy-2 prime-fluoro-2-aminoadenosine-3 prime-phosphate (2 prime-F-2,6-diaminopurine)
  modified_base order(1,21,35,37): mod_base = OTHER; note = 2 prime-deoxy-2 prime-fluoroadenosine-3 prime-phosphat
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 104 - RNA, 44 nt, synthetic construct; identical to SEQ ID NO: 94
  source 1..44: mol_type = other RNA
  modified_base 37: mod_base = OTHER; note = 2-amino-2 prime-O,4 prime-C-methylene adenosine-3 prime-phosphate (LNA-2,6-diaminopurine)
  modified_base order(1,21,31,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 105 - RNA, 44 nt, synthetic construct; identical to SEQ ID NO: 95
  source 1..44: mol_type = other RNA
  modified_base 35: mod_base = OTHER; note = 2-amino-2 prime-O,4 prime-C-methylene adenosine-3 prime-phosphate (LNA-2,6-diaminopurine)
  modified_base order(1,21,31,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 106 - RNA, 44 nt, synthetic construct; identical to SEQ ID NO: 96
  source 1..44: mol_type = other RNA
  modified_base 31: mod_base = OTHER; note = 2-amino-2 prime-O,4 prime-C-methylene adenosine-3 prime-phosphate (LNA-2,6-diaminopurine)
  modified_base order(1,21,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 107 - RNA, 44 nt, synthetic construct; identical to SEQ ID NO: 97
  source 1..44: mol_type = other RNA
  modified_base order(31,37): mod_base = OTHER; note = 2-amino-2 prime-O,4 prime-C-methylene adenosine-3 prime-phosphate (LNA-2,6-diaminopurine)
  modified_base order(1,21,35): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29,33): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 108 - RNA, 44 nt, synthetic construct; identical to SEQ ID NO: 98
  source 1..44: mol_type = other RNA
  modified_base order(31,35): mod_base = OTHER; note = 2-amino-2 prime-O,4 prime-C-methylene adenosine-3 prime-phosphate (LNA-2,6-diaminopurine)
  modified_base order(1,21,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 109 - RNA, 44 nt, synthetic construct
  source 1..44: mol_type = other RNA
  modified_base 34: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatagagcaagnacactgtttt

SEQ ID NO: 110 - RNA, 44 nt, synthetic construct
  source 1..44: mol_type = other RNA
  modified_base 32: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatagagcangaacactgtttt

SEQ ID NO: 111 - RNA, 44 nt, synthetic construct
  source 1..44: mol_type = other RNA
  modified_base 28: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatagngcaagaacactgtttt

SEQ ID NO: 112 - RNA, 44 nt, synthetic construct
  source 1..44: mol_type = other RNA
  modified_base 26: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattatngagcaagaacactgtttt

SEQ ID NO: 113 - RNA, 44 nt, synthetic construct
  source 1..44: mol_type = other RNA
  modified_base 24: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattntagagcaagaacactgtttt

SEQ ID NO: 114 - RNA, 44 nt, synthetic construct
  source 1..44: mol_type = other RNA
  modified_base order(24,26,28,32,34): mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattntngngcangnacactgtttt

SEQ ID NO: 115 - RNA, 44 nt, synthetic construct; identical to SEQ ID NO: 109
  source 1..44: mol_type = other RNA
  modified_base 34: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 116 - RNA, 44 nt, synthetic construct; identical to SEQ ID NO: 110
  source 1..44: mol_type = other RNA
  modified_base 32: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 117 - RNA, 44 nt, synthetic construct; identical to SEQ ID NO: 111
  source 1..44: mol_type = other RNA
  modified_base 28: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 118 - RNA, 44 nt, synthetic construct; identical to SEQ ID NO: 112
  source 1..44: mol_type = other RNA
  modified_base 26: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 119 - RNA, 44 nt, synthetic construct; identical to SEQ ID NO: 113
  source 1..44: mol_type = other RNA
  modified_base 24: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 120 - RNA, 44 nt, synthetic construct; identical to SEQ ID NO: 114
  source 1..44: mol_type = other RNA
  modified_base order(24,26,28,32,34): mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 121 - RNA, 44 nt, synthetic construct
  source 1..44: mol_type = other RNA
  modified_base order(24,26,32,34): mod_base = OTHER; note = 2 prime-O-methyl-2-aminoadenosine-3 prime-phosphate (2 primeOMe-2,6-diaminopurine)
  modified_base order(31,35,37): mod_base = OTHER; note = 2 prime-deoxy-2 prime-fluoro-2-aminoadenosine-3 prime-phosphate (2 prime-F-2,6-diaminopurine)
  modified_base 28: mod_base = OTHER; note = 2-amino-2 prime-O,4 prime-C-methylene adenosine-3 prime-phosphate (LNA-2,6-diaminopurine)
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctataattntngngcnngnncnctgtttt

SEQ ID NO: 122 - RNA, 44 nt, synthetic construct; identical to SEQ ID NO: 121
  source 1..44: mol_type = other RNA
  modified_base order(1,21): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base 31: mod_base = OTHER; note = 2 prime-deoxy-2-aminoadenosine-3 prime-phosphate (deoxy-2,6-diaminopurine)
  modified_base 24: mod_base = OTHER; note = 2-aminoadenosine-3 prime-phosphate (ribo-2,6-diaminopurine)
  modified_base order(35,37): mod_base = OTHER; note = 2 prime-deoxy-2 prime-fluoro-2-aminoadenosine-3 prime-phosphate (2 prime-F-2,6-diaminopurine)
  modified_base 28: mod_base = OTHER; note = 2-amino-2 prime-O,4 prime-C-methylene adenosine-3 prime-phosphate (LNA-2,6-diaminopurine)
  modified_base order(26,32,34): mod_base = OTHER; note = 2 prime-O-methyl-2-aminoadenosine-3 prime-phosphate (2 primeOMe-2,6-diaminopurine)
  modified_base order(1^2,2^3,22^23,23^24,42^43,43^44): mod_base = OTHER; note = Phosphorothioate linkage

SEQ ID NO: 123 - RNA, 44 nt, synthetic construct
  source 1..44: mol_type = other RNA
  modified_base 21: mod_base = OTHER; note = 2 prime-deoxy-2-aminoadenosine-3 prime-phosphate (deoxy-2,6-diaminopurine)
  modified_base order(1,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctatanttatagagcaagaacactgtttt

SEQ ID NO: 124 - RNA, 44 nt, synthetic construct
  source 1..44: mol_type = other RNA
  modified_base 1: mod_base = OTHER; note = 2 prime-deoxy-2-aminoadenosine-3 prime-phosphate (deoxy-2,6-diaminopurine)
  modified_base order(21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
nacagtgttcttgctctataattatagagcaagaacactgtttt

SEQ ID NO: 125 - RNA, 44 nt, synthetic construct
  source 1..44: mol_type = other RNA
  modified_base order(1,21): mod_base = OTHER; note = 2 prime-deoxy-2-aminoadenosine-3 prime-phosphate (deoxy-2,6-diaminopurine)
  modified_base order(31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
nacagtgttcttgctctatanttatagagcaagaacactgtttt

SEQ ID NO: 126 - RNA, 44 nt, synthetic construct; identical to SEQ ID NO: 123
  source 1..44: mol_type = other RNA
  modified_base 21: mod_base = OTHER; note = 2 prime-deoxy-2 prime-fluoro-2-aminoadenosine-3 prime-phosphate (2 prime-F-2,6-diaminopurine)
  modified_base order(1,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 127 - RNA, 44 nt, synthetic construct; identical to SEQ ID NO: 124
  source 1..44: mol_type = other RNA
  modified_base 1: mod_base = OTHER; note = 2 prime-deoxy-2 prime-fluoro-2-aminoadenosine-3 prime-phosphate (2 prime-F-2,6-diaminopurine)
  modified_base order(21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 128 - RNA, 44 nt, synthetic construct; identical to SEQ ID NO: 125
  source 1..44: mol_type = other RNA
  modified_base order(1,21): mod_base = OTHER; note = 2 prime-deoxy-2 prime-fluoro-2-aminoadenosine-3 prime-phosphate (2 prime-F-2,6-diaminopurine)
  modified_base order(31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 129 - RNA, 44 nt, synthetic construct; identical to SEQ ID NO: 123
  source 1..44: mol_type = other RNA
  modified_base order(1,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base 21: mod_base = OTHER; note = 2-amino-2 prime-O,4 prime-C-methylene adenosine-3 prime-phosphate (LNA-2,6-diaminopurine)
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 130 - RNA, 44 nt, synthetic construct; identical to SEQ ID NO: 124
  source 1..44: mol_type = other RNA
  modified_base order(21,31,35,37): mod_base = OTHER; note = 2 prime-deoxy-2 prime-fluoroadenosine-3 prime-phosphate
  modified_base 1: mod_base = OTHER; note = 2-amino-2 prime-O,4 prime-C-methylene adenosine-3 prime-phosphate (LNA-2,6-diaminopurine)
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 131 - RNA, 44 nt, synthetic construct; identical to SEQ ID NO: 125
  source 1..44: mol_type = other RNA
  modified_base order(1,2): mod_base = OTHER; note = 2-amino-2 prime-O,4 prime-C-methylene adenosine-3 prime-phosphate (LNA-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 132 - RNA, 44 nt, synthetic construct
  source 1..44: mol_type = other RNA
  modified_base 20: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctatnattatagagcaagaacactgtttt

SEQ ID NO: 133 - RNA, 44 nt, synthetic construct
  source 1..44: mol_type = other RNA
  modified_base 18: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacagtgttcttgctctntaattatagagcaagaacactgtttt

SEQ ID NO: 134 - RNA, 44 nt, synthetic construct
  source 1..44: mol_type = other RNA
  modified_base 4: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
aacngtgttcttgctctataattatagagcaagaacactgtttt

SEQ ID NO: 135 - RNA, 44 nt, synthetic construct
  source 1..44: mol_type = other RNA
  modified_base 2: mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
ancagtgttcttgctctataattatagagcaagaacactgtttt

SEQ ID NO: 136 - RNA, 44 nt, synthetic construct
  source 1..44: mol_type = other RNA
  modified_base order(2,4,18,20): mod_base = OTHER; note = 2-aminoadenosine-3 prime-phosphate (ribo-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
ancngtgttcttgctctntnattatagagcaagaacactgtttt

SEQ ID NO: 137 - RNA, 44 nt, synthetic construct; identical to SEQ ID NO: 132
  source 1..44: mol_type = other RNA
  modified_base 20: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 138 - RNA, 44 nt, synthetic construct; identical to SEQ ID NO: 133
  source 1..44: mol_type = other RNA
  modified_base 18: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 139 - RNA, 44 nt, synthetic construct; identical to SEQ ID NO: 134
  source 1..44: mol_type = other RNA
  modified_base 4: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 140 - RNA, 44 nt, synthetic construct; identical to SEQ ID NO: 135
  source 1..44: mol_type = other RNA
  modified_base 2: mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = e-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 141 - RNA, 44 nt, synthetic construct; identical to SEQ ID NO: 136
  source 1..44: mol_type = other RNA
  modified_base order(2,4,18,20): mod_base = OTHER; note = 2 prime-O-methyl-2-aminoadenosine-3 prime-phosphate (2 primeOMe-2,6-diaminopurine)
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 142 - RNA, 44 nt, synthetic construct; identical to SEQ ID NO: 93
  source 1..44: mol_type = other RNA
  modified_base order(1,21,31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(2,4,18,20,24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage

SEQ ID NO: 143 - RNA, 44 nt, synthetic construct
  source 1..44: mol_type = other RNA
  modified_base 1: mod_base = OTHER; note = 2-amino-2 prime-O,4 prime-C-methylene adenosine-3 prime-phosphate (LNA-2,6-diaminopurine)
  modified_base order(2,4,18,20): mod_base = OTHER; note = 2 prime-O-methyl-2-aminoadenosine-3 prime-phosphate (2 primeOMe-2,6-diaminopurine)
  modified_base 21: mod_base = OTHER; note = 2 prime-deoxy-2-aminoadenosine-3 prime-phosphate (deoxy-2,6-diaminopurine)
  modified_base order(31,35,37): mod_base = OTHER; note = 2 prime-deoxy-2 prime-fluoroadenosine-3 prime-phosphate
  modified_base order(3,10): mod_base = OTHER; note = 2 prime-deoxy-2 prime-fluorocytidine-3 prime-phosphate
  modified_base order(5,7,13,27,29): mod_base = OTHER; note = 2 prime-deoxy-2 prime-fluoroguanosine-3 prime-phosphate
  modified_base order(9,11,15,17,19,23,25,29,41): mod_base = OTHER; note = 2 prime-deoxy-2 prime-fluorouridine-3 prime-phosphate
  modified_base order(24,26,28,32,34): mod_base = OTHER; note = 2 prime-O-methyladenosine-3 prime-phosphate
  modified_base order(14,16,30,36,38): mod_base = OTHER; note = 2 prime-O-methylcytidine-3 prime-phosphate
  modified_base order(33,40): mod_base = OTHER; note = 2 prime-O-methylguanosine-3 prime-phosphate
  modified_base order(6,8,12,22,42,43,44): mod_base = OTHER; note = 2 prime-O-methyluridine-3 prime-phosphate
  modified_base order(1^2,2^3,22^23,23^24,42^43,43^44): mod_base = OTHER; note = Phosphorothioate linkage
nncngtgttcttgctctntnnttatagagcaagaacactgtttt

SEQ ID NO: 144 - RNA, 16 nt, synthetic construct
  source 1..16: mol_type = other RNA
  modified_base 9: mod_base = OTHER; note = 2 prime-deoxy-2 prime-fluoroadenosine-3 prime-phosphate
  modified_base 10: mod_base = OTHER; note = 2 prime-deoxy-2 prime-fluoroguanosine-3 prime-phosphate
  modified_base order(3,8,15,16): mod_base = OTHER; note = 2 prime-O-methyladenosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base order(1^2,2^3,3^4,4^5,5^6,6^7,8^9,9^10,10^11,11^12,12^13,13^14,14^15): mod_base = OTHER; note = Phosphorothioate linkage
  modified_base 12: mod_base = OTHER; note = 2 prime-deoxy-2 prime-fluorocytidine-3 prime-phosphate
tcatctcaagtcttaa

SEQ ID NO: 145 - RNA, 16 nt, synthetic construct
  source 1..16: mol_type = other RNA
  modified_base 12: mod_base = OTHER; note = 2 prime-deoxy-2 prime-fluorocytidine-3 prime-phosphate
  modified_base 10: mod_base = OTHER; note = 2 prime-deoxy-2 prime-fluoroguanosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base 16: mod_base = OTHER; note = 2 prime-deoxy-2-aminoadenosine-3 prime-phosphate (deoxy-2,6-diaminopurine)
  modified_base order(3,8,9,15): mod_base = OTHER; note = 2-amino-2 prime-O,4 prime-C-methylene adenosine-3 prime-phosphate (LNA-2,6-diaminopurine)
  modified_base order(1^2,2^3,3^4,4^5,5^6,6^7,8^9,9^10,10^11,11^12,12^13,13^14,14^15): mod_base = OTHER; note = Phosphorothioate linkage
tcntctcnngtcttnn

SEQ ID NO: 146 - RNA, 16 nt, synthetic construct
  source 1..16: mol_type = other RNA
  modified_base 12: mod_base = OTHER; note = 2 prime-deoxy-2 prime-fluorocytidine-3 prime-phosphate
  modified_base 10: mod_base = OTHER; note = 2 prime-deoxy-2 prime-fluoroguanosine-3 prime-phosphate
  modified_base order(2,5,7): mod_base = OTHER; note = 2 prime-O-methylcytidine-3 prime-phosphate
  modified_base order(1,4,6,13): mod_base = OTHER; note = 2 prime-O-methyluridine-3 prime-phosphate
  modified_base 16: mod_base = OTHER; note = 2 prime-deoxy-2-aminoadenosine-3 prime-phosphate (deoxy-2,6-diaminopurine)
  modified_base 9: mod_base = OTHER; note = 2-amino-2 prime-O,4 prime-C-methylene adenosine-3 prime-phosphate (LNA-2,6-diaminopurine)
  modified_base order(3,8,15): mod_base = OTHER; note = 2 prime-O-methyl-2-aminoadenosine-3 prime-phosphate (2 primeOMe-2,6-diaminopurine)
  modified_base order(1^2,2^3,3^4,4^5,5^6,6^7,8^9,9^10,10^11,11^12,12^13,13^14,14^15): mod_base = OTHER; note = Phosphorothioate linkage
tcntctcnngtcttnn

SEQ ID NO: 147 - RNA, 23 nt, synthetic construct
  source 1..23: mol_type = other RNA
  modified_base order(1,3..5,7,10,16,18,23): mod_base = OTHER; note = 2-prime-O-methyladenosine-3-prime-phosphate
  modified_base order(2,6,8..9,15,17,19,21..22): mod_base = OTHER; note = 2-prime-O-methylcytidine-3-prime-phosphate
  modified_base order(11..12,14,20): mod_base = OTHER; note = 2-prime-O-methyluridine-3-prime-phosphate
  modified_base 13: mod_base = OTHER; note = 2-prime-O-methylguanosine-3-prime-phosphate
  modified_base order(1^2,2^3,20^21,21^22,22^23,23): mod_base = OTHER; note = Phosphorothioate linkages
acaaacaccattgtcacactcca

SEQ ID NO: 148 - RNA, 23 nt, synthetic construct
  source 1..23: mol_type = other RNA
  modified_base order(1,3..5,7,10,18,23): mod_base = OTHER; note = 2-amino-2-prime-O,4-prime-C-methylene adenosine-3-prime-phosphate (LNA-2,6-diaminopurine)
  modified_base order(2,6,8..9,15,17,19,21..22): mod_base = OTHER; note = 2-prime-O-methylcytidine-3-prime-phosphate
  modified_base order(11..12,14,20): mod_base = OTHER; note = 2-prime-O-methyluridine-3-prime-phosphate
  modified_base 13: mod_base = OTHER; note = 2-prime-O-methylguanosine-3-prime-phosphate
  modified_base 16: mod_base = OTHER; note = 2-prime-O-methyladenosine-3-prime-phosphate
  modified_base order(1^2,2^3,20^21,21^22,22^23,23): mod_base = OTHER; note = Phosphorothioate linkages
ncnnncnccnttgtcacnctccn

SEQ ID NO: 149 - DNA, 12 bp, synthetic construct
  source 1..12: mol_type = other DNA
  modified_base order(2,4,10,12): mod_base = OTHER; note = 2-prime-deoxyguanosine-3-prime-phosphate (Deoxy-Guanosine)
  modified_base order(5,6): mod_base = OTHER; note = 2-prime-deoxyadenosine-3-prime-phosphate (Deoxy-Adenosine)
  modified_base order(7,8): mod_base = OTHER; note = 2-prime-deoxyuridine-3-prime-phosphate (Deoxy-Thymidine)
  modified_base order(1,3,9,11): mod_base = OTHER; note = 2-prime-deoxycytidine-3-prime-phosphate (Deoxy-Cytidine)
cgcgaattcgcg

SEQ ID NO: 150 - DNA, 12 bp, synthetic construct
  source 1..12: mol_type = other DNA
  modified_base 6: mod_base = OTHER; note = 2-prime-deoxyadenosine-3-prime-phosphate (Deoxy-Adenosine)
  modified_base order(1,3,9,11): mod_base = OTHER; note = 2-prime-deoxycytidine-3-prime-phosphate (Deoxy-Cytidine
  modified_base order(2,4,10,12): mod_base = OTHER; note = 2-prime-deoxyguanosine-3-prime-phosphate (Deoxy-Guanosine)
  modified_base order(7,8): mod_base = OTHER; note = 2-prime-deoxyuridine-3-prime-phosphate (Deoxy-Thymidine)
  modified_base 5: mod_base = OTHER; note = 2-prime-deoxy-2-aminoadenosine-3-prime-phosphate (deoxy-2,6-diaminopurine)
cgcgnattcgcg

SEQ ID NO: 151 - DNA, 12 bp, synthetic construct
  source 1..12: mol_type = other DNA
  modified_base order(1,3,9,11): mod_base = OTHER; note = 2-prime-deoxycytidine-3-prime-phosphate (Deoxy-Cytidine)
  modified_base order(2,4,10,12): mod_base = OTHER; note = 2-prime-deoxyguanosine-3-prime-phosphate (Deoxy-Guanosine)
  modified_base 5: mod_base = OTHER; note = 2-prime-deoxyadenosine-3-prime-phosphate (Deoxy-Adenosine)
  modified_base 6: mod_base = OTHER; note = 2-prime-deoxy-2-aminoadenosine-3-prime-phosphate (deoxy-2,6-diaminopurine)
  modified_base order(7,8): mod_base = OTHER; note = 2-prime-deoxyuridine-3-prime-phosphate (Deoxy-Thymidine)
cgcganttcgcg

SEQ ID NO: 152 - DNA, 12 bp, synthetic construct
  source 1..12: mol_type = other DNA
  modified_base order(1,3,9,11): mod_base = OTHER; note = 2-prime-deoxycytidine-3-prime-phosphate (Deoxy-Cytidine)
  modified_base order(2,4,10,12): mod_base = OTHER; note = 2-prime-deoxyguanosine-3-prime-phosphate (Deoxy-Guanosine)
  modified_base order(5,6): mod_base = OTHER; note = 2-prime-deoxy-2-aminoadenosine-3-prime-phosphate (deoxy-2,6-diaminopurine)
  modified_base order(7,8): mod_base = OTHER; note = 2-prime-deoxyuridine-3-prime-phosphate (Deoxy-Thymidine)
cgcgnnttcgcg

SEQ ID NO: 153 - DNA, 12 bp, synthetic construct
  source 1..12: mol_type = other DNA
  modified_base order(1,3,9,11): mod_base = OTHER; note = 2-prime-deoxycytidine-3-prime-phosphate (Deoxy-Cytidine)
  modified_base order(2,4,10,12): mod_base = OTHER; note = 2-prime-deoxyguanosine-3-prime-phosphate (Deoxy-Guanosine)
  modified_base 5: mod_base = OTHER; note = 2-prime-deoxy-2-(15N)aminoadenosine-3-prime-phospha te (deoxy-2,6-diaminopurine 2-15N labelling)
  modified_base 6: mod_base = OTHER; note = 2-prime-deoxyadenosine-3-prime-phosphate (Deoxy-Adenosine)
  modified_base order(7,8): mod_base = OTHER; note = 2-prime-deoxyuridine-3-prime-phosphate (Deoxy-Thymidine)
cgcgnattcgcg

SEQ ID NO: 154 - DNA, 12 bp, synthetic construct
  source 1..12: mol_type = other DNA
  modified_base order(1,3,9,11): mod_base = OTHER; note = 2-prime-deoxycytidine-3-prime-phosphate (Deoxy-Cytidine)
  modified_base order(2,4,10,12): mod_base = OTHER; note = 2-prime-deoxyguanosine-3-prime-phosphate (Deoxy-Guanosine)
  modified_base 5: mod_base = OTHER; note = 2-prime-deoxyadenosine-3-prime-phosphate (Deoxy-Adenosine)
  modified_base 6: mod_base = OTHER; note = 2-prime-deoxy-2-(15N)aminoadenosine-3-prime-phospha te (deoxy-2,6-diaminopurine 2-15N labelling)
  modified_base order(7,8): mod_base = OTHER; note = 2-prime-deoxyuridine-3-prime-phosphate (Deoxy-Thymidine)
cgcganttcgcg

SEQ ID NO: 155 - DNA, 12 bp, synthetic construct
  source 1..12: mol_type = other DNA
  modified_base order(1,3,9,11): mod_base = OTHER; note = 2-prime-deoxycytidine-3-prime-phosphate (Deoxy-Cytidine)
  modified_base order(2,4,10,12): mod_base = OTHER; note = 2-prime-deoxyguanosine-3-prime-phosphate (Deoxy-Guanosine)
  modified_base order(5,6): mod_base = OTHER; note = 2-prime-deoxy-2-(15N)aminoadenosine-3-prime-phospha te (deoxy-2,6-diaminopurine 2-15N labelling)
  modified_base order(7,8): mod_base = OTHER; note = 2-prime-deoxyuridine-3-prime-phosphate (Deoxy-Thymidine)
cgcgnnttcgcg

SEQ ID NO: 156 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base order(1^2,2^3): mod_base = OTHER; note = Phosphorothioate linkages
aacagtgttcttgctctataa

SEQ ID NO: 157 - RNA, 21 nt, synthetic construct
  source 1..21: mol_type = other RNA
  modified_base 1: mod_base = OTHER; note = 2-prime F-2-N-acetylgalactosamine-adenosine 3-prime-phosphate
  modified_base order(2,4): mod_base = OTHER; note = 2-prime OMe-2-N-acetylgalactosamine-adenosine 3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base order(18,20): mod_base = OTHER; note = 2-prime-O-methyladenosine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nncngtgttcttgctctataa

SEQ ID NO: 158 - RNA, 21 nt, synthetic construct
  source 1..21: mol_type = other RNA
  modified_base 1: mod_base = OTHER; note = 2-(N-(6-(O-cholesterol)oxcarbonylaminohexyl)amino)- 2-prime-deoxy-2-prime-fluoro-adenosine 3-prime-phosphate (2-prime-F-2-cholesterol-adenosine)
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nacagtgttcttgctctataa

SEQ ID NO: 159 - RNA, 21 nt, synthetic construct
  source 1..21: mol_type = other RNA
  modified_base 1: mod_base = OTHER; note = 2-prime F-2-N-acetylgalactosamine-adenosine 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(1^2,2^3,19^20,20^21): mod_base = OTHER; note = Phosphorothioate linkages
nacagtgttcttgctctataa

SEQ ID NO: 160 - RNA, 21 nt, synthetic construct
  source 1..21: mol_type = other RNA
  modified_base 1: mod_base = OTHER; note = 2-prime F-2-tri-N-acetylgalactosamine 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(1^2,2^3,19^20,20^21): mod_base = OTHER; note = Phosphorothioate linkages
nacagtgttcttgctctataa

SEQ ID NO: 161 - RNA, 21 nt, synthetic construct
  source 1..21: mol_type = other RNA
  modified_base 1: mod_base = OTHER; note = 2-prime F-2-cRGD Kessler peptide 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(1^2,2^3,19^20,20^21): mod_base = OTHER; note = Phosphorothioate linkages
nacagtgttcttgctctataa

SEQ ID NO: 162 - RNA, 21 nt, synthetic construct
  source 1..21: mol_type = other RNA
  modified_base 1: mod_base = OTHER; note = 2-(N-hexadecylamino)-2-prime-deoxy-2-prime-fluoro-a denosine-3-prime-phosphate (2-primeF-2-C16-adenosine)
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(1^2,2^3,19^20,20^21): mod_base = OTHER; note = Phosphorothioate linkages
nacagtgttcttgctctataa

SEQ ID NO: 163 - RNA, 21 nt, synthetic construct
  source 1..21: mol_type = other RNA
  modified_base 1: mod_base = OTHER; note = 2-prime-deoxy-2-prime-fluoroadenosine-3-prime-phosp hate
  modified_base order(2,4): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base order(18,20): mod_base = OTHER; note = 2-prime OMe-2-N-acetylgalactosamine-adenosine 3-prime-phosphate
  modified_base 21: mod_base = OTHER; note = 2-prime F-2-N-acetylgalactosamine-adenosine 3-prime-phosphate
  modified_base order(1^2,2^3): mod_base = OTHER; note = Phosphorothioate linkages
aacagtgttcttgctctntnn

SEQ ID NO: 164 - RNA, 21 nt, synthetic construct
  source 1..21: mol_type = other RNA
  modified_base 1: mod_base = OTHER; note = 2-prime-deoxy-2-prime-fluoroadenosine-3-prime-phosp hate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base 21: mod_base = OTHER; note = 2-prime F-2-tri-N-acetylgalactosamine 3-prime-phosphate
  modified_base order(1^2,2^3): mod_base = OTHER; note = Phosphorothioate linkages
aacagtgttcttgctctatan

SEQ ID NO: 165 - RNA, 21 nt, synthetic construct
  source 1..21: mol_type = other RNA
  modified_base order(1,6,8,13): mod_base = OTHER; note = 2-prime-O-methyluridine-3-prime-phosphate
  modified_base order(2,3..4,12,20): mod_base = OTHER; note = 2-prime-O-methylguanosine-3-prime-phosphate
  modified_base order(5,14..15,18..19,21): mod_base = OTHER; note = 2-prime-O-methyladenosine-3-prime-phosphate
  modified_base order(7,11): mod_base = OTHER; note = 2-prime-deoxy-2-prime-fluorouridine-3-prime-phospha te
  modified_base 9: mod_base = OTHER; note = 2-prime-deoxy-2-prime-fluorocytidine-3-prime-phosph ate
  modified_base 10: mod_base = OTHER; note = 2-prime-deoxy-2-prime-fluoroadenosine-3-prime-phosp hate
  modified_base order(16,17): mod_base = OTHER; note = 2-prime-O-methylcytidine-3-prime-phosphate
  modified_base order(1^2,2^3,19^20,20^21): mod_base = OTHER; note = Phosphorothioate linkages
tgggatttcatgtaaccaaga

SEQ ID NO: 166 - RNA, 21 nt, synthetic construct
  source 1..21: mol_type = other RNA
  modified_base order(1,6,8,13): mod_base = OTHER; note = 2-prime-O-methyluridine-3-prime-phosphate
  modified_base order(2,3..4,12,20): mod_base = OTHER; note = 2-prime-O-methylguanosine-3-prime-phosphate
  modified_base 5: mod_base = OTHER; note = 2-prime-O-methyl-2-aminoadenosine-3-prime-phosphate (2-prime-OMe-2,6-diaminopurine)
  modified_base order(7,11): mod_base = OTHER; note = 2-prime-deoxy-2-prime-fluorouridine-3-prime-phospha te
  modified_base 9: mod_base = OTHER; note = 2-prime-deoxy-2-prime-fluorocytidine-3-prime-phosph ate
  modified_base 9: mod_base = OTHER; note = 2-prime-deoxy-2-prime-fluorocytidine-3-prime-phosph ate
  modified_base 10: mod_base = OTHER; note = 2-prime-deoxy-2-prime-fluoroadenosine-3-prime-phosp hate
  modified_base order(14..15,18..19,21): mod_base = OTHER; note = 2-prime-O-methyladenosine-3-prime-phosphate
  modified_base order(16,17): mod_base = OTHER; note = 2-prime-O-methylcytidine-3-prime-phosphate
  modified_base order(1^2,2^3,19^20,20^21): mod_base = OTHER; note = Phosphorothioate linkages
tgggntttcatgtaaccaaga

SEQ ID NO: 167 - RNA, 21 nt, synthetic construct
  source 1..21: mol_type = other RNA
  modified_base order(1,6,8,13): mod_base = OTHER; note = 2-prime-O-methyluridine-3-prime-phosphate
  modified_base order(2,3..4,12,20): mod_base = OTHER; note = 2-prime-O-methylguanosine-3-prime-phosphate
  modified_base order(5,14): mod_base = OTHER; note = 2-(N-nonylamino)-2-prime-O-methyladenosine-3-prime- phosphate (2-prime OMe-2-C9-adenosine)
  modified_base order(7,11): mod_base = OTHER; note = 2-prime-deoxy-2-prime-fluorouridine-3-prime-phospha te
  modified_base 9: mod_base = OTHER; note = 2-prime-deoxy-2-prime-fluorocytidine-3-prime-phosph ate
  modified_base 10: mod_base = OTHER; note = 2-prime-deoxy-2-prime-fluoroadenosine-3-prime-phosp hate
  modified_base order(15,18..19,21): mod_base = OTHER; note = 2-prime-O-methyladenosine-3-prime-phosphate
  modified_base order(16,17): mod_base = OTHER; note = 2-prime-O-methylcytidine-3-prime-phosphate
  modified_base order(1^2,2^3,19^20,20^21): mod_base = OTHER; note = Phosphorothioate linkages
tgggntttcatgtnaccaaga

SEQ ID NO: 168 - RNA, 21 nt, synthetic construct
  source 1..21: mol_type = other RNA
  modified_base order(1,6,8,13): mod_base = OTHER; note = 2-prime-O-methyluridine-3-prime-phosphate
  modified_base order(2,3..4,12,20): mod_base = OTHER; note = 2-prime-O-methylguanosine-3-prime-phosphate
  modified_base order(5,14,18): mod_base = OTHER; note = 2-(N-hexylamino)-2-prime-O-methyladenosine-3-prime- phosphate (2-prime OMe-2-C6-adenosine)
  modified_base order(7,11): mod_base = OTHER; note = 2-prime-deoxy-2-prime-fluorouridine-3-prime-phospha te
  modified_base 9: mod_base = OTHER; note = 2-prime-deoxy-2-prime-fluorocytidine-3-prime-phosph ate
  modified_base 10: mod_base = OTHER; note = 2-prime-deoxy-2-prime-fluoroadenosine-3-prime-phosp hate
  modified_base order(15,19,21): mod_base = OTHER; note = 2-prime-O-methyladenosine-3-prime-phosphate
  modified_base order(1^2,2^3,19^20,20^21): mod_base = OTHER; note = Phosphorothioate linkages
tgggntttcatgtnaccnaga

SEQ ID NO: 169 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3..4,7..8,12,17,22): mod_base = OTHER; note = 2-prime-O-methyluridine-3-prime-phosphate
  modified_base 2: mod_base = OTHER; note = 2-prime-deoxy-2-prime-fluorocytidine-3-prime-phosph ate
  modified_base order(5,13): mod_base = OTHER; note = 2-prime-O-methylguanosine-3-prime-phosphate
  modified_base 6: mod_base = OTHER; note = 2-prime-deoxy-2-prime-fluoroguanosine-3-prime-phosp hate
  modified_base order(9,14,16): mod_base = OTHER; note = 2-prime-deoxy-2-prime-fluoroadenosine-3-prime-phosp hate
  modified_base order(10,18..20,23): mod_base = OTHER; note = 2-prime-O-methylcytidine-3-prime-phosphate
  modified_base order(11,15,21): mod_base = OTHER; note = 2-prime-O-methyladenosine-3-prime-phosphate
  modified_base order(1^2,2^3,21^22,22^23): mod_base = OTHER; note = Phosphorothioate linkages
tcttggttacatgaaatcccatc

SEQ ID NO: 170 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3..4,7..8,12,17,22): mod_base = OTHER; note = 2-prime-O-methyluridine-3-prime-phosphate
  modified_base 2: mod_base = OTHER; note = 2-prime-deoxy-2-prime-fluorocytidine-3-prime-phosph ate
  modified_base order(5,13): mod_base = OTHER; note = 2-prime-O-methylguanosine-3-prime-phosphate
  modified_base 6: mod_base = OTHER; note = 2-prime-deoxy-2-prime-fluoroguanosine-3-prime-phosp hate
  modified_base 9: mod_base = OTHER; note = 2-(N-hexadecylamino)-2-prime-deoxy-2-prime-fluoro-a denosine-3-prime-phosphate (2-primeF-2-C16-adenosine);
  modified_base order(10,18..20,23): mod_base = OTHER; note = 2-prime-O-methylcytidine-3-prime-phosphate
  modified_base order(11,15,21): mod_base = OTHER; note = 2-prime-O-methyladenosine-3-prime-phosphate
  modified_base order(14,16): mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
tcttggttncatgaaatcccatc

SEQ ID NO: 171 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3..4,7..8,12,17,22): mod_base = OTHER; note = 2-prime-O-methyluridine-3-prime-phosphate
  modified_base 2: mod_base = OTHER; note = 2-prime-deoxy-2-prime-fluorocytidine-3-prime-phosph ate
  modified_base order(5,13): mod_base = OTHER; note = 2-prime-O-methylguanosine-3-prime-phosphate
  modified_base 6: mod_base = OTHER; note = 2-prime-deoxy-2-prime-fluoroguanosine-3-prime-phosp hate
  modified_base 9: mod_base = OTHER; note = 2-(N-nonylamino)-2-prime-deoxy-2-prime-fluoro-adeno sine-3-prime-phosphate (2-prime F-2-C9-adenosine)
  modified_base order(10,18..20,23): mod_base = OTHER; note = 2-prime-O-methylcytidine-3-prime-phosphate
  modified_base 11: mod_base = OTHER; note = 2-(N-nonylamino)-2-prime-O-methyladenosine-3-prime- phosphate (2-prime OMe-2-C9-adenosine)
  modified_base order(14,16): mod_base = OTHER; note = 2-prime-deoxy-2-prime-fluoroadenosine-3-prime-phosp hate
  modified_base order(15,21): mod_base = OTHER; note = 2-prime-O-methyladenosine-3-prime-phosphate
  modified_base order(1^2,2^3,21^22,22^23): mod_base = OTHER; note = Phosphorothioate linkages
tcttggttncntgaaatcccatc

SEQ ID NO: 172 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3..4,7..8,12,17,22): mod_base = OTHER; note = 2-prime-O-methyluridine-3-prime-phosphate
  modified_base 2: mod_base = OTHER; note = 2-prime-deoxy-2-prime-fluorocytidine-3-prime-phosph ate
  modified_base order(5,13): mod_base = OTHER; note = 2-prime-O-methylguanosine-3-prime-phosphate
  modified_base 6: mod_base = OTHER; note = 2-prime-deoxy-2-prime-fluoroguanosine-3-prime-phosp hate
  modified_base order(9,14): mod_base = OTHER; note = 2-(N-hexylamino)-2-prime-deoxy-2-prime-fluoro-adeno sine-3-prime-phosphate (2-prime F-2-C6-adenosine)
  modified_base order(10,18..20,23): mod_base = OTHER; note = 2-prime-O-methylcytidine-3-prime-phosphate
  modified_base 11: mod_base = OTHER; note = 2-(N-hexylamino)-2-prime-O-methyladenosine-3-prime- phosphate (2-prime OMe-2-C6-adenosine)
  modified_base order(15,21): mod_base = OTHER; note = 2-prime-O-methyladenosine-3-prime-phosphate
  modified_base 16: mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
tcttggttncntgnaatcccatc

SEQ ID NO: 173 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20): mod_base = OTHER; note = 2-prime-O-methyladenosine-3-prime-phosphate
  modified_base order(1^2,2^3): mod_base = OTHER; note = Phosphorothioate linkages
tgacaaaataactcactataa

SEQ ID NO: 174 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(5,7): mod_base = OTHER; note = 2-prime F-2-N-acetylgalactosamine-adenosine 3-prime-phosphate
  modified_base 6: mod_base = OTHER; note = 2-prime OMe-2-N-acetylgalactosamine-adenosine 3-prime-phosphate
  modified_base order(8,18,20): mod_base = OTHER; note = 2-prime-O-methyladenosine-3-prime-phosphate
  modified_base order(1^2,2^3,19^20,20^21): mod_base = OTHER; note = Phosphorothioate linkages
tgacnnnataactcactataa

SEQ ID NO: 175 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base 3: mod_base = OTHER; note = 2-prime F-2-tri-N-acetylgalactosamine 3-prime-phosphate
  modified_base order(4,12,14,16): mod_base = OTHER; note = 2-prime-O-methylcytidine-3-prime-phosphate
  modified_base order(5,7,10..11,15,21): mod_base = OTHER; note = 2-prime-deoxy-2-prime-fluoroadenosine-3-prime-phosp hate
  modified_base order(6,8,18,20): mod_base = OTHER; note = 2-prime-O-methyladenosine-3-prime-phosphate
  modified_base order(1^2,2^3,19^20,20^21): mod_base = OTHER; note = Phosphorothioate linkages
tgncaaaataactcactataa

SEQ ID NO: 176 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20): mod_base = OTHER; note = 2-prime-O-methyladenosine-3-prime-phosphate
  modified_base 21: mod_base = OTHER; note = 2-prime F-2-tri-N-acetylgalactosamine 3-prime-phosphate
  modified_base order(1^2,2^3): mod_base = OTHER; note = Phosphorothioate linkages
tgacaaaataactcactatan

SEQ ID NO: 177 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base 3: mod_base = OTHER; note = 2-(N-hexadecylamino)-2-prime-deoxy-2-prime-fluoro-a denosine-3-prime-phosphate (2-primeF-2-C16-adenosine);
  modified_base order(4,12,14,16): mod_base = OTHER; note = 2-prime-O-methylcytidine-3-prime-phosphate
  modified_base order(5,7,10..11,15,21): mod_base = OTHER; note = 2-prime-deoxy-2-prime-fluoroadenosine-3-prime-phosp hate
  modified_base order(6,8,18,20): mod_base = OTHER; note = 2-prime-O-methyladenosine-3-prime-phosphate
  modified_base order(1^2,2^3,19^20,20^21): mod_base = OTHER; note = Phosphorothioate linkages
tgncaaaataactcactataa

SEQ ID NO: 178 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10): mod_base = OTHER; note = 2-(N-nonylamino)-2-prime-deoxy-2-prime-fluoro-adeno sine-3-prime-phosphate (2-prime F-2-C9-adenosine)
  modified_base order(4,12,14,16): mod_base = OTHER; note = 2-prime-O-methylcytidine-3-prime-phosphate
  modified_base order(5,7,11,15,21): mod_base = OTHER; note = 2-prime-deoxy-2-prime-fluoroadenosine-3-prime-phosp hate
  modified_base order(6,8,18,20): mod_base = OTHER; note = 2-prime-O-methyladenosine-3-prime-phosphate
  modified_base order(1^2,2^3,19^20,20^21): mod_base = OTHER; note = Phosphorothioate linkages
tgncaaaatnactcactataa

SEQ ID NO: 179 - RNA, 21 nt, synthetic construct
  source 1..21: mol_type = other RNA
  modified_base order(1,9,13,17,19): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10,15): mod_base = OTHER; note = 2-(N-hexylamino)-2-prime-deoxy-2-prime-fluoro-adeno sine-3-prime-phosphate (2-prime F-2-C6-adenosine)
  modified_base order(4,12,14,16): mod_base = OTHER; note = 2-prime-O-methylcytidine-3-prime-phosphate
  modified_base order(5,7,11,21): mod_base = OTHER; note = 2-prime-deoxy-2-prime-fluoroadenosine-3-prime-phosp hate
  modified_base order(6,8,18,20): mod_base = OTHER; note = 2-prime-O-methyladenosine-3-prime-phosphate
  modified_base order(1^2,2^3,19^20,20^21): mod_base = OTHER; note = Phosphorothioate linkages
tgncaaaatnactcnctataa

SEQ ID NO: 180 - RNA, 23 nt, synthetic construct
  source 1..23: mol_type = other RNA
  modified_base order(1,7,11..12,15,17,19,23): mod_base = OTHER; note = 2-prime-O-methyluridine-3-prime-phosphate
  modified_base order(2,4,14,16): mod_base = OTHER; note = 2-prime-deoxy-2-prime-fluorouridine-3-prime-phospha te
  modified_base order(3,5,9,13,21..22): mod_base = OTHER; note = 2-prime-O-methyladenosine-3-prime-phosphate
  modified_base order(6,8,10,18): mod_base = OTHER; note = 2-prime-deoxy-2-prime-fluoroguanosine-3-prime-phosp hate
  modified_base 20: mod_base = OTHER; note = 2-prime-deoxy-2-prime-fluorocytidine-3-prime-phosph ate
  modified_base order(1^2,2^3,21^22,22^23): mod_base = OTHER; note = Phosphorothioate linkages
ttatagtgagttattttgtcaat

SEQ ID NO: 181 - RNA, 23 nt, synthetic construct
  source 1..23: mol_type = other RNA
  modified_base order(1,7,11..12,15,17,19,23): mod_base = OTHER; note = 2-prime-O-methyluridine-3-prime-phosphate
  modified_base order(2,4,14,16): mod_base = OTHER; note = 2-prime-deoxy-2-prime-fluorouridine-3-prime-phospha te
  modified_base 3: mod_base = OTHER; note = 2-(N-hexadecylamino)-2-prime-O-methyladenosine-3-pr ime-phosphate (2-prime OMe-2-C16-adenosine)
  modified_base order(5,9,13,21,22): mod_base = OTHER; note = 2-prime-O-methyladenosine-3-prime-phosphate
  modified_base order(6,8,10,18): mod_base = OTHER; note = 2-prime-deoxy-2-prime-fluoroguanosine-3-prime-phosp hate
  modified_base 20: mod_base = OTHER; note = 2-prime-deoxy-2-prime-fluorocytidine-3-prime-phosph ate
  modified_base order(1^2,2^3,21^22,22^23): mod_base = OTHER; note = Phosphorothioate linkages
ttntagtgagttattttgtcaat

SEQ ID NO: 182 - RNA, 23 nt, synthetic construct
  source 1..23: mol_type = other RNA
  modified_base order(1,7,11..12,15,17,19,23): mod_base = OTHER; note = 2-prime-O-methyluridine-3-prime-phosphate
  modified_base order(2,4,14,16): mod_base = OTHER; note = 2-prime-deoxy-2-prime-fluorouridine-3-prime-phospha te
  modified_base order(3,9): mod_base = OTHER; note = 2-(N-nonylamino)-2-prime-O-methyladenosine-3-prime- phosphate (2-prime OMe-2-C9-adenosine)
  modified_base order(5,13,21..22): mod_base = OTHER; note = 2-prime-O-methyladenosine-3-prime-phosphate
  modified_base order(6,8,10,18): mod_base = OTHER; note = 2-prime-deoxy-2-prime-fluoroguanosine-3-prime-phosp hate
  modified_base 20: mod_base = OTHER; note = 2-prime-deoxy-2-prime-fluorocytidine-3-prime-phosph ate
  modified_base order(1^2,2^3,21^22,22^23): mod_base = OTHER; note = Phosphorothioate linkages
ttntagtgngttattttgtcaat

SEQ ID NO: 183 - RNA, 23 nt, synthetic construct
  source 1..23: mol_type = other RNA
  modified_base order(1,7,11..12,15,17,19,23): mod_base = OTHER; note = 2-prime-O-methyluridine-3-prime-phosphate
  modified_base order(2,4,14,16): mod_base = OTHER; note = 2-prime-deoxy-2-prime-fluorouridine-3-prime-phospha te
  modified_base order(3,9,13): mod_base = OTHER; note = 2-(N-hexylamino)-2-prime-O-methyladenosine-3-prime- phosphate (2-prime OMe-2-C6-adenosine)
  modified_base order(5,21..22): mod_base = OTHER; note = 2-prime-O-methyladenosine-3-prime-phosphate
  modified_base order(6,8,10,18): mod_base = OTHER; note = 2-prime-deoxy-2-prime-fluoroguanosine-3-prime-phosp hate
  modified_base 20: mod_base = OTHER; note = 2-prime-deoxy-2-prime-fluorocytidine-3-prime-phosph ate
  modified_base order(1^2,2^3,21^22,22^23): mod_base = OTHER; note = Phosphorothioate linkages
ttntagtgngttnttttgtcaat

SEQ ID NO: 184 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base order(2,14,20): mod_base = OTHER; note = 2-prime-O-methyladenosine-3-prime-phosphate
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base order(10,19,21): mod_base = OTHER; note = 2-prime-deoxy-2-prime-fluoroadenosine-3-prime-phosp hate
  modified_base order(1^2,2^3): mod_base = OTHER; note = Phosphorothioate linkages
tactgttggattgattcgaaa

SEQ ID NO: 185 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base order(2,14,20): mod_base = OTHER; note = 2-prime-O-methyladenosine-3-prime-phosphate
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base order(10,19): mod_base = OTHER; note = 2-prime-deoxy-2-prime-fluoroadenosine-3-prime-phosp hate
  modified_base 21: mod_base = OTHER; note = 2-prime F-2-tri-N-acetylgalactosamine 3-prime-phosphate
  modified_base order(1^2,2^3): mod_base = OTHER; note = Phosphorothioate linkages
tactgttggattgattcgaan

SEQ ID NO: 186 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base 2: mod_base = OTHER; note = 2-prime F-2-tri-N-acetylgalactosamine 3-prime-phosphate
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base order(10,19,21): mod_base = OTHER; note = 2-prime-deoxy-2-prime-fluoroadenosine-3-prime-phosp hate
  modified_base order(14,20): mod_base = OTHER; note = 2-prime-O-methyladenosine-3-prime-phosphate
  modified_base order(1^2,2^3,19^20,20^21): mod_base = OTHER; note = Phosphorothioate linkages
tnctgttggattgattcgaaa

SEQ ID NO: 187 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base 2: mod_base = OTHER; note = 2-(N-hexadecylamino)-2-prime-deoxy-2-prime-fluoro-a denosine-3-prime-phosphate (2-primeF-2-C16-adenosine);
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base order(10,19,21): mod_base = OTHER; note = 2-prime-deoxy-2-prime-fluoroadenosine-3-prime-phosp hate
  modified_base order(14,20): mod_base = OTHER; note = 2-prime-O-methyladenosine-3-prime-phosphate
  modified_base order(1^2,2^3,19^20,20^21): mod_base = OTHER; note = Phosphorothioate linkages
tnctgttggattgattcgaaa

SEQ ID NO: 188 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base order(2,10): mod_base = OTHER; note = 2-(N-nonylamino)-2-prime-deoxy-2-prime-fluoro-adeno sine-3-prime-phosphate (2-prime F-2-C9-adenosine)
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base order(14,20): mod_base = OTHER; note = 2-prime-O-methyladenosine-3-prime-phosphate
  modified_base order(19,21): mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
tnctgttggnttgattcgaaa

SEQ ID NO: 189 - RNA, 21 nt, synthetic construct
  source 1..21: mol_type = other RNA
  modified_base order(1,7,11,15): mod_base = OTHER; note = 2-prime-deoxy-2-prime-fluorouridine-3-prime-phospha te
  modified_base order(2,10): mod_base = OTHER; note = 2-(N-hexylamino)-2-prime-deoxy-2-prime-fluoro-adeno sine-3-prime-phosphate (2-prime F-2-C6-adenosine)
  modified_base order(3,17): mod_base = OTHER; note = 2-prime-deoxy-2-prime-fluorocytidine-3-prime-phosph ate
  modified_base order(4,6,12,16): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18): mod_base = OTHER; note = 2-prime-O-methylguanosine-3-prime-phosphate
  modified_base 14: mod_base = OTHER; note = 2-(N-hexylamino)-2-prime-O-methyladenosine-3-prime- phosphate (2-prime OMe-2-C6-adenosine)
  modified_base order(19,21): mod_base = OTHER; note = 2-prime-deoxy-2-prime-fluoroadenosine-3-prime-phosp hate
  modified_base 20: mod_base = OTHER; note = 2-prime-O-methyladenosine-3-prime-phosphate
  modified_base order(1^2,2^3,19^20,20^21): mod_base = OTHER; note = Phosphorothioate linkages
tnctgttggnttgnttcgaaa

SEQ ID NO: 190 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,12): mod_base = OTHER; note = 2-prime-O-methyluridine-3-prime-phosphate
  modified_base order(2,8,20): mod_base = OTHER; note = 2-prime-deoxy-2-prime-fluorouridine-3-prime-phospha te
  modified_base order(4,14): mod_base = OTHER; note = 2-prime-deoxy-2-prime-fluorocytidine-3-prime-phosph ate
  modified_base order(5,19,22): mod_base = OTHER; note = 2-prime-O-methylguanosine-3-prime-phosphate
  modified_base order(6,10,17,19): mod_base = OTHER; note = 2-prime-deoxy-2-prime-fluoroadenosine-3-prime-phosp hate
  modified_base order(7,11,15,21): mod_base = OTHER; note = 2-prime-O-methyladenosine-3-prime-phosphate
  modified_base order(9,13,17,23): mod_base = OTHER; note = 2-prime-O-methylcytidine-3-prime-phosphate
  modified_base order(1^2,2^3,21^22,22^23): mod_base = OTHER; note = Phosphorothioate linkages
tttcgaatcaatccaacagtagc

SEQ ID NO: 191 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,12): mod_base = OTHER; note = 2-prime-O-methyluridine-3-prime-phosphate
  modified_base order(2,8,20): mod_base = OTHER; note = 2-prime-deoxy-2-prime-fluorouridine-3-prime-phospha te
  modified_base order(4,14): mod_base = OTHER; note = 2-prime-deoxy-2-prime-fluorocytidine-3-prime-phosph ate
  modified_base order(5,19,22): mod_base = OTHER; note = 2-prime-O-methylguanosine-3-prime-phosphate
  modified_base 6: mod_base = OTHER; note = 2-(N-hexadecylamino)-2-prime-deoxy-2-prime-fluoro-a denosine-3-prime-phosphate (2-primeF-2-C16-adenosine);
  modified_base order(7,11,15,21): mod_base = OTHER; note = 2-prime-O-methyladenosine-3-prime-phosphate
  modified_base order(9,13,17,23): mod_base = OTHER; note = 2-prime-O-methylcytidine-3-prime-phosphate
  modified_base order(10,16,18): mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
tttcgnatcaatccaacagtagc

SEQ ID NO: 192 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,12): mod_base = OTHER; note = 2-prime-O-methyluridine-3-prime-phosphate
  modified_base order(2,8,20): mod_base = OTHER; note = 2-prime-deoxy-2-prime-fluorouridine-3-prime-phospha te
  modified_base order(4,14): mod_base = OTHER; note = 2-prime-deoxy-2-prime-fluorocytidine-3-prime-phosph ate
  modified_base order(5,19,22): mod_base = OTHER; note = 2-prime-O-methylguanosine-3-prime-phosphate
  modified_base order(6,10): mod_base = OTHER; note = 2-(N-nonylamino)-2-prime-deoxy-2-prime-fluoro-adeno sine-3-prime-phosphate (2-prime F-2-C9-adenosine)
  modified_base order(7,11,15,21): mod_base = OTHER; note = 2-prime-O-methyladenosine-3-prime-phosphate
  modified_base order(9,13,17,23): mod_base = OTHER; note = 2-prime-O-methylcytidine-3-prime-phosphate
  modified_base order(16,18): mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
tttcgnatcnatccaacagtagc

SEQ ID NO: 193 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,12): mod_base = OTHER; note = 2-prime-O-methyluridine-3-prime-phosphate
  modified_base order(2,8,20): mod_base = OTHER; note = 2-prime-deoxy-2-prime-fluorouridine-3-prime-phospha te
  modified_base order(4,14): mod_base = OTHER; note = 2-prime-deoxy-2-prime-fluorocytidine-3-prime-phosph ate
  modified_base order(5,19,22): mod_base = OTHER; note = 2-prime-O-methylguanosine-3-prime-phosphate
  modified_base order(6,10): mod_base = OTHER; note = 2-(N-hexylamino)-2-prime-deoxy-2-prime-fluoro-adeno sine-3-prime-phosphate (2-prime F-2-C6-adenosine)
  modified_base order(7,11,21): mod_base = OTHER; note = 2-prime-O-methyladenosine-3-prime-phosphate
  modified_base order(9,13,17,23): mod_base = OTHER; note = 2-prime-O-methylcytidine-3-prime-phosphate
  modified_base 15: mod_base = OTHER; note = 2-(N-hexylamino)-2-prime-O-methyladenosine-3-prime- phosphate (2-prime OMe-2-C6-adenosine)
  modified_base order(16,18): mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
tttcgnatcnatccnacagtagc

SEQ ID NO: 194 - RNA, 21 nt, synthetic construct
  source 1..21: mol_type = other RNA
  modified_base order(1,13,15,17): mod_base = OTHER; note = 2-prime-O-methylcytidine-3-prime-phosphate
  modified_base order(2,8,14,19..21): mod_base = OTHER; note = 2-prime-O-methyladenosine-3-prime-phosphate
  modified_base order(3..6,12,16,18): mod_base = OTHER; note = 2-prime-O-methyluridine-3-prime-phosphate
  modified_base 7: mod_base = OTHER; note = 2-prime-deoxy-2-prime-fluoroadenosine-3-prime-phosp hate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1^2,2^3,19^20,20^21): mod_base = OTHER; note = Phosphorothioate linkages
cattttaatcctcactctaaa

SEQ ID NO: 195 - RNA, 21 nt, synthetic construct
  source 1..21: mol_type = other RNA
  modified_base order(1,13,15,17): mod_base = OTHER; note = 2-prime-O-methylcytidine-3-prime-phosphate
  modified_base 2: mod_base = OTHER; note = 2-(N-hexadecylamino)-2-prime-O-methyladenosine-3-pr ime-phosphate (2-prime OMe-2-C16-adenosine)
  modified_base order(3..6,12,16,18): mod_base = OTHER; note = 2-prime-O-methyluridine-3-prime-phosphate
  modified_base 7: mod_base = OTHER; note = 2-prime-deoxy-2-prime-fluoroadenosine-3-prime-phosp hate
  modified_base order(8,14,19..21): mod_base = OTHER; note = 2-prime-O-methyladenosine-3-prime-phosphate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1^2,2^3,19^20,20^21): mod_base = OTHER; note = Phosphorothioate linkages
cnttttaatcctcactctaaa

SEQ ID NO: 196 - RNA, 21 nt, synthetic construct
  source 1..21: mol_type = other RNA
  modified_base order(1,13,15,17): mod_base = OTHER; note = 2-prime-O-methylcytidine-3-prime-phosphate
  modified_base 2: mod_base = OTHER; note = 2-(N-nonylamino)-2-prime-O-methyladenosine-3-prime- phosphate (2-prime OMe-2-C9-adenosine)
  modified_base order(3..6,12,16,18): mod_base = OTHER; note = 2-prime-O-methyluridine-3-prime-phosphate
  modified_base 7: mod_base = OTHER; note = 2-(N-nonylamino)-2-prime-deoxy-2-prime-fluoro-adeno sine-3-prime-phosphate (2-prime F-2-C9-adenosine)
  modified_base order(8,14,19..21): mod_base = OTHER; note = 2-prime-O-methyladenosine-3-prime-phosphate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1^2,2^3,19^20,20^21): mod_base = OTHER; note = Phosphorothioate linkages
cnttttnatcctcactctaaa

SEQ ID NO: 197 - RNA, 21 nt, synthetic construct
  source 1..21: mol_type = other RNA
  modified_base order(1,13,15,17): mod_base = OTHER; note = 2-prime-O-methylcytidine-3-prime-phosphate
  modified_base 2: mod_base = OTHER; note = 2-(N-hexylamino)-2-prime-O-methyladenosine-3-prime- phosphate (2-prime OMe-2-C6-adenosine)
  modified_base order(3..6,12,16,18): mod_base = OTHER; note = 2-prime-O-methyluridine-3-prime-phosphate
  modified_base 7: mod_base = OTHER; note = 2-(N-hexylamino)-2-prime-deoxy-2-prime-fluoro-adeno sine-3-prime-phosphate (2-prime F-2-C6-adenosine)
  modified_base order(8,19..21): mod_base = OTHER; note = 2-prime-O-methyladenosine-3-prime-phosphate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1^2,2^3,19^20,20^21): mod_base = OTHER; note = Phosphorothioate linkages
cnttttnatcctcnctctaaa

SEQ ID NO: 198 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,15,20): mod_base = OTHER; note = 2-prime-O-methyluridine-3-prime-phosphate
  modified_base order(2,8,14): mod_base = OTHER; note = 2-prime-deoxy-2-prime-fluorouridine-3-prime-phospha te
  modified_base order(4,10,13,17..19,22): mod_base = OTHER; note = 2-prime-O-methyladenosine-3-prime-phosphate
  modified_base order(5,7,11..12,21,23): mod_base = OTHER; note = 2-prime-O-methylguanosine-3-prime-phosphate
  modified_base order(6,16): mod_base = OTHER; note = 2-prime-deoxy-2-prime-fluoroadenosine-3-prime-phosp hate
  modified_base 9: mod_base = OTHER; note = 2-prime-deoxy-2-prime-fluoroguanosine-3-prime-phosp hate
  modified_base order(1^2,2^3,21^22,22^23): mod_base = OTHER; note = Phosphorothioate linkages
tttagagtgaggattaaaatgag

SEQ ID NO: 199 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,15,20): mod_base = OTHER; note = 2-prime-O-methyluridine-3-prime-phosphate
  modified_base order(2,8,14): mod_base = OTHER; note = 2-prime-deoxy-2-prime-fluorouridine-3-prime-phospha te
  modified_base 4: mod_base = OTHER; note = 2-(N-hexadecylamino)-2-prime-O-methyladenosine-3-pr ime-phosphate (2-prime OMe-2-C16-adenosine)
  modified_base order(5,7,11..12,21,23): mod_base = OTHER; note = 2-prime-O-methylguanosine-3-prime-phosphate
  modified_base order(6,16): mod_base = OTHER; note = 2-prime-deoxy-2-prime-fluoroadenosine-3-prime-phosp hate
  modified_base 9: mod_base = OTHER; note = 2-prime-deoxy-2-prime-fluoroguanosine-3-prime-phosp hate
  modified_base order(10,13,17..19,22): mod_base = OTHER; note = 2-prime-O-methyladenosine-3-prime-phosphate
  modified_base order(1^2,2^3,21^22,22^23): mod_base = OTHER; note = Phosphorothioate linkages
tttngagtgaggattaaaatgag

SEQ ID NO: 200 - RNA, 23 nt, synthetic construct
  source 1..23: mol_type = other RNA
  modified_base 1: mod_base = OTHER; note = Vinylphosphonate 2-prime-O-methyluridine-3-prime-phosphate
  modified_base order(3,15,20): mod_base = OTHER; note = 2-prime-O-methyluridine-3-prime-phosphate
  modified_base order(2,8,14): mod_base = OTHER; note = 2-prime-deoxy-2-prime-fluorouridine-3-prime-phospha te
  modified_base order(4,10): mod_base = OTHER; note = 2-(N-nonylamino)-2-prime-O-methyladenosine-3-prime- phosphate (2-prime OMe-2-C9-adenosine)
  modified_base order(5,7,11..12,21,23): mod_base = OTHER; note = 2-prime-O-methylguanosine-3-prime-phosphate
  modified_base order(6,16): mod_base = OTHER; note = 2-prime-deoxy-2-prime-fluoroadenosine-3-prime-phosp hate
  modified_base 9: mod_base = OTHER; note = 2-prime-deoxy-2-prime-fluoroguanosine-3-prime-phosp hate
  modified_base order(13,17..19,22): mod_base = OTHER; note = 2-prime-O-methyladenosine-3-prime-phosphate
  modified_base order(1^2,2^3,21^22,22^23): mod_base = OTHER; note = Phosphorothioate linkages
tttngagtgnggattaaaatgag

SEQ ID NO: 201 - RNA, 44 nt, synthetic construct; identical to SEQ ID NO: 93
  source 1..44: mol_type = other RNA
  modified_base order(1,21,31,35,37): mod_base = OTHER; note = 2-prime-deoxy-2-prime-fluoroadenosine-3-prime-phosp hate
  modified_base order(2,4,18,20,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(33,40): mod_base = OTHER; note = 2-prime-O-methylguanosine-3-prime-phosphate
  modified_base order(1^2,2^3,22^23,23^24,42^43,43^44): mod_base = OTHER; note = Phosphorothioate linkages

SEQ ID NO: 202 - RNA, 44 nt, synthetic construct
  source 1..44: mol_type = other RNA
  modified_base 1: mod_base = OTHER; note = 2-prime F-2-N-acetylgalactosamine-adenosine 3-prime-phosphate
  modified_base order(2,4): mod_base = OTHER; note = 2-prime OMe-2-N-acetylgalactosamine-adenosine 3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(18,20,24,26,28,32,34): mod_base = OTHER; note = 2-prime-O-methyladenosine-3-prime-phosphate
  modified_base order(21,31,35,37): mod_base = OTHER; note = 2-prime-deoxy-2-prime-fluoroadenosine-3-prime-phosp hate
  modified_base order(33,40): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages
nncngtgttcttgctctataattatagagcaagaacactgtttt

SEQ ID NO: 203 - RNA, 44 nt, synthetic construct; identical to SEQ ID NO: 124
  source 1..44: mol_type = other RNA
  modified_base 1: mod_base = OTHER; note = 2-(N-(6-(O-cholesterol)oxcarbonylaminohexyl)amino)- 2-prime-deoxy-2-prime-fluoro-adenosine 3-prime-phosphate(2-prime-F-2-cholesterol-adenosine)
  modified_base order(2,4,18,20,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(21,31,35,37): mod_base = OTHER; note = 2-prime-deoxy-2-prime-fluoroadenosine-3-prime-phosp hate
  modified_base order(33,40): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages

SEQ ID NO: 204 - RNA, 44 nt, synthetic construct; identical to SEQ ID NO: 124
  source 1..44: mol_type = other RNA
  modified_base 1: mod_base = OTHER; note = 2-prime F-2-tri-N-acetylgalactosamine 3-prime-phosphate
  modified_base order(2,4,18,20,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(21,31,35,37): mod_base = OTHER; note = 2-prime-deoxy-2-prime-fluoroadenosine-3-prime-phosp hate
  modified_base order(33,40): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages

SEQ ID NO: 205 - RNA, 44 nt, synthetic construct; identical to SEQ ID NO: 124
  source 1..44: mol_type = other RNA
  modified_base 1: mod_base = OTHER; note = 2-prime F-2-cRGD Kessler peptide 3-prime-phosphate
  modified_base order(2,4,18,20,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(21,31,35,37): mod_base = OTHER; note = 2-prime-deoxy-2-prime-fluoroadenosine-3-prime-phosp hate
  modified_base order(33,40): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages

SEQ ID NO: 206 - RNA, 44 nt, synthetic construct
  source 1..44: mol_type = other RNA
  modified_base order(1,31,35,37): mod_base = OTHER; note = 2-prime-deoxy-2-prime-fluoroadenosine-3-prime-phosp hate
  modified_base order(2,4,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base order(18,20): mod_base = OTHER; note = 2-prime OMe-2-N-acetylgalactosamine-adenosine 3-prime-phosphate
  modified_base 21: mod_base = OTHER; note = 2-prime F-2-N-acetylgalactosamine-adenosine 3-prime-phosphate
  modified_base order(33,40): mod_base = OTHER; note = 2-prime-O-methylguanosine-3-prime-phosphate
  modified_base order(1^2,2^3,22^23,23^24,42^43,43^44): mod_base = OTHER; note = Phosphorothioate linkages
aacagtgttcttgctctntnnttatagagcaagaacactgtttt

SEQ ID NO: 207 - RNA, 44 nt, synthetic construct; identical to SEQ ID NO: 123
  source 1..44: mol_type = other RNA
  modified_base order(1,31,35,37): mod_base = OTHER; note = 2-prime-deoxy-2-prime-fluoroadenosine-3-prime-phosp hate
  modified_base order(2,4,18,20,24,26,28,32,34): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13,27,29): mod_base = OTHER; note = 2-prime-deoxy-2-prime-fluoroguanosine-3-prime-phosp hate
  modified_base order(6,8,12,22,42..44): mod_base = OTHER; note = 2-prime-O-methyluridine-3-prime-phosphate
  modified_base order(9,11,15,17,19,23,25,39,41): mod_base = OTHER; note = 2-prime-deoxy-2-prime-fluorouridine-3-prime-phospha te
  modified_base order(14,16,30,36,38): mod_base = OTHER; note = 2-prime-O-methylcytidine-3-prime-phosphate
  modified_base 21: mod_base = OTHER; note = 2-prime F-2-tri-N-acetylgalactosamine 3-prime-phosphate
  modified_base order(33,40): mod_base = OTHER; note = 2-prime-O-methylguanosine-3-prime-phosphate
  modified_base order(1^2,2^3,22^23,23^24,42^43,43^44): mod_base = OTHER; note = Phosphorothioate linkages

SEQ ID NO: 208 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base order(2,3..4,12,20,26,34): mod_base = OTHER; note = 2-prime-O-methylguanosine-3-prime-phosphate
  modified_base order(5,14..15,18..19,21,32,36,42): mod_base = OTHER; note = 2-prime-O-methyladenosine-3-prime-phosphate
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,30,35,37): mod_base = OTHER; note = 2-prime-deoxy-2-prime-fluoroadenosine-3-prime-phosp hate
  modified_base order(16..17,31,39..41,44): mod_base = OTHER; note = 2-prime-O-methylcytidine-3-prime-phosphate
  modified_base 22: mod_base = OTHER; note = Vinylphosphonate 2-prime-O-methyluridine-3-prime-phosphate
  modified_base 27: mod_base = OTHER; note = 2-prime-deoxy-2-prime-fluoroguanosine-3-prime-phosp hate
  modified_base order(1^2,2^3,19^20,20^21,22^23,23^24,42^43,43^44): mod_base = OTHER; note = Phosphorothioate linkages
tgggatttcatgtaaccaagatcttggttacatgaaatcccatc

SEQ ID NO: 209 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base order(2..4,12,20,26,34): mod_base = OTHER; note = 2-prime-O-methylguanosine-3-prime-phosphate
  modified_base 5: mod_base = OTHER; note = 2-(N-hexadecylamino)-2-prime-deoxy-2-prime-fluoro-a denosine-3-prime-phosphate (2-primeF-2-C16-adenosine);
  modified_base order(7,11): mod_base = OTHER; note = 2-prime-deoxy-2-prime-fluorouridine-3-prime-phospha te
  modified_base 22: mod_base = OTHER; note = Vinylphosphonate 2-prime-O-methyluridine-3-prime-phosphate
  modified_base order(9,23): mod_base = OTHER; note = 2-prime-deoxy-2-prime-fluorocytidine-3-prime-phosph ate
  modified_base order(10,30,35,37): mod_base = OTHER; note = 2-prime-deoxy-2-prime-fluoroadenosine-3-prime-phosp hate
  modified_base order(14..15,18..19,21,32,36,42): mod_base = OTHER; note = 2-prime-O-methyladenosine-3-prime-phosphate
  modified_base order(16..17,31,39..41,44): mod_base = OTHER; note = 2-prime-O-methylcytidine-3-prime-phosphate
  modified_base 27: mod_base = OTHER; note = 2-prime-deoxy-2-prime-fluoroguanosine-3-prime-phosp hate
  modified_base order(1^2,2^3,19^20,20^21,22^23,23^24,42^43,43^44): mod_base = OTHER; note = Phosphorothioate linkages
tgggntttcatgtaaccaagatcttggttacatgaaatcccatc

SEQ ID NO: 210 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base 22: mod_base = OTHER; note = Vinylphosphonate 2-prime-O-methyluridine-3-prime-phosphate
  modified_base order(2..4,12,20,26,34): mod_base = OTHER; note = 2-prime-O-methylguanosine-3-prime-phosphate
  modified_base order(5,14): mod_base = OTHER; note = 2-(N-nonylamino)-2-prime-deoxy-2-prime-fluoro-adeno sine-3-prime-phosphate (2-prime F-2-C9-adenosine)
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,30,35,37): mod_base = OTHER; note = 2-prime-deoxy-2-prime-fluoroadenosine-3-prime-phosp hate
  modified_base order(15,18..19,21,32,36,42): mod_base = OTHER; note = 2-prime-O-methyladenosine-3-prime-phosphate
  modified_base order(16..17,31,39..41,44): mod_base = OTHER; note = 2-prime-O-methylcytidine-3-prime-phosphate
  modified_base 27: mod_base = OTHER; note = 2-prime-deoxy-2-prime-fluoroguanosine-3-prime-phosp hate
  modified_base order(1^2,2^3,19^20,20^21,22^23,23^24,42^43,43^44): mod_base = OTHER; note = Phosphorothioate linkages
tgggntttcatgtnaccaagatcttggttacatgaaatcccatc

SEQ ID NO: 211 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base order(2..4,12,20,26,34): mod_base = OTHER; note = 2-prime-O-methylguanosine-3-prime-phosphate
  modified_base order(5,14,18): mod_base = OTHER; note = 2-(N-hexylamino)-2-prime-O-methyladenosine-3-prime- phosphate (2-prime OMe-2-C6-adenosine)
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,30,35,37): mod_base = OTHER; note = 2-prime-deoxy-2-prime-fluoroadenosine-3-prime-phosp hate
  modified_base order(15,19,21,32,36,42): mod_base = OTHER; note = 2-prime-O-methyladenosine-3-prime-phosphate
  modified_base order(16..17,31,39..41,44): mod_base = OTHER; note = 2-prime-O-methylcytidine-3-prime-phosphate
  modified_base 22: mod_base = OTHER; note = Vinylphosphonate 2-prime-O-methyluridine-3-prime-phosphate
  modified_base order(1^2,2^3,19^20,20^21,22^23,23^24,42^43,43^44): mod_base = OTHER; note = Phosphorothioate linkages
tgggntttcatgtnaccnagatcttggttacatgaaatcccatc

SEQ ID NO: 212 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base 22: mod_base = OTHER; note = Vinylphosphonate 2-prime-O-methyluridine-3-prime-phosphate
  modified_base order(2..4,12,20,26,34): mod_base = OTHER; note = 2-prime-O-methylguanosine-3-prime-phosphate
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,35,37): mod_base = OTHER; note = 2-prime-deoxy-2-prime-fluoroadenosine-3-prime-phosp hate
  modified_base order(16..17,31,39..41,44): mod_base = OTHER; note = 2-prime-O-methylcytidine-3-prime-phosphate
  modified_base 27: mod_base = OTHER; note = 2-prime-deoxy-2-prime-fluoroguanosine-3-prime-phosp hate
  modified_base 30: mod_base = OTHER; note = 2-(N-hexadecylamino)-2-prime-deoxy-2-prime-fluoro-a denosine-3-prime-phosphate (2-primeF-2-C16-adenosine)
  modified_base order(5,14..15,18..19,21,32,36,42): mod_base = OTHER; note = 2-prime-O-methyladenosine-3-prime-phosphate
  modified_base order(1^2,2^3,19^20,20^21,22^23,23^24,42^43,43^44): mod_base = OTHER; note = Phosphorothioate linkages
tgggatttcatgtaaccaagatcttggttncatgaaatcccatc

SEQ ID NO: 213 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base order(2..4,12,20,26,34): mod_base = OTHER; note = 2-prime-O-methylguanosine-3-prime-phosphate
  modified_base order(5,14..15,18..19,21,36,42): mod_base = OTHER; note = 2-prime-O-methyladenosine-3-prime-phosphate
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,35,37): mod_base = OTHER; note = 2-prime-deoxy-2-prime-fluoroadenosine-3-prime-phosp hate
  modified_base order(9,23): mod_base = OTHER; note = 2-prime-deoxy-2-prime-fluorocytidine-3-prime-phosph ate
  modified_base order(16..17,31,39..41,44): mod_base = OTHER; note = 2-prime-O-methylcytidine-3-prime-phosphate
  modified_base 27: mod_base = OTHER; note = 2-prime-deoxy-2-prime-fluoroguanosine-3-prime-phosp hate
  modified_base 30: mod_base = OTHER; note = 2-(N-nonylamino)-2-prime-deoxy-2-prime-fluoro-adeno sine-3-prime-phosphate (2-prime F-2-C9-adenosine)
  modified_base 32: mod_base = OTHER; note = 2-(N-nonylamino)-2-prime-O-methyladenosine-3-prime- phosphate (2-prime OMe-2-C9-adenosine)
  modified_base order(1^2,2^3,19^20,20^21,22^23,23^24,42^43,43^44): mod_base = OTHER; note = Phosphorothioate linkages
tgggatttcatgtaaccaagatcttggttncntgaaatcccatc

SEQ ID NO: 214 - RNA, 44 nt, synthetic construct
  source 1..44: mol_type = other RNA
  modified_base order(1,6,8,13,24..25,28..29,33,38,43): mod_base = OTHER; note = 2-prime-O-methyluridine-3-prime-phosphate
  modified_base order(2..4,12,20,26,34): mod_base = OTHER; note = 2-prime-O-methylguanosine-3-prime-phosphate
  modified_base order(5,14..15,18..19,21,36,42): mod_base = OTHER; note = 2-prime-O-methyladenosine-3-prime-phosphate
  modified_base order(7,11): mod_base = OTHER; note = 2-prime-deoxy-2-prime-fluorouridine-3-prime-phospha te
  modified_base order(9,23): mod_base = OTHER; note = 2-prime-deoxy-2-prime-fluorocytidine-3-prime-phosph ate
  modified_base order(10,37): mod_base = OTHER; note = 2-prime-deoxy-2-prime-fluoroadenosine-3-prime-phosp hate
  modified_base order(16..17,31,39..41,44): mod_base = OTHER; note = 2-prime-O-methylcytidine-3-prime-phosphate
  modified_base 27: mod_base = OTHER; note = 2-prime-deoxy-2-prime-fluoroguanosine-3-prime-phosp hate
  modified_base 22: mod_base = OTHER; note = Vinylphosphonate 2-prime-O-methyluridine-3-prime-phosphate
  modified_base order(30,35): mod_base = OTHER; note = 2-(N-hexylamino)-2-prime-deoxy-2-prime-fluoro-adeno sine-3-prime-phosphate (2-prime F-2-C6-adenosine)
  modified_base 32: mod_base = OTHER; note = 2-(N-hexylamino)-2-prime-O-methyladenosine-3-prime- phosphate (2-prime OMe-2-C6-adenosine)
  modified_base order(1^2,2^3,19^20,20^21,22^23,23^24,42^43,43^44): mod_base = OTHER; note = Phosphorothioate linkages
tgggatttcatgtaaccaagatcttggttncntgnaatcccatc

SEQ ID NO: 215 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base order(1^2,2^3,22^23,23^24,42^43,43^44): mod_base = OTHER; note = Phosphorothioate linkages
tgacaaaataactcactataattatagtgagttattttgtcaat

SEQ ID NO: 216 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(5,7): mod_base = OTHER; note = 2-prime F-2-N-acetylgalactosamine-adenosine 3-prime-phosphate
  modified_base 6: mod_base = OTHER; note = 2-prime OMe-2-N-acetylgalactosamine-adenosine 3-prime-phosphate
  modified_base order(8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgacnnnataactcactataattatagtgagttattttgtcaat

SEQ ID NO: 217 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base 3: mod_base = OTHER; note = 2-prime F-2-tri-N-acetylgalactosamine 3-prime-phosphate
  modified_base order(4,12,14,16): mod_base = OTHER; note = 2-prime-O-methylcytidine-3-prime-phosphate
  modified_base order(5,7,10..11,15,21): mod_base = OTHER; note = 2-prime-deoxy-2-prime-fluoroadenosine-3-prime-phosp hate
  modified_base order(6,8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgncaaaataactcactataattatagtgagttattttgtcaat

SEQ ID NO: 218 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20,24,26,30,34,42..43): mod_base = OTHER; note = 2-prime-O-methyladenosine-3-prime-phosphate
  modified_base 21: mod_base = OTHER; note = 2-prime F-2-tri-N-acetylgalactosamine 3-prime-phosphate
  modified_base order(27,29,31,39): mod_base = OTHER; note = 2-prime-deoxy-2-prime-fluoroguanosine-3-prime-phosp hate
  modified_base order(22,28,32..33,36,38,40,44): mod_base = OTHER; note = 2-prime-O-methyluridine-3-prime-phosphate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgacaaaataactcactatanttatagtgagttattttgtcaat

SEQ ID NO: 219 - RNA, 44 nt, synthetic construct; identical to SEQ ID NO: 217
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base 3: mod_base = OTHER; note = 2-(N-hexadecylamino)-2-prime-deoxy-2-prime-fluoro-a denosine-3-prime-phosphate (2-primeF-2-C16-adenosine)
  modified_base order(4,12,14,16): mod_base = OTHER; note = 2-prime-O-methylcytidine-3-prime-phosphate
  modified_base order(5,7,10..11,15,21): mod_base = OTHER; note = 2-prime-deoxy-2-prime-fluoroadenosine-3-prime-phosp hate
  modified_base order(6,8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base order(1^2,2^3,19^20,20^21,22^23,23^24,42^43,43^44): mod_base = OTHER; note = Phosphorothioate linkages

SEQ ID NO: 220 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10): mod_base = OTHER; note = 2-(N-nonylamino)-2-prime-deoxy-2-prime-fluoro-adeno sine-3-prime-phosphate (2-prime F-2-C9-adenosine)
  modified_base order(4,12,14,16): mod_base = OTHER; note = 2-prime-O-methylcytidine-3-prime-phosphate
  modified_base order(5,7,11,15,21): mod_base = OTHER; note = 2-prime-deoxy-2-prime-fluoroadenosine-3-prime-phosp hate
  modified_base order(6,8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,19^20,20^21,22^23,23^24,42^43,43^44): mod_base = OTHER; note = Phosphorothioate linkages
tgncaaaatnactcactataattatagtgagttattttgtcaat

SEQ ID NO: 221 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,10,15): mod_base = OTHER; note = 2-(N-hexylamino)-2-prime-deoxy-2-prime-fluoro-adeno sine-3-prime-phosphate (2-prime F-2-C6-adenosine)
  modified_base order(4,12,14,16): mod_base = OTHER; note = 2-prime-O-methylcytidine-3-prime-phosphate
  modified_base order(5,7,11): mod_base = OTHER; note = 2-prime-deoxy-2-prime-fluoroadenosine-3-prime-phosp hate
  modified_base order(6,8,18,20,24,26,30,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,19^20,20^21,22^23,23^24,42^43,43^44): mod_base = OTHER; note = Phosphorothioate linkages
tgncaaaatnactcnctataattatagtgagttattttgtcaat

SEQ ID NO: 222 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20,26,30,34,42..43): mod_base = OTHER; note = 2-prime-O-methyladenosine-3-prime-phosphate
  modified_base 24: mod_base = OTHER; note = 2-(N-hexadecylamino)-2-prime-O-methyladenosine-3-pr ime-phosphate (2-prime OMe-2-C16-adenosine)
  modified_base order(27,29,31,39): mod_base = OTHER; note = 2-prime-deoxy-2-prime-fluoroguanosine-3-prime-phosp hate
  modified_base order(22,28,32..33,36,38,40,44): mod_base = OTHER; note = 2-prime-O-methyluridine-3-prime-phosphate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgacaaaataactcactataattntagtgagttattttgtcaat

SEQ ID NO: 223 - RNA, 44 nt, synthetic construct
  source 1..44: mol_type = other RNA
  modified_base order(1,9,13,17,19,23,25,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20,26,34,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(24,30): mod_base = OTHER; note = 2-(N-nonylamino)-2-prime-O-methyladenosine-3-prime- phosphate (2-prime OMe-2-C9-adenosine)
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgacaaaataactcactataattntagtgngttattttgtcaat

SEQ ID NO: 224 - RNA, 44 nt, synthetic construct
  source 1..44: mol_type = other RNA
  modified_base order(1,9,13,17,19,23,25,35,37): mod_base = OTHER; note = 2-prime-deoxy-2-prime-fluorouridine-3-prime-phospha te
  modified_base 2: mod_base = OTHER; note = 2-prime-O-methylguanosine-3-prime-phosphate
  modified_base order(3,5,7,10..11,15,21): mod_base = OTHER; note = 2-prime-deoxy-2-prime-fluoroadenosine-3-prime-phosp hate
  modified_base order(4,12,14,16): mod_base = OTHER; note = 2-prime-O-methylcytidine-3-prime-phosphate
  modified_base order(6,8,18,20,26,42..43): mod_base = OTHER; note = 2-prime-O-methyladenosine-3-prime-phosphate
  modified_base order(22,28,32..33,36,38,40,44): mod_base = OTHER; note = 2-prime-O-methyluridine-3-prime-phosphate
  modified_base order(24,30,34): mod_base = OTHER; note = 2-(N-hexylamino)-2-prime-O-methyladenosine-3-prime- phosphate (2-prime OMe-2-C6-adenosine)
  modified_base order(27,29,31,39): mod_base = OTHER; note = 2-prime-deoxy-2-prime-fluoroguanosine-3-prime-phosp hate
  modified_base 41: mod_base = OTHER; note = 2-prime-deoxy-2-prime-fluorocytidine-3-prime-phosph ate
  modified_base order(1^2,2^3,22^23,23^24,42^43,43^44): mod_base = OTHER; note = Phosphorothioate linkages
tgacaaaataactcactataattntagtgngttnttttgtcaat

SEQ ID NO: 225 - RNA, 44 nt, synthetic construct
  source 1..44: mol_type = other RNA
  modified_base order(1,7,11,15,23,29): mod_base = OTHER; note = 2-prime-deoxy-2-prime-fluorouridine-3-prime-phospha te
  modified_base order(2,14,20,28,32,36,42): mod_base = OTHER; note = 2-prime-O-methyladenosine-3-prime-phosphate
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base 22: mod_base = OTHER; note = Vinylphosphonate 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18,26,40,43): mod_base = OTHER; note = 2-prime-O-methylguanosine-3-prime-phosphate
  modified_base order(10,19,21,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(1^2,2^3,22^23,23^24,42^43,43^44): mod_base = OTHER; note = Phosphorothioate linkages
tactgttggattgattcgaaatttcgaatcaatccaacagtagc

SEQ ID NO: 226 - RNA, 44 nt, synthetic construct
  source 1..44: mol_type = other RNA
  modified_base order(1^2,2^3,22^23,23^24,42^43,43^44): mod_base = OTHER; note = Phosphorothioate linkages
  modified_base 22: mod_base = OTHER; note = Vinylphosphonate 2-prime-O-methyluridine-3-prime-phosphate
  modified_base order(1,7,11,15,23,29,41): mod_base = OTHER; note = 2-prime-deoxy-2-prime-fluorouridine-3-prime-phospha te
  modified_base order(2,14,20,28,32,36,42): mod_base = OTHER; note = 2-prime-O-methyladenosine-3-prime-phosphate
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18,26,40,43): mod_base = OTHER; note = 2-prime-O-methylguanosine-3-prime-phosphate
  modified_base order(10,19,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base 21: mod_base = OTHER; note = 2-prime F-2-tri-N-acetylgalactosamine 3-prime-phosphate
tactgttggattgattcgaantttcgaatcaatccaacagtagc

SEQ ID NO: 227 - RNA, 44 nt, synthetic construct
  source 1..44: mol_type = other RNA
  modified_base order(1^2,2^3,19^20,20^21,22^23,23^24,42^43,43^44): mod_base = OTHER; note = Phosphorothioate linkages
  modified_base order(1,7,11,15,23,29,41): mod_base = OTHER; note = 2-prime-deoxy-2-prime-fluorouridine-3-prime-phospha te
  modified_base order(3,17,25,35): mod_base = OTHER; note = 2-prime-deoxy-2-prime-fluorocytidine-3-prime-phosph ate
  modified_base 22: mod_base = OTHER; note = Vinylphosphonate 2-prime-O-methyluridine-3-prime-phosphate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(10,19,21,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(14,20,28,32,36,42): mod_base = OTHER; note = 2-prime-O-methyladenosine-3-prime-phosphate
  modified_base order(8,18,26,40,43): mod_base = OTHER; note = 2-prime-O-methylguanosine-3-prime-phosphate
  modified_base 2: mod_base = OTHER; note = 2-prime F-2-tri-N-acetylgalactosamine 3-prime-phosphate
tnctgttggattgattcgaaatttcgaatcaatccaacagtagc

SEQ ID NO: 228 - RNA, 44 nt, synthetic construct; identical to SEQ ID NO: 227
  source 1..44: mol_type = other RNA
  modified_base order(1^2,2^3,19^20,20^21,22^23,23^24,42^43,43^44): mod_base = OTHER; note = Phosphorothioate linkages
  modified_base 22: mod_base = OTHER; note = Vinylphosphonate 2-prime-O-methyluridine-3-prime-phosphate
  modified_base order(1,7,11,15,23,29,41): mod_base = OTHER; note = 2-prime-deoxy-2-prime-fluorouridine-3-prime-phospha te
  modified_base 2: mod_base = OTHER; note = 2-(N-hexadecylamino)-2-prime-deoxy-2-prime-fluoro-a denosine-3-prime-phosphate (2-primeF-2-C16-adenosine)
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(10,19,21,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(14,20,28,32,36,42): mod_base = OTHER; note = 2-prime-O-methyladenosine-3-prime-phosphate
  modified_base order(8,18,26,40,43): mod_base = OTHER; note = 2-prime-O-methylguanosine-3-prime-phosphate

SEQ ID NO: 229 - RNA, 44 nt, synthetic construct
  source 1..44: mol_type = other RNA
  modified_base order(1^2,2^3,19^20,20^21,22^23,23^24,42^43,43^44): mod_base = OTHER; note = Phosphorothioate linkages
  modified_base 22: mod_base = OTHER; note = Vinylphosphonate 2-prime-O-methyluridine-3-prime-phosphate
  modified_base order(1,7,11,15,23,29,41): mod_base = OTHER; note = 2-prime-deoxy-2-prime-fluorouridine-3-prime-phospha te
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(14,20,28,32,36,42): mod_base = OTHER; note = 2-prime-O-methyladenosine-3-prime-phosphate
  modified_base order(8,18,26,40,43): mod_base = OTHER; note = 2-prime-O-methylguanosine-3-prime-phosphate
  modified_base order(19,21,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(2,10): mod_base = OTHER; note = 2-(N-nonylamino)-2-prime-deoxy-2-prime-fluoro-adeno sine-3-prime-phosphate (2-prime F-2-C9-adenosine)
tnctgttggnttgattcgaaatttcgaatcaatccaacagtagc

SEQ ID NO: 230 - RNA, 44 nt, synthetic construct
  source 1..44: mol_type = other RNA
  modified_base order(1^2,2^3,19^20,20^21,22^23,23^24,42^43,43^44): mod_base = OTHER; note = Phosphorothioate linkages
  modified_base order(2,10): mod_base = OTHER; note = 2-(N-nonylamino)-2-prime-deoxy-2-prime-fluoro-adeno sine-3-prime-phosphate (2-prime F-2-C9-adenosine)
  modified_base 14: mod_base = OTHER; note = 2-(N-hexylamino)-2-prime-O-methyladenosine-3-prime- phosphate (2-prime OMe-2-C6-adenosine)
  modified_base 22: mod_base = OTHER; note = Vinylphosphonate 2-prime-O-methyluridine-3-prime-phosphate
  modified_base order(1,7,11,15,23,29,41): mod_base = OTHER; note = 2-prime-deoxy-2-prime-fluorouridine-3-prime-phospha te
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(20,28,32,36,42): mod_base = OTHER; note = 2-prime-O-methyladenosine-3-prime-phosphate
  modified_base order(8,18,26,40,43): mod_base = OTHER; note = 2-prime-O-methylguanosine-3-prime-phosphate
  modified_base order(19,21,27,31,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
tnctgttggnttgnttcgaaatttcgaatcaatccaacagtagc

SEQ ID NO: 231 - RNA, 44 nt, synthetic construct
  source 1..44: mol_type = other RNA
  modified_base order(30,34,38,44): mod_base = OTHER; note = 2-prime-O-methylcytidine-3-prime-phosphate
  modified_base 22: mod_base = OTHER; note = Vinylphosphonate 2-prime-O-methyluridine-3-prime-phosphate
  modified_base order(10,19,21,31,37,39): mod_base = OTHER; note = 2-prime-deoxy-2-prime-fluoroadenosine-3-prime-phosp hate
  modified_base order(3,17,25,35): mod_base = OTHER; note = 2-prime-deoxy-2-prime-fluorocytidine-3-prime-phosph ate
  modified_base order(8,18,26,40,43): mod_base = OTHER; note = 2-prime-O-methylguanosine-3-prime-phosphate
  modified_base order(1,7,11,15,23,29,41): mod_base = OTHER; note = 2-prime-deoxy-2-prime-fluorouridine-3-prime-phospha te
  modified_base order(1^2,2^3,19^20,20^21,22^23,23^24,42^43,43^44): mod_base = OTHER; note = Phosphorothioate linkages
  modified_base order(4,6,12,16,24,33): mod_base = OTHER; note = 2-prime-O-methyluridine-3-prime-phosphate
  modified_base order(2,14,20,28,32,36,42): mod_base = OTHER; note = 2-prime-O-methyladenosine-3-prime-phosphate
  modified_base order(5,9,13): mod_base = OTHER; note = 2-prime-deoxy-2-prime-fluoroguanosine-3-prime-phosp hate
  modified_base 27: mod_base = OTHER; note = 2-(N-hexadecylamino)-2-prime-deoxy-2-prime-fluoro-a denosine-3-prime-phosphate (2-primeF-2-C16-adenosine)
tactgttggattgattcgaaatttcgnatcaatccaacagtagc

SEQ ID NO: 232 - RNA, 44 nt, synthetic construct
  source 1..44: mol_type = other RNA
  modified_base order(1^2,2^3,19^20,20^21,22^23,23^24,42^43,43^44): mod_base = OTHER; note = Phosphorothioate linkages
  modified_base order(1,7,11,15,23,29,41): mod_base = OTHER; note = 2-prime-deoxy-2-prime-fluorouridine-3-prime-phospha te
  modified_base order(2,14,20,28,32,36,42): mod_base = OTHER; note = 2-prime-O-methyladenosine-3-prime-phosphate
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18,26,40,43): mod_base = OTHER; note = 2-prime-O-methylguanosine-3-prime-phosphate
  modified_base order(10,19,21,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(27,31): mod_base = OTHER; note = 2-(N-nonylamino)-2-prime-deoxy-2-prime-fluoro-adeno sine-3-prime-phosphate (2-prime F-2-C9-adenosine)
  modified_base 22: mod_base = OTHER; note = Vinylphosphonate 2-prime-O-methyluridine-3-prime-phosphate
tactgttggattgattcgaaatttcgnatcnatccaacagtagc

SEQ ID NO: 233 - RNA, 44 nt, synthetic construct
  source 1..44: mol_type = other RNA
  modified_base order(1,7,11,15,23,29,41): mod_base = OTHER; note = 2-prime-deoxy-2-prime-fluorouridine-3-prime-phospha te
  modified_base order(2,14,20,28,32,42): mod_base = OTHER; note = 2-prime-O-methyladenosine-3-prime-phosphate
  modified_base 22: mod_base = OTHER; note = Vinylphosphonate 2-prime-O-methyluridine-3-prime-phosphate
  modified_base order(3,17,25,35): mod_base = OTHER; note = 2-prime-deoxy-2-prime-fluorocytidine-3-prime-phosph ate
  modified_base order(4,6,12,16,24,33): mod_base = OTHER; note = 2-prime-O-methyluridine-3-prime-phosphate
  modified_base order(5,9,13): mod_base = OTHER; note = 2-prime-deoxy-2-prime-fluoroguanosine-3-prime-phosp hate
  modified_base order(8,18,26,40,43): mod_base = OTHER; note = 2-prime-O-methylguanosine-3-prime-phosphate
  modified_base order(10,19,21,37,39): mod_base = OTHER; note = 2-prime-deoxy-2-prime-fluoroadenosine-3-prime-phosp hate
  modified_base order(30,34,38,44): mod_base = OTHER; note = 2-prime-O-methylcytidine-3-prime-phosphate
  modified_base order(27,31): mod_base = OTHER; note = 2-(N-hexylamino)-2-prime-deoxy-2-prime-fluoro-adeno sine-3-prime-phosphate (2-prime F-2-C6-adenosine)
  modified_base 36: mod_base = OTHER; note = 2-(N-hexylamino)-2-prime-O-methyladenosine-3-prime- phosphate (2-prime OMe-2-C6-adenosine)
  modified_base order(1^2,2^3,19^20,20^21,22^23,23^24,42^43,43^44): mod_base = OTHER; note = Phosphorothioate linkages
tactgttggattgattcgaaatttcgnatcnatccnacagtagc

SEQ ID NO: 234 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base order(1,13,15,17): mod_base = OTHER; note = 2-prime-O-methylcytidine-3-prime-phosphate
  modified_base order(2,8,14,19,20,21,25,31,34,38,39,40,43): mod_base = OTHER; note = 2-prime-O-methyladenosine-3-prime-phosphate
  modified_base order(3,4,5,6,12,16,24,36,41): mod_base = OTHER; note = 2-prime-O-methyluridine-3-prime-phosphate
  modified_base order(7,27,37): mod_base = OTHER; note = 2-prime-deoxy-2-prime-fluoroadenosine-3-prime-phosp hate
  modified_base order(9,23,29,35): mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base 30: mod_base = OTHER; note = 2-prime-deoxy-2-prime-fluoroguanosine-3-prime-phosp hate
  modified_base order(25,28,32,33,42,44): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages
cattttaatcctcactctaaatttagagtgaggattaaaatgag

SEQ ID NO: 235 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base order(1,13,15,17): mod_base = OTHER; note = 2-prime-O-methylcytidine-3-prime-phosphate
  modified_base 2: mod_base = OTHER; note = 2-(N-hexadecylamino)-2-prime-O-methyladenosine-3-pr ime-phosphate (2-prime OMe-2-C16-adenosine)
  modified_base order(3,4,5,6,12,16,18,24,36,41): mod_base = OTHER; note = 2-prime-O-methyluridine-3-prime-phosphate
  modified_base order(7,27,37): mod_base = OTHER; note = 2-prime-deoxy-2-prime-fluoroadenosine-3-prime-phosp hate
  modified_base order(8,14,19,20,21,25,31,34,38,39,40,43): mod_base = OTHER; note = 2-prime-O-methyladenosine-3-prime-phosphate
  modified_base order(9,23,29,35): mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base 30: mod_base = OTHER; note = 2-prime-deoxy-2-prime-fluoroguanosine-3-prime-phosp hate
  modified_base order(26,28,32,33,42,44): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages
cnttttaatcctcactctaaatttagagtgaggattaaaatgag

SEQ ID NO: 236 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base 30: mod_base = OTHER; note = 2-prime-deoxy-2-prime-fluoroguanosine-3-prime-phosp hate
  modified_base order(10,11): mod_base = OTHER; note = 2-prime-deoxy-2-prime-fluorocytidine-3-prime-phosph ate
  modified_base order(1,13,15,17): mod_base = OTHER; note = 2-prime-O-methylcytidine-3-prime-phosphate
  modified_base order(3,4,5,6,12,16,18,24,36,41): mod_base = OTHER; note = 2-prime-O-methyluridine-3-prime-phosphate
  modified_base order(8,14,19,20,21,25,31,34,38,39,40,43): mod_base = OTHER; note = 2-prime-O-methyladenosine-3-prime-phosphate
  modified_base order(9,23,29,35): mod_base = OTHER; note = 2-prime-deoxy-2-prime-fluorouridine-3-prime-phospha te
  modified_base order(37,37): mod_base = OTHER; note = 2-prime-deoxy-2-prime-fluoroadenosine-3-prime-phosp hate
  modified_base order(26,28,32,33,42,44): mod_base = OTHER; note = 2-prime-O-methylguanosine-3-prime-phosphate
  modified_base 2: mod_base = OTHER; note = 2-(N-nonylamino)-2-prime-O-methyladenosine-3-prime- phosphate (2-prime OMe-2-C9-adenosine)
  modified_base 7: mod_base = OTHER; note = 2-(N-nonylamino)-2-prime-deoxy-2-prime-fluoro-adeno sine-3-prime-phosphate (2-prime F-2-C9-adenosine)
  modified_base order(1^2,2^3,19^20,20^21,22^23,23^24,42^43,43^44): mod_base = OTHER; note = Phosphorothioate linkages
cnttttnatcctcactctaaatttagagtgaggattaaaatgag

SEQ ID NO: 237 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base order(3,4,5,6,12,16,18,24,36,41): mod_base = OTHER; note = 2-prime-O-methyluridine-3-prime-phosphate
  modified_base order(10,11): mod_base = OTHER; note = 2-prime-deoxy-2-prime-fluorocytidine-3-prime-phosph ate
  modified_base order(9,23,29,35): mod_base = OTHER; note = 2-prime-deoxy-2-prime-fluorouridine-3-prime-phospha te
  modified_base order(8,19,20,21,25,31,34,38,39,40,43): mod_base = OTHER; note = 2-prime-O-methyladenosine-3-prime-phosphate
  modified_base order(27,37): mod_base = OTHER; note = 2-prime-deoxy-2-prime-fluoroadenosine-3-prime-phosp hate
  modified_base 30: mod_base = OTHER; note = 2-prime-deoxy-2-prime-fluoroguanosine-3-prime-phosp hate
  modified_base order(1,13,15,17): mod_base = OTHER; note = 2-prime-O-methylcytidine-3-prime-phosphate
  modified_base order(26,28,32,33,42,44): mod_base = OTHER; note = 2-prime-O-methylguanosine-3-prime-phosphate
  modified_base order(2,14): mod_base = OTHER; note = 2-(N-hexylamino)-2-prime-O-methyladenosine-3-prime- phosphate (2-prime OMe-2-C6-adenosine)
  modified_base 7: mod_base = OTHER; note = 2-(N-hexylamino)-2-prime-deoxy-2-prime-fluoro-adeno sine-3-prime-phosphate (2-prime F-2-C6-adenosine)
  modified_base order(1^2,2^3,19^20,20^21,22^23,23^24,42^43,43^44): mod_base = OTHER; note = Phosphorothioate linkages
cnttttnatcctcnctctaaatttagagtgaggattaaaatgag

SEQ ID NO: 238 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base order(10,11): mod_base = OTHER; note = 2-prime-deoxy-2-prime-fluorocytidine-3-prime-phosph ate
  modified_base order(3,4,5,6,12,16,18,24,36,41): mod_base = OTHER; note = 2-prime-O-methyluridine-3-prime-phosphate
  modified_base order(2,8,14,19,20,21,31,34,38,39,40,43): mod_base = OTHER; note = 2-prime-O-methyladenosine-3-prime-phosphate
  modified_base order(1,13,15,17): mod_base = OTHER; note = 2-prime-O-methylcytidine-3-prime-phosphate
  modified_base order(7,27,37): mod_base = OTHER; note = 2-prime-deoxy-2-prime-fluoroadenosine-3-prime-phosp hate
  modified_base order(9,23,29,35): mod_base = OTHER; note = 2-prime-deoxy-2-prime-fluorouridine-3-prime-phospha te
  modified_base 30: mod_base = OTHER; note = 2-prime-deoxy-2-prime-fluoroguanosine-3-prime-phosp hate
  modified_base order(26,28,32,33,42,44): mod_base = OTHER; note = 2-prime-O-methylguanosine-3-prime-phosphate
  modified_base 25: mod_base = OTHER; note = 2-(N-hexadecylamino)-2-prime-O-methyladenosine-3-pr ime-phosphate (2-prime OMe-2-C16-adenosine)
  modified_base order(1^2,2^3,19^20,20^21,22^23,23^24,42^43,43^44): mod_base = OTHER; note = Phosphorothioate linkages
cattttaatcctcactctaaatttngagtgaggattaaaatgag

SEQ ID NO: 239 - RNA, 44 nt, synthetic construct
  source 1..44: mol_type = other RNA
  modified_base order(25,31): mod_base = OTHER; note = 2-(N-nonylamino)-2-prime-O-methyladenosine-3-prime- phosphate (2-prime OMe-2-C9-adenosine)
  modified_base 22: mod_base = OTHER; note = Vinylphosphonate 2-prime-O-methyluridine-3-prime-phosphate
  modified_base order(3,4,5,6,12,16,18,24,36,41): mod_base = OTHER; note = 2-prime-O-methyluridine-3-prime-phosphate
  modified_base order(10,11): mod_base = OTHER; note = 2-prime-deoxy-2-prime-fluorocytidine-3-prime-phosph ate
  modified_base order(7,27,37): mod_base = OTHER; note = 2-prime-deoxy-2-prime-fluoroadenosine-3-prime-phosp hate
  modified_base order(1,13,15,17): mod_base = OTHER; note = 2-prime-O-methylcytidine-3-prime-phosphate
  modified_base order(2,8,14,19,20,21,34,38,39,40,43): mod_base = OTHER; note = 2-prime-O-methyladenosine-3-prime-phosphate
  modified_base order(9,23,29,35): mod_base = OTHER; note = 2-prime-deoxy-2-prime-fluorouridine-3-prime-phospha te
  modified_base 30: mod_base = OTHER; note = 2-prime-deoxy-2-prime-fluoroguanosine-3-prime-phosp hate
  modified_base order(26,28,32,33,42,44): mod_base = OTHER; note = 2-prime-O-methylguanosine-3-prime-phosphate
  modified_base order(1^2,2^3,19^20,20^21,22^23,23^24,42^43,43^44): mod_base = OTHER; note = Phosphorothioate linkages
cattttaatcctcactctaaatttngagtgnggattaaaatgag

SEQ ID NO: 240 - RNA, 44 nt, synthetic construct
  source 1..44: mol_type = other RNA
  modified_base 22: mod_base = OTHER; note = Vinylphosphonate 2-prime-O-methyluridine-3-prime-phosphate
  modified_base order(9,23,29,35): mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1,13,15,17): mod_base = OTHER; note = 2-prime-O-methylcytidine-3-prime-phosphate
  modified_base order(2,8,14,19,20,21,34,38,39,40,43): mod_base = OTHER; note = 2-prime-O-methyladenosine-3-prime-phosphate
  modified_base order(3,4,5,6,12,16,18,24,36,41): mod_base = OTHER; note = 2-prime-O-methyluridine-3-prime-phosphate
  modified_base 30: mod_base = OTHER; note = 2-prime-deoxy-2-prime-fluoroguanosine-3-prime-phosp hate
  modified_base order(7,27): mod_base = OTHER; note = 2-prime-deoxy-2-prime-fluoroadenosine-3-prime-phosp hate
  modified_base order(26,28,32,33,42,44): mod_base = OTHER; note = 2-prime-O-methylguanosine-3-prime-phosphate
  modified_base order(25,31): mod_base = OTHER; note = 2-(N-hexylamino)-2-prime-O-methyladenosine-3-prime- phosphate (2-prime OMe-2-C6-adenosine)
  modified_base 37: mod_base = OTHER; note = 2-(N-hexylamino)-2-prime-deoxy-2-prime-fluoro-adeno sine-3-prime-phosphate (2-prime F-2-C6-adenosine)
  modified_base order(1^2,2^3,19^20,20^21,22^23,23^24,42^43,43^44): mod_base = OTHER; note = Phosphorothioate linkages
cattttaatcctcactctaaatttngagtgnggattnaaatgag

SEQ ID NO: 241 - RNA, 44 nt, synthetic construct; identical to SEQ ID NO: 93
  source 1..44: mol_type = other RNA

SEQ ID NO: 242 - RNA, 44 nt, synthetic construct; identical to SEQ ID NO: 202
  source 1..44: mol_type = other RNA
  modified_base 1: mod_base = OTHER; note = 2prime F-2-N-acetylgalactosamine-adenosine 3-prime-phosphate
  modified_base order(21,31,35,37): mod_base = OTHER; note = 2-prime-deoxy-2-prime-fluoroadenosine-3-prime-phosp hate
  modified_base order(2,4): mod_base = OTHER; note = 2-prime OMe-2-N-acetylgalactosamine-adenosine 3-prime-phosphate
  modified_base order(5,7,13,27,29): mod_base = OTHER; note = 2-prime-deoxy-2-prime-fluoroguanosine-3-prime-phosp hate
  modified_base order(3,10): mod_base = OTHER; note = 2-prime-deoxy-2-prime-fluorocytidine-3-prime-phosph ate
  modified_base order(18,20,24,26,28,32,34): mod_base = OTHER; note = 2-prime-O-methyladenosine-3-prime-phosphate
  modified_base order(9,11,15,17,19,23,25,38,41): mod_base = OTHER; note = 2-prime-deoxy-2-prime-fluorouridine-3-prime-phospha te
  modified_base order(6,8,12,22,42,43,44): mod_base = OTHER; note = 2-prime-O-methyluridine-3-prime-phosphate
  modified_base order(33,40): mod_base = OTHER; note = 2-prime-O-methylguanosine-3-prime-phosphate
  modified_base order(14,16,30,36,38): mod_base = OTHER; note = 2-prime-O-methylcytidine-3-prime-phosphate
  modified_base order(1^2,2^3,19^20,20^21,22^23,23^24,42^43,43^44): mod_base = OTHER; note = Phosphorothioate linkages

SEQ ID NO: 243 - DNA, 19 bp, synthetic construct
  source 1..19: mol_type = other DNA
  modified_base 1: mod_base = OTHER; note = 2-(N-(6-(O-cholesterol)oxcarbonylaminohexyl)amino)- 2-prime-deoxy-2-prime-fluoro-adenosine 3-prime-phosphate(2-prime-F-2-cholesterol-adenosine)
  modified_base order(2,19): mod_base = OTHER; note = 2-prime-deoxyuridine-3-prime-phosphate (Deoxy-Thymidine)
ntttttttttttttttttt

SEQ ID NO: 244 - DNA, 19 bp, synthetic construct
  source 1..19: mol_type = other DNA
  modified_base 1: mod_base = OTHER; note = 2-prime F-2-N-acetylgalactosamine-adenosine 3-prime-phosphate
  modified_base order(2,19): mod_base = OTHER; note = 2-prime-deoxyuridine-3-prime-phosphate (Deoxy-Thymidine)
ntttttttttttttttttt

SEQ ID NO: 245 - DNA, 19 bp, synthetic construct
  source 1..19: mol_type = other DNA
  modified_base 1: mod_base = OTHER; note = 2-prime F-2-tri-N-acetylgalactosamine 3-prime-phosphate
  modified_base order(2,19): mod_base = OTHER; note = 2-prime-deoxyuridine-3-prime-phosphate (Deoxy-Thymidine)
ntttttttttttttttttt

SEQ ID NO: 246 - DNA, 19 bp, synthetic construct
  source 1..19: mol_type = other DNA
  modified_base 1: mod_base = OTHER; note = 2-prime F-2-cRGD Kessler peptide 3-prime-phosphate
  modified_base order(2,19): mod_base = OTHER; note = 2-prime-deoxyuridine-3-prime-phosphate (Deoxy-Thymidine)
ntttttttttttttttttt

SEQ ID NO: 247 - DNA, 19 bp, synthetic construct
  source 1..19: mol_type = other DNA
  modified_base 1: mod_base = OTHER; note = 2-(N-hexadecylamino)-2-prime-deoxy-2-prime-fluoro-a denosine-3-prime-phosphate (2-primeF-2-C16-adenosine)
  modified_base order(2,19): mod_base = OTHER; note = 2-prime-deoxyuridine-3-prime-phosphate (Deoxy-Thymidine)
ntttttttttttttttttt

SEQ ID NO: 248 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base order(1^2,2^3): mod_base = OTHER; note = Phosphorothioate linkages
aacagtgttcttgctctataa

SEQ ID NO: 249 - RNA, 21 nt, synthetic construct
  source 1..21: mol_type = other RNA
  modified_base 1: mod_base = OTHER; note = 2-(N-(6-(O-cholesterol)oxcarbonylaminohexyl)amino)- 2-prime-deoxy-2-prime-fluoro-adenosine 3-prime-phosphate(2-prime-F-2-cholesterol-adenosine)
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nacagtgttcttgctctataa

SEQ ID NO: 250 - RNA, 21 nt, synthetic construct
  source 1..21: mol_type = other RNA
  modified_base 1: mod_base = OTHER; note = 2-prime F-2-N-acetylgalactosamine-adenosine 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nacagtgttcttgctctataa

SEQ ID NO: 251 - RNA, 21 nt, synthetic construct
  source 1..21: mol_type = other RNA
  modified_base 1: mod_base = OTHER; note = 2-prime F-2-tri-N-acetylgalactosamine 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nacagtgttcttgctctataa

SEQ ID NO: 252 - RNA, 21 nt, synthetic construct
  source 1..21: mol_type = other RNA
  modified_base 1: mod_base = OTHER; note = 2-prime F-2-cRGD Kessler peptide 3-prime-phosphate
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(14,16): mod_base = OTHER; note = 2-prime-O-methylcytidine-3-prime-phosphate
  modified_base order(9,11,15,17,19): mod_base = OTHER; note = 2-prime-deoxy-2-prime-fluorouridine-3-prime-phospha te
  modified_base order(6,8,12): mod_base = OTHER; note = 2-prime-O-methyluridine-3-prime-phosphate
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
nacagtgttcttgctctataa

SEQ ID NO: 253 - RNA, 21 nt, synthetic construct
  source 1..21: mol_type = other RNA
  modified_base 1: mod_base = OTHER; note = 2-(N-hexadecylamino)-2-prime-deoxy-2-prime-fluoro-a denosine-3-prime-phosphate (2-primeF-2-C16-adenosine);
  modified_base order(2,4,18,20): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base order(1^2,2^3,19^20,20^21): mod_base = OTHER; note = Phosphorothioate linkages
nacagtgttcttgctctataa

SEQ ID NO: 254 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(5,6): mod_base = OTHER; note = Deoxy-Adenosine
  modified_base order(7,8): mod_base = OTHER; note = Deoxy-Thymidine
cgcgaattcgcg

SEQ ID NO: 255 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base 6: mod_base = OTHER; note = Deoxy-Adenosine
  modified_base order(7,8): mod_base = OTHER; note = Deoxy-Thymidine
  modified_base 5: mod_base = OTHER; note = deoxy-2,6-diaminopurine
cgcgnattcgcg

SEQ ID NO: 256 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base 5: mod_base = OTHER; note = Deoxy-Adenosine
  modified_base 6: mod_base = OTHER; note = deoxy-2,6-diaminopurine
  modified_base order(7,8): mod_base = OTHER; note = Deoxy-Thymidine
cgcganttcgcg

SEQ ID NO: 257 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(5,6): mod_base = OTHER; note = deoxy-2,6-diaminopurine
  modified_base order(7,8): mod_base = OTHER; note = Deoxy-Thymidine
cgcgnnttcgcg

SEQ ID NO: 258 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(7,8): mod_base = OTHER; note = Deoxy-Thymidine
  modified_base 6: mod_base = OTHER; note = Deoxy-Adenosine
  modified_base 5: mod_base = OTHER; note = 2-prime-deoxy-2-(15N)aminoadenosine-3-prime-phospha te
cgcgnattcgcg

SEQ ID NO: 259 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(7,8): mod_base = OTHER; note = Deoxy-Thymidine
  modified_base 5: mod_base = OTHER; note = Deoxy-Adenosine
  modified_base 6: mod_base = OTHER; note = 2-prime-deoxy-2-(15N)aminoadenosine-3-prime-phospha te
cgcganttcgcg

SEQ ID NO: 260 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(7,8): mod_base = OTHER; note = Deoxy-Thymidine
  modified_base order(5,6): mod_base = OTHER; note = 2-prime-deoxy-2-(15N)aminoadenosine-3-prime-phospha te
cgcgnnttcgcg

SEQ ID NO: 261 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base 5: mod_base = OTHER; note = Deoxy-Adenosine
  modified_base 6: mod_base = OTHER; note = 2-fluoro-deoxy-adenosine
  modified_base order(7,8): mod_base = OTHER; note = Deoxy-Thymidine
cgcganttcgcg

SEQ ID NO: 262 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(7,8): mod_base = OTHER; note = Deoxy-Thymidine
  modified_base order(5,6): mod_base = OTHER; note = 2-fluoro-deoxy-adenosine
cgcgnnttcgcg

SEQ ID NO: 263 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base 5: mod_base = OTHER; note = Deoxy-Adenosine
  modified_base 6: mod_base = OTHER; note = deoxy-isoguanosine
  modified_base order(7,8): mod_base = OTHER; note = Deoxy-Thymidine
cgcganttcgcg

SEQ ID NO: 264 - RNA, 12 nt, synthetic construct
  source 1..12: mol_type = other RNA
  modified_base order(1,3,9,11): mod_base = OTHER; note = Deoxy-Cytidine
  modified_base order(2,4,10,12): mod_base = OTHER; note = Deoxy-Guanosine
  modified_base order(5,6): mod_base = OTHER; note = deoxy-isoguanosine
  modified_base order(7,8): mod_base = OTHER; note = Deoxy-Thymidine
cgcgnnttcgcg

SEQ ID NO: 265 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(9,11,15,17,19): mod_base = OTHER; note = 2-prime-deoxy-2-prime-fluorouridine-3-prime-phospha te
  modified_base order(2,4,18,20): mod_base = OTHER; note = 2-prime-O-methyladenosine-3-prime-phosphate
  modified_base order(6,8,12): mod_base = OTHER; note = 2-prime-O-methyluridine-3-prime-phosphate
  modified_base order(14,16): mod_base = OTHER; note = 2-prime-O-methylcytidine-3-prime-phosphate
aacagtgttcttgctctataa

SEQ ID NO: 266 - RNA, 23 nt, synthetic construct
  source 1..23: mol_type = other RNA
  modified_base order(1,21..23): mod_base = OTHER; note = 2-prime-O-methyluridine-3-prime-phosphate
  modified_base order(3,5,7,11,13): mod_base = OTHER; note = 2-prime-O-methyladenosine-3-prime-phosphate
  modified_base order(9,15,17): mod_base = OTHER; note = 2-prime-O-methylcytidine-3-prime-phosphate
  modified_base order(12,19): mod_base = OTHER; note = 2-prime-O-methylguanosine-3-prime-phosphate
  modified_base order(2,4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(10,14,16): mod_base = OTHER; note = 2-prime-deoxy-2-prime-fluoroadenosine-3-prime-phosp hate
  modified_base order(1^2,21^22,22^23): mod_base = OTHER; note = Phosphorothioate linkages
ttatagagcaagaacactgtttt

SEQ ID NO: 267 - RNA, 12 nt, synthetic construct
  source 1..12: mol_type = other RNA
  modified_base 7: mod_base = OTHER; note = 2-fluoro-deoxy-adenosine
gcgcttnagcgc

SEQ ID NO: 268 - RNA, 12 nt, synthetic construct
  source 1..12: mol_type = other RNA
  modified_base 7..8: mod_base = OTHER; note = 2-fluoro-deoxy-adenosine
gcgcttnngcgc

SEQ ID NO: 269 - RNA, 12 nt, synthetic construct
  source 1..12: mol_type = other RNA
  modified_base 7..8: mod_base = OTHER; note = deoxy-isoguanosine
gcgcttnngcgc

SEQ ID NO: 270 - RNA, 12 nt, synthetic construct
  source 1..12: mol_type = other RNA
  modified_base 7: mod_base = OTHER; note = deoxy-isoguanosine
gcgcttnagcgc

SEQ ID NO: 271 - DNA, 18 bp, synthetic construct
  source 1..18: mol_type = other DNA
tttttttttttttttttt

SEQ ID NO: 272 - RNA, 23 nt, synthetic construct
  source 1..23: mol_type = other RNA
  modified_base order(1,21..23): mod_base = OTHER; note = 2-prime-O-methyluridine-3-prime-phosphate
  modified_base order(3,5,7,11,13): mod_base = OTHER; note = 2-prime-O-methyladenosine-3-prime-phosphate
  modified_base order(12,19): mod_base = OTHER; note = 2-prime-O-methylguanosine-3-prime-phosphate
  modified_base order(9,15,17): mod_base = OTHER; note = 2-prime-O-methylcytidine-3-prime-phosphate
  modified_base order(2,4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(10,16): mod_base = OTHER; note = 2-prime-deoxy-2-prime-fluoroadenosine-3-prime-phosp hate
  modified_base 14: mod_base = OTHER; note = 2-prime F DAP
  modified_base order(1^2,2^3,21^22,22^23): mod_base = OTHER; note = Phosphorothioate linkages
ttatagagcaagaacactgtttt

SEQ ID NO: 273 - RNA, 23 nt, synthetic construct
  source 1..23: mol_type = other RNA
  modified_base order(1,21..23): mod_base = OTHER; note = 2-prime-O-methyluridine-3-prime-phosphate
  modified_base order(3,5,7,11,13): mod_base = OTHER; note = 2-prime-O-methyladenosine-3-prime-phosphate
  modified_base order(9,15,17): mod_base = OTHER; note = 2-prime-O-methylcytidine-3-prime-phosphate
  modified_base order(12,19): mod_base = OTHER; note = 2-prime-O-methylguanosine-3-prime-phosphate
  modified_base order(2,4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(10,16): mod_base = OTHER; note = 2-prime-deoxy-2-prime-fluoroadenosine-3-prime-phosp hate
  modified_base order(1^2,2^3,21^22,22^23): mod_base = OTHER; note = Phosphorothioate linkages
  modified_base 11: mod_base = OTHER; note = 2-prime OMe DAP
ttatagagcaagaacactgtttt

SEQ ID NO: 274 - RNA, 21 nt, synthetic construct
  source 1..21: mol_type = other RNA
  modified_base order(2,4,18,20): mod_base = OTHER; note = 2-prime-O-methyladenosine-3-prime-phosphate
  modified_base order(6,8,12): mod_base = OTHER; note = 2-prime-O-methyluridine-3-prime-phosphate
  modified_base order(14,16): mod_base = OTHER; note = 2-prime-O-methylcytidine-3-prime-phosphate
  modified_base order(3,10,13,19): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(9,11,15,17): mod_base = OTHER; note = 2-prime-deoxy-2-prime-fluorouridine-3-prime-phospha te
  modified_base order(1,21): mod_base = OTHER; note = 2-prime-F DAP
aacagtgttcttgctctataa

SEQ ID NO: 275 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(9,11,15,17,19): mod_base = OTHER; note = 2-prime-deoxy-2-prime-fluorouridine-3-prime-phospha te
  modified_base order(6,8,12): mod_base = OTHER; note = 2-prime-O-methyluridine-3-prime-phosphate
  modified_base order(14,16): mod_base = OTHER; note = 2-prime-O-methylcytidine-3-prime-phosphate
  modified_base order(1^2,2^3): mod_base = OTHER; note = Phosphorothioate linkages
  modified_base order(2,4,18,20): mod_base = OTHER; note = 2-prime-OMe DAP
aacagtgttcttgctctataa

SEQ ID NO: 276 - RNA, 23 nt, synthetic construct
  source 1..23: mol_type = other RNA
  modified_base order(1,21..23): mod_base = OTHER; note = 2-prime-O-methyluridine-3-prime-phosphate
  modified_base order(3,5,7,11,13): mod_base = OTHER; note = 2-prime-O-methyladenosine-3-prime-phosphate
  modified_base order(9,15,17): mod_base = OTHER; note = 2-prime-O-methylcytidine-3-prime-phosphate
  modified_base order(12,19): mod_base = OTHER; note = 2-prime-O-methylguanosine-3-prime-phosphate
  modified_base order(2,4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(10,14,16): mod_base = OTHER; note = 2-prime-F DAP
  modified_base order(1^2,2^3,21^22,22^23): mod_base = OTHER; note = Phosphorothioate linkages
ttatagagcaagaacactgtttt

SEQ ID NO: 277 - RNA, 23 nt, synthetic construct
  source 1..23: mol_type = other RNA
  modified_base order(1,21..23): mod_base = OTHER; note = 2-prime-O-methyluridine-3-prime-phosphate
  modified_base order(9,15,17): mod_base = OTHER; note = 2-prime-O-methylcytidine-3-prime-phosphate
  modified_base order(12,19): mod_base = OTHER; note = 2-prime-O-methylguanosine-3-prime-phosphate
  modified_base order(2,4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(10,14,16): mod_base = OTHER; note = 2-prime-deoxy-2-prime-fluoroadenosine-3-prime-phosp hate
  modified_base order(3,5,7,11,13): mod_base = OTHER; note = 2-prime-OMe DAP
  modified_base order(1^2,2^3,21^22,22^23): mod_base = OTHER; note = Phosphorothioate linkages
ttatagagcaagaacactgtttt

SEQ ID NO: 278 - RNA, 21 nt, synthetic construct
  source 1..21: mol_type = other RNA
  modified_base 1: mod_base = OTHER; note = 2-prime-FA Conjugate
  modified_base order(2,4,18,20): mod_base = OTHER; note = 2-prime-O-methyladenosine-3-prime-phosphate
  modified_base order(6,8,12): mod_base = OTHER; note = 2-prime-O-methyluridine-3-prime-phosphate
  modified_base order(14,16): mod_base = OTHER; note = 2-prime-O-methylcytidine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(9,11,15,17,19): mod_base = OTHER; note = 2-prime-deoxy-2-prime-fluorouridine-3-prime-phospha te
  modified_base 21: mod_base = OTHER; note = 2-prime-deoxy-2-prime-fluoroadenosine-3-prime-phosp hate
  modified_base order(1^2,2^3,19^20,20^21): mod_base = OTHER; note = Phosphorothioate linkages
aacagtgttcttgctctataa

(19) United States
(12) Patent Application Publication
(10) Pub. No.: US 2025/0025564 A1
(43) Pub. Date: Jan. 23, 2025

(54) SIMPLE CHEMICAL APPROACHES TO INTRODUCE 2,6-DIAMINOPURINE AND 2-AMINOADENINE CONJUGATES INTO OLIGONUCLEOTIDES

(71) Applicant: ALNYLAM PHARMACEUTICALS, INC., Cambridge, MA (US)

(72) Inventors: Muthiah MANOHARAN, Cambridge, MA (US); Dhrubajyoti DATTA, Cambridge, MA (US); Mimouna MADAOUI, Cambridge, MA (US)

(73) Assignee: ALNYLAM PHARMACEUTICALS, INC., Cambridge, MA (US)

(21) Appl. No.: 18/577,888

(22) PCT Filed: Jul. 15, 2022
(86) PCT No.: PCT/US2022/037260

§ 371 Date: Jan. 9, 2024

Related U.S. Application Data
(60) Provisional application No. 63/222,269, filed on Jul. 15, 2021.
(60) Provisional application No. 63/236,022, filed on Aug. 23, 2021.
(60) Provisional application No. 63/246,479, filed on Sep. 21, 2021.

(51) Int. Cl. A61K 47/54; A61K 49/00; C07H 21/02; C07H 21/04

(52) U.S. Cl. CPC A61K 47/549; A61K 49/0054; C07H 21/02; C07H 21/04

(57) ABSTRACT

The present disclosure relates monomers and methods for synthesizing oligonucleotides comprising 2,6-diaminopurine (DAP) and 2-aminoadenine conjugates. The methodology employs simple aromatic nucleophilic substitution of halogen atom at the 2-position in 2-haloadenosine derivatives with amines or alkali earth hydroxides obviating the need for any protecting group on adenine.

### CROSS-REFERENCE TO RELATED APPLICATIONS

This application is a 35 U.S.C. § 371 National Phase Entry Application of International Application No. PCT/US2022/037260 filed Jul. 15, 2022, which designates the U.S. and claims benefit under claims benefit under 119 (e) of U.S. Provisional Application No. 63/246,479 filed Sep. 21, 2021, U.S. Provisional Application No. 63/236,022 filed Aug. 23, 2021, and U.S. Provisional Application No. 63/222,269 filed Jul. 15, 2021, contents of all of which are incorporated herein by reference in their entireties.

### SEQUENCE LISTING

The instant application contains a Sequence Listing which has been submitted electronically in XML format and is hereby incorporated by reference in its entirety. Said XML copy, created on Sep. 2, 2022, is named 051058-190510WOPT.xml and is 1,000,000 bytes in size.

### TECHNICAL FIELD

The present disclosure relates generally to methods and compositions for facile synthesis of oligonucleotide comprising 2,6-diaminopurine and 2-aminoadenine conjugates.

### BACKGROUND

The 2,6-diaminopurine (DAP) nucleobase can form Watson-Crick base pairs with thymine in DNA and uracil in RNA. These pairs are stabilized by three hydrogen bonds resulting in improved thermodynamic duplex stability relative to base pairing with adenine. Previously reported strategies for synthesis of DAP building blocks for oligonucleotide synthesis require multiple steps and have —NH₂ protecting group issues.

The inventors have developed a post-synthetic strategy using the 2-fluoro-6-amino-adenine as the key intermediate to make DAP-containing oligonucleotides and their conjugates.

### SUMMARY

In one aspect, provided herein is a method for preparing an oligonucleotide comprising a nucleoside of Formula (I):

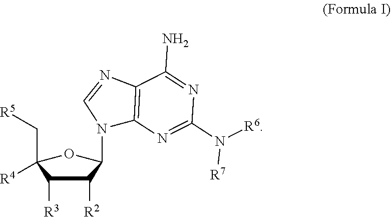

The method comprises reacting an oligonucleotide comprising a nucleoside of Formula (II):

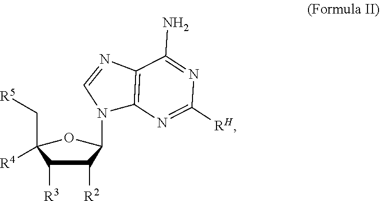

with an amine of formula HNR⁶R⁷ under conditions to convert the halogen group R^{H} to an amine of formula R⁶R⁷.

It is noted that the oligonucleotide comprising a nucleotide of Formula II can be linked to a solid support when reacting with an amine of formula HNR⁶R⁷. Alternatively, the oligonucleotide comprising a nucleotide of Formula II is not linked to a solid support when reacting with an amine of formula HNR⁶R⁷.

In some embodiments of any one of the aspects, the N atom in the amine of formula HNR⁶R⁷ can be ¹⁵N.

In another aspect, provided herein is a method for preparing an oligonucleotide comprising a nucleoside of Formula (X):

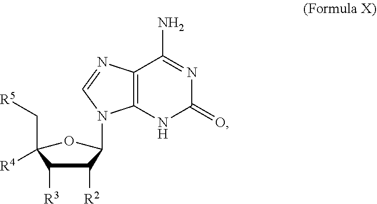

The method comprises reacting an oligonucleotide comprising a nucleoside of Formula (XI):

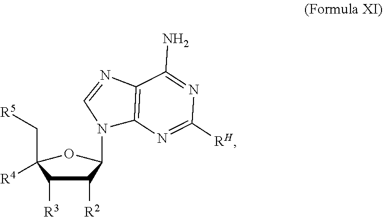

with an alkali hydroxide or alkali earth hydroxide (e.g., NaOH).

It is noted that the oligonucleotide comprising a nucleotide of Formula XI can be linked to a solid support when reacting with an alkali hydroxide or alkali earth hydroxide (e.g., NaOH). Alternatively, the oligonucleotide comprising a nucleotide of Formula XI is not linked to a solid support when reacting with an alkali hydroxide or alkali earth hydroxide (e.g., NaOH).

In some embodiments, the oligonucleotide comprising a nucleotide of Formula XI is linked to a solid support and the method comprises a step of cleaving the oligonucleotide from the solid support prior to reacting with an alkali hydroxide or alkali earth hydroxide (e.g., NaOH).

In Formulas (I), (II), (X) and (XI):
R^{H} is halogen (e.g., chloro or fluoro);
R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R³ is a bond to an internucleotide linkage to a subsequent nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R⁴ is hydrogen, optionally substituted C₁-6alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
   Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
   R¹⁰ and R¹¹ independently are H, optionally substituted C₁-C₆alkyl, optionally substituted C₂-C₆ alkenyl or optionally substituted C₂-C₆ alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵ represents a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}—5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
   X is 0 or S;
   a and b are each independently 1-10;
each R⁶ and R⁷ is independently hydrogen or -L-R^{L}, provided that at least one of R⁶ and R⁷ is not H;
or R⁶ and R⁷taken together with the nitrogen atom to which they are attached form a 3-10 membered heterocyclyl or a 3-10 membered heteroaryl group, the heterocyclyl or heteroaryl comprising one -L-R^{L} group,
   wherein
   L is absent or a linker;
   each R^{L} is a ligand, (e.g., selected independently from the group consisting of carbohydrates, peptides, lipids, therapeutic agents, diagnostic agents, detectable labels, antibodies or fragments thereof, vitamins, optionally substituted C₁₋₃₀ alkyl, optionally substituted C₁₋₃₀ alkenyl, or optionally substituted C₁₋₃₀ alkynyl);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl,
provided that,
(i) no more than one of R² and R³ is a bond to an internucleotide linkage to a subsequent nucleotide; and
   (ii) when both of R² and R³ are not a bond, then R⁵ is a bond to an internucleotide linkage to a preceding nucleotide.

In another aspect, provided herein is a compound of Formula (III):

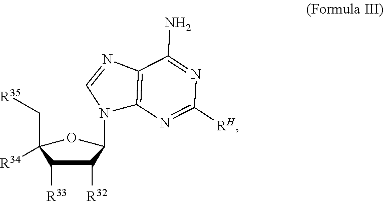

In Formula (III):
R^{H} is halogen;
R³² is hydrogen, hydroxy, halogen protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support;
R³³ is hydrogen, hydroxy, halogen protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support, and optionally, only one of R³² and R³³ is a phosphate group, a reactive phosphorous group, a solid support or a linker to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R³² taken together are 4′-C(R¹⁰R¹¹)ᵥ—Y-2′ or 4′-Y—C(R¹⁰R¹¹)ᵥ-2′;
   Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R²)—;
   R¹⁰ and R¹¹ independently are H, optionally substituted C₁-C₆alkyl, optionally substituted C₂-C₆alkenyl or optionally substituted C₂-C₆alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10); and
   each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of compounds of Formula (III), R³⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic; and R³³ is a reactive phosphorous group.

In some embodiments of compounds of Formula (III), R³⁵ is a vinylphosphonate (VP) group, cyclopropylphosphonate, or a phosphate mimic; and R³³ is a reactive phosphorous group.

In some embodiments of compounds of Formula (III), R³⁵ is a vinylphosphonate (VP) group (e.g., E-vinylphosphonate), cyclopropylphosphonate, or a phosphate mimic; and R³³ is a phosphoramidite group.

In some embodiments of compounds of Formula (III), R³⁵ is a triphosphate group and R³³ is allyloxy, azidomethoxy, or aminooxy.

In some embodiments of compounds of Formula (III), R³³ is a reactive phosphorous group.

In some embodiments of compounds of Formula (III), R³⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic; and R³³ is a reactive phosphorous group.

In some embodiments of compounds of Formula (III), R³⁵ is a vinylphosphonate (VP) group, cyclopropylphosphonate, or a phosphate mimic; and R³³ is a reactive phosphorous group.

In some embodiments of compounds of Formula (III), R³⁵ is a vinylphosphonate (VP) group (e.g., E-vinylphosphonate), cyclopropylphosphonate, or a phosphate mimic; and R³³ is a phosphoramidite group.

In some embodiments of compounds of Formula (III), R³⁵ is a triphosphate group and R³³ is allyloxy, azidomethoxy, or aminooxy.

In some embodiments of compounds of Formula (III), R³³ is a reactive phosphorous group.

Some exemplary compounds of Formula (III) include, but are not limited to the following:

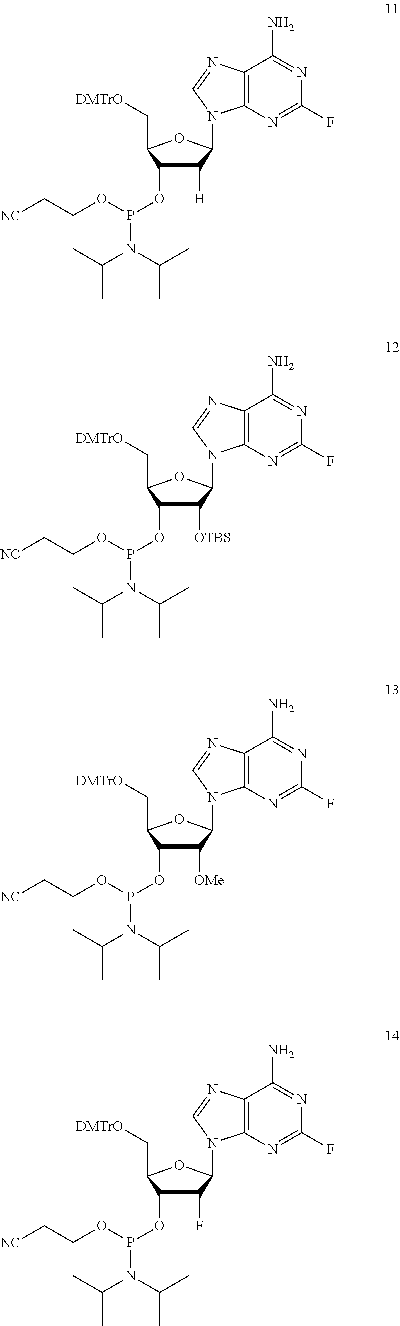

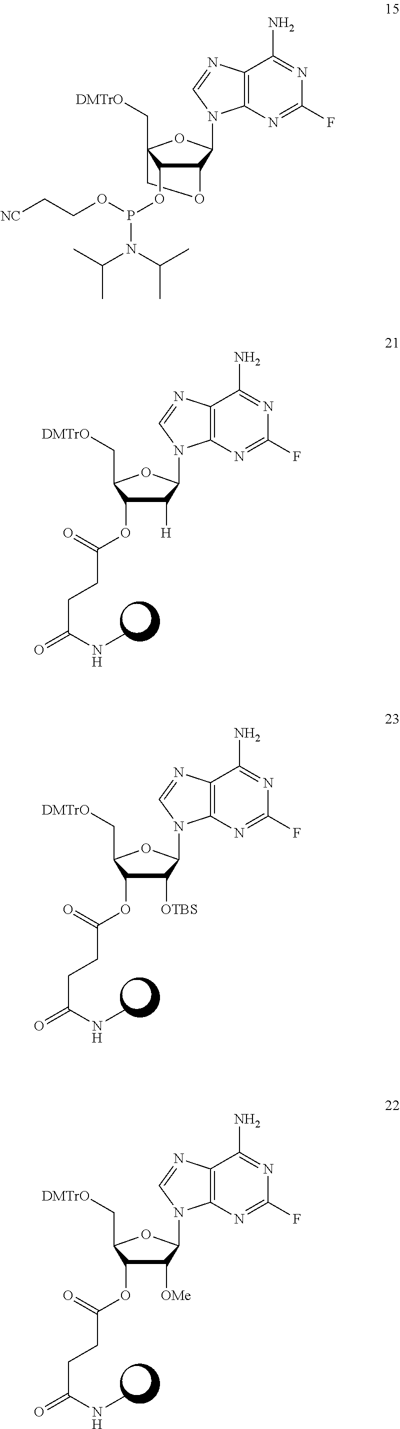

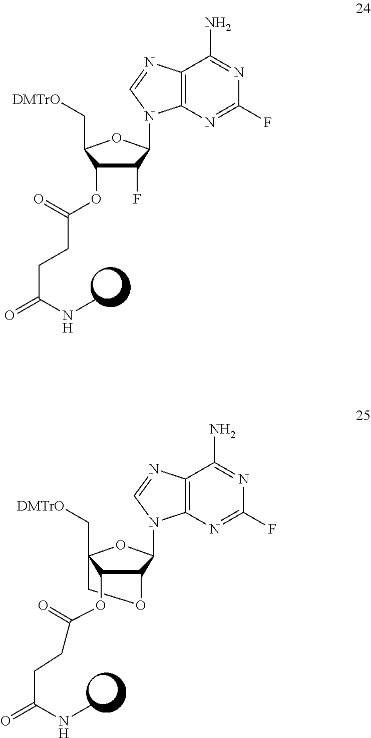

Additional exemplary compounds of Formula (III) include, but are not limited to,

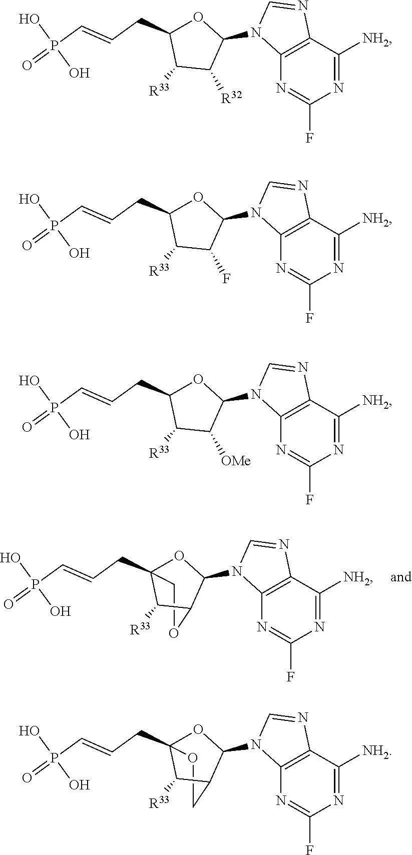

In yet another aspect, provided herein is an oligonucleotide prepared using a method described herein. For example, the disclosure provides an oligonucleotide comprising nucleoside of Formula (I), (II), (X) and/or (XI).

It is noted that the nucleoside of Formula (I), (II), (X) or (XI) can be present in any position in the oligonucleotide. For example, a nucleoside of Formula (I), (II), (X) or (XI) can be at the 5′-end of the oligonucleotide. In some other non-limiting example, a nucleoside of Formula (I), (II), (X) or (XI) can be at the 3′-end of the oligonucleotide. In yet some other non-limiting examples, a nucleoside of Formula (I), (II), (X) or (XI) can be present at an internal position of the oligonucleotide.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1 is a schematic representation of exemplary aspects of 2,6-diaminopurine (DAP) nucleobase.

FIG. 2 shows exemplary 2-fluoro-6-aminopurine monomers for oligonucleotide synthesis.

FIG. 3 shows exemplary monomer residues after incorporation into oligonucleotides.

FIGS. 4 and 5 are schematic representation of post-synthesis conjugation according to exemplary embodiments of the invention.

FIG. 6 shows exemplary ligands the could be conjugated at the 2-amino of the DAP.

FIGS. 7A and 7B show exemplary post-synthesis conjugation monomer residues in oligonucleotides according to some exemplary embodiments of the disclosure.

FIG. 8 show exemplary post-synthesis conjugation monomer residues in oligonucleotides according to some exemplary embodiments of the disclosure.

FIG. 9 is a synthetic scheme for synthesis of 2-fluoro-6-aminopurine nucleosides. References for compounds 1, 3, 5, 7, 8 and 9 are Morris et. al. J. Med. Chem. 1972, 15, 735; Vorbrueggen et. al. Nucleosides & Nucleotides 1994, 13, 673; Iribarren et. al. Nucleic Acids Research 1990, 18, 41; Ross et. al. Nucleosides, Nucleotides & Nucleic Acids 2008, 27, 67; Koch et. al. Bioorg Med Chem 2004, 12, 2385; Ross & Manoharan. 2000, WO2000012563; Manoharan et. al. in PCT Int. Appl. 2009, WO 2009091982; and Montgomery et. al. Nucleosides & Nucleotides 1994, 13, 309.

FIG. 10 is a schematic representation of synthesis of exemplary oligonucleotide comprising DAP nucleoside.

FIG. 11 shows ion-exchange HPLC analysis of crude and purified oligonucleotides after site-specific single DAP incorporation.

FIG. 12 shows LCMS analysis of purified oligonucleotides after site-specific single DAP incorporation.

FIG. 13 is a schematic representation of post-synthetic conjugation according to an embodiment of the invention.

FIGS. 14-16 shows LCMS analysis of crude oligonucleotides after post-synthetic conjugation on solid support (FIGS. 14 and 15) and solution phase (FIG. 16).

FIG. 17 is a schematic representation of some exemplary DAP conjugates.

FIG. 18 shows ¹⁹F NMR analysis of any oligonucleotide prepared according to an exemplary embodiment of the method described herein.

FIG. 19 is a schematic representation showing conversion of 2-F A to iso-G according to an exemplary embodiment of the method described herein.

FIG. 20 shows LC-MS analysis of iso-G comprising oligonucleotides prepared according to an exemplary embodiment of the method described herein.

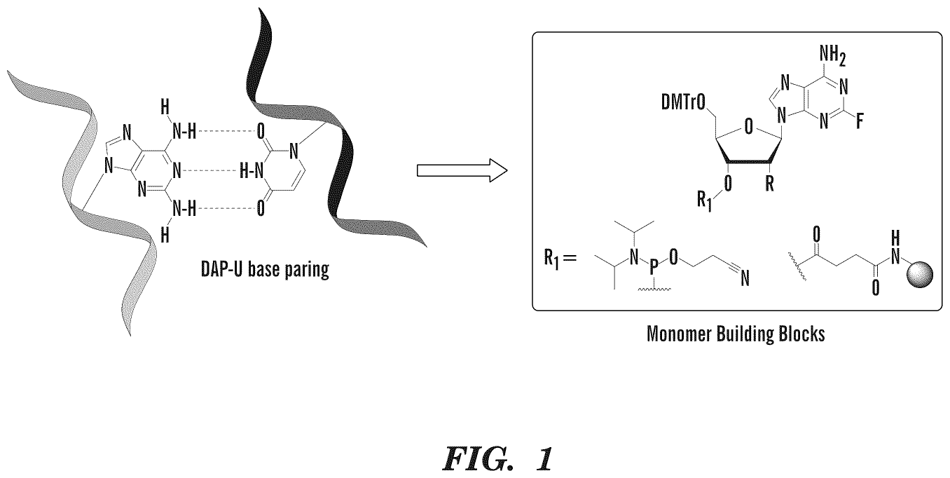

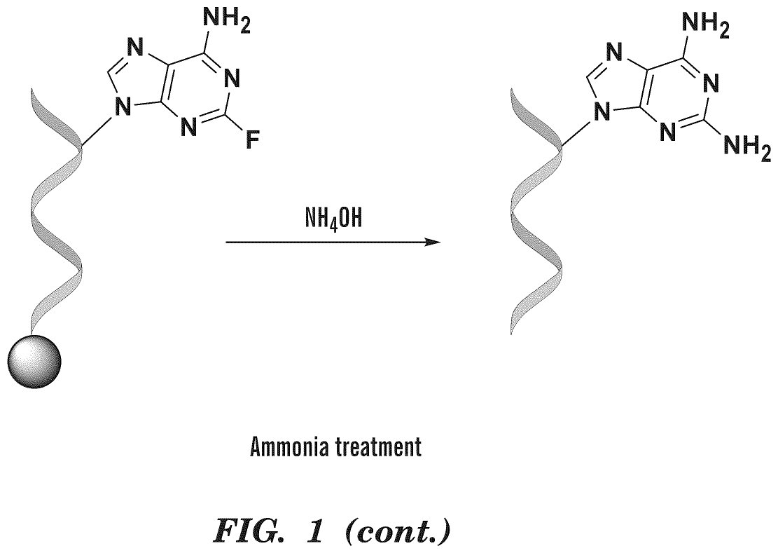

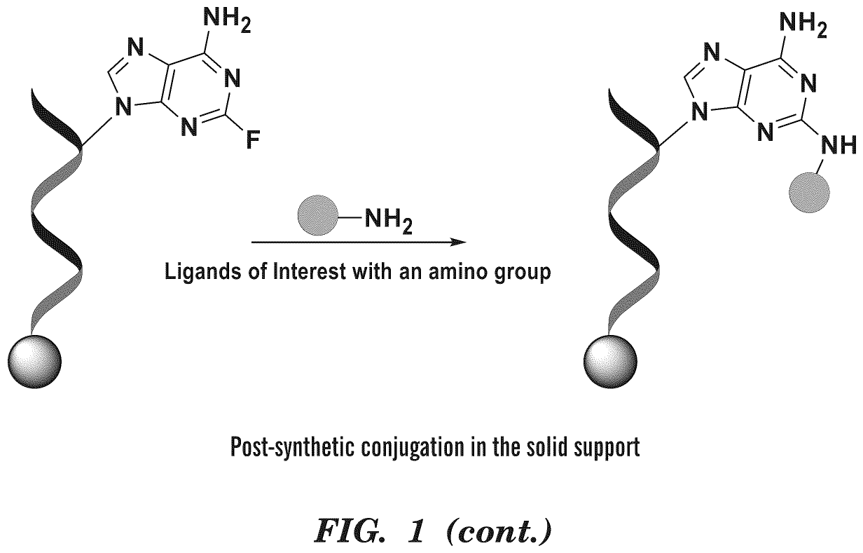

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

In one aspect, provided herein is a method for preparing an oligonucleotide comprising at least one nucleotide of Formula (I):

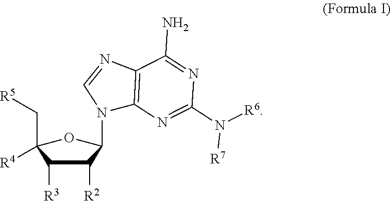

The method comprises reacting an oligonucleotide comprising a nucleoside of Formula (II):

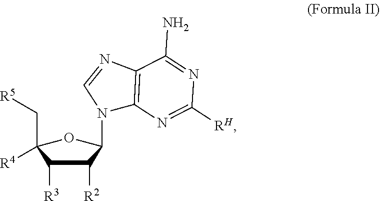

with an amine of formula HNR⁶R⁷ under conditions to convert the halogen group R^{H} to an amine of formula R⁶R⁷.

In another aspect, provided herein is a method for preparing an oligonucleotide comprising a nucleoside of Formula (X):

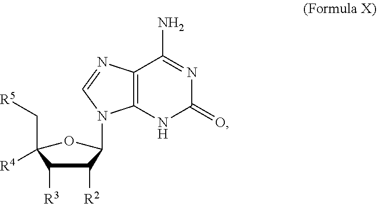

The method comprises reacting an oligonucleotide comprising a nucleoside of Formula (XI):

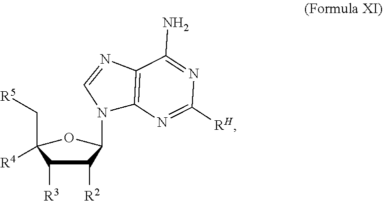

with an alkali hydroxide or alkali earth hydroxide (e.g., NaOH).

In another aspects, the disclosure provides a compound of Formula (III):

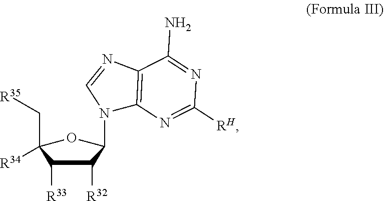

The various R groups and variables present in Formula (I), (II), (III), (X) and/or (XI) are described herein.

### R^{H}

In some embodiments of any one of the aspects described herein, R^{H} is halogen. For example, R^{H} can be fluoro, chloro, bromo or iodo. In some embodiments of any one of the aspects described herein, R^{H} is fluoro.

### R^{L}

In embodiments of the various aspects described herein, each R^{L} can be independently selected from the groups consisting of H, carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

In some embodiments of any one of the aspects described herein, R^{L} is a ligand. Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid, O3-(oleoyl)lithocholic acid, 03-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; u, (3, or y peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

(SEQ ID NO: 1)
AALEALAEALEALAEALEALAEAAAAGGC (GALA);
(SEQ ID NO: 2)
AALAEALAEALAEALAEALAEALAAAAGGC (EALA);
(SEQ ID NO: 3)
ALEALAEALEALAEA;
(SEQ ID NO: 4)
GLFEAIEGFIENGWEGMIWDYG (INF-7);
(SEQ ID NO: 5)
GLFGAIAGFIENGWEGMIDGWYG (Inf HA-2);
(SEQ ID NO: 6)
GLFEAIEGFIENGWEGMIDGWYGCGLFEAIEGFIENGWEGMID
GWYGC (diINF-7);
(SEQ ID NO: 7)
GLFEAIEGFIENGWEGMIDGGCGLFEAIEGFIENGWEGMIDGGC
(diINF-3);
(SEQ ID NO: 8)
GLFGALAEALAEALAEHLAEALAEALEALAAGGSC (GLF);
(SEQ ID NO: 9)
GLFEAIEGFIENGWEGLAEALAEALEALAAGGSC (GALA-INF3);
(SEQ ID NO: 10)
GLF EAI EGFI ENGW EGnI DG K GLF EAI EGFI ENGW
EGnI DG (INF-5, n is norleucine);
(SEQ ID NO: 11)
LFEALLELLESLWELLLEA (JTS-1);
(SEQ ID NO: 12)
GLFKALLKLLKSLWKLLLKA (ppTG1);
(SEQ ID NO: 13)
GLFRALLRLLRSLWRLLLRA (ppTG20);
(SEQ ID NO: 14)
WEAKLAKALAKALAKHLAKALAKALKACEA (KALA);
(SEQ ID NO: 15)
GLFFEAIAEFIEGGWEGLIEGC (HA);
(SEQ ID NO: 16)
GIGAVLKVLTTGLPALISWIKRKRQQ (Melittin);
(SEQ ID NO: 17)
H₅WYG;
and
(SEQ ID NO: 18)
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

(SEQ ID NO: 19)
RQIKIWFQNRRMKWKK (penetratin);
(SEQ ID NO: 20)
GRKKRRQRRRPPQC (Tat fragment 48-60);
(SEQ ID NO: 21)
GALFLGWLGAAGSTMGAWSQPKKKRKV (signal sequence
based peptide);
(SEQ ID NO: 22)
LLIILRRRIRKQAHAHSK (PVEC);
(SEQ ID NO: 23)
GWTLNSAGYLLKINLKALAALAKKIL (transportan);
(SEQ ID NO: 24)
KLALKLALKALKAALKLA (amphiphilic model peptide);
(SEQ ID NO: 25)
RRRRRRRRR (Arg9);
(SEQ ID NO: 26)
KFFKFFKFFK (Bacterial cell wall permeating
peptide);
(SEQ ID NO: 27)
LLGDFFRKSKEKIGKEFKRIVQRIKDFLRNLVPRTES (LL-37);
(SEQ ID NO: 28)
SWLSKTAKKLENSAKKRISEGIAIAIQGGPR (cecropin P1);
(SEQ ID NO: 29)
ACYCRIPACIAGERRYGTCIYQGRLWAFCC (α-defensin);
(SEQ ID NO: 30)
DHYNCVSSGGQCLYSACPIFTKIQGTCYRGKAKCCK (β-
defensin);
(SEQ ID NO: 31)
RRRPRPPYLPRPRPPPFFPPRLPPRIPPGFPPRFPPRFPGKR-NH2
(PR-39);
(SEQ ID NO: 32)
ILPWKWPWWPWRR-NH2 (indolicidin);
(SEQ ID NO: 33)
AAVALLPAVLLALLAP (RFGF);
(SEQ ID NO: 34)
AALLPVLLAAP (RFGF analogue);
and
(SEQ ID NO: 35)
RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)—(VII):

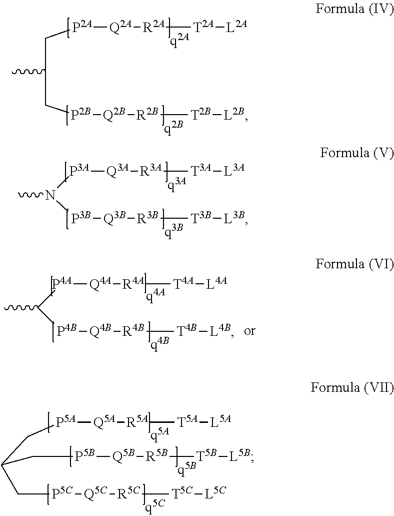

wherein:
q^{2A}, q^{2B}, q^{3A}, q^{3B}, q⁴^{^{A}}, q^{4B}, q^{5A}, q^{5B} and q^{5C} represent independently for each occurrence O-20 and wherein the repeating unit can be the same or different; P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5C}, Q^{5C} are independently for each occurrence absent, alkylene, substituted alkylene wherein one or more methylenes can be interrupted or terminated by one or more of O, S, S(O), SO₂, N(R^{N}), C(R′)═C(R¹¹), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

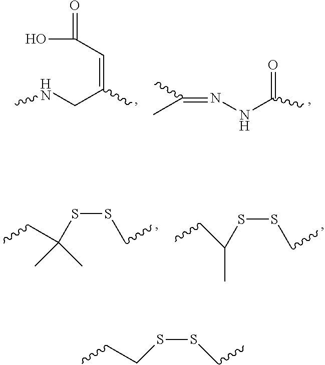

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

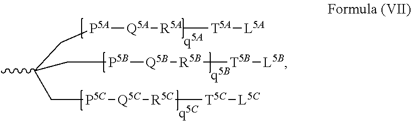

wherein L^{5A}, L^{5B} and L^{5C} represent a monosaccharide, such as GalNAc derivative.

Exemplary ligands include, but are not limited to, the following:

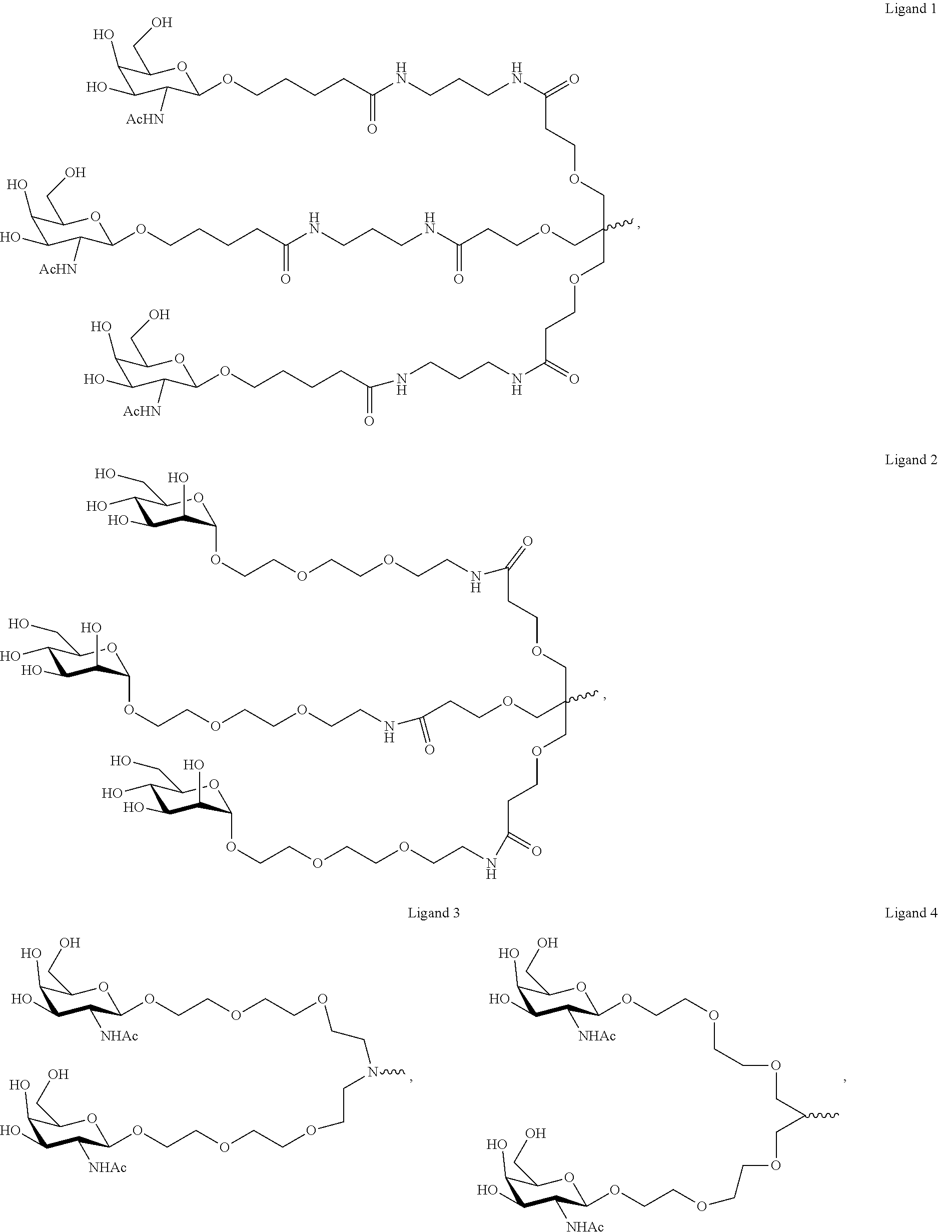

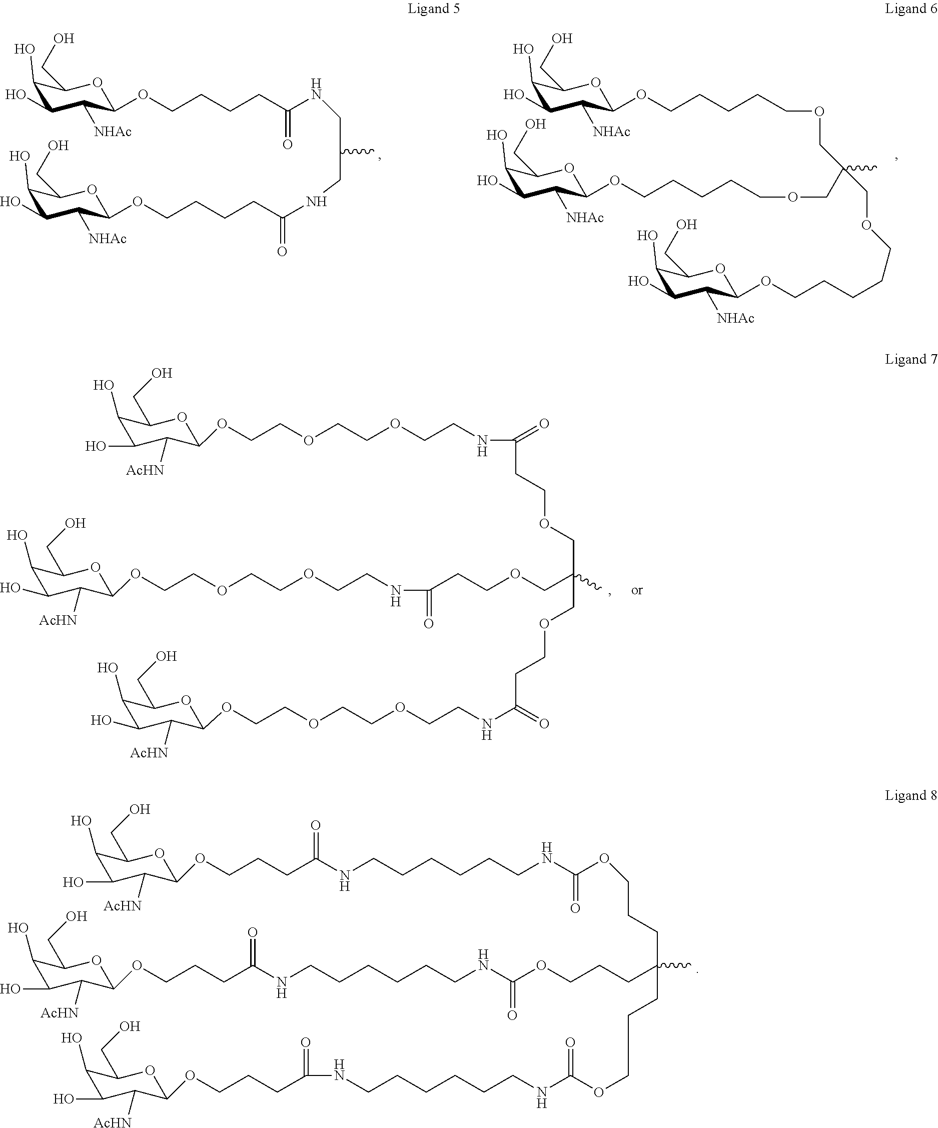

In some embodiments of any one of the aspects described herein, the ligand is a ligand described in U.S. Pat. No. 5,994,517 or U.S. Pat. No. 6,906,182, content of each of which is incorporated herein by reference in its entirety.

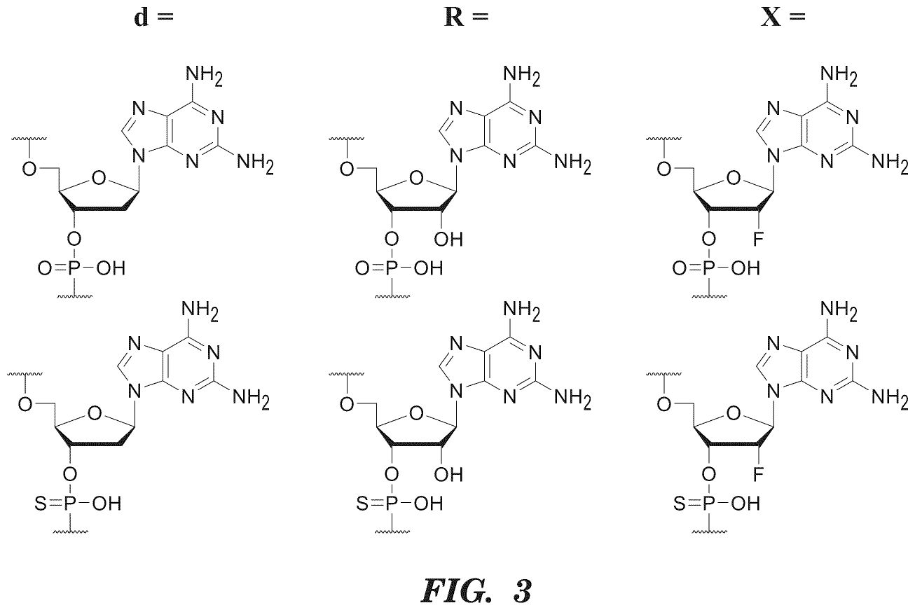

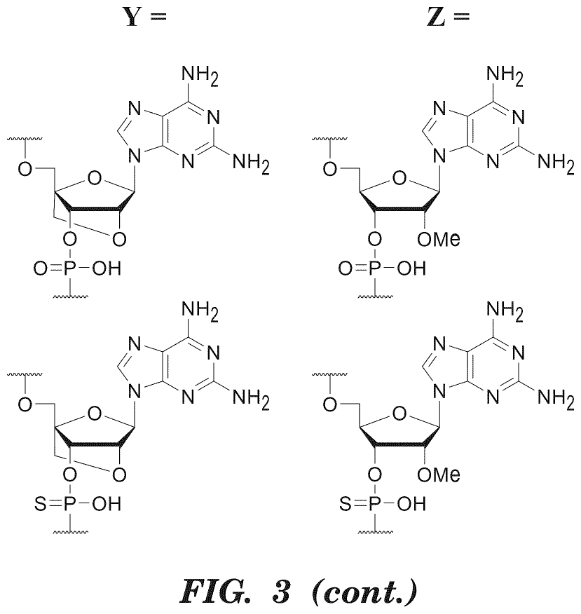

In some embodiments, the ligand can be a tri-antennary ligand described in FIG. 3 of U.S. Pat. No. 6,906,182. For example, the ligand is selected from the following tri-antennary ligands:

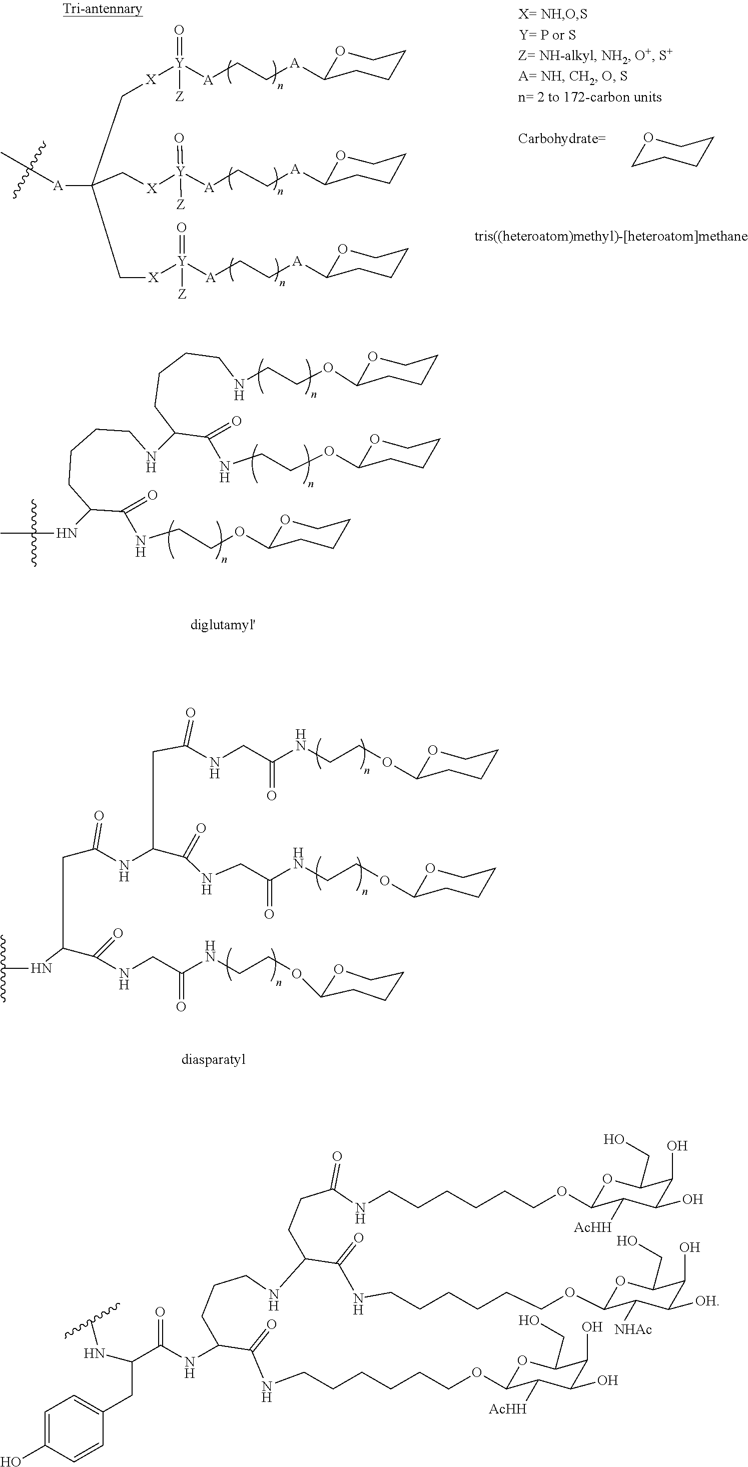

In some embodiments of any one of the aspects described herein, R^{L} is a ligand. It is noted that when more than one R^{L} are present, they can be same or different. Accordingly, in some embodiments of any one of the aspects described herein, all R^{L} are same. In some other embodiments of any one of the aspects described herein, R^{L} are different.

R⁶ and R⁷

In some embodiments of any one of the aspects described herein, at least one of R⁶ and R⁷ is not H. For example, at least one of R⁶ and R⁷ is -L-R^{L}.

It is noted that R⁶ and R⁷ can be same or different. Accordingly, in some embodiments of any one of the aspects described herein, R⁶ and R⁷ are the same. In some other embodiments of any one of the aspects described herein, R⁶ and R⁷ are different.

In some embodiments, one of R⁶ and R⁷ is H.

In some embodiments of any one of the aspects described herein, at least one of R⁶ and R⁷ is selected independently from the group consisting of:

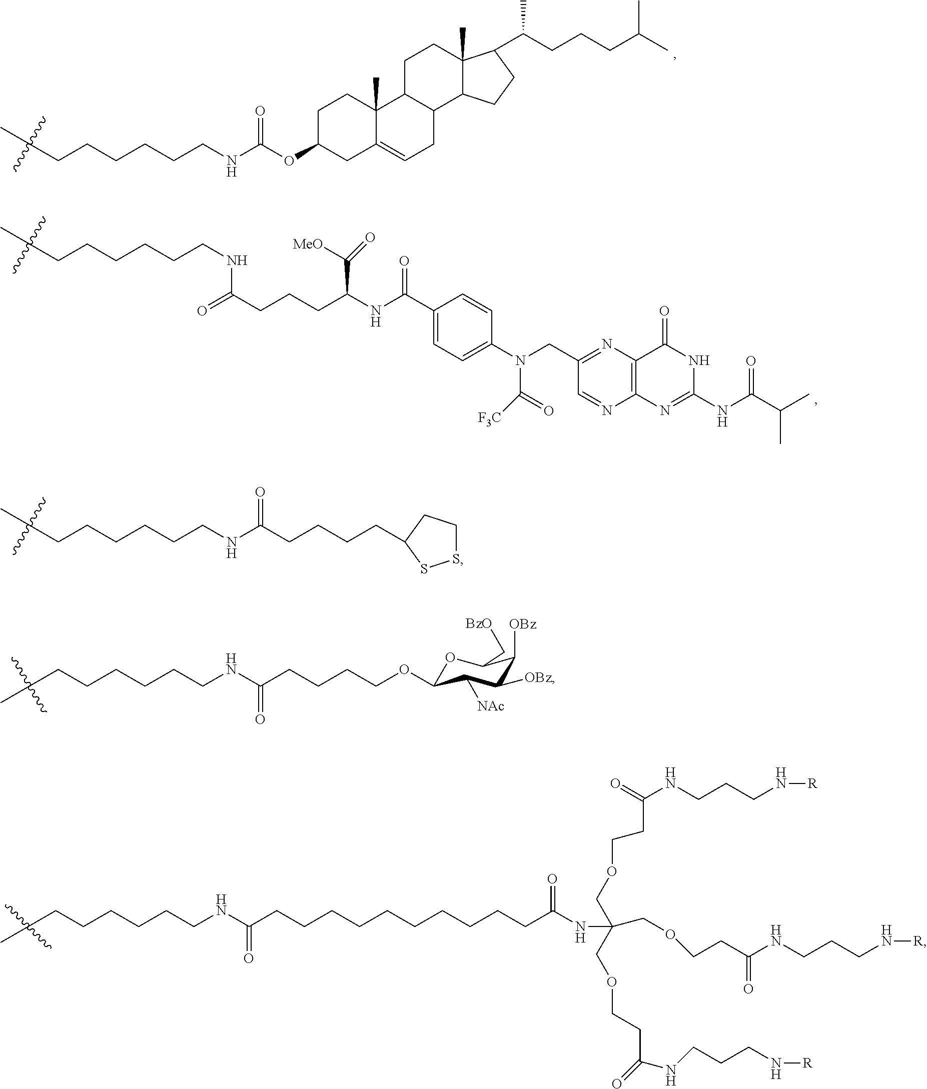

cyclo [Phe-Arg-Gly-Asp-Leu-Ala-Phe-D-Pro-N-Me-Lys(PEG4-NH₂) (SEQ ID NO:36)](cyclic RGD peptide) or optionally substituted C₁-C₃₀alkyl, and wherein R is

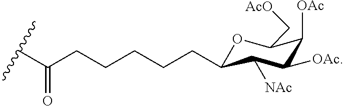

In some embodiments, at least one of R⁶ and R⁷ is an optionally substituted, branched C₄-C₃₀alkyl. For example, at least one of R⁶ and R⁷ is —CHR^{L1}R^{L2}, where R^{L1} and R^{L2} are independently selected from optionally substituted C₁-C₁₅alkyls.

In some embodiments, R^{L1} is optionally substituted C₆-C₁₂alkyl, e.g., C₇, C₈, C₉, C₁₀, or C₁₁ alkyl, each of which can be optionally substituted. In some embodiments, R^{L1} is optionally substituted C₈alkyl.

In some embodiments, R^{L2} is optionally substituted C₆-C₁₂alkyl, e.g., C₇, C₈, C₉, C₁₀, or C₁₁ alkyl, each of which can be optionally substituted. In some embodiments, R^{L2} is optionally substituted C₇alkyl.

In some embodiments, R⁶ and R⁷ independently are optionally substituted C₁-C₃₀alkyl. For example, R⁶ and R⁷ independently are optionally substituted C₄-C₂₀alkyl, e.g., R⁶ and R⁷ independently are optionally substituted C₇, C₈, C₉, C₁₀, C₁₁, C₁₂, C₁₂, C₁₄, C₁₅, C₁₆, C₁₇, C₁₈, C₁₉, or C₂₀ alkyl.

In some embodiments, at least one of R⁶ and R⁷ is a C₁-C₃₀alkyl optionally substituted with substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carboxyl, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{a′} is C₁-C₃₀alkoxy optionally substituted with a NH₂, OH, C(O)NH₂, COOH, halo, SH, or C₁-C₆alkoxy.

In some embodiments, at least one of R⁶ and R⁷ is —CH₂(CH₂)₁₄CO₂H.

### R²

In some embodiments of any one of the aspects described herein, R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R² is hydrogen, hydroxy, protected hydroxy, halogen, optionally substituted C₁₋₃₀ alkoxy, or alkoxyalkyl (e.g., methoxyethyl. In some embodiments of any one of the aspects, R² is hydrogen, hydroxy, protected hydroxy, fluoro or methoxy.

In some embodiments of any one of the aspects R² is halogen. For example, R² can be fluoro, chloro, bromo or iodo. In some embodiments of any one of the aspects described herein, R² is fluoro.

In some embodiments of any one of the aspects described herein, R² and R⁴

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2.

In some embodiments, Y is O. For example, R² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃.

In some embodiments of any one of the aspects, R¹⁰ and R¹¹ attached to the same C are the same. For example, R¹⁰ and R¹¹ attached to the same C are H.

In some embodiments of any one of the aspects, R² and R⁴ taken together are 4′-CH₂—O-2′, 4′-CH(CH₃)—O-2′, 4′-CH(CH₂OCH₃)—O-2′, or 4′-CH₂CH₂—O-2′. For example, R² and R⁴ taken together are 4′-CH₂CH₂—O-2′.

In some embodiments of any one of the aspects described herein, R² is a bond to an internucleotide linkage to a subsequent nucleotide. It is noted that only one of R² and R³ can be a bond to an internucleotide linkage to a subsequent nucleotide.

### R³

In some embodiments of any one of the aspects described herein, R³ can be a bond to an internucleotide linkage to a subsequent nucleotide, hydroxy, protected hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide, hydroxy, protected hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R³ is a bond to an internucleotide linkage to a subsequent nucleotide, hydroxy, a solid support, or a linker covalently bonded (e.g., —C(O)CH₂CH₂C(O)—) to a solid support. In some embodiments of any one of the aspects described herein, R³ is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide.

In some embodiments of any one of the aspects described herein, R³ is a solid support, or a linker covalently bonded to a solid support.

In some embodiments of any one of the aspects described herein, R³ is hydroxyl.

In some embodiments of any one of the aspects described herein, R³ and R⁴ taken together with the atoms to which they are attached form an optionally substituted C₃₋₈cycloalkyl, optionally substituted C₃₋₈cycloalkenyl, or optionally substituted 3-8 membered heterocyclyl.

### R⁴

In some embodiments of any one of the aspects described herein, R⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, optionally substituted C₁₋₆alkyl or optionally substituted C₁₋₆alkoxy.

In some embodiments of any one of the aspects described herein, R⁴ is H.

### R⁵

In some embodiments of any one of the aspects described herein, R⁵ can be a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)2(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate; phosphoramidates ((HO)2(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)2(X)P—O[—(CH₂)ₐ—O—P(X)(OH)—O]_{b}-5′ or (HO)2(X)P—O[—(CH₂)ₐ—P(X)(OH)—O]_{b}-5′ or (HO)2(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10).

In some embodiments of any one of the aspects described herein, R⁵ can be a bond to an internucleotide linkage to a preceding nucleotide, hydroxy, protected hydroxy, optionally substituted C₂₋₃₀alkenyl, optionally substituted C₂₋₃₀alkynyl, optionally substituted C₁₋₃₀ alkoxy, vinylphosphonate (VP) group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidates, or alkylphosphonates.

In some embodiments of any one of the aspects described herein, R⁵ is a bond to an internucleotide linkage to a preceding nucleotide, hydroxy, protected hydroxy, optionally substituted C₂₋₃₀alkenyl, optionally substituted C₁₋₃₀ alkoxy or a vinylphosphonate (VP) group.

In some embodiments of any one of the aspects described herein, R⁵ is a bond to an internucleotide linkage to a preceding nucleotide.

In some embodiments of any one of the aspects described herein, R⁵ is a hydroxyl or protected hydroxyl.

In some embodiments of any one of the aspects described herein, R⁵ is optionally substituted C₂₋₃₀alkenyl or optionally substituted C₁₋₃₀ alkoxy.

In some embodiments of any one of the aspects described herein, R⁵ is a vinylphosphonate group.

In some embodiments of any one of the aspects descried herein, R⁵ can be —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of any one of the aspects described herein, X⁵ is O or a bond. For example, X⁵ is O. In some other embodiments of any one of the aspects described herein, X⁵ is absent, i.e., R⁵ is —CH(R⁵¹)R⁵².

In some embodiments of the various aspects described herein, R⁵ can be —CH(R⁵¹)—R⁵² or —C(R⁵¹)═CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the various aspects described herein, R⁵ is —CH(R⁵¹)—X⁵—R⁵². For example, R⁵ is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m (CH₂)ₚ—OH, CH₂—[CH(OH)]ₘ—(CH₂)ₚ—NH₂ or CH₂-aryl-alkoxy, where “in” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of the various aspects described herein, R⁵ is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m (CH₂)ₚ—OH, CH₂—[CH(OH)]ₘ—(CH₂)ₚ—NH₂ or CH₂-aryl-alkoxy, where “in” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R⁵ is —C(R⁵¹)═CHR⁵². It is noted that the double bond in —C(R⁵¹)═CHR⁵² can be in the cis or trans configuration. Accordingly, in some embodiments of any one of the aspects, R⁵ is —C(R⁵¹)═CHR⁵² and wherein the double bond is in the cis configuration. In some other embodiments of any one of the aspects, R⁵ is —C(R⁵¹)═CHR⁵² and wherein the double bond is in the trans configuration. In some embodiments of any one of the aspects described herein, R⁵ is —CH═CHR⁵².

In some embodiments of any one of the aspects described herein, R⁵² is a bond to an internucleoside linkage to the preceding nucleotide.

In embodiments of the various aspects described herein, R⁵ is optionally substituted C₁₋₆alkyl-R⁵³, optionally substituted —C₂₋₆alkenyl-R⁵³, or optionally substituted —C₂₋₆alkynyl-R⁵³. In embodiments of the various aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R⁵ is optionally substituted —C₂₋₆alkenyl-R⁵³. For example, R⁵ is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R⁵ is —CH═CHR⁵³. It is noted that a double bond in the optionally substituted —C₂₋₆alkenyl-R⁵³ can be in the cis or trans configuration. Accordingly, in some embodiments of any one of the aspects, R⁵ is —CH═CHR⁵³ and wherein the double bond is in the cis configuration. In some other embodiments of any one of the aspects, R⁵ is —CH═CHR⁵³ and wherein the double bond is in the trans configuration.

In some embodiments of any one of the aspects, R⁵ is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R⁵ is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R⁵ is optionally substituted —C₁₋₆alkenyl-R⁵³. For example, R⁵ is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R⁵ can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)— alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In one non-limiting example, R⁵⁸ is H. In some other non-limiting examples, R⁵⁸ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R⁵ is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R⁵ is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R⁵ is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

### R³²

In some embodiments of any one of the aspects described herein, R³² is hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁶, a lipid, a linker covalently attached to a lipid, a ligand, a linker covalently attached to a ligand, a solid support, a linker covalently attached to a solid support, or a reactive phosphorus group.

R³²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R³²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R³² is R³² is hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴

In some embodiments of any one of the aspects described herein, R³² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R³² is hydrogen, hydroxy, protected hydroxy, halogen, optionally substituted C₁₋₃₀ alkoxy, or alkoxyalkyl (e.g., methoxyethyl. In some embodiments of any one of the aspects, R² is hydrogen, hydroxy, protected hydroxy, fluoro or methoxy.

In some embodiments of any one of the aspects R³² is halogen. For example, R³² can be fluoro, chloro, bromo or iodo. In some embodiments of any one of the aspects described herein, R³² is fluoro.

In some embodiments of any one of the aspects described herein, R³² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group. In some embodiments of any one of the aspects, v is 1. In some other embodiments of any one of the aspects, v is 2. In some embodiments, Y is O. For example, R³² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃. In some embodiments of any one of the aspects, R¹⁰ and R¹¹ attached to the same C are the same. For example, R¹⁰ and R¹¹ attached to the same C are H.

In some embodiments of any one of the aspects, R³² and R⁴ taken together are 4′-CH₂—O-2′, 4′-CH(CH₃)—O-2′, 4′-CH(CH₂OCH₃)—O-2′, or 4′-CH₂CH₂—O-2′. For example, R³² and R⁴ taken together are 4′-CH₂CH₂—O-2′.

In some embodiments of any one of the aspects described herein, R³² is a reactive phosphorus group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})(N(R^{P2})₂).

In some embodiments of any one of the aspects, R is an optionally substituted C₁₋₆alkyl. For example, R is a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In some embodiments, R^{p} is a C₁₋₆alkyl, optionally substituted with a CN or —SC(O)Ph. For example, R^{p} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)— alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)— alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In the reactive phosphorous groups, each R³ is independently optionally substituted C₁₋₆alkyl. For example, R³ can be a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁—C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR^{P})(N(R^{P2})₂). For example, the reactive phosphorous group is —OP(OR^{P})(N(R^{P2})₂), where R is cyanoethyl (—CH₂CH₂CN) and each R^{P2} is isopropyl.

In some embodiments of any one of the aspects described herein, R³² is —OP(OR^{P})(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR)H, —OP(O)(OR^{P})R^{P3}, —OP(S)(OR^{P})R^{P3}, or —OP(O)(SR^{P})R^{P3}.

In some embodiments of any one of the aspects, R³² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R² is independently optionally substituted C₁₋₆alkyl; and each R^{P3} is independently optionally substituted C₁₋₆alkyl.

In some embodiments of any one of the aspects, R³² is —OP(OR^{P})(N(R^{P2})₂). For example, the R³² is —OP(OR^{P})(N(R^{P2})₂), where R is cyanoethyl (—CH₂CH₂CN) and each R² is isopropyl.

In some embodiments of any one of the aspects descried herein, R³² is a solid support or a linker covalently attached to a solid support. For example, R³² is —OC(O)CH₂CH₂C(O)NH—Z, where Z is a solid support. In some embodiments, R³² is —OC(O)CH₂CH₂CO₂H.

In some embodiments of any one of the aspects, when R³² is —OR³²², R³²² can be hydrogen or a hydroxyl protecting group.

When R³² is —SR³²³, R³²³ can be hydrogen or a sulfur protecting group. Accordingly, in some embodiments of any one of the aspects, R³²³ is hydrogen.

When R³² is —O(CH₂CH₂O)ᵣCH₂CH₂OR³²⁴, r can be 1-50; R³²⁴ is independently for each occurrence H, C₁-C₃₀alkyl, cyclyl, heterocyclyl, aryl, heteroaryl, aralkyl, sugar or R³²⁵; and R³²⁵ is independently for each occurrence amino (NH₂), alkylamino, dialkylamino, arylamino, diarylamino, heteroarylamino, or diheteroaryl amino.

When R³² is —NH(CH₂CH₂NH)ₛCH₂CH₂—R³²⁵, s can be 1-50 and R³²⁵ can be independently for each occurrence amino (NH₂), alkylamino, dialkylamino, arylamino, diarylamino, heteroarylamino, or diheteroaryl amino.

In some embodiments of any one of the aspects described herein, R³² is hydrogen, halogen, —OR³²², or optionally substituted C₁-C₃₀alkoxy. For example, R³² is halogen, —OR³²², or optionally substituted C₁-C₃₀alkoxy. In some embodiments of any one of the aspects described herein, R³² is F, OH or optionally substituted C₁-C₃₀alkoxy.

In some embodiments of any one of the aspects described herein, R³² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R³² is C₁-C₃₀alkoxy optionally substituted with a NH₂, OH, C(O)NH₂, COOH, halo, SH, or C₁-C₆alkoxy. In some embodiments of any one of the aspects described herein, R³² is —O(CH₂)ₜCH₃, where t is 1-21. For example, t is 14, 15, 16, 17 or 18. In one non-limiting example, t is 16.

In some embodiments of any one of the aspects, R³² is —O(CH₂)ᵤR³²⁷, where u is 2-10; R³²⁷ is C₁-C₆alkoxy, amino (NH₂), CO₂H, OH or halo. For example, R³²⁷ is —CH₃ or NH₂. Accordingly, in some embodiments of any one of the aspects described herein, R³² is —O(CH₂)ᵤ—OMe or R³² is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³² is a C₁-C₆haloalkyl. For example, R³² is a C₁-C₄haloalkyl. In some embodiments of any one of the aspects described herein, R³² is —CF₃, —CF₂CF₃, —CF₂CF₂CF₃ or —CF₂(CF₃)₂.

In some embodiments of any one of the aspects described herein, R³² is —OCH(CH₂OR³²⁸)CH₂OR³²⁹, where R³²⁸ and R³²⁹ independently are H, optionally substituted C₁-C₃₀alkyl, optionally substituted C₂-C₃₀alkenyl or optionally substituted C₂-C₃₀alkynyl. For example, R³²⁸ and R³²⁹ independently are optionally substituted C₁-C₃₀alkyl.

In some embodiments of any one of the aspects described herein, R³² is —CH₂C(O)NHR³²¹⁰, where R³²¹⁰ is H, optionally substituted C₁-C₃₀alkyl, optionally substituted C₂-C₃₀alkenyl or optionally substituted C₂-C₃₀alkynyl. For example, R³²¹⁰ is H or optionally substituted C₁-C₃₀alkyl. In some embodiments, R³²¹⁰ is optionally substituted C₁-C₆alkyl.

### R³³

In some embodiments of any one of the aspects described herein, R³³ is hydrogen, halogen, —OR³³², —SR³³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³³⁴, cyano, alkyl-thio-alkyl, thioalkoxy, cycloalkyl, aryl, heteroaryl, —NH(CH₂CH₂NH)ₛCH₂CH₂—R³³⁵, NHC(O)R³³⁶, a lipid, a linker covalently attached to a lipid, a ligand, a linker covalently attached to a ligand, a solid support, a linker covalently attached to a solid support, or a reactive phosphorus group.

R³³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R³³⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R³³ is a reactive phosphorus group. For example, R³³ is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂, —OP(O)(OR^{P})H, —OP(S)(OR^{P})H, —OP(O)(SR)H, —OP(O)(OR^{P})R^{P3}, —OP(S)(OR^{P})R^{P3}, or —OP(O)(SR^{P})R^{P3}.

In some embodiments of any one of the aspects, R³³ is —OP(OR^{P})(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆alkyl.

In some embodiments of any one of the aspects, R³³ is —OP(OR^{P})(N(R^{P2})₂). For example, the R³³ is —OP(OR^{P})(N(R^{P2})₂), where R is cyanoethyl (—CH₂CH₂CN) and each R² is isopropyl.

Optionally, only one of R³² and R³³ is a reactive phosphorous group.

In some embodiments of any one of the aspects descried herein, R³³ is a solid support or a linker covalently attached to a solid support. For example, R³³ is —OC(O)CH₂CH₂C(O)NH—Z, where Z is a solid support.

Optionally, only one of R³² and R³³ is a solid support or a linker covalently attached to a solid support.

In some embodiments of any one of the aspects, when R³³ is —OR³³², R³³² can be hydrogen or a hydroxyl protecting group. For example, R³³² can be hydrogen in some embodiments of any one of the aspects described herein. In some embodiments, R³³ is —OC(O)CH₂CH₂CO₂H.

When R³³ is —SR³³³, R³³³ can be hydrogen or a sulfur protecting group. Accordingly, in some embodiments of any one of the aspects, R³³³ is hydrogen.

When R³³ is —O(CH₂CH₂O)ᵣCH₂CH₂OR³³⁴, r can be 1-50; R³³⁴ is independently for each occurrence H, C₁-C₃₀alkyl, cyclyl, heterocyclyl, aryl, heteroaryl, aralkyl, sugar or R³³⁵; and R³³⁵ is independently for each occurrence amino (NH₂), alkylamino, dialkylamino, arylamino, diarylamino, heteroarylamino, or diheteroaryl amino.

When R³³ is —NH(CH₂CH₂NH)ₛCH₂CH₂—R³³⁵, s can be 1-50 and R³³⁵ can be independently for each occurrence amino (NH₂), alkylamino, dialkylamino, arylamino, diarylamino, heteroarylamino, or diheteroaryl amino.

In some embodiments of any one of the aspects described herein, R³³ is hydrogen, halogen, —OR³³², or optionally substituted C₁-C₃₀alkoxy. For example, R³³ is halogen, —OR³³², or optionally substituted C₁-C₃₀alkoxy. In some embodiments of any one of the aspects described herein, R³³ is F, OH or optionally substituted C₁-C₃₀alkoxy.

In some embodiments of any one of the aspects described herein, R³³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂ [CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R³³ is C₁-C₃₀alkoxy optionally substituted with a NH₂, OH, C(O)NH₂, COOH, halo, SH, or C₁-C₆alkoxy. In some embodiments of any one of the aspects described herein, R³³ is —O(CH₂)ₜCH₃, where t is 1-21. For example, t is 14, 15, 16, 17 or 18. In one non-limiting example, t is 16.

In some embodiments of any one of the aspects, R³³ is —O(CH₂)ᵤR³³⁷, where u is 2-10; R³³⁷ is C₁-C₆alkoxy, amino (NH₂), CO₂H, OH or halo. For example, R³³⁷ is —CH₃ or NH₂. Accordingly, in some embodiments of any one of the aspects described herein, R³³ is —O(CH₂)ᵤ—OMe or R³³ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³³ is a C₁-C₆haloalkyl. For example, R³³ is a C₁-C₄haloalkyl. In some embodiments of any one of the aspects described herein, R³³ is —CF₃, —CF₂CF₃, —CF₂CF₂CF₃ or —CF₂(CF₃)₂.

In some embodiments of any one of the aspects described herein, R³³ is —OCH(CH₂OR³³⁸)CH₂OR³³⁹, where R³³⁸ and R³³⁹ independently are H, optionally substituted C₁-C₃₀alkyl, optionally substituted C₂-C₃₀alkenyl or optionally substituted C₂-C₃₀alkynyl. For example, R³³⁸ and R³³⁹ independently are optionally substituted C₁-C₃₀alkyl.

In some embodiments of any one of the aspects described herein, R³³ is —CH₂C(O)NHR³³¹⁰, where R³³¹⁰ is H, optionally substituted C₁-C₃₀alkyl, optionally substituted C₂-C₃₀alkenyl or optionally substituted C₂-C₃₀alkynyl. For example, R³³¹⁰ is H or optionally substituted C₁-C₃₀alkyl. In some embodiments, R³³¹⁰ is optionally substituted C₁-C₆alkyl.

In some embodiments of any one of the aspected described herein, R³³ and R⁴ taken together with the atoms to which they are attached form an optionally substituted C₃₋₈cycloalkyl, optionally substituted C₃₋₈cycloalkenyl, or optionally substituted 3-8 membered heterocyclyl.

### R³⁵

In some embodiments of the various aspects described herein, R³⁵ is R⁵⁵¹, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵⁵³, hydrogen, a phosphorous group, a solid support or a linker to a solid support. When R⁵⁵¹ is —OR⁵⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects described herein, R³⁵ is —OR⁵⁵² or —SR⁵⁵³

In some embodiments of any one of the aspects described herein, R⁵⁵² is a hydroxyl protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R³⁵ is —OR⁵⁵² and R⁵⁵² is 4,4′-dimethoxytrityl (DMT), e.g., R³⁵ is —O-DMT.

In some embodiments of any one of the aspects described herein, R³⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R³⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is —CH(R⁵⁵⁴)—O—R⁵⁵² where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is optionally substituted C₁₋₆alkyl-R⁵⁵¹ or optionally substituted —C₂₋₆alkenyl-R⁵⁵¹,

In some embodiments of any one of the aspects described herein, R³⁵ is —C(R⁵⁵⁴)═CH₁R⁵⁵. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)═CHR⁵⁵¹ and wherein the double bond is in the cis configuration. In some other embodiments of any one of the aspects, R^{d} is —C(R⁵⁵⁴)═CHR⁵⁵¹ and wherein the double bond is in the trans configuration.

In some embodiments of any one of the aspects described herein, R³⁵ is —CH═CHR⁵⁵.

In some embodiments of any one of the aspects, when R³⁵ is —C(R⁵⁵⁴)═CHR⁵⁵, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R³⁵ is —CH═CHR⁵⁵.

In some embodiments of any one of the aspects described herein, R⁵⁵¹ is a reactive phosphorous group.

In some embodiments of any one of the aspects, R³⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R³⁵ is —CH═CH—P(O)(OR⁵⁵⁵)₂, where each R⁵⁵⁵ is H or an oxygen protecting group.

In some embodiments of any one of the aspects, R³³ is a reactive phosphorous group, a solid support, a linker to a solid support, and R³⁵ is a protected hydroxyl.

In some other embodiments of any one of the aspects, R³² is a reactive phosphorous group, a solid support, a linker to a solid support, and R³⁵ is a protected hydroxyl.

### L

In embodiments of the various aspects described herein, L is a linker.

As used herein, the term “linker” means an organic moiety that connects two parts of a compound. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original form after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the dsRNA molecule according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the dsRNA molecule according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, hydrogen, C1-C20 alkyl, C1-C20 haloalkyl, C6-C10 aryl, C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the dsRNA molecule according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the dsRNA molecule according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids.

In some embodiments of any one of the aspects, L is a bond.

In some embodiments of any one of the aspects, L is absent, e.g., R⁶ or R⁷ is —R^{L}.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′—CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides described herein comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside linkage is

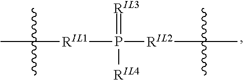

where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R^{IL4} are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL3} and R^{IL4} all are O.

In some embodiments, R^{IL1} and R^{IL2} are O and at least one of R^{IL3} and R^{IL4} is other than O. For example, one of R^{IL3} and R^{IL4} is S and the other is O or both of R^{IL3} and R^{IL4} are S.

In some embodiments of any one of the aspects described herein, one of R³ or R⁵ is a bond to a modified internucleoside linkage, e.g., an internucleoside linkage of structure:

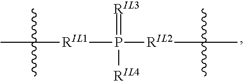

where at least one of R^{IL1}, R^{IL2}, R^{IL3} and R^{IL4} is not O. For example, at least one of R^{IL3} and R^{IL4} is S.

In some embodiments of any one of the aspects described herein, both of R³ and R⁵ are a bond to a modified internucleoside linkage.

In some embodiments of any one of the aspects described herein R³ is a bond to phosphodiester internucleoside linkage.

In some embodiments of any one of the aspects described herein R⁵ is a bond to phosphodiester internucleoside linkage.

In some embodiments of any one of the aspects described herein, R³ is a bond to a modified internucleoside linkage and R⁵ is a bond to phosphodiester internucleoside linkage.

In some embodiments of any one of the aspects described herein, R⁵ is a bond to a modified internucleoside linkage and R³ is a bond to phosphodiester internucleoside linkage.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 modified internucleoside linkages. For example, the oligonucleotide comprises 1, 2, 3 or 4 modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 phosphorothioate internucleoside linkages. For example, the oligonucleotide comprises 1, 2, 3 or 4 phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

### Oxygen Protecting Groups

Some embodiments of the various aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2})N(R^{OP2})₂, —S(═O)R^{OP1}, —SO⁺₂R^{OP1}, —Si(R^{OP2})₃, —P(R^{OP3})₂, —P(R^{OP3})⁺₃X⁻, —P(OR^{OP3})₂, —P(OR^{OP3})₃ X⁻, —P(═O)(R^{OP1})₂, —P(═O)(OR^{OP3})₂, and —P(═O)(N(R^{OP2})₂)₂; wherein each X⁻ is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, —SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂ [CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuSP3inoate, (E)-2-methyl-2-butenoate, o-(methoxyacyl)benzoate, u-naphthoate, nitrate, alkylN, N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In certain embodiments, the hydroxyl protecting group is selected from acetyl, benzyl, t-butyldimethylsilyl, t-butyldiphenylsilyl and dimethoxytrityl wherein a more preferred hydroxyl protecting group is 4,4′-dimethoxytrityl.

The terms “protected hydroxyl” and “protected hydroxy” as used herein mean a group of the formula —OR^{Pro}, wherein R^{Pro} is an oxygen protecting group as defined herein. Nitrogen protecting groups

Some embodiments of the various aspects described herein include a nitrogen protecting group (also referred to as an amino protecting group herein). Nitrogen protecting groups include, but are not limited to, —OH, —OR^{NP1}, —N(R^{NP2})₂, —C(═O)R^{NP1}, —C(═O)N(R^{NP2})₂, —CO₂R^{NP1}, —SO₂R^{NP1}, —C(═NR^{NP2})R^{NP1}, —C(═NR^{NP2})OR^{NP1}, —C(═NR^{NP2})N(R^{NP2})₂, SO₂N(R^{NP2})₂—SO₂R^{NP2}, —SO₂OR^{NP2}, —SOR^{NP1}, —C(═S)N(R^{NP2})₂, —C(═O)SR^{NP2}, —C(═S)SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆-14 aryl, and 5-14 membered heteroaryl groups, where each R^{NP1} is independently C₁₋₁₀ alkyl, C₁-10 perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N—2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl] methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNPlcylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}, —CO₂R^{SP1}, —C(═O)N(R^{SP2})₂, —C(═NR^{SP2})R^{SP1}, —C(═NR^{SP2})OR^{SP1}, —C(═NR^{SP2})N(R^{SP2})₂, —S(═O)R^{SP1}, —SO₂R^{SP1}, —Si(R^{SP1})₃, —P(R^{SP3})₂, —P(R^{SP3})⁺₃ X⁻, —P(OR^{SP3})₂, —P(OR^{SP3})⁺₃ X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, —OR^{SP1}, —N(R^{SP3})₂, —CN, —C(═O)R^{SP1}, —C(═O)N(R^{SP3})₂, —CO₂R^{SP1}, SO₂R^{SP1}, —C(═NR^{SP3})OR^{SP1}, —C(═NR^{SP3})N(R^{SP3})₂, —SO₂N(R^{SP3})₂, —SO₂R^{SP3}, —SO₂OR^{SP3}, —SOR^{SP1}, —C(═S)N(R^{SP3})₂, —C(═O)SR^{SP3}, —C(═S)SR^{SP3}, —P(═O)(R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Sulfur protecting groups are well known in the art and include those described in detail in Greene's Protecting Groups in Organic Synthesis, P. G. M. Wuts, 5^{th} Edition, John Wiley & Sons, 2014, incorporated herein by reference.

### Oligonucleotides

It is noted that the nucleoside of Formula (I) or (II) can be located anywhere in the oligonucleotide. In some embodiments, the nucleoside of Formula (I) or (II) is present at the 5′- or 3′-terminus of the oligonucleotide. In some embodiments, the nucleoside of Formula (I) or (II) is present at an internal position of the oligonucleotide.

In some embodiments of any one of the aspects described herein, the oligonucleotide further comprises, i.e., in addition to a nucleoside of Formula (I) or (II), a nucleoside with a modified sugar. By a “modified sugar” is meant a sugar or moiety other than 2′-deoxy (i.e. 2′-H) or 2′-OH ribose sugar. Some exemplary nucleotides comprising a modified sugar are 2′-F ribose, 2′-OMe ribose, 2′-0,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxypropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′-F nucleotides. It is noted that the 2′-F nucleotides can be present at any position of the oligonucleotide.

In some embodiments, the oligonucleotide comprises, e.g., solely comprises 2′-nucleosides of Formula (I)/(II) and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′-OMe nucleotides. It is noted that the 2′-OMe nucleotides can be present at any position of the oligonucleotide.

In some embodiments, the oligonucleotide comprises, e.g., solely comprises solely comprises solely comprises 2′-nucleosides of Formula (I)/(II) and 2′-OMe nucleosides. In some other embodiments, the oligonucleotide comprises, e.g., solely comprises solely comprises 2′-nucleosides of Formula (I)/(II), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises solely comprises nucleosides of Formula (I)/(II) and 2′-deoxy (2′-H) nucleotides. In some embodiments, the oligonucleotide comprises, e.g., solely comprises nucleosides of Formula (I)/(II), 2′-OMe nucleosides, and 2′-deoxy (2′-H) nucleotides. In some embodiments, the oligonucleotide comprises, e.g., solely comprises nucleosides of Formula (I)/(II), 2′-F nucleosides and 2′-deoxy (2′-H) nucleotides. In some embodiments, the oligonucleotide comprises, e.g., solely comprises nucleosides of Formula (I)/(II), 2′-OMe nucleosides, 2′-F nucleosides and 2′-deoxy (2′-H) nucleotides.

In some embodiments of any one of the aspects described herein, the oligonucleotide further comprises, i.e., in addition to a nucleoside of Formula (I) or (II), a non-natural nucleobase. In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²- and N⁶- with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-natural nucleobase is a protected nucleobase. As used herein, a “protected nucleobase” refers to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hunderes of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

In some embodiments, the oligonucleotide described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, the oligonucleotide described herein comprises a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, the oligonucleotide described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, oligonucleotide described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments of any one of the aspects described herein, the oligonucleotides described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., RP(OH)(O)—O-5′-, R=alkyl, e.g., methyl, ethyl, isopropyl, propyl, etc.), 5′-alkenylphosphonates (i.e. vinyl, substituted vinyl, e.g., OH)₂(0)P-5′-CH═ or (OH)₂(0)P-5′-CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

In some embodiments of any one of the aspects described herein, the oligonucleotide comprises a 5′-vinylphosphonate group. For example, the oligonucleotide comprises a 5′-E-vinyl phosphonate group. In some other non-limiting example, the oligonucleotide comprises a 5′-Z-vinylphosphonate group.

In some embodiments of any one of the aspects, the oligonucleotide described herein comprises a 5′-morpholino, a 5′-dimethylamino, a 5′-deoxy, an inverted abasic, or an inverted abasic locked nucleic acid modification at the 5′-end.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification is located at position 2, 3, 4, 5, 6, 7, 8 or 9, counting from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

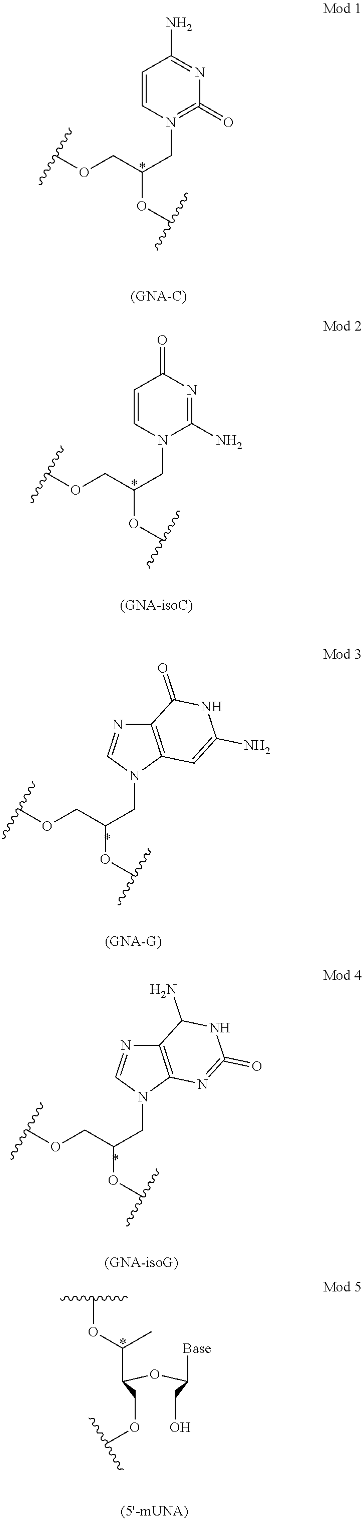

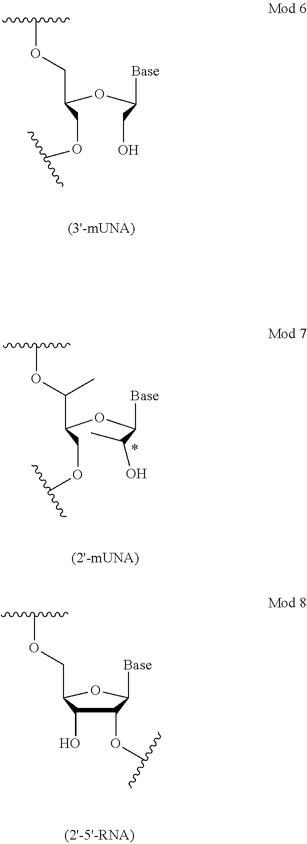

*Both stereoisomers tested

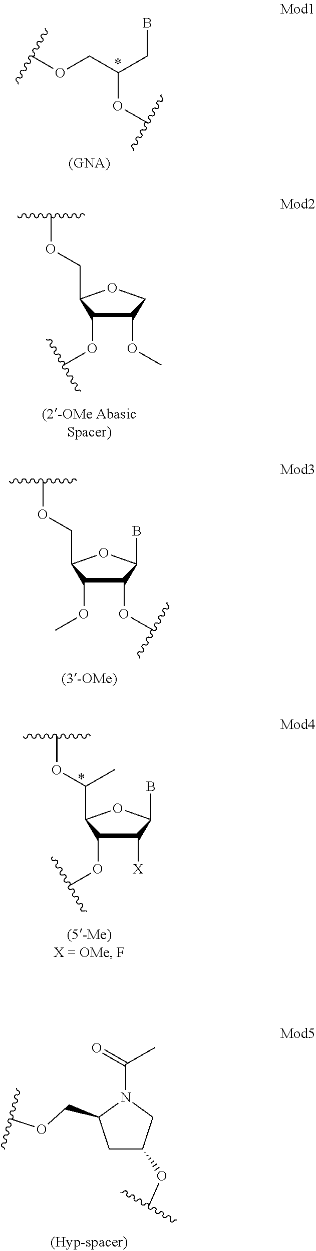

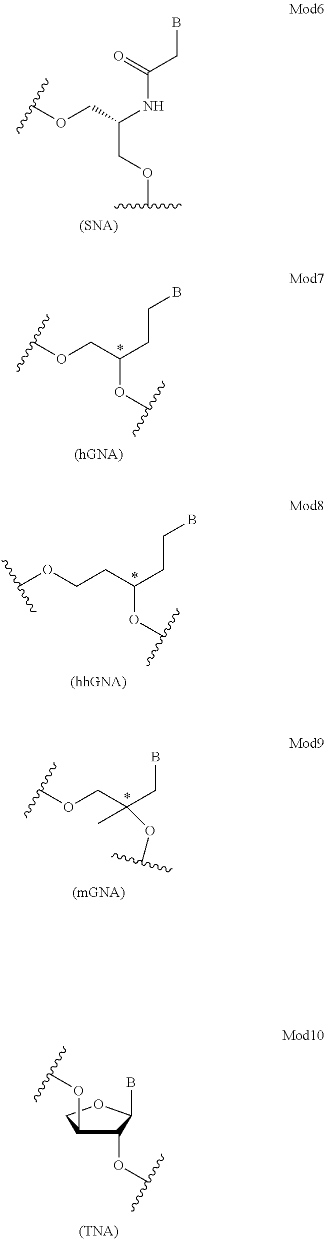

*Both stereoisomers tested

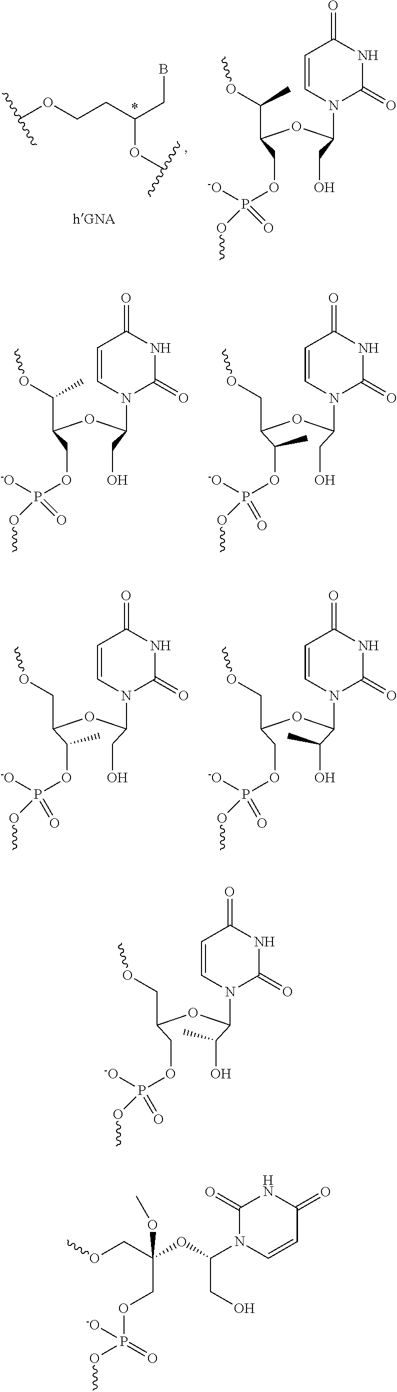

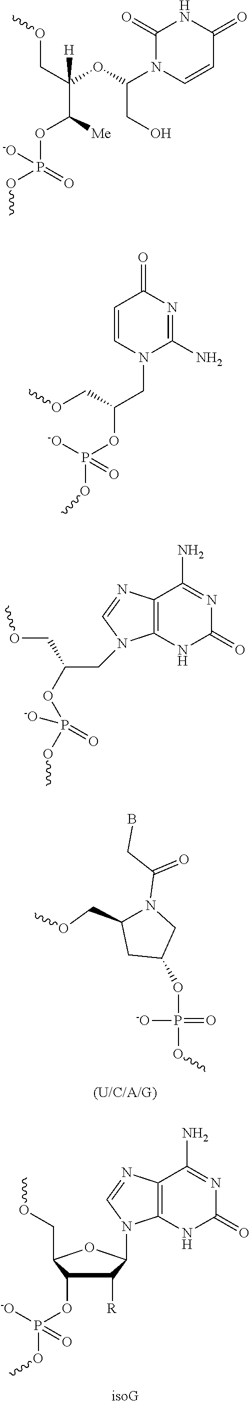

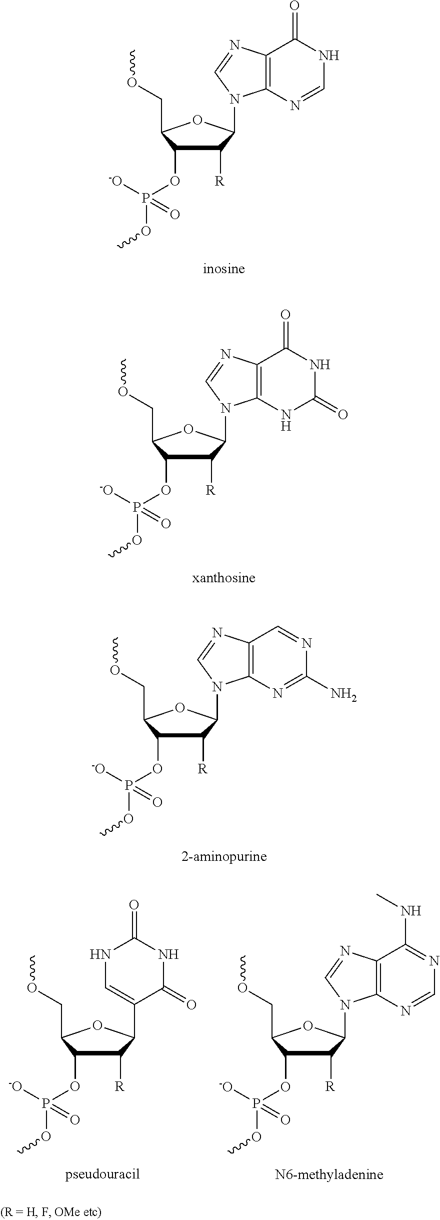

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

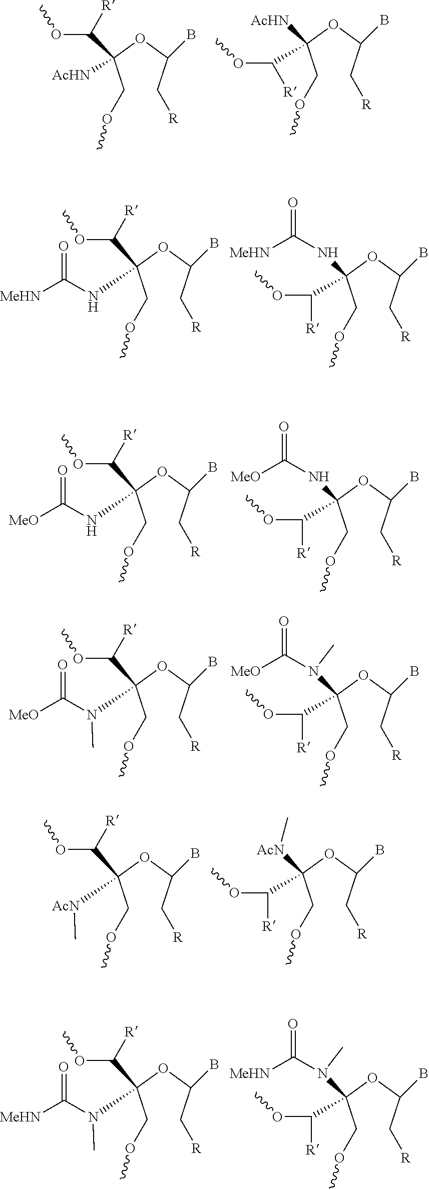

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and Stereochemistry is R or S and combination of R and S for the unspecified chiral centers.

In some embodiments, the destabilizing modification mUNA is selected from the group consisting of

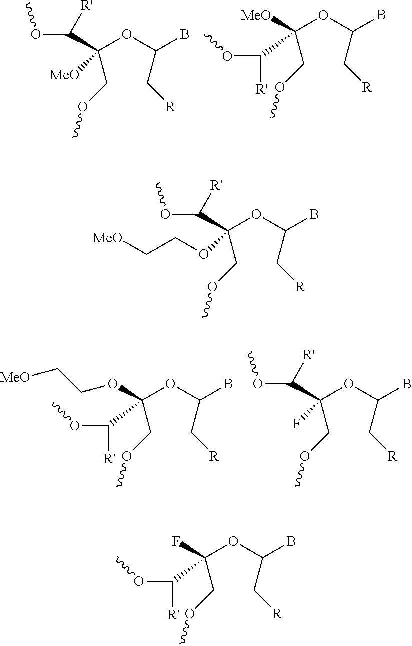

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and Stereochemistry is R or S and combination of R and S for the unspecified chiral centers.

In some embodiments, the destabilizing modification mUNA is selected from the group consisting of

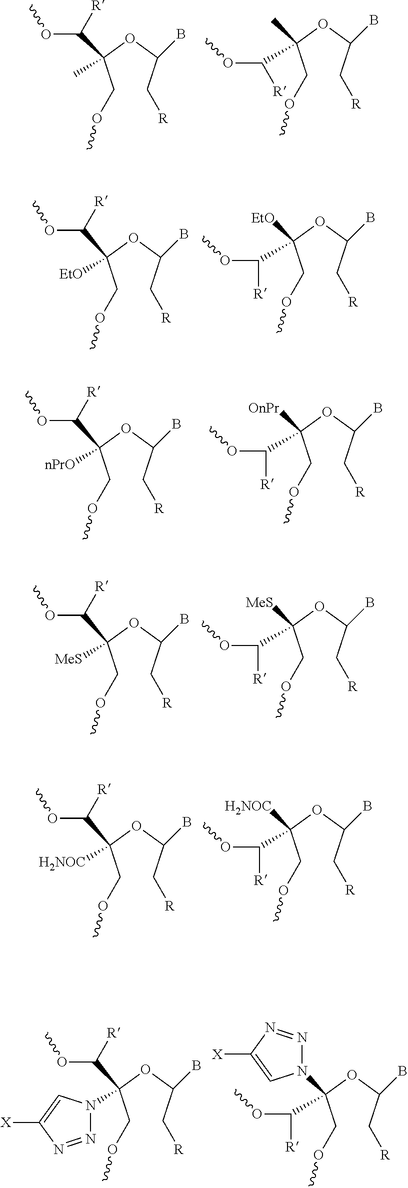

R═H, OMe; F; OH; O—(CH₂)₂OMe; SMe, NMe₂; NH₂; Me; O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 7-deazapurines; and Stereochemistry is R or S and combination of R and S for the unspecified chiral centers.

In some embodiments, the destabilizing modification mUNA is selected from the group consisting of

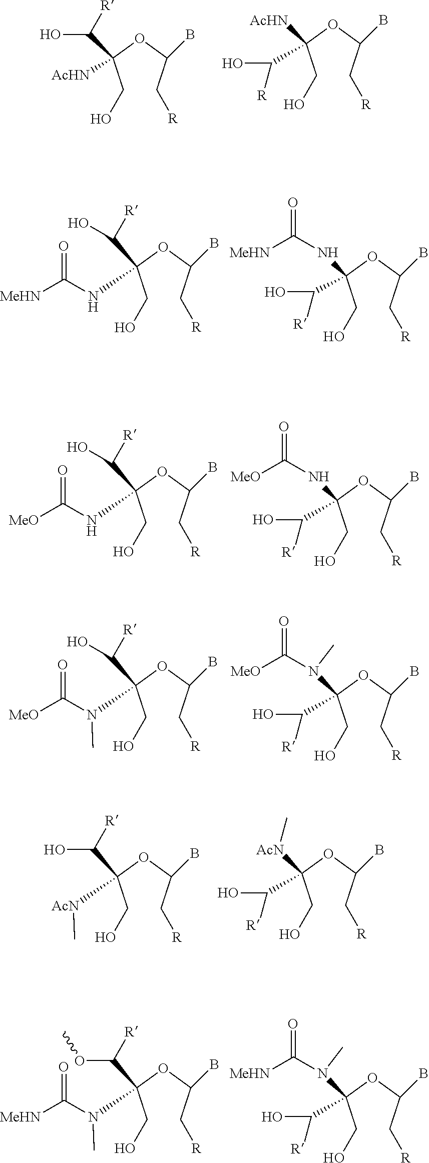

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and Stereochemistry is R or S and combination of R and S for the unspecified chiral centers

In some embodiments, the destabilizing modification mUNA is selected from the group consisting of

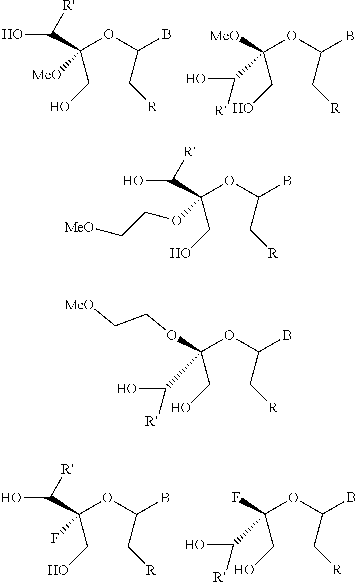

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 2-thiouridine; 4-thiouridine; C5-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

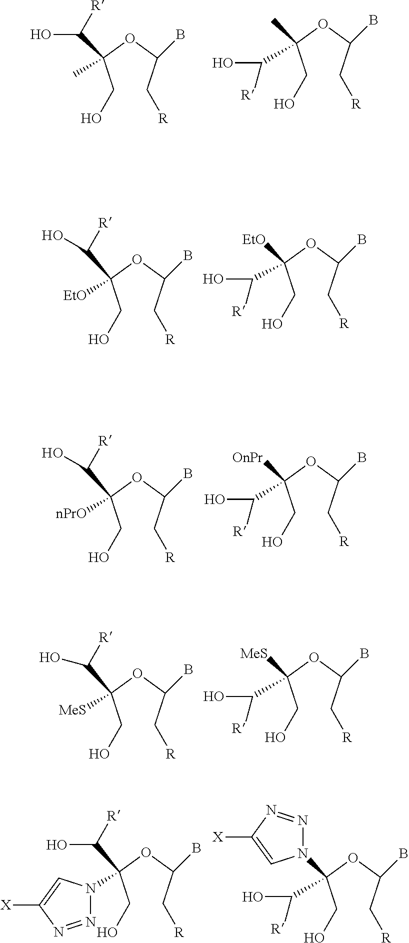

R═H, OMe; F; OH; O—(CH₂)₂OMe; SMe, NMe₂; NH₂; Me; O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; 06-alkyl-G; 7-deazapurines; and
Stereochemistry is R or S and combination of R and S for the unspecified chiral centers

Exemplary abasic modifications include, but are not limited to the following:

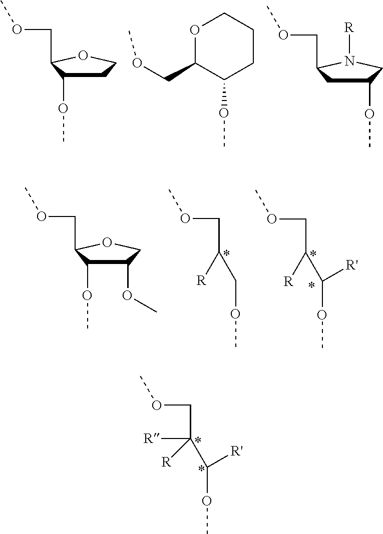

Wherein R═H, Me, Et or OMe; R′═H, Me, Et or OMe; R″═H, Me, Et or OMe

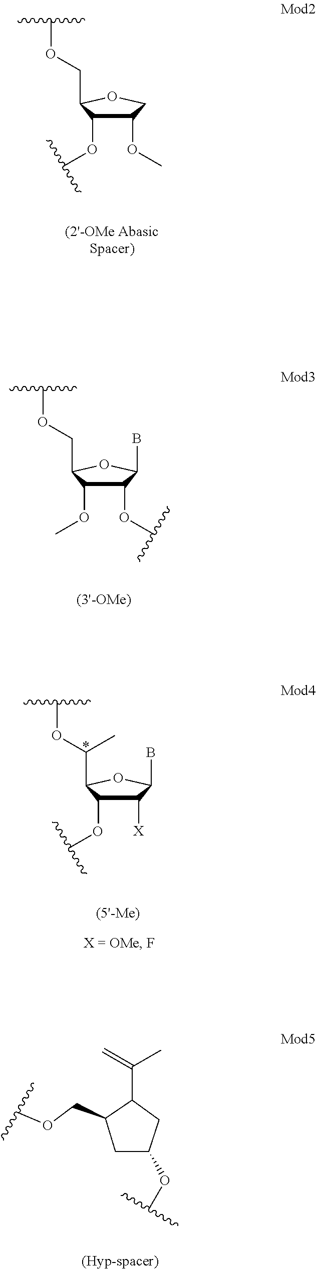

wherein B is a modified or unmodified nucleobase and the asterisk on each structure represents either R, S or racemic.

Exemplified sugar modifications include, but are not limited to the following:

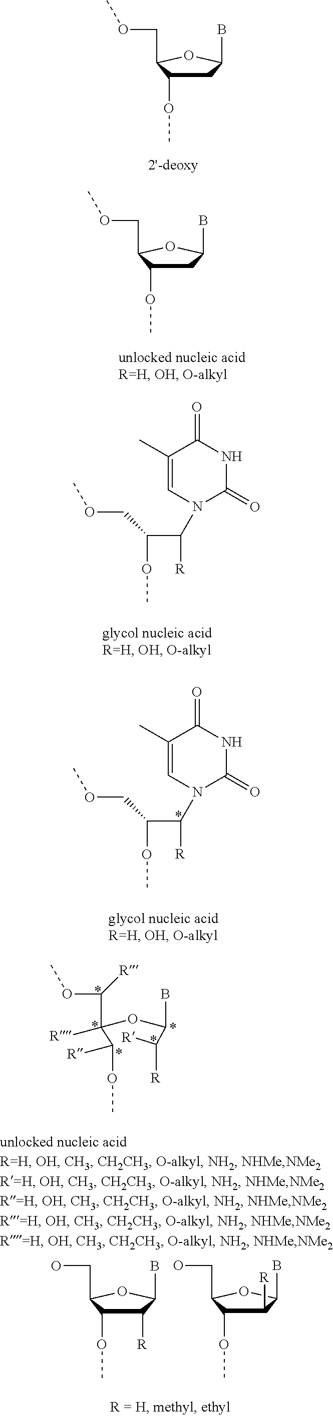

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h′GNA (Mod A-Mod K).

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

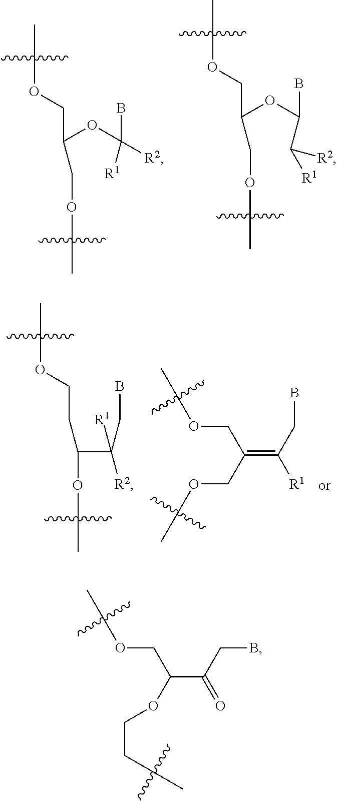

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

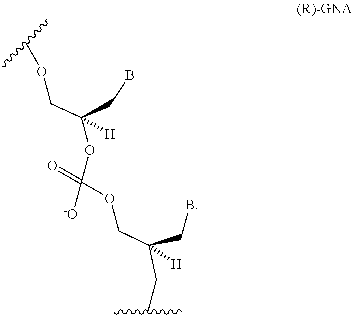

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

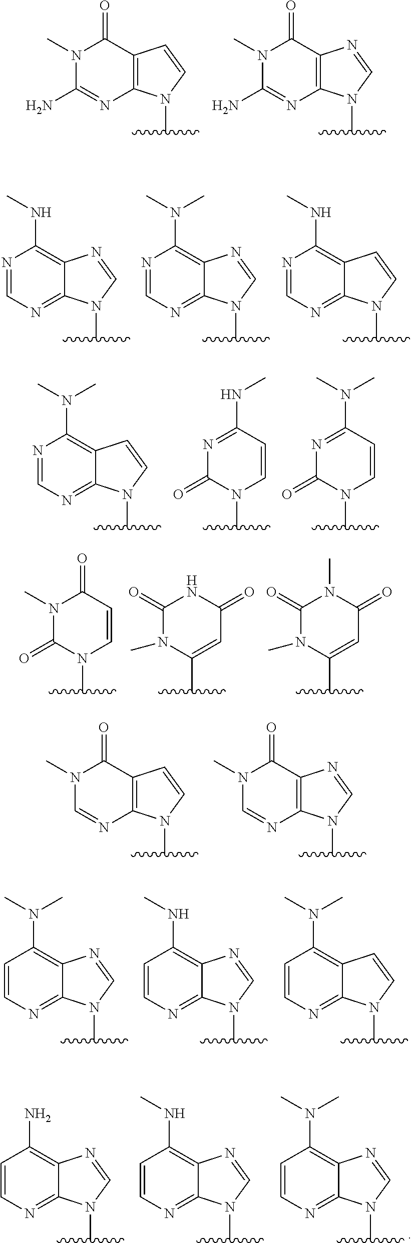

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

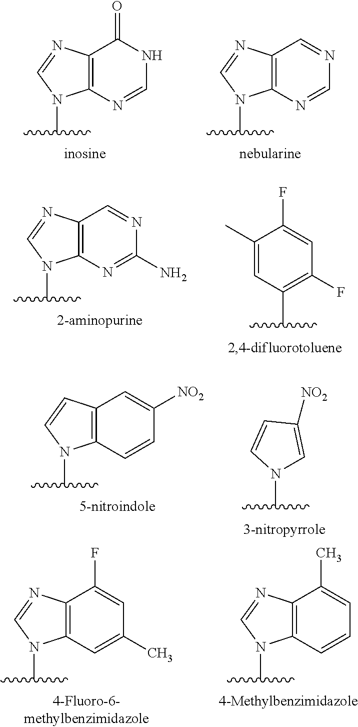

In some embodiments, the thermally destabilizing modification of the duplex in the seed region of the antisense strand includes one or more a-nucleotide complementary to the base on the target mRNA, such as:

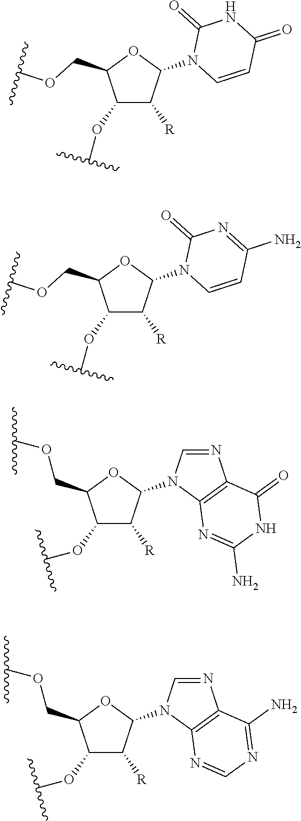

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

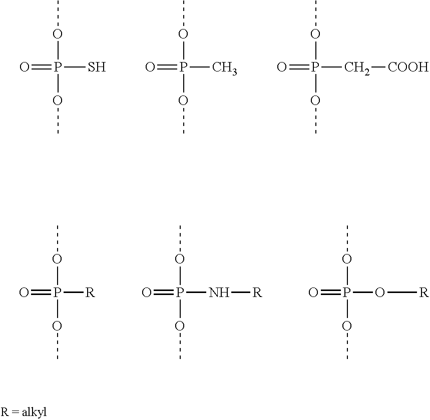

The alkyl for the R group can be a C1-C6alkyl. Specific alkyls for the R group include, but are not limited to methyl, ethyl, propyl, isopropyl, butyl, pentyl and hexyl.

In some embodiments of any one of the aspects described herein, the oligonucleotide can comprise one or more stabilizing modifications. For example, the oligonucleotide can comprise at least two (e.g., two, three, four, five, six, seven, eight, nine, ten or more) stabilizing modifications.

In some embodiments, the oligonucleotide comprises at least two (e.g., two, three, four, five, six, seven, eight, nine, ten or more) stabilizing modifications. Without limitations, a stabilizing modification in the oligonucleotide can be present at any positions. In some embodiments, the oligonucleotide comprises stabilizing modifications at positions 2, 6, 8, 9, 14 and 16, counting from the 5′-end. In some other embodiments, the oligonucleotide comprises stabilizing modifications at positions 2, 6, 14 and 16, counting from the 5′-end. In still some other embodiments, the oligonucleotide comprises stabilizing modifications at positions 2, 14 and 16, counting from the 5′-end. In some embodiments, the oligonucleotide comprises stabilizing modifications at positions 7, 10 and 11, counting from the 5′-end. In some other embodiments, the oligonucleotide comprises stabilizing modifications at positions 7, 9, 10 and 11, counting from the 5′-end.

In some embodiments, the oligonucleotide comprises at least one stabilizing modification adjacent to a destabilizing modification. For example, the stabilizing modification can be the nucleotide at the 5′-end or the 3′-end of the destabilizing modification, i.e., at position −1 or +1 from the position of the destabilizing modification. In some embodiments, the oligonucleotide comprises a stabilizing modification at each of the 5′-end and the 3′-end of the destabilizing modification, i.e., positions −1 and +1 from the position of the destabilizing modification.

In some embodiments, the oligonucleotide comprises at least two stabilizing modifications at the 3′-end of a destabilizing modification, i.e., at positions +1 and ⁺₂ from the position of the destabilizing modification.

Exemplary thermally stabilizing modifications include, but are not limited to 2′-fluoro modifications. Other thermally stabilizing modifications include, but are not limited to LNA.

### Uses of Oligonucleotides

The oligonucleotides described herein can be used for any use known in the art for oligonucleotides. For example, the oligonucleotides described herein can be used RNA interference based gene silencing techniques. Some exemplary uses for the oligonucleotides described herein include, but are not limited to, RNA interference agents, antisense oligonucleotides, aptamers, miRNAs, ribozymes, triplex forming oligonucleotides and the like.

In some embodiments of any one of the aspects described herein, an oligonucleotide described herein is comprised in a double-stranded nucleic acid. For example, an olignucleotide described herein can be one strand of a dsRNA molecule.

Generally, the dsRNA molecule comprises a sense strand (also referred to as passenger strand) and an antisense strand (also referred to as guide strand). An oligonucleotide described herein can be used as the sense and/or the antisense strand of the dsRNA molecule. In some embodiments, an oligonucleotide described herein is the sense strand of the dsRNA molecule. In some embodiments, an oligonucleotide described herein is the antisense strand of the dsRNA molecule.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the duplex region is 20, 21 or 22 nucleotide pairs in length. For example, the dsRNA molecule has a duplex region of 21 base pairs.

In some embodiments of any one of the aspects, the oligonucleotide described herein or the antisense strand of the dsRNA molecule described herein comprises a nucleotide sequence substantially complementary to a target nucleic acid, e.g., a target gene or mRNA.

Accordingly, in another aspect, the disclosure is directed to a use of an oligonucleotide and/or dsRNA molecule described herein for inhibiting expression of a target gene. In some embodiments, the present invention further relates to a use of an oligonucleotide and/or dsRNA molecule described herein for inhibiting expression of a target gene in vitro.

In another aspect, the disclosure is directed to a use of an oligonucleotide and/or dsRNA molecule described herein for use in inhibiting expression of a target gene in a subject. The subject may be any animal, such as a mammal, e.g., a mouse, a rat, a sheep, a cattle, a dog, a cat, or a human

In some embodiments, the oligonucleotide and/or dsRNA molecule described herein is administered in buffer.

In some embodiments, oligonucleotide and/or dsRNA molecule described herein described herein can be formulated for administration to a subject. A formulated oligonucleotide and/or dsRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide and/or dsRNA preparation can be formulated in combination with another agent, e.g., another therapeutic agent or an agent that stabilizes an oligonucleotide and/or dsRNA, e.g., a protein that complexes with oligonucleotide and/or dsRNA. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the oligonucleotide and/or dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the oligonucleotide and/or dsRNA preparation includes at least a second therapeutic agent (e.g., an agent other than a RNA or a DNA). For example, a oligonucleotide and/or dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the oligonucleotide and/or dsRNA according to the present invention are discussed below.

### Liposomes.

A oligonucleotide and/or dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide and/or dsRNA composition. The lipophilic material isolates the aqueous interior from an aqueous exterior, which typically does not include the oligonucleotide and/or dsRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide and/or dsRNA are delivered into the cell where the dsRNA can specifically bind to a target RNA and can mediate RNAi. In some embodiments, the liposomes are also specifically targeted, e.g., to direct the oligonucleotide and/or dsRNA to particular cell types.

A liposome containing oligonucleotide and/or dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of oligonucleotide and/or dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging oligonucleotide and/or dsRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2, 405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include oligonucleotide and/or dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

### Surfactants.

The oligonucleotide and/or dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

### Micelles and Other Membranous Formulations.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the oligonucleotide and/or dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the oligonucleotide and/or dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### Particles.

In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotide and/or dsRNA described herein can be formulated for pharmaceutical use. The present invention further relates to a pharmaceutical composition comprising the oligonucleotide and/or dsRNA described herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a dsRNA molecule described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of oligonucleotide and/or dsRNA composition described herein.

### Methods of Inhibiting Expression of a Target Gene

Aspects of the disclosure also relate to methods for inhibiting the expression of a target gene. The method comprises administering to the subject in an amount sufficient to inhibit expression of the target gene: (i) a double-stranded RNA described herein, where the wherein the first strand is complementary to a target gene; and/or (ii) an oligonucleotide described herein, wherein the oligonucleotide is complementary to a target gene.

The present disclosure further relates to a use of an oligonucleotide and/or dsRNA molecule described herein for inhibiting expression of a target gene in a target cell. The present disclosure further relates to a use of an oligonucleotide and/or dsRNA molecule described herein for inhibiting expression of a target gene in a target cell in vitro.

Another aspect the invention relates to a method of modulating the expression of a target gene in a cell, comprising administering to said cell an oligonucleotide and/or dsRNA molecule described herein. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, INK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

Some embodiments of the various aspects described herein are described by the following numbered embodiments:

Embodiment 1: A method for preparing an oligonucleotide comprising a nucleoside of Formula (I), the method comprising reacting an oligonucleotide comprising a nucleoside of Formula (II) with an amine of formula HNR⁶R⁷, wherein: R^{H} is halogen (e.g., chloro or fluoro); R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support; R³ is a bond to an internucleotide linkage to a subsequent nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support; R⁴ is hydrogen, optionally substituted C₁₋₆alkyl, optionally substituted C₂₋₆alkenyl, optionally substituted C₂₋₆alkynyl, or optionally substituted C₁₋₆alkoxy; or R⁴ and R² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆ alkenyl or optionally substituted C₂-C₆ alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; v is 1, 2 or 3; or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl; R⁵ represents a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(RP)(OH)(O)P—O-5′, R is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′; X is 0 or S; a and b are each independently 1-10; each R⁶ and R⁷ is independently hydrogen or -L-R^{L}, provided that at least one of R⁶ and R⁷ is not H; or R⁶ and R⁷ taken together with the nitrogen atom to which they are attached form a 3-10 membered heterocyclyl or a 3-10 membered heteroaryl group, the heterocyclyl or heteroaryl comprising one -L-R^{L} group; L is absent or a linker; each R^{L} is a ligand, (e.g., selected independently from the group consisting of carbohydrates, peptides, lipids, therapeutic agents, diagnostic agents, detectable labels, antibodies or fragments thereof, vitamins, optionally substituted C₁₋₃₀ alkyl, optionally substituted C₁₋₃₀ alkenyl, or optionally substituted C₁₋₃₀ alkynyl); each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl, and provided that: (i) no more than one of R² and R³ is a bond to an internucleotide linkage to a subsequent nucleotide; and (ii) when both of R² and R³ are not a bond, then R⁵ is a bond to an internucleotide linkage to a preceding nucleotide.

Embodiment 2: The method of claim 1, wherein R^{H} is fluoro.

Embodiment 3: The method of claim 1 or 2, wherein at least one L is a bond.

Embodiment 4: The method of any one of claims 1-3, wherein at least one L is a linker.

Embodiment 5: The method of any one of claims 1-4, wherein at least one R^{L} is selected independently from the group consisting of carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

Embodiment 6: The method of any one of claims 1-5, wherein at least one R^{L} is a ligand selected from targeting ligands, endosomolytic ligands and PK modulating ligands.

Embodiment 7: The method of any one of claims 1-6, wherein R⁶ and R⁷ are same.

Embodiment 8: The method of any one of claims 1-7, wherein R² is hydrogen, hydroxyl, protected hydroxyl, halogen, optionally substituted C₁₋₆ alkoxy (e.g., methyl) or alkoxyalkyl (e.g. 2-methoxyethyl); or R⁴ and R² taken together are 4′-C(R¹⁰R¹¹)ᵥ—Y-2′ or 4′-Y—C(R¹⁰R¹¹)ᵥ-2′.

Embodiment 9: The method of any one of claims 1-8, wherein R² is hydrogen, hydroxyl, F, methoxy, or R⁴ and R² taken together are 4′-C(R¹⁰R¹¹)ᵥ—Y-2′.

Embodiment 10: The method of any one claims 1-9, wherein R⁴ and R² taken together are 4′-C(R¹⁰R¹¹)ᵥ—Y-2′.

Embodiment 11: The method of any one of claims 1-10, wherein R⁴ is H.

Embodiment 12: The method of any one of claims 1-11, wherein R³ is a bond to an internucleotide linkage to a subsequent nucleotide, hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

Embodiment 13: The method of any one of claims 1-12, wherein R³ is a bond to an internucleotide linkage to a subsequent nucleotide, hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support.

Embodiment 14: The method of any one of claims 1-13, wherein R³ is a bond to an internucleotide linkage to a subsequent nucleotide.

Embodiment 15: The method of any one of claims 1-14, wherein R³ is hydroxyl.

Embodiment 16: The method of any one of claims 1-15, wherein R⁵ is a bond to an internucleotide linkage to a preceding nucleotide, hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates and phosphate mimics.

Embodiment 17: The method of any one of claims 1-16, wherein R⁵ is a bond to an internucleotide linkage to a preceding nucleotide.

Embodiment 18: The method of any one of claims 1-17, wherein R⁵ is hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

Embodiment 19: The method of any one of claims 1-18, wherein the oligonucleotide comprises from 3 to 50 nucleotides.

Embodiment 20: The method of any one of claims 1-19, wherein the oligonucleotide comprises at least one ribonucleotide.

Embodiment 21: The method of any one of claims 1-20, wherein the oligonucleotide comprises at least one 2′-deoxyribonucleotide.

Embodiment 22: The method of any one of claims 1-21, wherein the oligonucleotide comprises at least one nucleotide with a modified or non-natural nucleobase.

Embodiment 23: The method of any one of claims 1-22, wherein the oligonucleotide comprises at least one nucleotide with a modified ribose sugar.

Embodiment 24: The method of any one of claims 1-23, wherein the oligonucleotide comprises at least one nucleotide comprising a group other than H or OH at the 2′-position of the ribose sugar.

Embodiment 25: The method of any one of claims 1-24, wherein the oligonucleotide comprises at least one nucleotide with a 2′-F ribose.

Embodiment 26: The method of any one of claims 1-25, wherein the oligonucleotide comprises at least one nucleotide with a 2′-OMe ribose.

Embodiment 27: The method of any one of claims 1-26, wherein the oligonucleotide comprises at least one nucleotide comprising a moiety other than a ribose sugar.

Embodiment 28: The method of any one of claims 1-27, wherein the oligonucleotide comprises at least one modified internucleotide linkage.

Embodiment 29: The method of any one of claims 1-28, wherein the oligonucleotide comprises at least 2, e.g., 3, 4 or 5 consecutive independently selected monomers of the Formula (I) and/or (II).

Embodiment 30: The method of any one of claims 1-29, wherein the oligonucleotide is attached to a solid support.

Embodiment 31: The method of any one of claims 1-30, wherein the oligonucleotide comprises at least one hydroxyl, phosphate or amino protecting group.

Embodiment 32: An oligonucleotide prepared by a method of any one of claims 1-31.

Embodiment 33: An oligonucleotide comprising a nucleoside of Formula (II).

Embodiment 34: A compound of Formula (III), wherein: R^{H} is halogen; R³² is hydrogen, hydroxy, halogen protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support; R³³ is hydrogen, hydroxy, halogen protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support, and optionally, only one of R³² and R³³ is a phosphate group, a reactive phosphorous group, a solid support or a linker to a solid support; R⁴ is hydrogen, optionally substituted C₁₋₆alkyl, optionally substituted C₂₋₆alkenyl, optionally substituted C₂₋₆alkynyl, or optionally substituted C₁₋₆alkoxy; or R⁴ and R³² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group; v is 1, 2 or 3; or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl; R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}—5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10); and each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

Embodiment 35: The compound of claim 34, wherein R^{H} is fluoro.

Embodiment 36: The compound of claim 34 or 35, wherein R³² is halogen; or R⁴ and R³² taken together are 4′-C(R¹⁰R¹¹)ᵥ—Y-2′ or 4′-Y—C(R¹⁰R¹¹)ᵥ-2′.

Embodiment 37: The compound of any one of claims 34-36, wherein R³² is F, or R⁴ and R² taken together are 4′-C(R¹⁰R¹¹)ᵥ—Y-2′.

Embodiment 38: The compound of any one claims 34-37, wherein R⁴ and R³² taken together are 4′-C(R¹⁰R¹¹)ᵥ—Y-2′.

Embodiment 39: The compound of any one of claims 34-37, wherein R⁴ is H.

Embodiment 40: The compound of any one of claims 34-39, wherein R³³ is hydroxy, protected hydroxy, a phosphate group, a solid support, or a linker covalently bonded (e.g., —C(O)CH₂CH₂C(O)—) to a solid support.

Embodiment 41: The compound of any one of claims 34-40, wherein R³³ is hydroxy, a phosphate group, a solid support, or a linker covalently bonded (e.g., —C(O)CH₂CH₂C(O)—) to a solid support.

Embodiment 42: The compound of any one of claims 34-41, wherein R³⁵ is hydroxy, protected hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates and phosphate mimics.

Embodiment 43: The compound of any one of claims 34-42, wherein R³⁵ is hydroxyl or protected hydroxyl.

Embodiment 44: The compound of any one of claims 34-43, wherein R³³ is a reactive phosphorous group and R³⁵ is a protected hydroxyl.

Embodiment 45: The compound of any one of claims 34-43, wherein R³² is a reactive phosphorous group and R³⁵ is a protected hydroxyl.

Embodiment 46: The compound of any one of claims 34-41, wherein R³⁵ is a vinylphosphonate (VP) group, cyclopropylphosphonate, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates, or a phosphate mimic; and R³³ is a reactive phosphorous group.

Embodiment 47: The compound of any one of claims 34-41, wherein R³⁵ is a vinylphosphonate (VP) group, cyclopropylphosphonate, or a phosphate mimic; and R³³ is a reactive phosphorous group.

Embodiment 48: The compound of any one of claims 34-41, wherein R³⁵ is a vinylphosphonate (VP) group (e.g., E-vinylphosphonate), cyclopropylphosphonate, or a phosphate mimic; and R³³ is a phosphoramidite group.

Embodiment 49: The compound of any one of claims 34-41, wherein R³⁵ is a triphosphate group and R³³ is allyloxy, azidomethoxy, or aminooxy.

Embodiment 50: The compound of claim 34, wherein the compound is

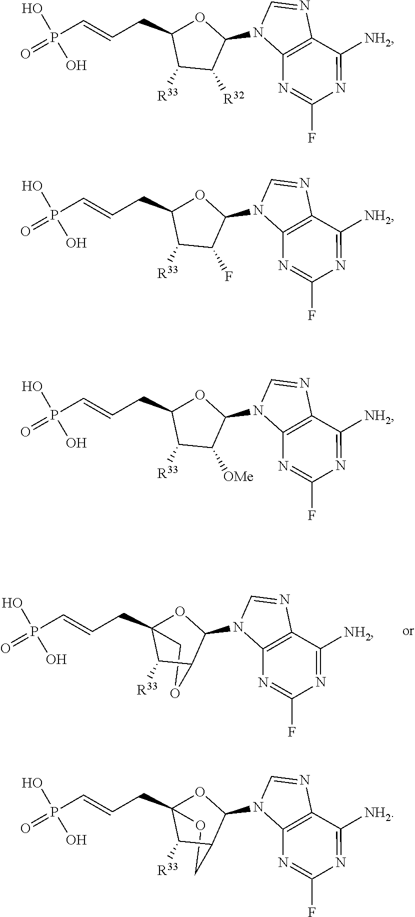

Embodiment 51: The compound of claim 50, wherein R³³ is a reactive phosphorous group.

Embodiment 52: A method for preparing an oligonucleotide comprising a nucleoside of Formula (X), the method comprising reacting an oligonucleotide comprising a nucleoside of Formula (XI) with an alkali hydroxide or alkali earth hydroxide (e.g., NaOH), wherein: R^{H} is halogen (e.g., chloro or fluoro); R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support; R³ is a bond to an internucleotide linkage to a subsequent nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support; R⁴ is hydrogen, optionally substituted C₁₋₆alkyl, optionally substituted C₂₋₆alkenyl, optionally substituted C₂₋₆alkynyl, or optionally substituted C₁₋₆alkoxy; or
R⁴ and R² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R^{a13})C(O)—, or —C(O)N(R^{a13})—; R¹⁰ and R¹¹ and R^{a13} independently are H, optionally substituted C₁-C₆alkyl, optionally substituted C₂-C₆ alkenyl or optionally substituted C₂-C₆ alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; v is 1, 2 or 3; or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl; R⁵ represents a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′; X is O or S; a and b are each independently 1-10; each R⁶ and R⁷ is independently hydrogen or -L-R^{L}, provided that at least one of R⁶ and R⁷ is not H; or R⁶ and R⁷ taken together with the nitrogen atom to which they are attached form a 3-10 membered heterocyclyl or a 3-10 membered heteroaryl group, the heterocyclyl or heteroaryl comprising one -L-R^{L} group; L is absent or a linker;
each R^{L} is a ligand, (e.g., selected independently from the group consisting of carbohydrates, peptides, lipids, therapeutic agents, diagnostic agents, detectable labels, antibodies or fragments thereof, vitamins, optionally substituted C₁₋₃₀ alkyl, optionally substituted C₁₋₃₀ alkenyl, or optionally substituted C₁₋₃₀ alkynyl); each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl, provided that: (i) no more than one of R² and R³ is a bond to an internucleotide linkage to a subsequent nucleotide; and (ii) when both of R² and R³ are not a bond, then R⁵ is a bond to an internucleotide linkage to a preceding nucleotide.

Embodiment 53: The method of claim 52, wherein the oligonucleotide comprising a nucleoside of formula (XI) is linked to a solid support.

Embodiment 54: method of claim 52 or 53, wherein the oligonucleotide comprising a nucleoside of formula (XI) is linked to a solid support and the oligonucleotide comprising a nucleoside of formula (X) is not linked to a solid support.

Embodiment 55: The method of any one of claims 52-54, wherein the oligonucleotide comprising a nucleoside of formula (XI) is not linked to a solid support.

Embodiment 56: The method of claim 55, wherein the oligonucleotide comprising a nucleoside of formula (XI) and the oligonucleotide comprising a nucleoside of formula (X) are not linked to a solid support.

Embodiment 57: The method of claim 52, wherein the oligonucleotide comprising a nucleoside of formula (XI) is linked to a solid support and the method comprises a step of cleaving the oligonucleotide from the solid support prior to reacting with the alkali hydroxide or alkali earth hydroxide (e.g., NaOH).

Embodiment 58: The method of claim 57, wherein said step of cleaving the oligonucleotide comprising a nucleoside of formula (XI) from the solid support comprises contacting the oligonucleotide linked to a solid support with an ammonium hydroxide at a temperature less than 60° C. (e.g., about room temperature or between about 20° C. and 40° C.).

Embodiment 59: The method of any one of claims 52-58, wherein R^{H} is fluoro.

Embodiment 60: The method of any one of claims 52-59, wherein at least one L is a bond.

Embodiment 61: The method of any one of claims 52-60, wherein at least one L is a linker.

Embodiment 62: The method of any one of claims 52-61, wherein at least one R^{L} is selected independently from the group consisting of carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

Embodiment 63: The method of any one of claims 52-62, wherein at least one R^{L} is a ligand selected from targeting ligands, endosomolytic ligands and PK modulating ligands.

Embodiment 64: The method of any one of claims 52-63, wherein R⁶ and R⁷ are same.

Embodiment 65: The method of any one of claims 52-64, wherein R² is hydrogen, hydroxyl, protected hydroxyl, halogen, optionally substituted C₁₋₆ alkoxy (e.g., methyl) or alkoxyalkyl (e.g. 2-methoxyethyl); or R⁴ and R² taken together are 4′-C(R¹⁰R¹¹)ᵥ—Y-2′ or 4′-Y—C(R¹⁰R¹¹)ᵥ-2′.

Embodiment 66: The method of any one of claims 52-65, wherein R² is hydrogen, hydroxyl, F, methoxy, or R⁴ and R² taken together are 4′-C(R¹⁰R¹¹)ᵥ—Y-2′.

Embodiment 67: The method of any one claims 52-66, wherein R⁴ and R² taken together are 4′-C(R¹⁰R¹¹)ᵥ—Y-2′.

Embodiment 68: The method of any one of claims 52-67, wherein R⁴ is H.

Embodiment 69: The method of any one of claims 52-68, wherein R³ is a bond to an internucleotide linkage to a subsequent nucleotide, hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

Embodiment 70: The method of any one of claims 52-69, wherein R³ is a bond to an internucleotide linkage to a subsequent nucleotide, hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support.

Embodiment 71: The method of any one of claims 52-70, wherein R³ is a bond to an internucleotide linkage to a subsequent nucleotide.

Embodiment 72: The method of any one of claims 52-71, wherein R³ is hydroxyl.

Embodiment 73: The method of any one of claims 52-72, wherein R⁵ is a bond to an internucleotide linkage to a preceding nucleotide, hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates and phosphate mimics.

Embodiment 74: The method of any one of claims 52-73, wherein R⁵ is a bond to an internucleotide linkage to a preceding nucleotide.

Embodiment 75: The method of any one of claims 52-74, wherein R⁵ is hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

Embodiment 76: The method of any one of claims 52-76, wherein the oligonucleotide comprises at least one ribonucleotide.

Embodiment 77: The method of any one of claims 52-77, wherein the oligonucleotide comprises at least one 2′-deoxyribonucleotide.

Embodiment 78 The method of any one of claims 52-78, wherein the oligonucleotide comprises at least one nucleotide with a modified or non-natural nucleobase.

Embodiment 79: The method of any one of claims 52-79, wherein the oligonucleotide comprises at least one nucleotide with a modified ribose sugar.

Embodiment 80: The method of any one of claims 52-80, wherein the oligonucleotide comprises at least one nucleotide comprising a group other than H or OH at the 2′-position of the ribose sugar.

Embodiment 81: The method of any one of claims 52-81, wherein the oligonucleotide comprises at least one nucleotide with a 2′-F ribose.

Embodiment 82: The method of any one of claims 52-82, wherein the oligonucleotide comprises at least one nucleotide with a 2′-OMe ribose.

Embodiment 83: The method of any one of claims 52-83, wherein the oligonucleotide comprises at least one nucleotide comprising a moiety other than a ribose sugar.

Embodiment 84: The method of any one of claims 52-84, wherein the oligonucleotide comprises at least one modified internucleotide linkage.

Embodiment 85: The method of any one of claims 52-85, wherein the oligonucleotide comprising a nucleoside of formula (XI) comprises at least one hydroxyl, phosphate or amino protecting group.

Embodiment 86: The method of any one of claims 52-86, wherein the oligonucleotide comprising a nucleoside of formula (XI) does not comprise a hydroxyl, phosphate or amino protecting group.

### Some Selected Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-l,l-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, -NHaryl, and N(aryl)₂. The term “heteroarylamino” means a nitrogen moiety having at least one heteroaryl radical attached to the nitrogen. For example —NHheteroaryl, and N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O— alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, OS(O)₂, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl-group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

As used herein, the term “nucleoside” means a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosomine comprising a nucleobase and a sugar having a phosphate group covalently linked to the sugar. Nucleotides may be modified with any of a variety of substituents.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to β-D-methyleneoxy LNA.

As used herein, the term “MOE” refers to a 2′-O-methoxyethyl substituent.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric compounds are gapmers having 2′-deoxynucleotides in the gap and nucleotides with high-affinity modifications in the wing.

The term ‘BNA’ refers to bridged nucleic acid, and is often referred as constrained or inaccessible RNA. BNA can contain a 5-, 6-membered, or even a 7-membered bridged structure with a “fixed” C₃′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

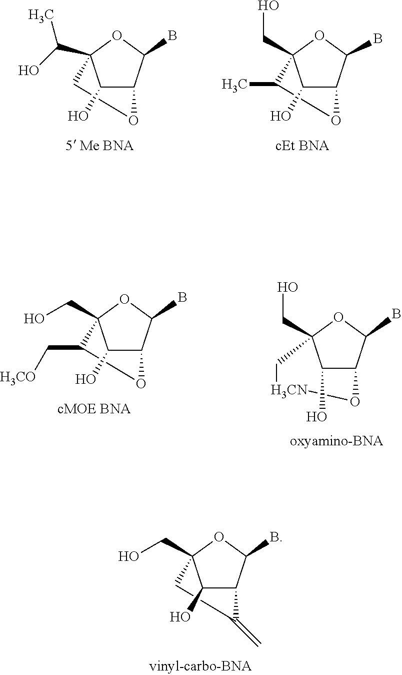

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

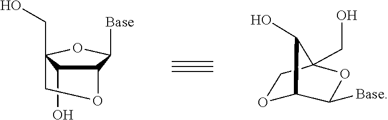

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized antisense oligonucleotide. In certain embodiments, a dosage unit is a vial comprising reconstituted antisense oligonucleotide.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Simple Chemical Approaches to Introduce 2,6-Diaminopurine and 2-Aminoadenine Conjugates into Oligonucleotides

2,6-Diaminopurine (DAP) is a nucleobase discovered in S-L2 cyanophage in 1977 by Kirnos et al [1]. Previous studies showed that DAP can form three hydrogen bonds Watson-crick base-pairing with thymidine in DNA [2, 3] and uridine in RNA [4, 5] resulting on increasing duplex stability by approximately 1-2 deg/modification. However, for DAP:2-thiothymidine [6, 7] or DAP:2-thiouridine [8] Tm duplex is decreased because of the steric hindrance between N2-nitrogen of DAP and sulfur of the pyrimidine base. NMR and circular dichroism studies showed the A form [9, 10] of DNA:RNA duplex containing DAP and transition of B to A form in high salt conditions in case of DNA:DNA duplex [11, 12]. In order to understand the influence of chemical modification on duplex incorporating DAP, a series of modified oligonucleotides were investigated including: sugar modifications (2′OMe [13-15], 2′-O-allyl [16] or alkyne [17], anhydrohexitol [18, 19], TNA [20], LNA [15, 21, 22], UNA [23], dimer double headed [24]), PO linkage (2′-5′[25-27], N3′-P5′ [28, 29]) and, backbone nature (PNA [30], SNA [8, 31]). Historically, DAP building blocks were obtained from guanosine analogue but usually with a low yield [2, 3, 32-35]. Later, other synthetic routes were developed using halogenated purine such as the 6-chloro-2-aminopurine [13, 15, 16, 18, 19, 22, 36] or 2,6-dicholoropurine [37-39] analogues or the commercially available 2,6-diaminopurine [31]. Nevertheless, these synthesis strategies still involve tedious chemical synthesis steps to introduce diamino function, placement of protecting groups and deprotection and purification. As the deprotection efficiency is not always perfect, the purification of the oligonucleotide is also an issue. Following an earlier communication, herein, we describe a synthetic route for easy incorporation of DAP in siRNAs using a post-synthetic strategy. The biophysical studies and RNA silencing activity are also discussed. In the same post-synthetic treatment, if an amine functionality is used, it leads to a conjugate at the 2-position of adenine, still leaving an RN—H for hydrogen bonding.

Currently used protecting groups for synthesising DAP comprising oligonucleotides are shown in Table 1.

TABLE 1
Protecting groups used in the art for DAP.

| | | |
|---|---|---|
| | Deprotection | |
| Structure | condition | References |

Hetero protecting groups

| | | |
|---|---|---|
| 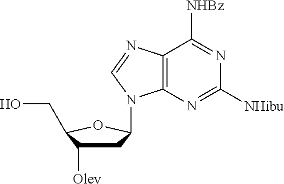 | Ammonia 65° C. 5-7 days | [3] |
| 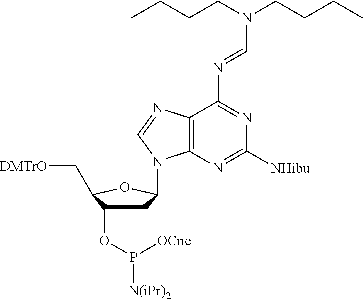 | | [40] |
| 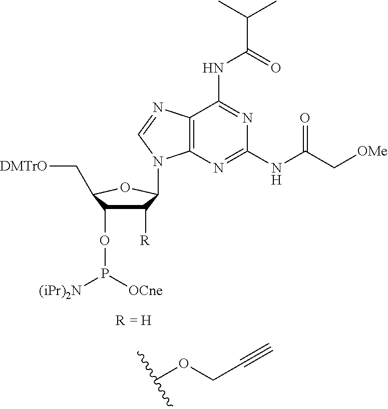 | Ammonia and methylamine (AMA) 60° C., 1 h i-butyryl difficult to remove | [17] |
| 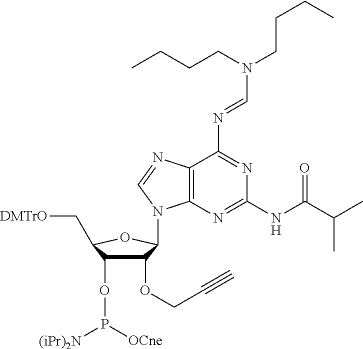 | | |
| 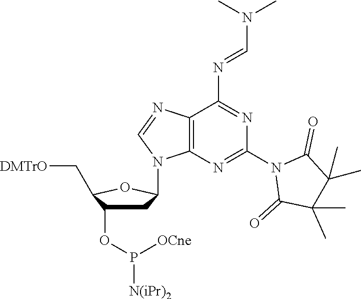 | Ammonia | [41] |

Homo protecting groups

| | | |
|---|---|---|
| 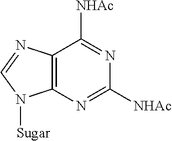 | Ammonia 55° C., 18 h | Ribose phosphate triester [10] LNA amidite [15] |
| 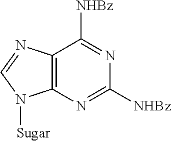 | AMA 70° C., 30 min to 18 h Ammonia 55° C., 4-6 h 24% hydrazine 4° C., 18 h | Ribose amidite [4] Deoxy amidite [25-27] HNA amidite [18, 19] TNA amidite [20] LNA amidite [21, 22] UNA amidite [23] Double headed nucleotide [24] |
| 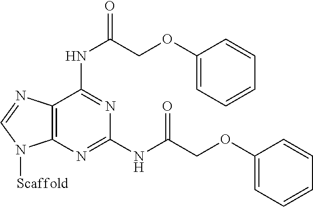 | Ammonia 60° C., 6-16 h | Ribo 2′OMe amidite [13, 14, 16, 42] Deoxy amidite [5, 6, 28, 32, 43, 44] Ribo amidite [29] PNA [30] |
| 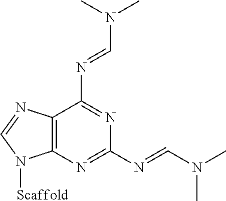 | Ammonia 55° C., 4-6 h | Deoxy amidite [44] SNA [31] |
| 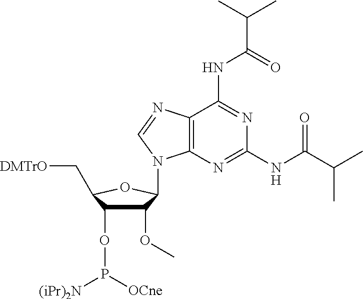 | Ammonia 55° C., 18 h | [15] |
| 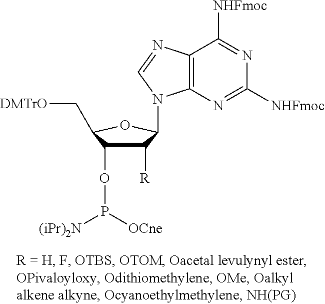 | Secondary or tertiary amine Room temperature | [45] |

### Synthesis of Required Monomers to Avoid the Need for Protecting Groups:

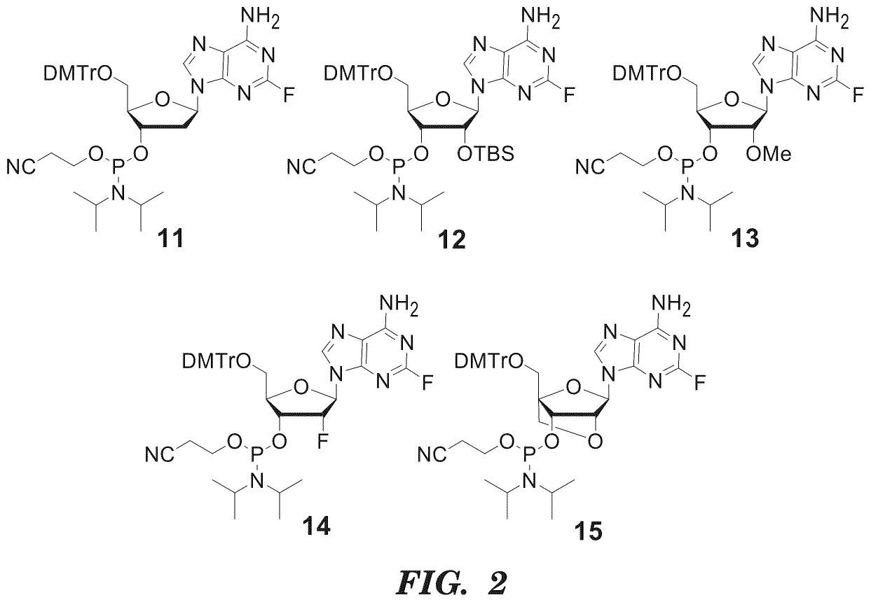

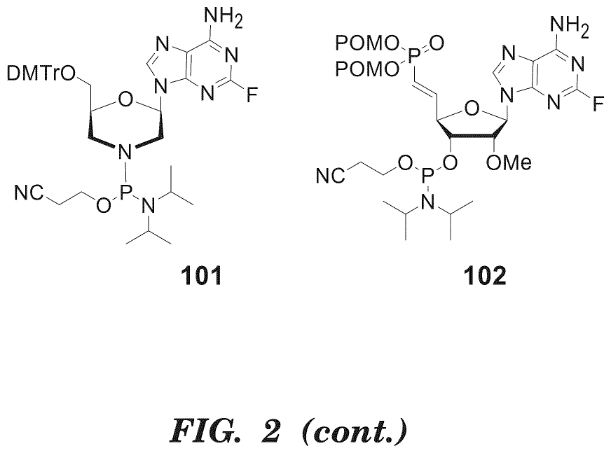

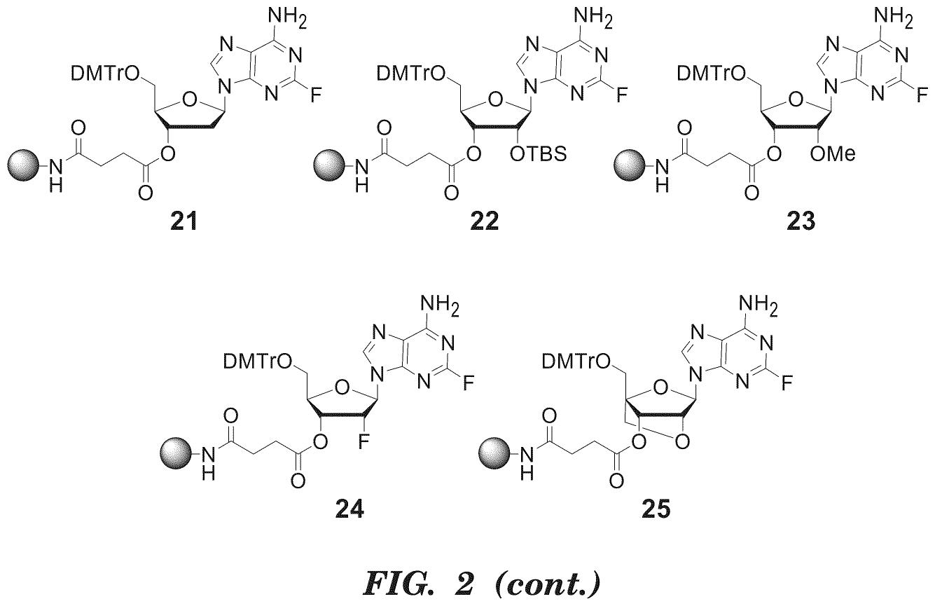

Following known methods reported in the literature, Exemplary monomers shown in FIG. 2 were synthesized.

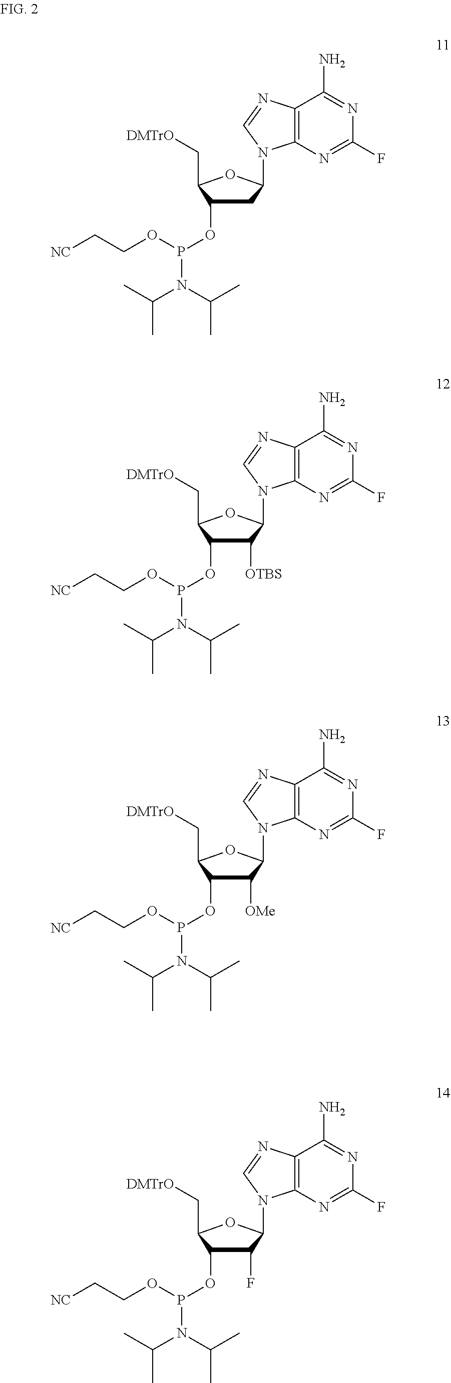

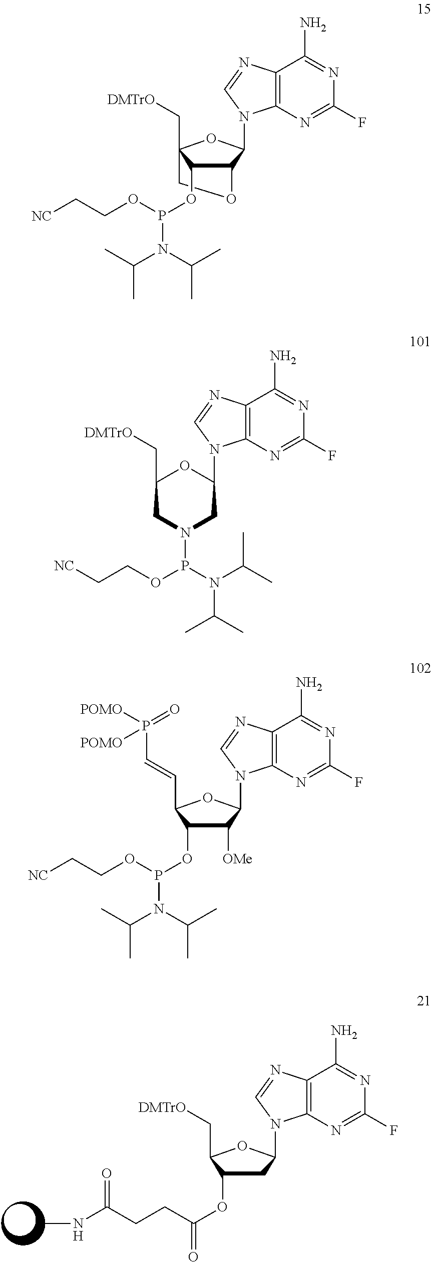

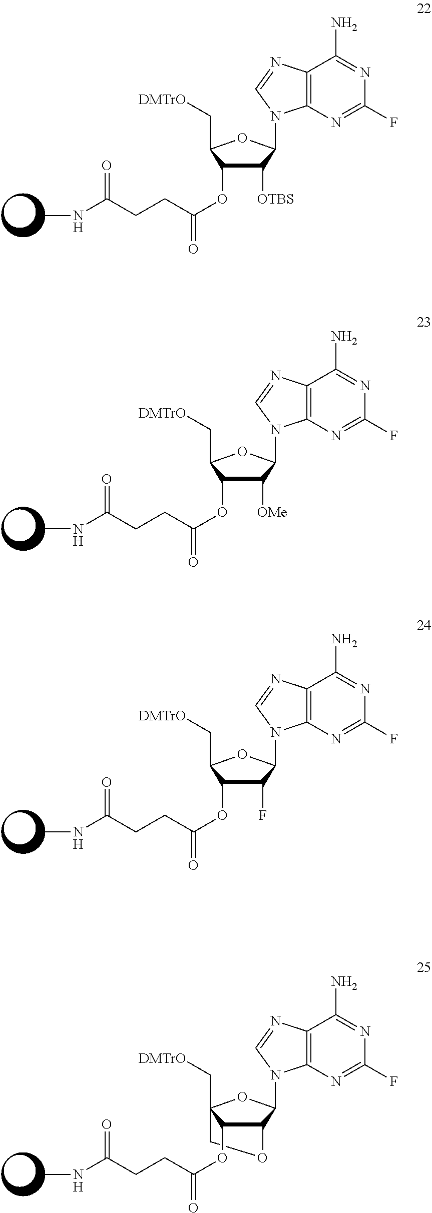

Using the monomers shown in FIG. 2, oligonucleotides comprising the DAP monomers residues show in FIG. 3.

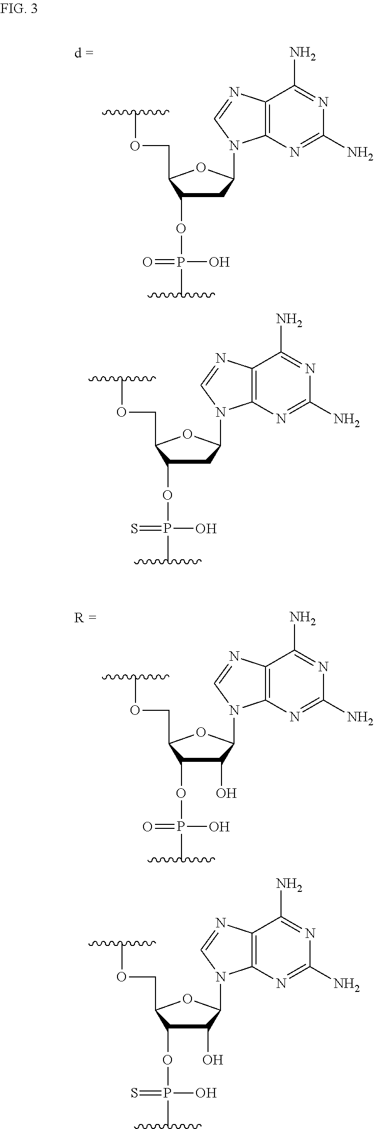

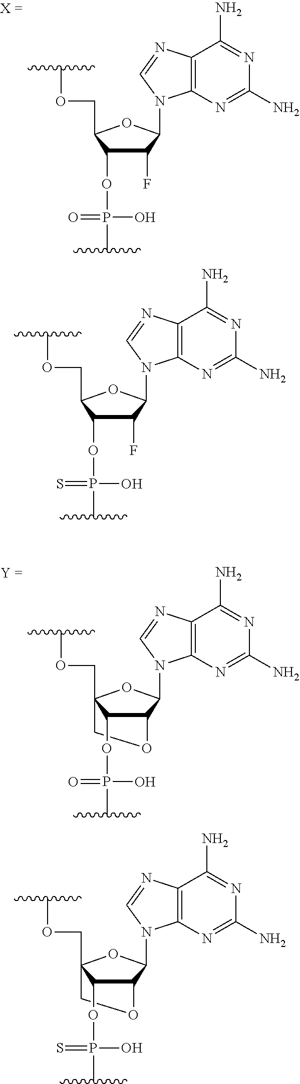

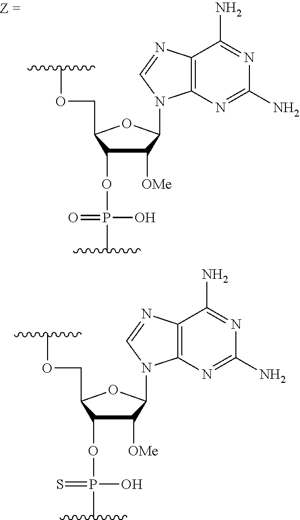

Sequences and mass spectroscopy analysis of the prepared oligonucleotides comprising the exemplary DAP monomers of FIG. 3 are shown in Table 2.

TABLE 2
Sequences and mass spectroscopy characterization of siRNA strandsª

| | | | | Mass (m/z) | |
|---|---|---|---|---|---|
| | Target | Strand | Sequence 5′-3′ | Calc. | Obs. |
| 1 | mTTR | AS Parent | u•U•aUaGaGcAagaAcAcUgUu•u•u (SEQ ID | 7595.94 | |
| | | A-117800 | NO: 37) | | |
| 2 | mTTR | A-3144501 | u•U•aUaGaGcAagaAcdcUgUu•u•u (SEQ ID | 7592.94 | 7591.41 |
| | | | NO: 38) | | |
| 3 | mTTR | A-3144502 | u•U•aUaGaGcAagadcAcUgUu•u•u (SEQ ID | 7592.94 | 7591.66 |
| | | | NO: 39) | | |
| 4 | mTTR | A-3144503 | u•U•aUaGaGcdagaAcAcUgUu•u•u (SEQ ID | 7592.94 | 7591.50 |
| | | | NO: 40) | | |
| 5 | mTTR | A-3144504 | u•U•aUaGaGcdagaAcdcUgUu•u•u (SEQ ID | 7588.98 | 7588.97 |
| | | | NO: 41) | | |
| 6 | mTTR | A-3144505 | u•U•aUaGaGcdagadcdcUgUu•u•u (SEQ ID | 7587.00 | 7585.87 |
| | | | NO: 42) | | |
| 7 | mTTR | A-3116238 | u•U•aUaGaGcAagaAcXcUgUu•u•u (SEQ ID | 7610.95 | 7609.54 |
| | | | NO: 43) | | |
| 8 | mTTR | A-3116239 | u•U•aUaGaGcAagaXcAcUgUu•u•u (SEQ ID | 7610.95 | 7609.62 |
| | | | NO: 44) | | |
| 9 | mTTR | A-3116240 | u•U•aUaGaGcXagaAcAcUgUu•u•u (SEQ ID | 7610.95 | 7609.75 |
| | | | NO: 45) | | |
| 10 | mTTR | A-3116244 | u•U•aUaGaGcXagaAcXcUgUu•u•u (SEQ ID | 7625.96 | 7624.71 |
| | | | NO: 46) | | |
| 11 | mTTR | A-3116242 | u•U•aUaGaGcXagaXcXcUgUu•u•u (SEQ ID NO: 47) | 7640.98 | 7639.72 |
| 12 | mTTR | A-3144493 | u•U•aUaGaGcAagaAcYcUgUu•u•u (SEQ ID | 7620.97 | 7619.54 |
| | | | NO: 48) | | |
| 13 | mTTR | A-3144494 | u•U•aUaGaGcAagaYcAcUgUu•u•u (SEQ ID | 7620.97 | 7619.70 |
| | | | NO: 49) | | |
| 14 | mTTR | A-3144495 | u•U•aUaGaGcYagaAcAcUgUu•u•u (SEQ ID | 7620.97 | 7619.73 |
| | | | NO: 50) | | |
| 15 | mTTR | A-3144496 | u•U•aUaGaGcYagaAcYcUgUu•u•u (SEQ ID | 7646.00 | 7644.77 |
| | | | NO: 51) | | |
| 16 | mTTR | A-3144497 | u•U•aUaGaGcYagaYcYcUgUu•u•u (SEQ ID | 7671.04 | 7669.57 |
| | | | NO: 52) | | |
| 17 | mTTR | A-3146178 | u•U•aUaGaGcAagRAcAcUgUu•u•u (SEQ ID | 7596.92 | |
| | | | NO: 53) | | |
| 18 | mTTR | A-3146179 | u•U•aUaGaGcARgaAcAcUgUu•u•u (SEQ ID | 7596.92 | |
| | | | NO: 54) | | |
| 19 | mTTR | A-3146180 | u•U•aUaGRGcAagaAcAcUgUu•u•u (SEQ ID | 7596.92 | |
| | | | NO: 55) | | |
| 20 | mTTR | A-3146181 | u•U•aURGaGcAagaAcAcUgUu•u•u (SEQ ID | 7596.92 | |
| | | | NO: 56) | | |
| 21 | mTTR | A-3146182 | u•U•RUaGaGcAagaAcAcUgUu•u•u (SEQ ID | 7596.92 | |
| | | | NO: 57) | | |
| 22 | mTTR | A-3146183 | u•U•RURGRGcARgRAcAcUgUu•u•u (SEQ ID | 7600.87 | |
| | | | NO: 58) | | |
| 23 | mTTR | A-3144509 | u•U•aUaGaGcAagZAcAcUgUu•u•u (SEQ ID | 7610.95 | 7609.86 |
| | | | NO: 59) | | |
| 24 | mTTR | A-3144510 | u•U•aUaGaGcAZgaAcAcUgUu•u•u (SEQ ID | 7610.95 | 7609.94 |
| | | | NO: 60) | | |
| 25 | mTTR | A-3144511 | u•U•aUaGZGcAagaAcAcUgUu•u•u (SEQ ID | 7610.95 | 7609.79 |
| | | | NO: 61) | | |
| 26 | mTTR | A-3144512 | u•U•aUZGaGcAagaAcAcUgUu•u•u (SEQ ID | 7610.95 | 7609.80 |
| | | | NO: 62) | | |
| 27 | mTTR | A-3144513 | u•U•ZUaGaGcAagaAcAcUgUu•u•u (SEQ ID | 7610.95 | 7609.78 |
| | | | NO: 63) | | |
| 28 | mTTR | A-3144514 | u•U•ZUZGZGcAZgZAcAcUgUu•u•u (SEQ ID | 7671.02 | 7669.86 |
| | | | NO: 64) | | |
| 29 | mTTR | A- | u•U•ZUZGYGcXZgZXcXcUgUu•u•u (SEQ ID | 7714.04 | 7712.78 |
| | | | NO: 65) | | |
| 30 | mTTR | A- | u•U•RUZGYGcdZgZXcXcUgUu•u•u (SEQ ID | | |
| | | | NO: 66) | | |
| 31 | mTTR | S Parent | A•a•CaGuGuUCUuGcUcUaUaA(L) (SEQ ID | 8590.17 | |
| | | A-117799 | NO: 67) | | |
| 32 | mTTR | A-3144506 | A•a•CaGuGuUCUuGcUcUaUad (SEQ ID NO: 68) | 6798.23 | 6797.12 |
| 33 | mTTR | A-3144507 | d•a•CaGuGuUCUuGcUcUaUaA (SEQ ID NO: 69) | 6798.23 | 6796.95 |
| 34 | mTTR | A-3144508 | d•a•CaGuGuUCUuGcUcUaUad (SEQ ID NO: 70) | 6795.25 | 6794.25 |
| 35 | mTTR | A-3116243 | A•a•CaGuGuUCUuGcUcUaUaX (SEQ ID NO: 71) | 6816.22 | 6814.94 |
| 36 | mTTR | A-3116245 | X•a•CaGuGuUCUuGcUcUaUaA (SEQ ID NO: 72) | 6816.22 | 6814.64 |
| 37 | mTTR | A-3116246 | X•a•CaGuGuUCUuGcUcUaUaX (SEQ ID NO: 73) | 6831.23 | 6829.91 |
| 38 | mTTR | A-3144498 | A•a•CaGuGuUCUuGcUcUaUaY (SEQ ID NO: 74) | 6829.22 | 6824.95 |
| 39 | mTTR | A-3144499 | Y•a•CaGuGuUCUuGcUcUaUaA (SEQ ID NO: 75) | 6826.23 | 6824.85 |
| 40 | mTTR | A-3144500 | Y•a•CaGuGuUCUuGcUcUaUaY (SEQ ID NO: 76) | 6851.27 | 6866.08 |
| 41 | mTTR | A-3144515 | A•a•CaGuGuUCUuGcUcUaUZA (SEQ ID NO: 77) | 6816.23 | 6815.08 |
| 42 | mTTR | A-3144516 | A•a•CaGuGuUCUuGcUcUZUaA (SEQ ID NO: 78) | 6816.23 | 6815.04 |
| 43 | mTTR | A-3144517 | A•a•CZGuGuUCUuGcUcUaUaA (SEQ ID NO: 79) | 6816.23 | 6815.03 |
| 44 | mTTR | A-3144518 | A•Z•CaGuGuUCUuGcUcUaUaA (SEQ ID NO: 80) | 6816.23 | 6815.03 |
| 45 | mTTR | A-3144519 | A•Z•CZGuGuUCUuGcUcUZUZA (SEQ ID | 6861.27 | 6860.15 |
| | | | NO: 81) | | |
| 46 | mTTR | A-3146184 | A•a•CaGuGuUCUuGcUcUaURA (SEQ ID NO: 82) | 6802.2 | |
| 47 | mTTR | A-3146185 | A•a•CaGuGuUCUuGcUcURUaA (SEQ ID NO: 83) | 6802.2 | |
| 48 | mTTR | A-3146186 | A•a•CRGuGuUCUuGcUcUaUaA (SEQ ID NO: 84) | 6802.2 | |
| 49 | mTTR | A-3146187 | A•R•CaGuGuUCUuGcUcUaUaA (SEQ ID NO: 85) | 6802.19 | |
| 50 | mTTR | A-3146188 | A•R•CRGuGuUCUuGcUcURURA (SEQ ID | 6805.15 | |
| | | | NO: 86) | | |
| 51 | mTTR | A- | Y•Z•CZGuGuUCUuGcUcUZUZd(L) (SEQ ID | 8672.28 | |
| | | | NO: 87) | | |
| 52 | Coagulation | S Parent | u•c•a•u•c•u•c•a•A•G•T•C•u•T•aa(L) | 7227.27 | |
| | factor 7 | A- | (SEQ ID NO: 88) | | |
| 53 | Coagulation | A- | u•c•Y•u•c•u•c•Y•Y•G•T•C•u•T•Yd(L) | 7280.21 | |
| | factor 7 | | (SEQ ID NO: 89) | | |
| 54 | Coagulation | A- | u•c•Z•u•c•u•c•Z•Y•G•T•C•u•T•Zd(L) | 7276.25 | |
| | factor 7 | | (SEQ ID NO: 90) | | |
| 55 | AntagomiR- | S Parent | a·c·aaacaccauugucacacu•c•c•a•(L) (SEQ ID | 9425.35 | |
| | 122 | A- | NO: 91) | | |
| 56 | AntagomiR- | A- | Y•c•YYYcYccYuugucZcYcu•c•c•Y•(L) (SEQ | 9544.34 | |
| | 122 | | ID NO: 92) | | |

Italicized uppercase, lowercase, and uppercase red bold letters represent 2′-F-RNA, 2′-OMe sugar modifications and 2,6-diaminopurine nucleoside, respectively, to Adenosine (A), Cytidine (C), Guanosine (G), Uridine (U), deoxy-2,6-diaminopurine (d), ribo-2,6-diaminopurine (R), 2′-F-2,6-diaminopurine (X), LNA-2,6-diaminopurine (Y) and 2′OMe-2,6-diaminopurine (Z). Phosphorothioate linkages are indicated by the “•” symbol.

Double-stranded RNA molecules, i.e., siRNA duplex comprising the exemplary DAP monomer residues FIG. 2 and their biophysical studies are summarized in Table 3.

TABLE 3
siRNA duplexes and biophysical studies

| | | Tm | |
|---|---|---|---|
| Duplexes | S/AS duplex 5′-3′ | (° C.) | CD |
| Control | A•a•CaGuGuUCUuGcUcUaUaA(L) | | |
| AD-57727 | u•U•aUaGaGcAagaAcAcUgUu•u•u | | |
| | (SEQ ID NO: 93) | | |
| AD- | A•a•CaGuGuUCUuGcUcUaUaA(L) | | |
| | u•U•aUaGaGcAagaAcdcUgUu•u•u | | |
| | (SEQ ID NO: 94) | | |
| AD- | A•a•CaGuGuUCUuGcUcUaUaA(L) | | |
| | u•U•aUaGaGcAagadcAcUgUu•u•u | | |
| | (SEQ ID NO: 95) | | |
| AD- | A•a•CaGuGuUCUuGcUcUaUaA(L) | | |
| | u•U•aUaGaGcdagaAcAcUgUu•u•u | | |
| | (SEQ ID NO: 96) | | |
| AD- | A•a•CaGuGuUCUuGcUcUaUaA(L) | | |
| | u•U•aUaGaGcdagaAcdcUgUu•u•u | | |
| | (SEQ ID NO: 97) | | |
| AD- | A•a•CaGuGuUCUuGcUcUaUaA(L) | | |
| | u•U•aUaGaGcdagadcdcUgUu•u•u | | |
| | (SEQ ID NO: 98) | | |
| AD- | A•a•CaGuGuUCUuGcUcUaUaA(L) | | |
| | u•U•aUaGaGcAagaAcXcUgUu•u•u | | |
| | (SEQ ID NO: 99) | | |
| AD- | A•a•CaGuGuUCUuGcUcUaUaA(L) | | |
| | u•U•aUaGaGcAagaXcAcUgUu•u•u | | |
| | (SEQ ID NO: 100) | | |
| AD- | A•a•CaGuGuUCUuGcUcUaUaA(L) | | |
| | u•U•aUaGaGcXagaAcAcUgUu•u•u | | |
| | (SEQ ID NO: 101) | | |
| AD- | A•a•CaGuGuUCUuGcUcUaUaA(L) | | |
| | u•U•aUaGaGcXagaAcXcUgUu•u•u | | |
| | (SEQ ID NO: 102) | | |
| AD- | A•a•CaGuGuUCUuGcUcUaUaA(L) | | |
| | u•U•aUaGaGcXagaXcXcUgUu•u•u | | |
| | (SEQ ID NO: 103) | | |
| AD- | A•a•CaGuGuUCUuGcUcUaUaA(L) | | |
| | u•U•aUaGaGcAagaAcYcUgUu•u•u | | |
| | (SEQ ID NO: 104) | | |
| AD- | A•a•CaGuGuUCUuGcUcUaUaA(L) | | |
| | u•U•aUaGaGcAagaYcAcUgUu•u•u | | |
| | (SEQ ID NO: 105) | | |
| AD- | A•a•CaGuGuUCUuGcUcUaUaA(L) | | |
| | u•U•aUaGaGcYagaAcAcUgUu•u•u | | |
| | (SEQ ID NO: 106) | | |
| AD- | A•a•CaGuGuUCUuGcUcUaUaA(L) | | |
| | u•U•aUaGaGcYagaAcYcUgUu•u•u | | |
| | (SEQ ID NO: 107) | | |
| AD- | A•a•CaGuGuUCUuGcUcUaUaA(L) | | |
| | u•U•aUaGaGcYagaYcYcUgUu•u•u | | |
| | (SEQ ID NO: 108) | | |
| AD- | A•a•CaGuGuUCUuGcUcUaUaA(L) | | |
| | u•U•aUaGaGcAagRAcAcUgUu•u•u | | |
| | (SEQ ID NO: 109) | | |
| AD- | A•a•CaGuGuUCUuGcUcUaUaA(L) | | |
| | u•U•aUaGaGcARgaAcAcUgUu•u•u | | |
| | (SEQ ID NO: 110) | | |
| AD- | A•a•CaGuGuUCUuGcUcUaUaA(L) | | |
| | u•U•aUaGRGcAagaAcAcUgUu•u•u | | |
| | (SEQ ID NO: 111) | | |
| AD- | A•a•CaGuGuUCUuGcUcUaUaA(L) | | |
| | u•U•aURGaGcAagaAcAcUgUu•u•u | | |
| | (SEQ ID NO: 112) | | |
| AD- | A•a•CaGuGuUCUuGcUcUaUaA(L) | | |
| | u•U•RUaGaGcAagaAcAcUgUu•u•u | | |
| | (SEQ ID NO: 113) | | |
| AD- | A•a•CaGuGuUCUuGcUcUaUaA(L) | | |
| | u•U•RURGRGCARgRACAcUgUu•u•u | | |
| | (SEQ ID NO: 114) | | |
| AD- | A•a•CaGuGuUCUuGcUcUaUaA(L) | | |
| | u•U•aUaGaGcAagZAcAcUgUu•u•u | | |
| | (SEQ ID NO: 115) | | |
| AD- | A•a•CaGuGuUCUuGcUcUaUaA(L) | | |
| | u•U•aUaGaGcAZgaAcAcUgUu•u•u | | |
| | (SEQ ID NO: 116) | | |
| AD- | A•a•CaGuGuUCUuGcUcUaUaA(L) | | |
| | u•U•aUaGZGcAagaAcAcUgUu•u•u | | |
| | (SEQ ID NO: 117) | | |
| AD- | A•a•CaGuGuUCUuGcUcUaUaA(L) | | |
| | u•U•aUZGaGcAagaAcAcUgUu•u•u | | |
| | (SEQ ID NO: 118) | | |
| AD- | A•a•CaGuGuUCUuGcUcUaUaA(L) | | |
| | u•U•ZUaGaGcAagaAcAcUgUu•u•u | | |
| | (SEQ ID NO: 119) | | |
| AD- | A•a•CaGuGuUCUuGcUcUaUaA(L) | | |
| | u•U•ZUZGZGcAZgZAcAcUgUu•u•u | | |
| | (SEQ ID NO: 120) | | |
| AD- | A•a•CaGuGuUCUuGcUcUaUaA(L) | | |
| | u•U•ZUZGYGcXZgZXcXcUgUu•u•u | | |
| | (SEQ ID NO: 121) | | |
| AD- | A•a•CaGuGuUCUuGcUcUaUaA(L) | | |
| | u•U•RUZGYGcdZgZXcXcUgUu•u•u | | |
| | (SEQ ID NO: 122) | | |
| AD- | A•a•CaGuGuUCUuGcUcUaUad | | |
| | u•U•aUaGaGcAagaAcAcUgUu•u•u | | |
| | (SEQ ID NO: 123) | | |
| AD- | d•a•CaGuGuUCUuGcUcUaUaA | | |
| | u•U•aUaGaGcAagaAcAcUgUu•u•u | | |
| | (SEQ ID NO: 124) | | |
| AD- | d•a•CaGuGuUCUuGcUcUaUad | | |
| | u•U•aUaGaGcAagaAcAcUgUu•u•u | | |
| | (SEQ ID NO: 125) | | |
| AD- | A•a•CaGuGuUCUuGcUcUaUaX | | |
| | u•U•aUaGaGcAagaAcAcUgUu•u•u | | |
| | (SEQ ID NO: 126) | | |
| AD- | X•a•CaGuGuUCUuGcUcUaUaA | | |
| | u•U•aUaGaGcAagaAcAcUgUu•u•u | | |
| | (SEQ ID NO: 127) | | |
| AD- | X•a•CaGuGuUCUuGcUcUaUaX | | |
| | u•U•aUaGaGcAagaAcAcUgUu•u•u | | |
| | (SEQ ID NO: 128) | | |
| AD- | A•a•CaGuGuUCUuGcUcUaUaY | | |
| | u•U•aUaGaGcAagaAcAcUgUu•u•u | | |
| | (SEQ ID NO: 129) | | |
| AD- | Y•a•CaGuGuUCUuGcUcUaUaA | | |
| | u•U•aUaGaGcAagaAcAcUgUu•u•u | | |
| | (SEQ ID NO: 130) | | |
| AD- | Y•a•CaGuGuUCUuGcUcUaUaY | | |
| | u•U•aUaGaGcAagaAcAcUgUu•u•u | | |
| | (SEQ ID NO: 131) | | |
| AD- | A•a•CaGuGuUCUuGcUcUaURA | | |
| | u•U•aUaGaGcAagaAcAcUgUu•u•u | | |
| | (SEQ ID NO: 132) | | |
| AD- | A.a.CaGuGuUCUuGcUcURUaA | | |
| | u•U•aUaGaGcAagaAcAcUgUu•u•u | | |
| | (SEQ ID NO: 133) | | |
| AD- | A•a•CRGuGuUCUuGcUcUaUaA | | |
| | u•U•aUaGaGcAagaAcAcUgUu•u•u | | |
| | (SEQ ID NO: 134) | | |
| AD- | A•R•CaGuGuUCUuGcUcUaUaA | | |
| | u•U•aUaGaGcAagaAcAcUgUu•u•u | | |
| | (SEQ ID NO: 135) | | |
| AD- | A•R•CRGuGuUCUuGcUcURURA | | |
| | u•U•aUaGaGcAagaAcAcUgUu•u•u | | |
| | (SEQ ID NO: 136) | | |
| AD- | A•a•CaGuGuUCUuGcUcUaUZA | | |
| | u•U•aUaGaGcAagaAcAcUgUu•u•u | | |
| | (SEQ ID NO: 137) | | |
| AD- | A•a•CaGuGuUCUuGcUcUZUaA | | |
| | u•U•aUaGaGcAagaAcAcUgUu•u•u | | |
| | (SEQ ID NO: 138) | | |
| AD- | A•a•CZGuGuUCUuGcUcUaUaA | | |
| | u•U•aUaGaGcAagaAcAcUgUu•u•u | | |
| | (SEQ ID NO: 139) | | |
| AD- | A•Z•CaGuGuUCUuGcUcUaUaA | | |
| | u•U•aUaGaGcAagaAcAcUgUu•u•u | | |
| | (SEQ ID NO: 140) | | |
| AD- | A•Z•CZGuGuUCUuGcUcUZUZA | | |
| | u•U•aUaGaGcAagaAcAcUgUu•u•u | | |
| | (SEQ ID NO: 141) | | |

Italicized uppercase, lowercase, and uppercase red bold letters represent 2′-F-RNA, 2′-OMe sugar modifications and 2,6-diaminopurine nucleoside, respectively, to Adenosine (A), Cytidine (C), Guanosine (G), Uridine (U), deoxy-2,6-diaminopurine (d), ribo-2,6-diaminopurine (R), 2′-F-2,6-diaminopurine (X), LNA-2,6-diaminopurine (Y) and 2′OMe-2,6-diaminopurine (Z). Phosphorothioate linkages are indicated by the “•” symbol.

Exemplary dsRNAs comprising the DAP monomer residues of FIG. 2 for RNAi studies are shown in Table 4.

TABLE 4
Duplexes for RNAi silencing study

| Target | Duplexes | S/AS duplex 5′-3′ |
|---|---|---|
| mTTR | Control | A•a•CaGuGuUCUuGcUcUaUaA(L) |

| | | |
|---|---|---|
| | AD-57727 | u•U•aUaGaGcAagaAcAcUgUu•u•u |
| | | (SEQ ID NO: 142) |
| mTTR | AD- | Y•Z•CZGuGuUCUuGcUcUZUZd(L) |
| | | u•U•aUaGaGcAagaAcAcUgUu•u•u |
| | | (SEQ ID NO: 143) |

Italicized uppercase, lowercase, uppercase, uppercase red bold letters and lowercase bold letters represent 2′-F-RNA, 2′-OMe, LNA sugar modifications, 2,6-diaminopurine and deoxy nucleoside, respectively, to Adenosine (A), Cytidine (C), Guanosine (G), Uridine (U), deoxy-2,6-diaminopurine (d), ribo-2,6-diaminopurine (R), 2′-F-2,6-diaminopurine (X), LNA-2,6-diaminopurine (Y) and 2′OMe-2,6-diaminopurine (Z). (L) represents the tri-N-acetylgalactosamine (tri-GalNAc) ligand. Phosphorothioate linkages are indicated by the “•” symbol

Exemplary single-stranded Antagomirs and Reversirs comprising the DAP monomer residues of FIG. 2 are shown in Table 5.

TABLE 5
Oligonucleotide with single stranded Antagomir and Reversir

| Target | Duplexes | S/AS duplex 5′-3′ |
|---|---|---|
| Coagulation | Control | u•c•a•u•c•u•c•a•A•G•T•C•u•T•aa(L) |
| factor VII | A- | (SEQ ID NO: 144) |
| Coagulation | A- | u•c•Y•u•c•u•c•Y•Y•G•T•C•u•T•Yd(L) |
| factor VII | | (SEQ ID NO: 145) |
| Coagulation | A- | u•c•Z•u•c•u•c•Z•Y•G•T•C•u•T•Zd(L) |
| factor VII | | (SEQ ID NO: 146) |
| AntogomiR- | Control | a•c•aaacaccauugucacacu•c•c•a•(L) |
| 122 | A- | (SEQ ID NO: 147) |
| AntagomiR- | A- | Y•c•YYYcYccYuugucacYcu•c•c•Y•(L) |
| 122 | | (SEQ ID NO: 148) |

Uppercase, lowercase, and uppercase red bold letters represent LNA, 2′-OMe sugar modifications and 2,6-diaminopurine nucleoside, respectively, to Adenosine (A), Cytidine (C), Guanosine (G), Uridine (U), deoxy-2,6-diaminopurine (d), ribo-2,6-diaminopurine (R), 2′-F-2,6-diaminopurine (X), LNA-2,6-diaminopurine (Y) and 2′OMe-2,6-diaminopurine (Z). Phosphorothioate linkages are indicated by the “.” symbol.

Sequences and mass spectroscopy characterization of exemplary oligonucleotides comprising the DAP monomer residues of FIG. 3 for Dikerson-Dodecamer NMR studies are shown in Table 5.

TABLE 5
Sequences and mass spectroscopy characterization of oligonucleotides
made for Dickerson-Dodecamer NMR study

| | | Mass (m/z) | |
|---|---|---|---|
| Strand | Sequence 5′-3′ | Calc. | Obs. |
| A-69532 | dCdGdCdGdAdAdTdTdCdGdCdG | 3646.393 | 3645.58 |
| | (SEQ ID NO: 149) | | |
| A-3154596 | dCdGdCdGddAdTdTdCdGdCdG | 3661.410 | 3660.62 |
| | (SEQ ID NO: 150) | | |
| A-3154597 | dCdGdCdGdAddTdTdCdGdCdG | 3661.410 | 3660.89 |
| | (SEQ ID NO: 151) | | |
| A-3154598 | dCdGdCdGdddTdTdCdGdCdG | 3676.428 | 3675.61 |
| | (SEQ ID NO: 152) | | |
| ON101 | dCdGdCdGd¹⁵dAdTdTdCdGdCdG | 3662.41 | 3661.61 |
| | (SEQ ID NO: 153) | | |
| ON102 | dCdGdCdGdAd¹⁵dTdTdCdGdCdG | 3662.41 | 3661.35 |
| | (SEQ ID NO: 154) | | |
| ON103 | dCdGdCdGd¹⁵d¹⁵dTdTdCdGdCdG | 3677.42 | 3677.56 |
| | (SEQ ID NO: 155) | | |

Deoxy-Adenosine (dA), deoxy-Cytidine (dC), deoxy-Guanosine (dG), deoxy-Thymidine (dT), deoxy-2,6-diaminopurine (d), deoxy-2,6-diaminopurine 2-¹⁵N labelling (d¹⁵)

### Post-Synthesis Conjugation

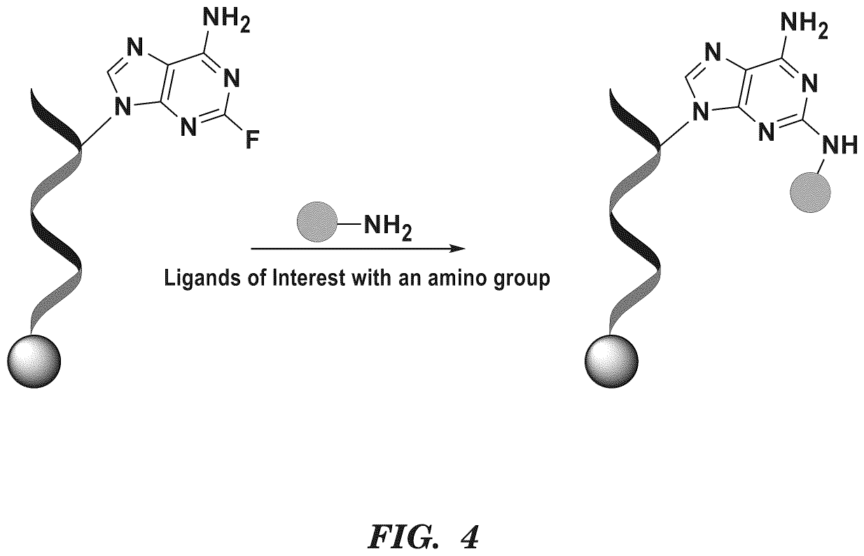

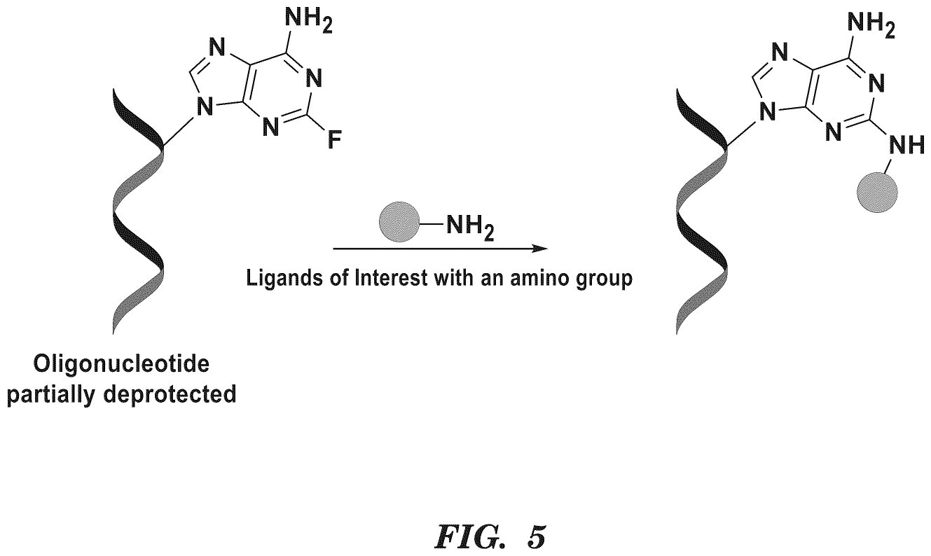

Exemplary post-synthetic conjugation process for post-synthesis conjugation with 2-F,6-NH2 nucleotides in solid support is shown in FIG. 4A and in solution phase in FIG. 5B.

Exemplary ligands used for post-synthetic conjugation include, but are not limited to, carbohydrates, peptides, lipids, diagnostic agents (Biotin), fluorescent dyes, PEGs, antibodies, antibody fragments (Nanobodies), folic acid, RGD-peptide and DUPA ligand.

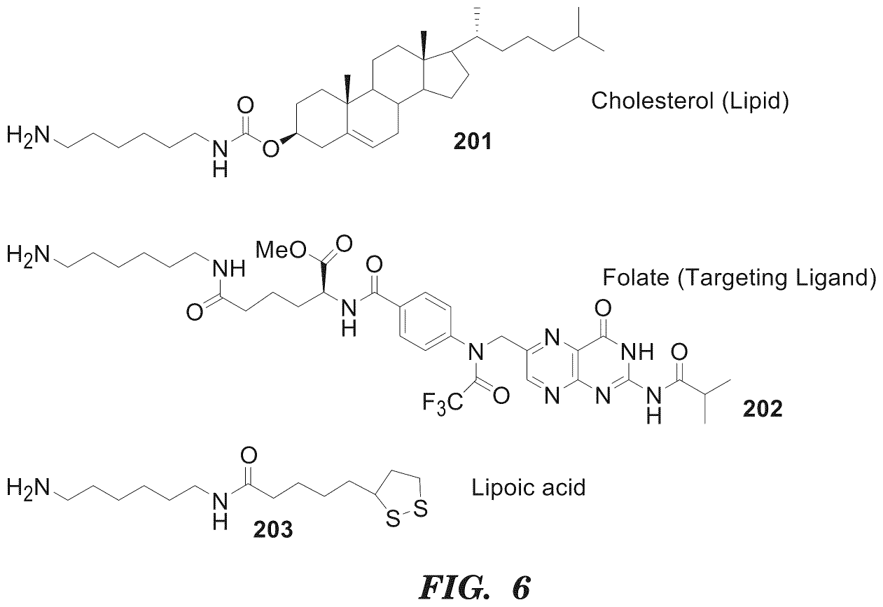

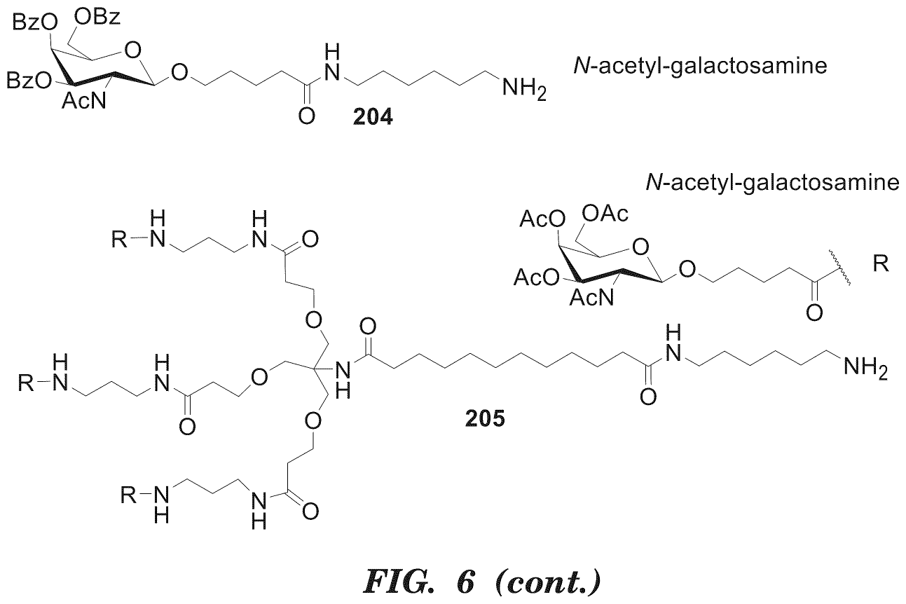

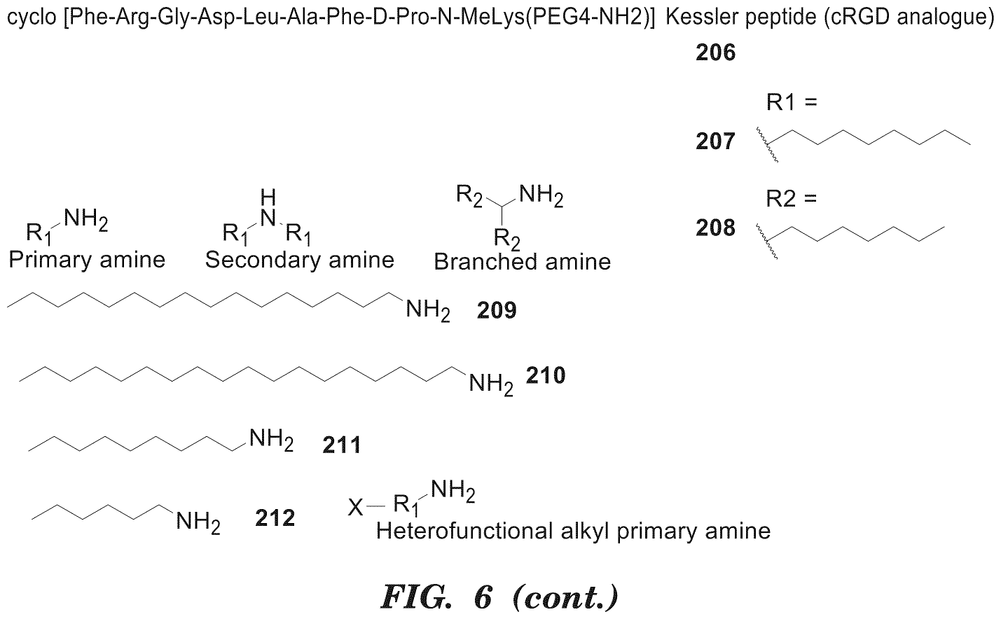

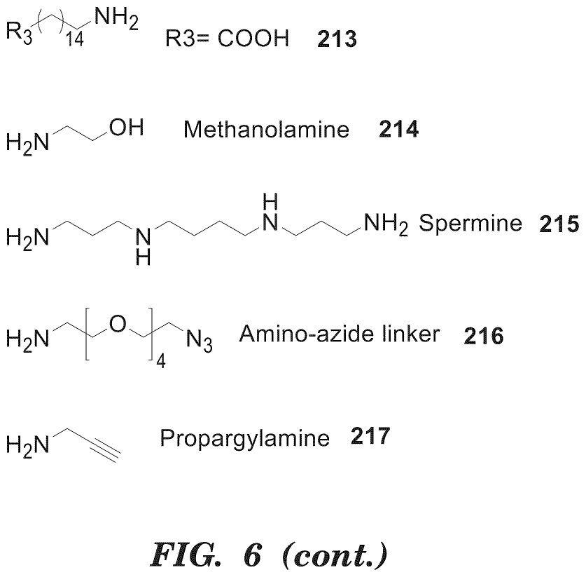

Exemplary ligands With amine linker used for the post-synthetic conjugation process are shown in FIG. 6.

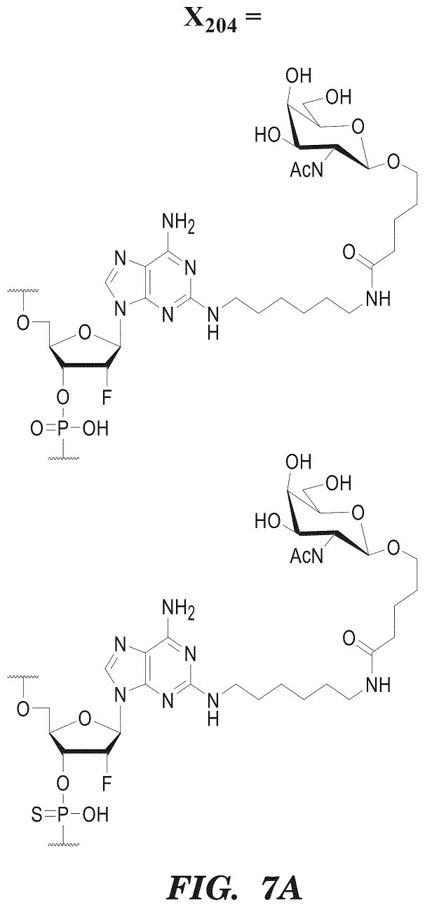

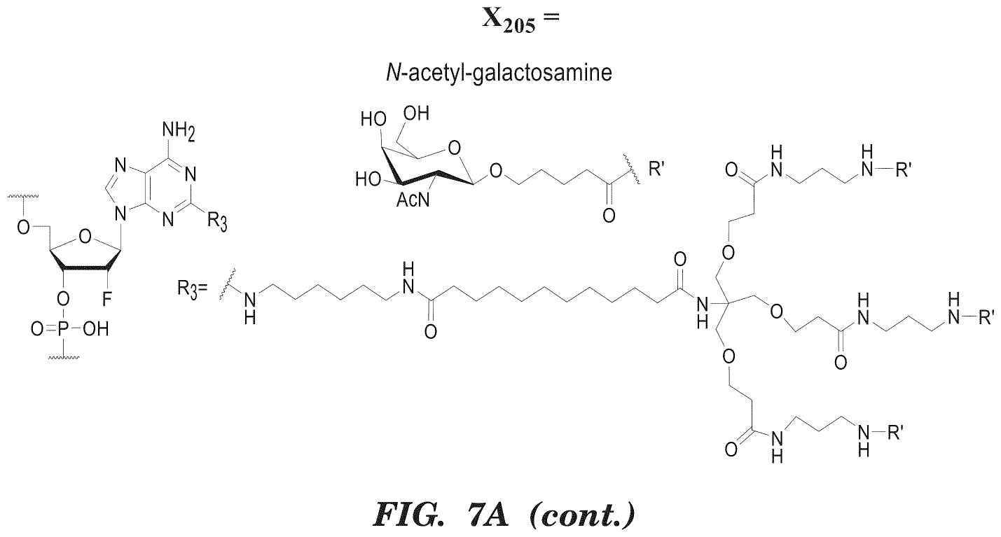

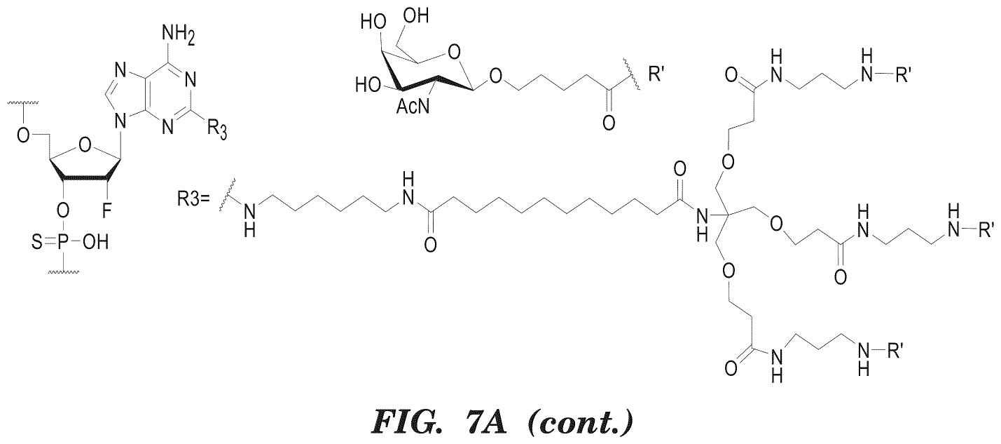

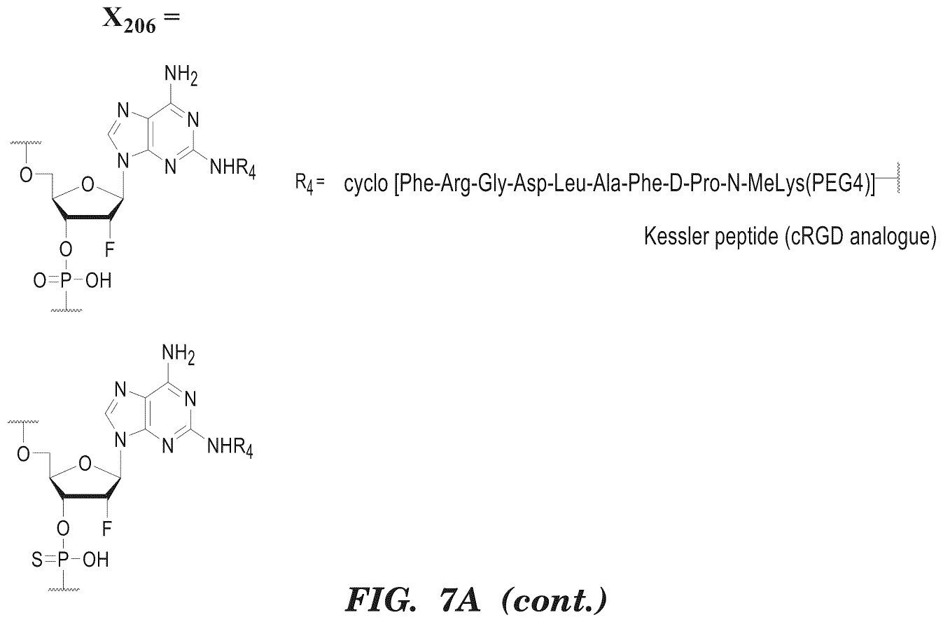

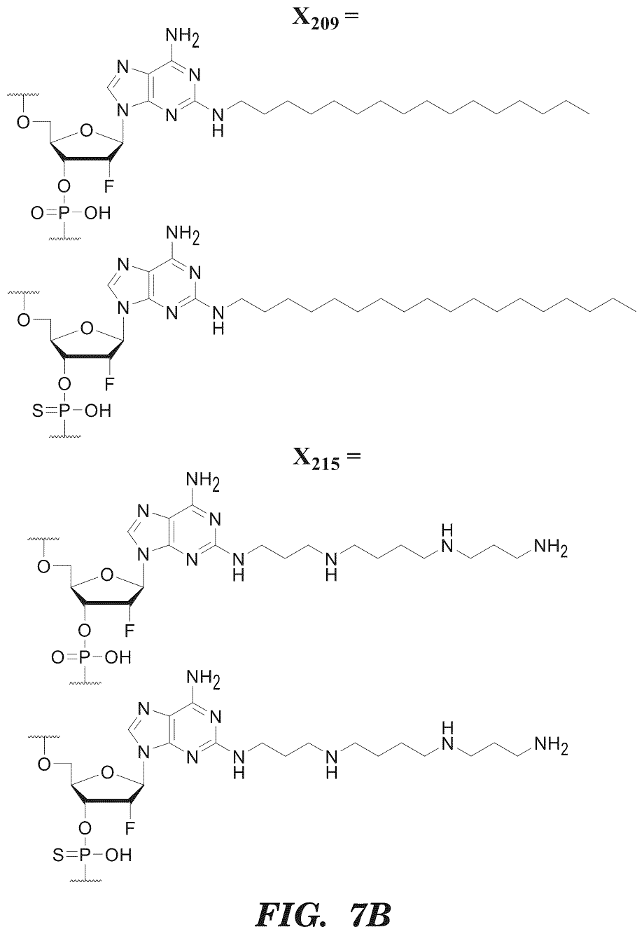

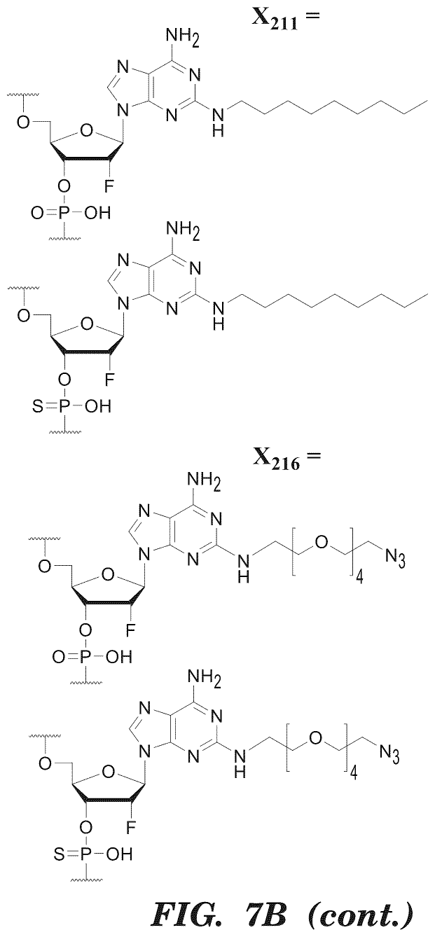

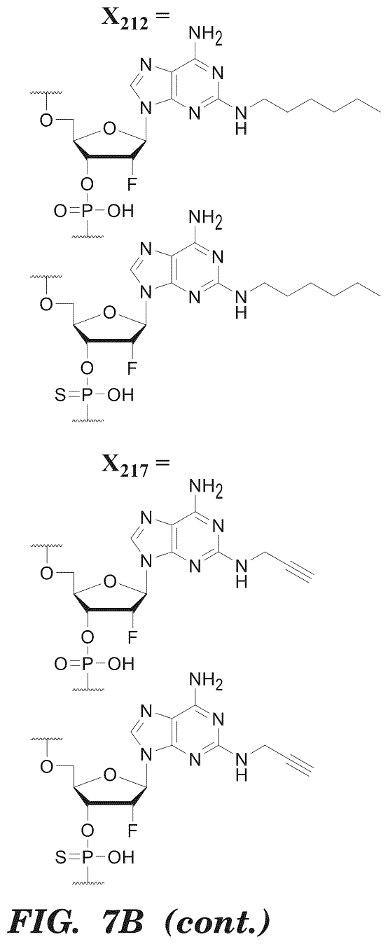

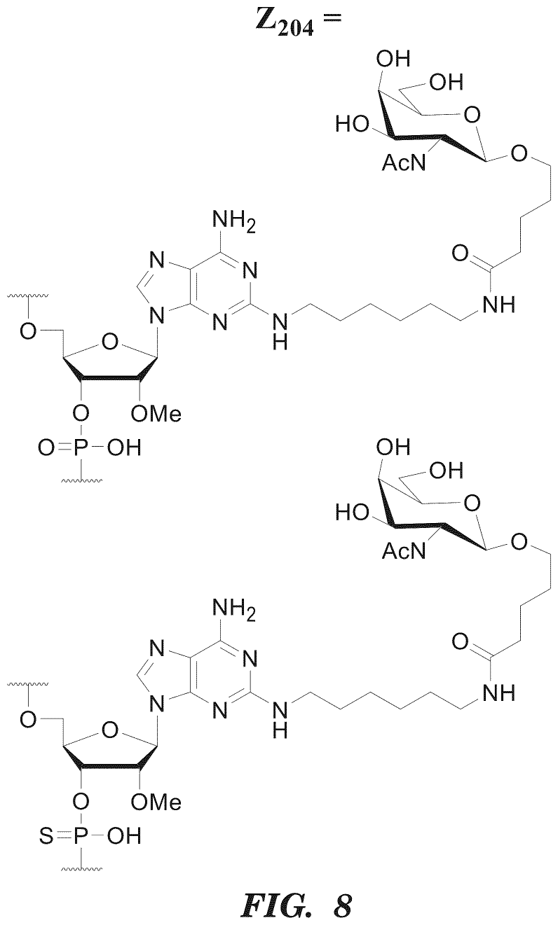

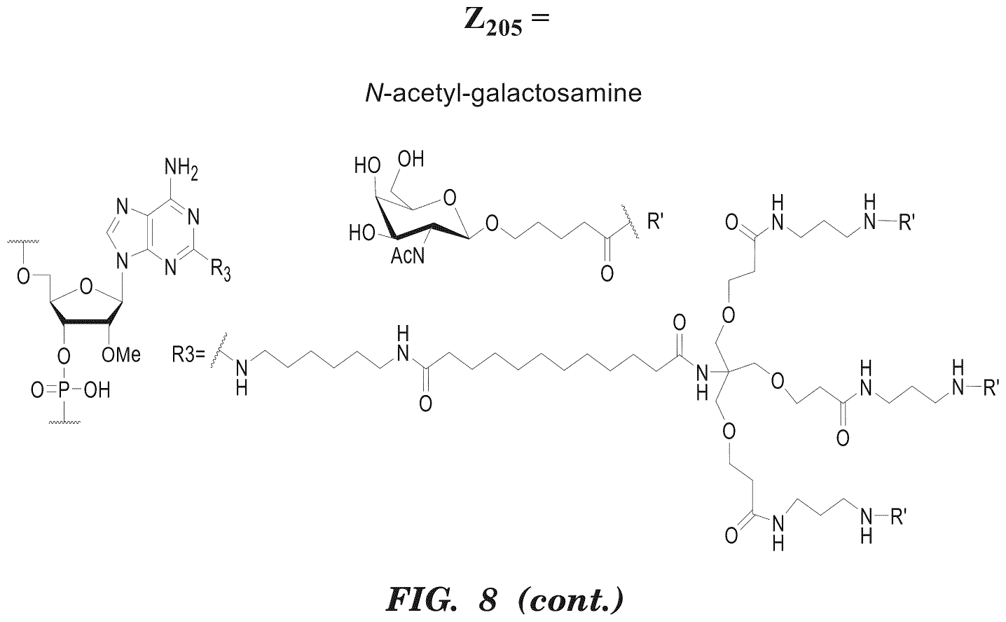

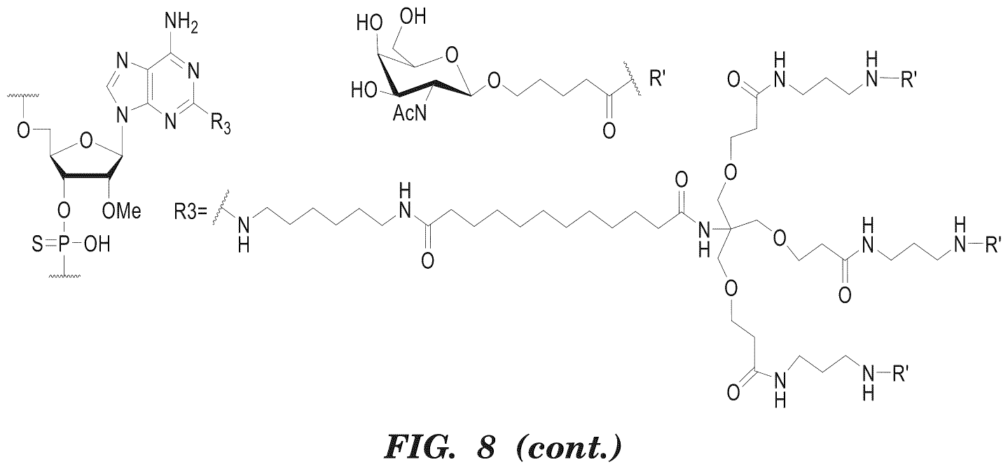

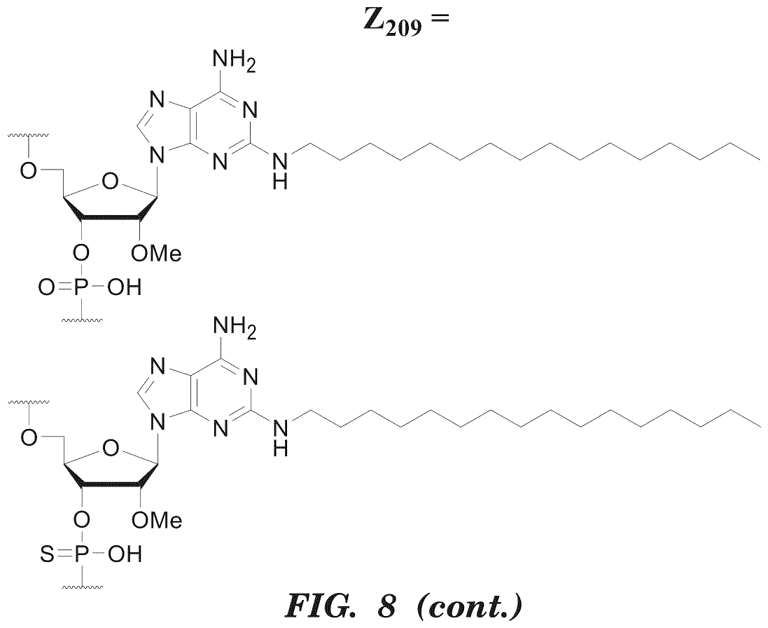

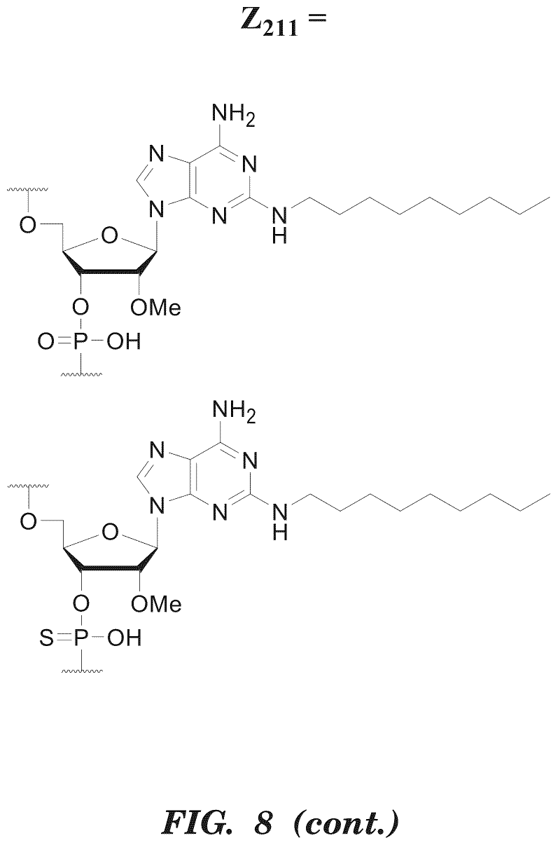

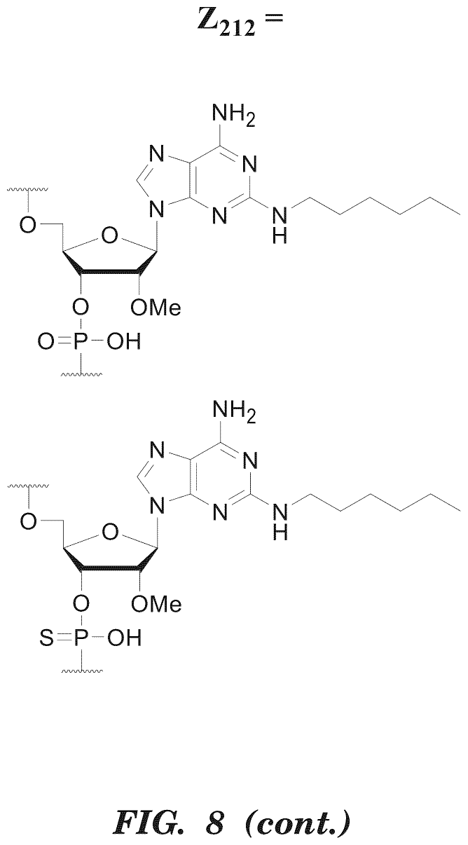

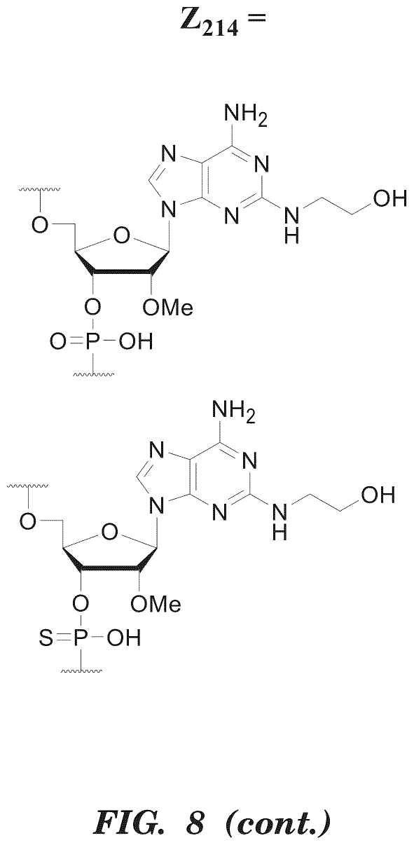

Exemplary conjugates prepared with the post-synthetic conjugation process are shown in FIGS. 7 and 8.

Sequences and mass spectroscopy analysis of the siRNA strands comprising the post-synthesis conjugates of FIGS. 7 and 8 are shown in Table 6.

TABLE 6
Sequences and mass spectroscopy characterization of siRNA conjugate strands

| | Target | | | Mass (m/z) | |
|---|---|---|---|---|---|
| Target | tissue | Strand | Sequence 5′-3′ | Calc. | Obs. |
| mTTR | liver | S Parent | A•a•CaGuGuUCUuGcUcUaUaA(L) | — | — |
| | | A-117799 | (SEQ ID NO: 156) | | |
| mTTR | liver | A- | X₂₀₄•Z₂₀₄CZ₂₀₄GuGuUCUuGcUcUaU•a•A | 8085.85 | |
| | | | (SEQ ID NO: 157) | | |
| mTTR | liver | A- | X₂₀₁•a•CaGuGuUCUuGcUcUaU•a•A | 7360.21 | |
| | | | (SEQ ID NO: 158) | | |
| mTTR | liver | A- | X₂₀₄•a•CaGuGuUCUuGcUcUaU•a•A | 7250.86 | 7248.92 |
| | | | (SEQ ID NO: 159) | | |
| mTTR | liver | A- | X₂₀₅•a•CaGuGuUCUuGcUcUaU•a•A | 8557.42 | 8554.31 |
| | | | (SEQ ID NO: 160) | | |
| mTTR | liver | A- | X₂₀₆•a•CaGuGuUCUuGcUcUaU•a•A | 8124.88 | 8122.72 |
| | | | (SEQ ID NO: 161) | | |
| mTTR | liver | A- | X₂₀₉•a•CaGuGuUCUuGcUcUaU•a•A | 7072.80 | |
| | | | (SEQ ID NO: 162) | | |
| mTTR | liver | A- | A•a•CaGuGuUCUuGcUCUZ₂₀₄UZ₂₀₄X₂₀₄ | 8053.72 | |
| | | | (SEQ ID NO: 163) | | |
| mTTR | liver | A- | A•a•CaGuGuUCUuGcUcUaUaX₂₀₅ | 8555.3 | |
| | | | (SEQ ID NO: 164) | | |
| h/cTTR | ocular | S Parent | u•g•ggauUuCAUguaaccaa•g•a | | |
| | | A- | (SEQ ID NO: 165) | | |
| h/cTTR | ocular | A- | u•g•ggZ₂₀₉uUuCAUguaaccaa•g•a | | |
| | | | (SEQ ID NO: 166) | | |
| h/cTTR | ocular | A- | u•g•ggZ₂₁₁uUuCAUguZ₂₁₁accaa•g•a | | |
| | | | (SEQ ID NO: 167) | | |
| h/cTTR | ocular | A- | u•g•ggZ₂₁₂uUuCAUguZ₂₁₂accZ₂₁₂a•g•a | | |
| | | | (SEQ ID NO: 168) | | |
| h/cTTR | ocular | AS Parent | VPu•C•uugGuuAcaugAaAuccca•u•c | | |
| | | A-131902 | (SEQ ID NO: 169) | | |
| h/cTTR | ocular | A- | VPu•C•uugGuuX₂₀₉caugAaAuccca•u•c | | |
| | | | (SEQ ID NO: 170) | | |
| h/cTTR | ocular | A- | VPu•C•uugGuuX₂₁₁CZ₂₁₁ugAaAuccca•u•c | | |
| | | | (SEQ ID NO: 171) | | |
| h/cTTR | ocular | A- | VPu•C•uugGuuX₂₁₂CZ₂₁₂ugX₂₁₂aAuccca•u•c | | |
| | | | (SEQ ID NO: 172) | | |
| C5 | liver | S Parent | U•g•AcAaAaUAAcUcAcUaUaA | | |
| | | A-119322 | (SEQ ID NO: 173) | | |
| C5 | liver | A- | U•g•AcX₂₀₄Z₂₀₄X₂₀₄aUAAcUcAcUaU•a•A | | |
| | | | (SEQ ID NO: 174) | | |
| C5 | liver | A- | U•g•X₂₀₅cAaAaUAAcUcAcUaU•a•A | | |
| | | | (SEQ ID NO: 175) | | |
| C5 | liver | A- | U•g•AcAaAaUAAcUcAcUaUaX₂₀₅ | | |
| | | | (SEQ ID NO: 176) | | |
| C5 | liver | A- | U•g•X₂₀₉CAaAaUAAcUcAcUaU•a•A | | |
| | | | (SEQ ID NO: 177) | | |
| C5 | liver | A- | U•g•X₂₁₁CAaAaUX₂₁₁AcUcAcUaU•a•A | | |
| | | | (SEQ ID NO: 178) | | |
| C5 | liver | A- | U•g•X₂₁₂CAaAaUX₂₁₂AcUcX₂₁₂CUaU•a•A | | |
| | | | (SEQ ID NO: 179) | | |
| C5 | liver | AS Parent | u•U•aUaGuGaGuuaUuUuGuCa•a•u | | |
| | | A-119322 | (SEQ ID NO: 180) | | |
| C5 | liver | A- | u•U•Z₂₀₉UaGuGaGuuaUuUuGuCa•a•u | | |
| | | | (SEQ ID NO: 181) | | |
| C5 | liver | A- | u•U•Z₂₁₁UaGuGZ₂₁₁GuuaUuUuGuCa•a•u | | |
| | | | (SEQ ID NO:182) | | |
| C5 | liver | A- | u•U•Z₂₁₂UaGuGZ₂₁₂GuuZ₂₁₂UuUuGuCa•a•u | | |
| | | | (SEQ ID NO: 183) | | |
| β-Cat | liver | S Parent | U•a•CuGuUgGAUuGaUuCgAaA | | |
| | | A-129065 | (SEQ ID NO: 184) | | |
| β-Cat | liver | A- | U•a•CuGuUgGAUuGaUuCgAaX₂₀₅ | | |
| | | | (SEQ ID NO: 185) | | |
| β-Cat | liver | A- | U•X₂₀₅•CuGuUgGAUuGaUuCgA•a•A | | |
| | | | (SEQ ID NO: 186) | | |
| β-Cat | liver | A- | U•X₂₀₉•CuGuUgGAUuGaUuCgA•a•A | | |
| | | | (SEQ ID NO: 187) | | |
| β-Cat | liver | A- | U•X₂₁₁•CuGuUgGX₂₁₁UuGaUuCgA•a•A | | |
| | | | (SEQ ID NO: 188) | | |
| β-Cat | liver | A- | U•X₂₁₂•CuGuUgGX₂₁₂UuGZ₂₁₂UuCgA•a•A | | |
| | | | (SEQ ID NO: 189) | | |
| β-Cat | liver | AS Parent | VPu•U•uCgAaUcAaucCaAcAgUa•g•c | | |
| | | A-129066 | (SEQ ID NO: 190) | | |
| β-Cat | liver | A- | VPu•U•uCgX₂₀₉aUcAaucCaAcAgUa•g•c | | |
| | | | (SEQ ID NO: 191) | | |
| β-Cat | liver | A- | VPu•U•uCgX₂₁₁aUcX₂₁₁aucCaAcAgUa•g•c | | |
| | | | (SEQ ID NO: 192) | | |
| β-Cat | liver | A- | VPu•U•uCgX₂₁₂aUcX₂₁₂aucCZ₂₁₂AcAgUa•g•c | | |
| | | | (SEQ ID NO: 193) | | |
| SOD1 | brain | S Parent | c•a•uuuuAaUCCucacucua•a•a | | |
| | | A-637448 | (SEQ ID NO: 194) | | |
| SOD1 | brain | A- | C•Z₂₀₉•uuuuAaUCCucacucua•a•a | | |
| | | | (SEQ ID NO: 195) | | |
| SOD1 | brain | A- | c•Z₂₁₁•uuuuX₂₁₁aUCCucacucua•a•a | | |
| | | | (SEQ ID NO: 196) | | |
| SOD1 | brain | A- | C•Z₂₁₂•uuuuX₂₁₂aUCCucZ₂₁₂cucua•a•a | | |
| | | | (SEQ ID NO: 197) | | |
| SOD1 | brain | AS Parent | VPu•U•uagAgUGaggaUuAaaaug•a•g | | |
| | | A-444402 | (SEQ ID NO: 198) | | |
| SOD1 | brain | A- | VPu•U•uZ₂₀₉gAgUGaggaUuAaaaug•a•g | | |
| | | | (SEQ ID NO: 199) | | |
| SOD1 | brain | A- | VPu•U•uZ₂₁1gAgUGZ₂₁₁ggaUuAaaaug•a•g | | |
| | | | (SEQ ID NO: 200) | | |

Sequences exemplary dsRNA comprising the post-synthesis conjugates of FIGS. 6 and 7 for siRNA silencing studies are shown in Table 7.

TABLE 7
Duplexes for siRNA silencing study

| | Target | | | Silencing |
|---|---|---|---|---|
| Target | tissue | Duplexes | S/AS duplex 5′-3′ | activity |
| mTTR | liver | Control | A•a•CaGuGuUCUuGcUcUaUaA(L) | |
| | | AD- | u•U•aUaGaGcAagaAcAcUgUu•u•u | |
| | | | (SEQ ID NO: 201) | |
| mTTR | liver | AD- | X₂₀₄•Z₂₀₄•CZ₂₀₄GuGuUCUuGcUcUaU•a•A | |
| | | | u•U•aUaGaGcAagaAcAcUgUu•u•u | |
| | | | (SEQ ID NO: 202) | |
| mTTR | liver | AD- | X₂₀₁•a•CaGuGuUCUuGcUcUaU•a•A | |
| | | | u•U•aUaGaGcAagaAcAcUgUu•u•u | |
| | | | (SEQ ID NO: 203) | |
| mTTR | liver | AD- | X₂₀₅•a•CaGuGuUCUuGcUcUaU•a•A | |
| | | | u•U•aUaGaGcAagaAcAcUgUu•u•u | |
| | | | (SEQ ID NO: 204) | |
| mTTR | liver | AD- | X₂₀₆•a•CaGuGuUCUuGcUcUaU•a•A | |
| | | | u•U•aUaGaGcAagaAcAcUgUu•u•u | |
| | | | (SEQ ID NO: 205) | |
| mTTR | liver | AD- | A•a•CaGuGuUCUuGcUcUZ₂₀₄UZ₂₀₄X₂₀₄ | |
| | | | u•U•aUaGaGcAagaAcAcUgUu•u•u | |
| | | | (SEQ ID NO: 206) | |
| mTTR | liver | AD- | A•a•CaGuGuUCUuGcUcUaUaX₂₀₅ | |
| | | | u•U•aUaGaGcAagaAcAcUgUu•u•u | |
| | | | (SEQ ID NO: 207) | |
| mTTR | liver | A- | u•g•ggauUuCAUguaaccaa•g•a | |
| | | | VPu•C•uugGuuAcaugAaAuccca•u•c | |
| | | | (SEQ ID NO: 208) | |
| mTTR | liver | A- | u•g•ggZ₂₀₉uUuCAUguaaccaa•g•a | |
| | | | VPu•C•uugGuuAcaugAaAuccca•••c | |
| | | | (SEQ ID NO: 209) | |
| h/cTTR | ocular | Control | u•g•ggZ₂₁₁u•uCAUguZ₂₁₁accaa•g•a | |
| | | AD- | VPu•C•uugGuuAcaugAaAuccca•u•c | |
| | | | (SEQ ID NO: 210) | |
| h/cTTR | ocular | AD- | VPu•C•uugGuuAcaugAaAuccca•u•c | |
| | | | (SEQ ID NO: 211) | |
| h/cTTR | ocular | AD- | u•g•ggauUuCAUguaaccaa•g•a | |
| | | | VPu•CouugGuuX₂₀₉caugAaAuccca•u•c | |
| | | | (SEQ ID NO: 212) | |
| h/cTTR | ocular | AD- | u•g•ggauUuCAUguaaccaa•g•a | |
| | | | VPu•C•uugGuuX₂₁₁CZ₂₁₁ugAaAuccca•u•c | |
| | | | (SEQ ID NO: 213) | |
| h/cTTR | ocular | AD- | u•g•ggauUuCAUguaaccaa•g•a | |
| | | | VPu•C•uugGuuX₂₁₂CZ₂₁₂ugX₂₁₂aAuccca•u•c | |
| | | | (SEQ ID NO: 214) | |
| h/cTTR | ocular | AD- | U•g•AcAaAaUAAcUcAcUaUaA | |
| | | | u•U•aUaGuGaGuuaUuUuGuCa•a•u | |
| | | | (SEQ ID NO: 215) | |
| h/cTTR | ocular | AD- | U•g•AcX₂₀₄Z₂₀₄X₂₀₄aUAAcUcAcUaUaA | |
| | | | u•U•aUaGuGaGuuaUuUuGuCa•a•u | |
| | | | (SEQ ID NO: 216) | |
| C5 | liver | Control | U•g•X₂₀₅CAaAaUAAcUcAcUaUaA | |
| | | AD- | u•U•aUaGuGaGuuaUuUuGuCa•a•u | |
| | | | (SEQ ID NO: 217) | |
| C5 | liver | AD- | U•g•AcAaAaUAAcUcAcUaUaX₂₀₅ | |
| | | | u•U•aUaGuGaGuuaUuUuGuCa•a•u | |
| | | | (SEQ ID NO: 218) | |
| C5 | liver | AD- | U•g•X₂₀₉CAaAaUAAcUcAcUaU•a•A | |
| | | | u•U•aUaGuGaGuuaUuUuGuCa•a•u | |
| | | | (SEQ ID NO: 219) | |
| C5 | liver | A- | U•g•X₂₁₁CAaAaUX₂₁₁AcUcAcUaU•a•A | |
| | | | u•U•aUaGuGaGuuaUuUuGuCa•a•u | |
| | | | (SEQ ID NO: 220) | |
| C5 | liver | AD- | u•U•aUaGuGaGuuaUuUuGuCa•a•u | |
| | | | (SEQ ID NO: 221) | |
| C5 | liver | AD- | U•g•AcAaAaUAAcUcAcUaUaA(L) | |
| | | | u•U•Z₂₀₉UaGuGaGuuaUuUuGuCa•a•u | |
| | | | (SEQ ID NO: 222) | |
| C5 | liver | AD- | U•g•AcAaAaUAAcUcAcUaUaA(L) | |
| | | | u•U•Z₂₁₁UaGuGZ₂₁₁GuuaUuUuGuCa•a•u | |
| | | | (SEQ ID NO: 223) | |
| C5 | liver | AD- | U•g•AcAaAaUAAcUcAcUaUaA(L) | |
| | | | u•U•Z₂₁₂UaGuGZ₂₁₂GuuZ₂₁₂UuUuGuCa•a•u | |
| | | | (SEQ ID NO: 224) | |
| C5 | liver | AD- | U•a•CuGuUgGAUuGaUuCgAaA(L) | |
| | | | VPu•U•uCgAaUcAaucCaAcAgUa•g•c | |
| | | | (SEQ ID NO: 225) | |
| C5 | liver | AD- | U•a•CuGuUgGAUuGaUuCgAaX₂₀₅ | |
| | | | VPu•U•uCgAaUcAaucCaAcAgUa•g•c | |
| | | | (SEQ ID NO: 226) | |
| β-Cat | liver | Control | U•X₂₀₅•CuGuUgGAUuGaUuCgA•a•A | |
| | | AD- | VPu•U•uCgAaUcAaucCaAcAgUa•g•c | |
| | | | (SEQ ID NO: 227) | |
| β-Cat | liver | AD- | U•X₂₀₉•CuGuUgGAUuGaUuCgA•a•A | |
| | | | VPu•U•uCgAaUcAaucCaAcAgUa•g•c | |
| | | | (SEQ ID NO: 228) | |
| β-Cat | liver | AD- | U•X₂₁₁•CuGuUgGX₂₁₁UuGaUuCgA•a•A | |
| | | | VPu•U•uCgAaUcAaucCaAcAgUa•g•c | |
| | | | (SEQ ID NO: 229) | |
| β-Cat | liver | AD- | U•X₂₁₂•CuGuUgGX₂₁₂UuGZ₂₁₂UuCgA•a•A | |
| | | | VPu•U•uCgAaUcAaucCaAcAgUa•g•c | |
| | | | (SEQ ID NO: 230) | |
| β-Cat | liver | AD- | U•a•CuGuUgGAUuGaUuCgA•a•A | |
| | | | VPu•U•uCgX₂₀₉aUcAaucCaAcAgUa•g•c | |
| | | | (SEQ ID NO: 231) | |
| β-Cat | liver | AD- | U•a•CuGuUgGAUuGaUuCgA•a•A | |
| | | | VPu•U•uCgX₂₁₁aUcX₂₁₁aucCaAcAgUa•g•c | |
| | | | (SEQ ID NO: 232) | |
| β-Cat | liver | AD- | U•a•CuGuUgGAUuGaUuCgA•a•A | |
| | | | VPu•U•uCgX₂₁₂aUcX₂₁₂aucCZ₂₁₂AcAgUa•g•c | |
| | | | (SEQ ID NO: 233) | |
| β-Cat | liver | AD- | c•a•uuuuAaUCCucacucua•a•a | |
| | | | VPu•U•uagAgUGaggaUuAaaaug•a•g | |
| | | | (SEQ ID NO: 234) | |
| β-Cat | liver | AD- | c•Z₂₀₉•uuuuAaUCCucacucua•a•a | |
| | | | VPu•U•uagAgUGaggaUuAaaaug•a•g | |
| | | | (SEQ ID NO: 235) | |
| SOD1 | brain | Control | c•Z₂₁₁•uuuuX₂₁₁aUCCucacucua•a•a | |
| | | AD- | VPu•U•uagAgUGaggaUuAaaaug•a•g | |
| | | | (SEQ ID NO: 236) | |
| SOD1 | brain | AD- | c•Z₂₁₂•uuuuX₂₁₂aUCCucZ₂₁₂cucua•a•a | |
| | | | VPu•U•uagAgUGaggaUuAaaaug•a•g | |
| | | | (SEQ ID NO: 237) | |
| SOD1 | brain | AD- | c•a•uuuuAaUCCucacucua•a•a | |
| | | | VPu•U•uZ₂₀₉gAgUGaggaUuAaaaug•a•g | |
| | | | (SEQ ID NO: 238) | |
| SOD1 | brain | AD- | c•a•uuuuAaUCCucacucua•a•a | |
| | | | VPu•U•uZ₂₁₁gAgUGZ₂₁₁ggaUuAaaaug•a•g | |
| | | | (SEQ ID NO: 239) | |
| SOD1 | brain | AD- | c•a•uuuuAaUCCucacucua•a•a | |
| | | | VPu•U•uZ₂₁₂gAgUGZ₂₁₂ggaVuX₂₁₂aaaug•a•g | |
| | | | (SEQ ID NO: 240) | |
| SOD1 | brain | AD- | A•a•CaGuGuUCUuGcUcUaUaA(L) | |
| | | | u•U•aUaGaGcAagaAcAcUgUu•u•u | |
| | | | (SEQ ID NO: 241) | |
| SOD1 | brain | AD- | X₂₀₄•Z₂₀₄•CZ₂₀₄GuGuUCUuGcUcUaU•a•A | |
| | | | u•U•aUaGaGcAagaAcAcUgUu•u•u | |
| | | | (SEQ ID NO: 242) | |

Italicized uppercase, lowercase, uppercase, uppercase red bold letters and lowercase bold letters represent 2′-F-RNA, 2′-OMe, LNA sugar modifications, 2,6-diaminopurine and deoxy nucleoside, respectively, to Adenosine (A), Cytidine (C), Guanosine (G), Uridine (U), 2'F-2-cholesterol-adenosine (X₂₀₁), 2′F-2-N-acetylgalactosamine-adenosine (X₂₀₄), 2′F-2-tri-N-acetylgalactosamine (X₂₀₅), 2′F-2-cRGD Kessler peptide (X₂₀₆), 2′F-2-C16-adenosine (X₂₀₉), 2′F-2-C9-adenosine (X₂₁₁), 2′F-2-C6-adenosine (X₂₁₂), 2′OMe-2-N-acetylgalactosamine-adenosine (Z₂₀₄), 2′OMe-2-C16-adenosine (Z₂₀₉), 2′OMe-2-C9-adenosine (Z₂₁₁), 2′OMe-2-C6-adenosine (Z₂₁₂),Phosphorothioate linkages are indicated by the “•” symbol

Sequences and pourcentage conversion analysis of the siRNA strands comprising the solid phase or solution phase post-synthesis conjugates of FIGS. 7 and 8 are shown in Table 8 and Table 9 respectively.

TABLE 8
Sequences and pourcentage conversion of siRNA conjugate strands obtain by
solid phase conjugation

| | Target | Strand | Sequence 5′-3′ | % Conversion |
|---|---|---|---|---|
| 307 | — | A- | X₂₀₁dTdTdTdTdTdTdTdTdTdTdTdTdTdTdTdTdTdT | 100 |
| | | | (SEQ ID NO:243) | |
| 308 | — | A- | X₂₀₄dTdTdTdTdTdTdTdTdTdTdTdTdTdTdTdTdTdT | 78 |
| | | | (SEQ ID NO:244) | |
| 309 | — | A- | X₂₀₅dTdTdTdTdTdTdTdTdTdTdTdTdTdTdTdTdTdT | |
| | | | (SEQ ID NO:245) | |
| 310 | — | A- | X₂₀₆dTdTdTdTdTdTdTdTdTdTdTdTdTdTdTdTdTdT | 62 |
| | | | (SEQ ID NO:246) | |
| 311 | — | A- | X₂₀₉dTdTdTdTdTdTdTdTdTdTdTdTdTdTdTdTdTdT | 100 |
| | | | (SEQ ID NO:247) | |
| | mTTR | S Parent | A•a•CaGuGuUCUuGcUcUaUaA(L) | — |
| | | A-117799 | (SEQ ID NO: 248) | |
| 318 | mTTR | A- | X₂₀₁•a•CaGuGuUCUuGcUcUaU•a•A | 100 |
| | | | (SEQ ID NO: 249) | |
| 319 | mTTR | A- | X₂₀₄•a•CaGuGuUCUuGcUcUaU•a•A | 100 |
| | | | (SEQ ID NO: 250) | |
| 320 | mTTR | A- | X₂₀₅•a•CaGuGuUCUuGcUcUaU•a•A | 43 |
| | | | (SEQ ID NO: 251) | |
| 321 | mTTR | A- | X₂₀₆•a•CaGuGuUCUuGcUcUaU•a•A | 87 |
| | | | (SEQ ID NO: 252) | |
| 322 | mTTR | A- | X₂₀₉•a•CaGuGuUCUuGcUcUaU•a•A | 100 |
| | | | (SEQ ID NO: 253) | |

Italicized uppercase, lowercase, uppercase, uppercase red bold letters and lowercase bold letters represent 2′-F-RNA, 2′-OMe, LNA sugar modifications, 2,6-diaminopurine and deoxy nucleoside, respectively, to Adenosine (A), Cytidine (C), Guanosine (G), Uridine (U), 2′F-2-cholesterol-adenosine (X₂₀₁), 2′F-2-N-acetylgalactosamine-adenosine (X₂₀₄), 2′F-2-tri-N-
acetylgalactosamine (X₂₀₅), 2′F-2-cRGD Kessler peptide (X₂₀₆), 2′F-2-C16-adenosine (X₂₀₉), 2′F-2-C9-adenosine (X₂₁₁), 2′F-2-C6-adenosine (X₂₁₂), 2′OMe-2-N-acetylgalactosamine-adenosine (Z₂₀₄), 2′OMe-2-C16-adenosine (Z₂₀₉), 2′OMe-2-C9-adenosine (Z₂₁₁), 2′OMe-2-C6-adenosine (Z₂₁₂), deoxy-thymidine (dT), Phosphorothioate linkages are indicated by the “•” symbol

TABLE 9
Sequences and percentage conversion of siRNA conjugate strands obtain by
solution phase conjugation

| | | | | % Conversion | |
|---|---|---|---|---|---|
| | | | | Reported | Current |
| | Target | Strand | Sequence 5′-3′ | method | method |
| | mTTR | S Parent | A•a•CaGuGuUCUuGcUcUaUaA(L) | — | — |
| | | A-117799 | (SEQ ID NO: 248) | | |
| 320 | mTTR | A- | X₂₀₅•a•CaGuGuUCUuGcUcUaU•a•A | | |
| | | | (SEQ ID NO: 251) | | |
| 322 | mTTR | A- | X₂₀₉•a•CaGuGuUCUuGcUcUaU•a•A | 74 | 91 |
| | | | (SEQ ID NO: 253) | | |

Italicized uppercase, lowercase, uppercase, uppercase red bold letters and lowercase bold letters represent 2′-F-RNA, 2′-OMe, LNA sugar modifications, 2,6-diaminopurine and deoxy nucleoside, respectively, to Adenosine (A), Cytidine (C), Guanosine (G), Uridine (U), 2′F-2-cholesterol-adenosine (X201), 2′F-2-N-acetylgalactosamine-adenosine (X₂₀₄), 2′F-2-tri-N-acetylgalactosamine (X₂₀₅), 2′F-2-cRGD Kessler peptide (X₂₀₆), 2′F-2-C16-adenosine (X₂₀₉), 2′F-2-C9-adenosine (X₂₁₁), 2′F-2-C6-adenosine (X₂₁₂), 2′OMe-2-N-acetylgalactosamine-adenosine (Z₂₀₄), 2'OMe-2-C16-adenosine (Z₂₀₉), 2′OMe-2-C9-adenosine (Z₂₁₁), 2′OMe-2-C6-adenosine (Z₂₁₂), Phosphorothioate linkages are indicated by the ″.″ symbol

### Results and Discussion

Synthesis of monomers (amidites) for DAP containing oligonucleotides has always been problematic and challenging. See, for example, Sproat, B. S., et al. (1990), Nucleic Acids Research 18(1): 41-49; Rosenbohm, C., et al. (2004), Bioorganic & Medicinal Chemistry, 12(9), 2385-2396; Boudou, V., et al. (1999), Nucleic Acids Research, 27(6), 1450-1456; Wu, X., et al. (2002), Organic Letters, 4(8), 1283-1286; Langkjxer, N., et al. (2015), Bioorganic & Medicinal Chemistry Letters, 25(22), 5064-5066; Matray, T. J. and S. M. Gryaznov (1999), Nucleic Acids Research, 27(20), 3976-3985; Haaiima, G., et al. (1997), Nucleic Acids Res, 25(22), 4639-4643; and Kamiya, Y., et al. (2017), ChemBioChem 18(19), 1917-1922, contents of all which are incorporated herein by reference in their entirety. The methods used in the prior art, the DAP-modified building blocks (amidites used) mostly consist of orthogonal protecting groups for 2- and 6-amino groups. See, for example, WO2010062404A2, ChemGenes Corporation, (2010); Nucleic Acids Research, (1982), 10(14), 4351-4361; Nucleosides and Nucleotides, (1989), 8(5-6), 1051-1051; The Journal of Organic Chemistry, (2010), 75(5), 1360-1365; The Journal of Organic Chemistry, (2014), 79(10), 4423-4437; Nucleic Acids Research (1990), 18(1): 41-49; Nucleic Acids Research, (1999), 27(20), 3976-3985; ChemBioChem, (2017), 18(19), 1917-1922; Zeitschrift für Naturforschung B, (1988), 43(5), 623-630 and Nucleic Acids Research, (2007), 35(12), 4055-4063, contents of all which are incorporated herein by reference in their entirety.

Ross and Manoharan introduced 2-fluoro-6-amino purines as a novel precursor of DAP and applied it in oligonucleotide synthesis. See, for example, B. S. Ross, R. H. Springer, R. Bharadwaj, A. M. Symons, & M. Manoharan (1999) Nucleosides and Nucleotides, 18, 1203-1204 and International patent publication WO2000012563 to B. S. Ross & M. Manoharan, contents of both of which are incorporated herein by reference in their entirety. Without wishing to be bound by a theory, F atom at 2-position of the purine, e.g. adenine works as a sink of electron through —I of −F and +R effect of amino and completely inactivate the 6-NH₂ group and stabilize the intermediate after F loss. This results in a “no requirement of protecting group” for the 6-NH₂ group.

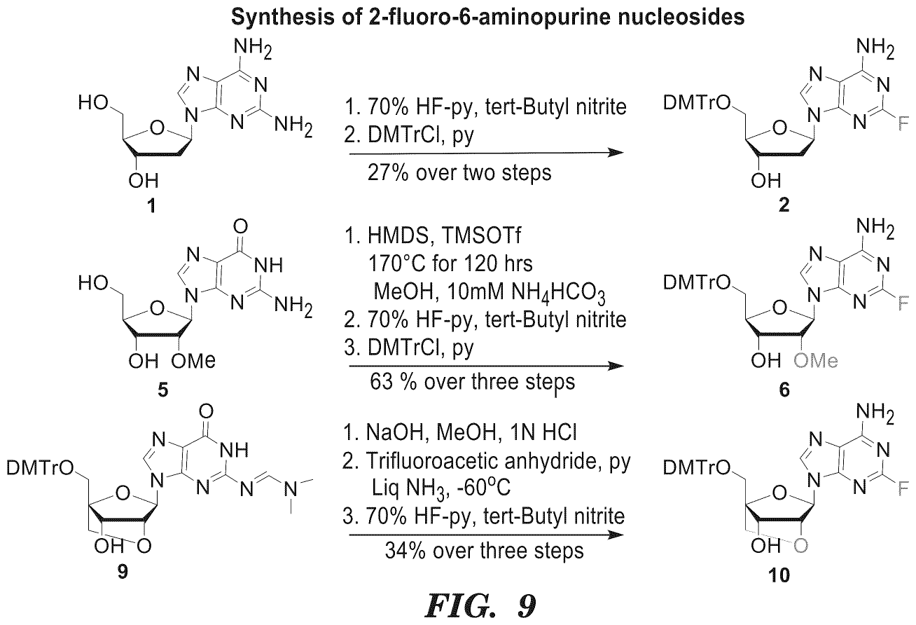

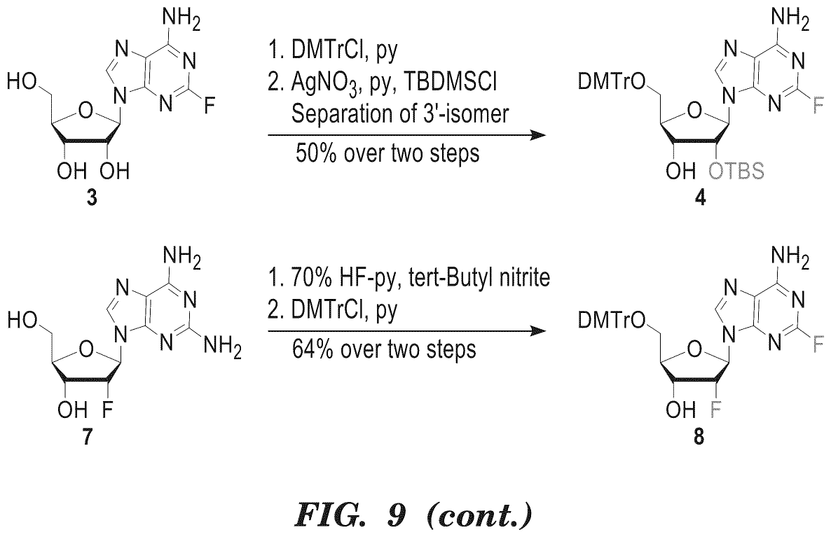

In this study, we have described the use of five different 2-fluoro-6-amino purines as building blocks to avoid and resolve synthetic problems. Starting 2-fluoro-6-aminopurine nucleosides were prepared according to the method shown FIG. 9.

The 2-fluoro-6-aminopurine nucleosides were used to prepare the amidites and CPGs according to the methods shown in Scheme 1 and Scheme 2.

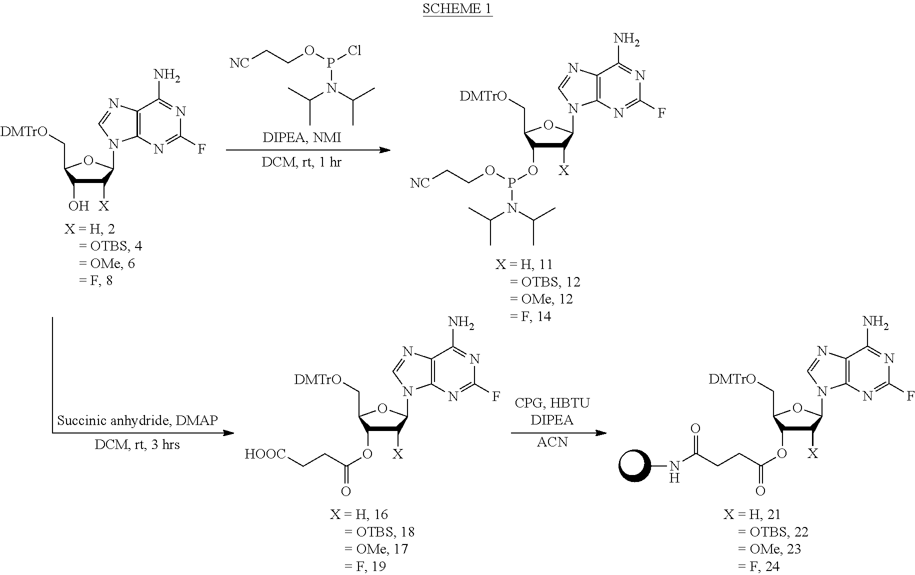

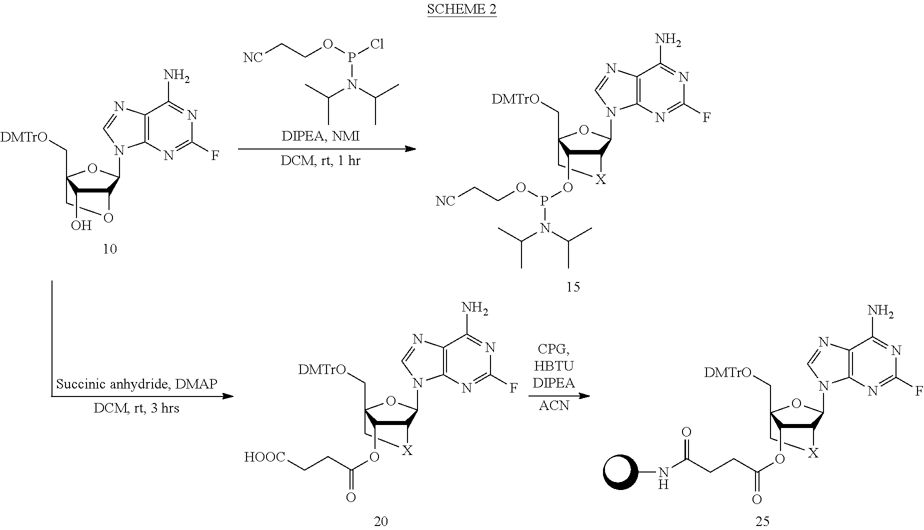

### Materials and Methods

General conditions: TLC was performed on Merck silica gel 60 plates coated with F254. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QTof Premier instrument using the direct flow injection mode. ¹H NMR spectra were recorded at 500 and 600 MHz. ¹³C NMR spectra were recorded at 126 and 151 MHz. ³¹P NMR spectra were recorded at 202 and 243 MHz. ¹⁹F NMR spectra were recorded at 565 MHz. Chemical shifts are given in ppm referenced to the solvent residual peak (DMSO-d₆—1H: δ at 2.50 ppm and 13C δ at 39.5 ppm; CDCl₃—1H: δ at 7.26 ppm and 13C δ at 77.16 ppm). Coupling constants are given in Hertz. Signal splitting patterns are described as singlet (s), doublet (d), triplet (t), septet (sept), broad signal (brs), or multiplet (m).

### Oligomer Synthesis:

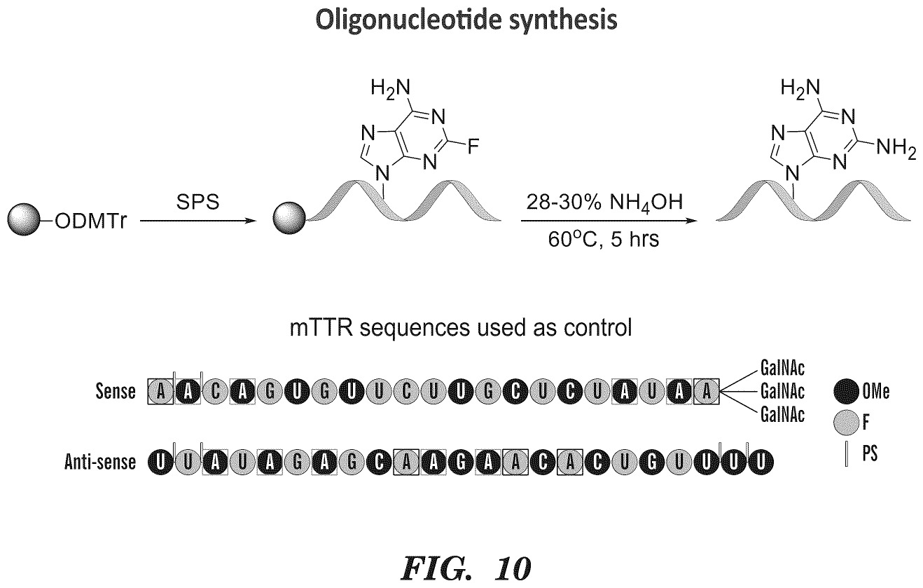

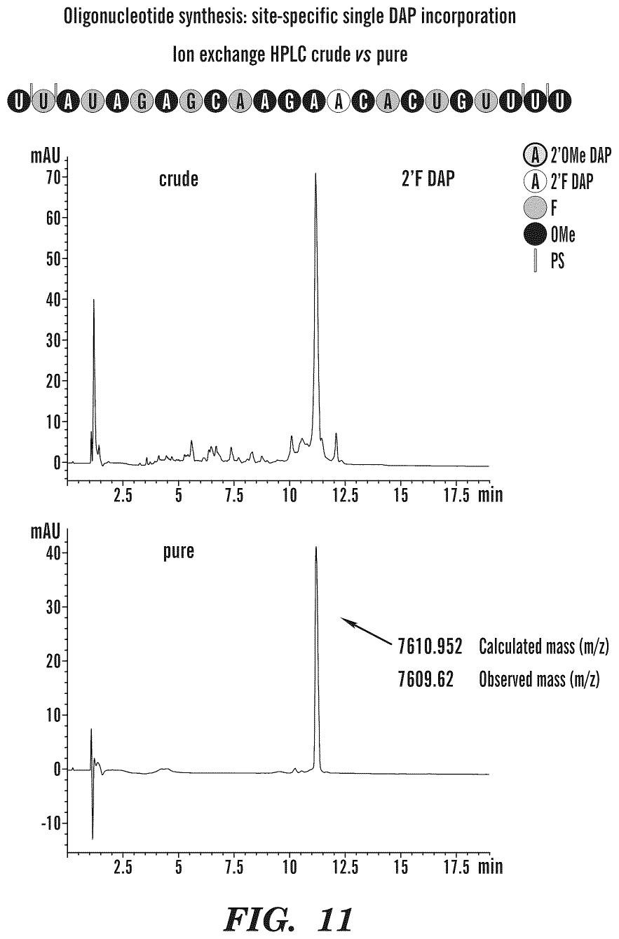

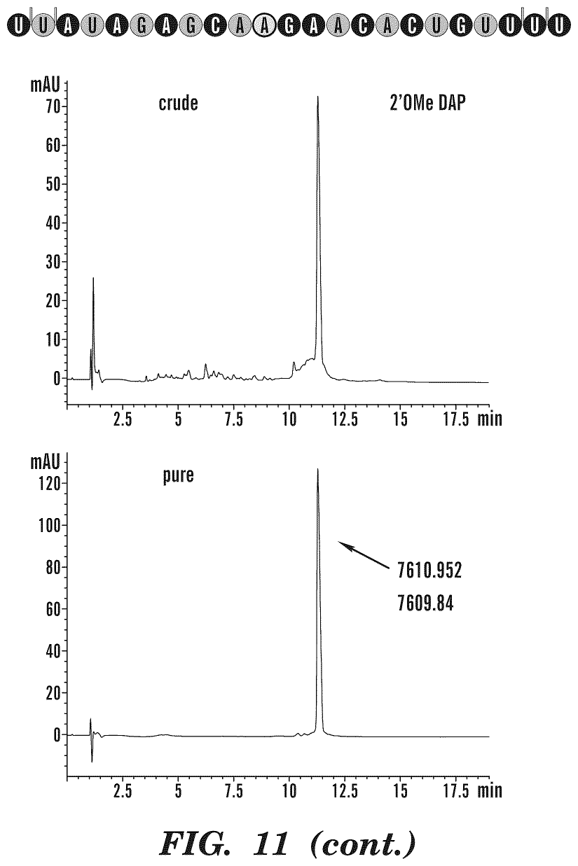

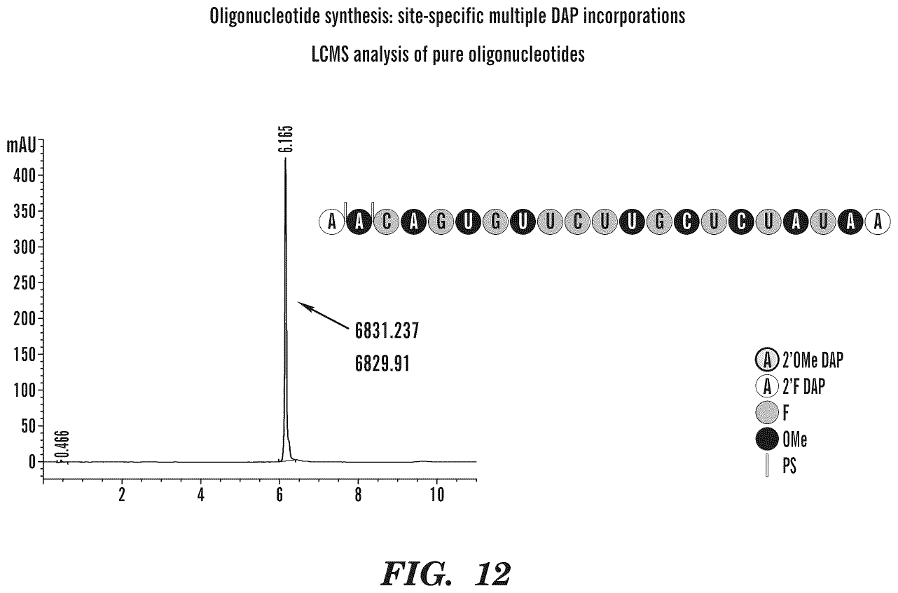

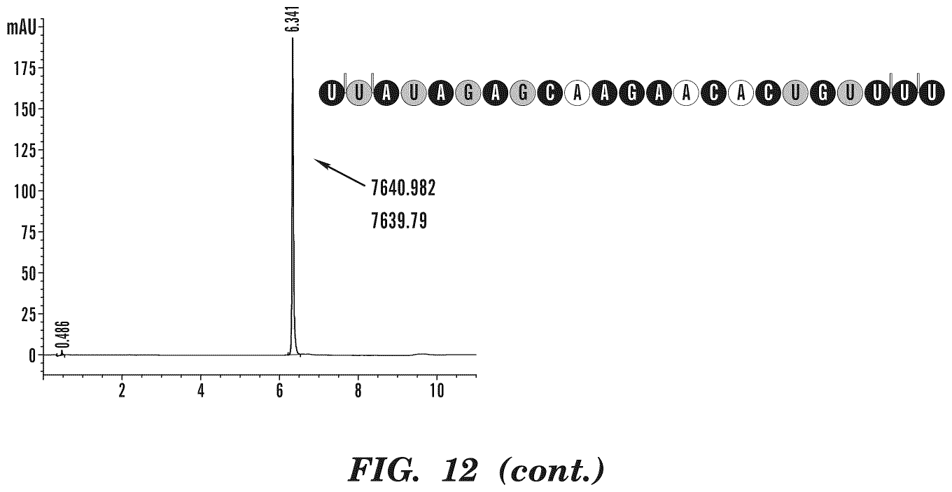

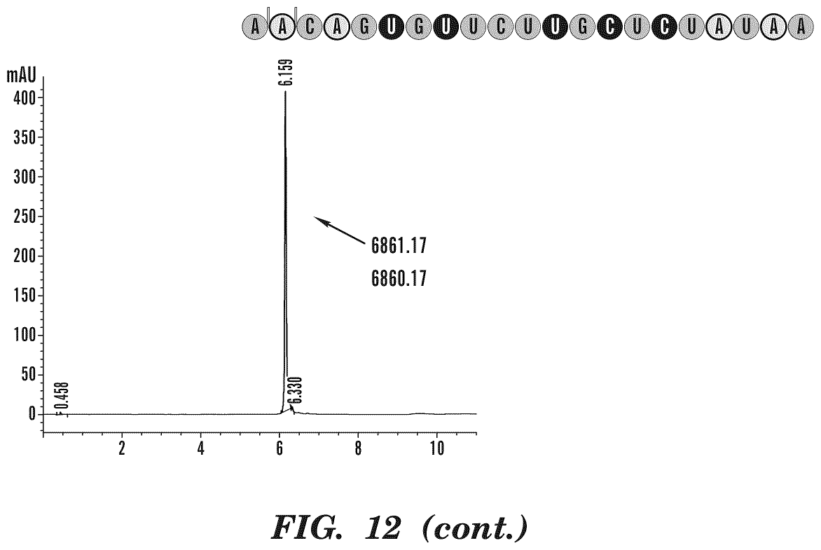

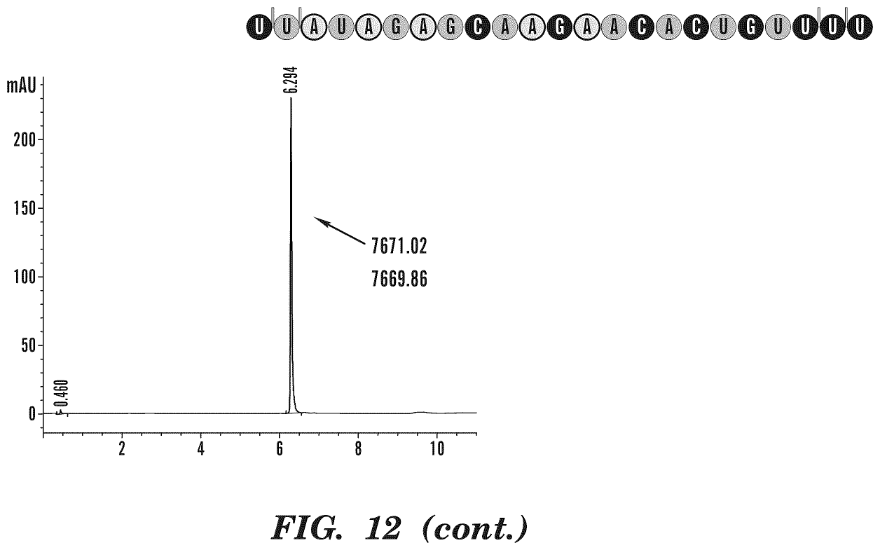

Oligonucleotides were synthesized on an ABI-394 DNA/RNA synthesizer using modified synthesis cycles based on those provided with the instrument. A solution of 0.25 M 5-(S-ethylthio)-1H-tetrazole in acetonitrile was used as the activator. The phosphoramidite solutions were 0.15 M in anhydrous acetonitrile. The oxidizing reagent was 0.02 M 12 in THF/pyridine/H₂O. N,N-Dimethyl-N′-(3-thioxo-3H-1,2,4-dithiazol-5-yl) methanimidamide (DDTT), 0.1 M in pyridine, was used as the sulfurizing reagent. The detritylation reagent was 3% dichloroacetic acid (DCA) in DCM. After completion of the automated synthesis, the solid support was washed with 0.1 M piperidine in acetonitrile for 10 min, then washed with anhydrous acetonitrile and dried with argon. The oligonucleotide was then manually released from support and deprotected using a mixture of 30% NH₄OH for 5 h at 60° C. Solvent was collected by filtration and the support was rinsed with deionized water (6 mL). About oligonucleotides containing 2′-OTBS protecting group were release from support and deprotected using NH₄OH/ethanol (3:1, v/v) or 40% methylamine (0.5 mL/μmol of solid support) for 6 h at 55° C. or 15 min at 60° C., respectively. For all oligonucleotide, solvent was collected by filtration and the support was rinsed with DMSO (1.5 mL/μmol of solid support) according to the method shown in FIG. 10. Crude oligonucleotides were purified by anion exchange glass column (10×mm) packed with TSK Gel Super Q-5PW and using 20 mM sodium phosphate, 15% ACN as buffer A and 20 mM sodium phosphate 15% ACN, 1M NaBr as buffer B. Purification gradient started from 20% to 60% B in 240 min at 60° C. All single strands were purified to >90% IEX HPLC and >98% C18 HPLC (260 nm) purity and then desalted by size exclusion chromatography using a custom packed with Sephadex G25 (GE Healthcare), eluted with sterile nuclease-free water. Some examples are shown in FIGS. 11 and 12.

Oligonucleotide concentrations were calculated based on absorbance at 260 nm and the following extinction coefficients: A/2,6-diaminoA, 13.86 M⁻¹ cm⁻¹; T/U, 7.92 M⁻¹ cm⁻¹; C, 6.57 M⁻¹ cm⁻¹; and G, 10.53 M⁻¹ cm⁻¹. The purity and identity of oligonucleotides were verified by analytical anion exchange chromatography and mass spectrometry, respectively.

### Post-Synthesis Oligonucleotide Conjugation

### On Solid Support

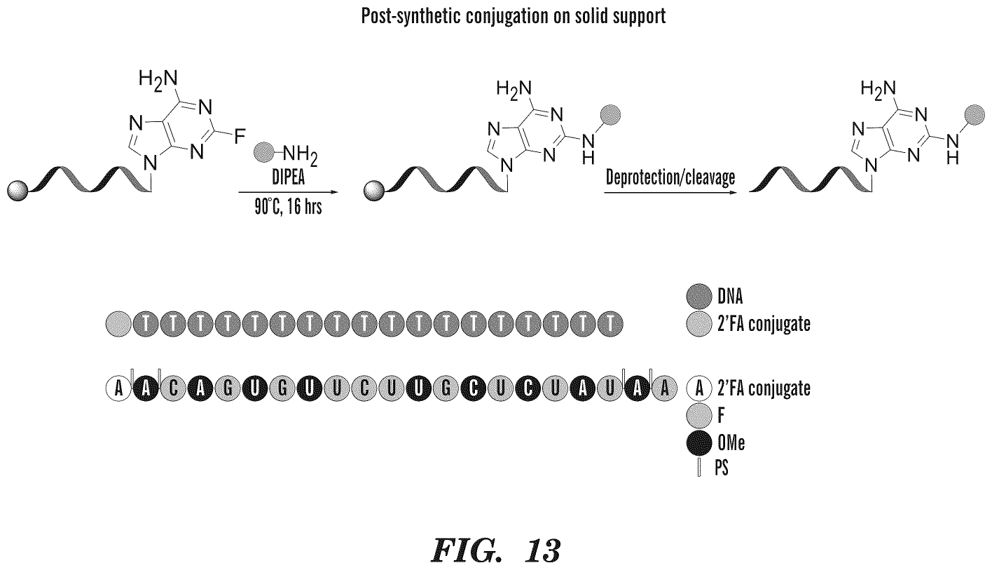

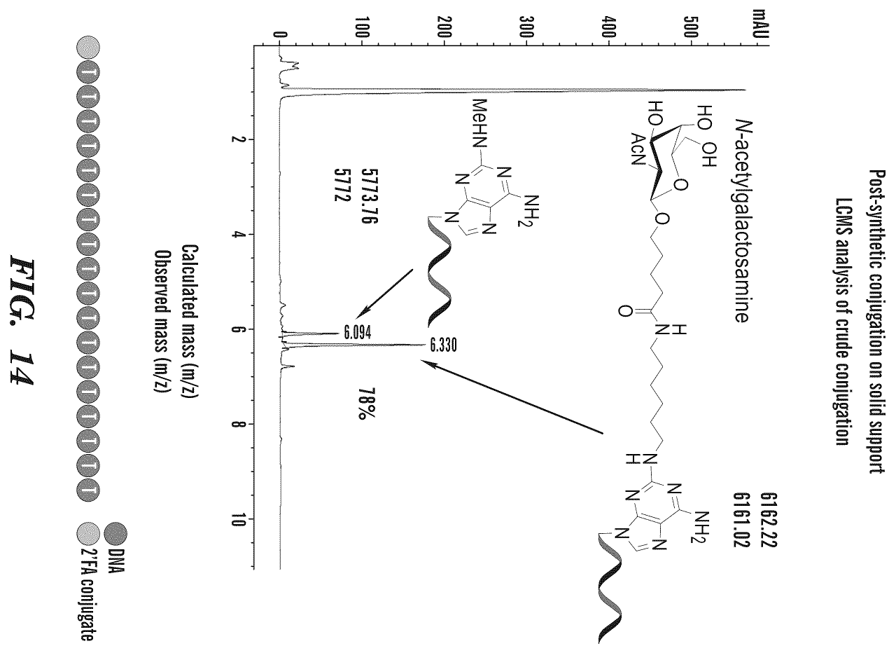

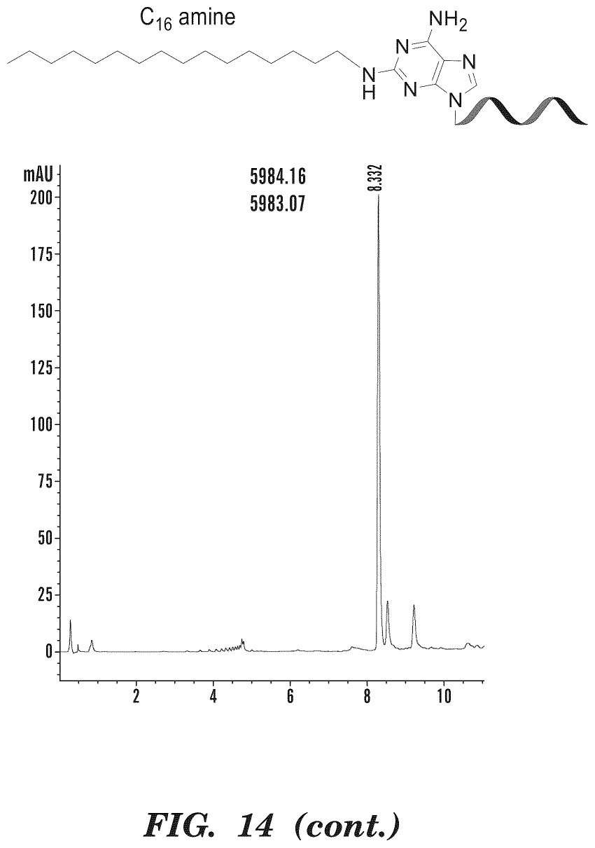

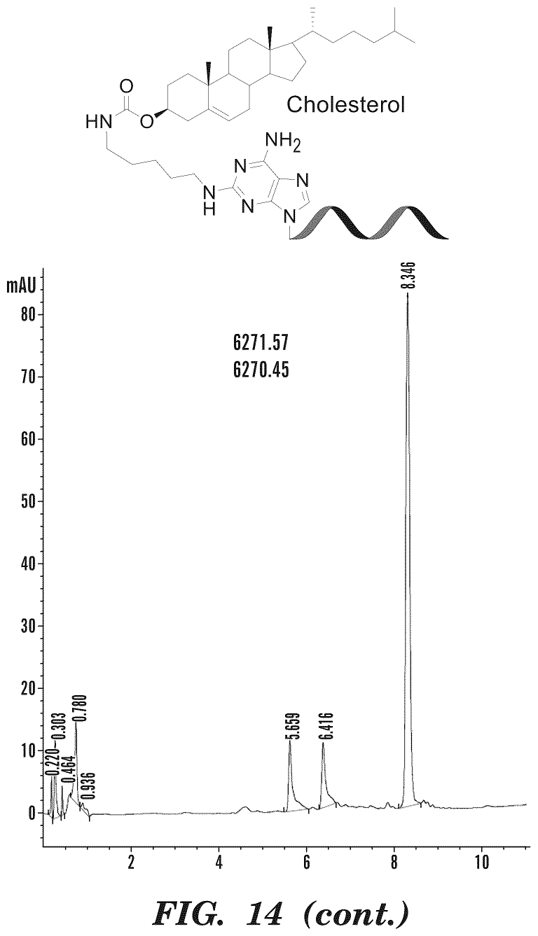

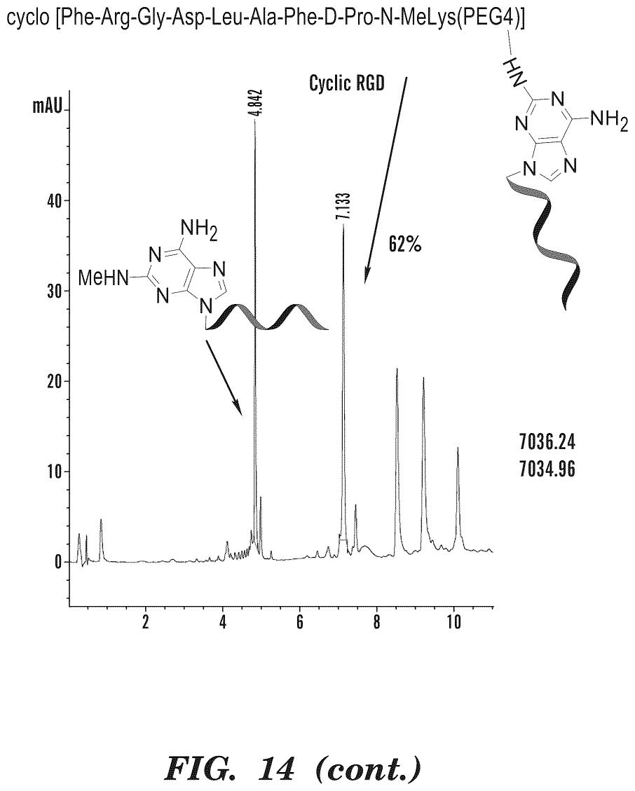

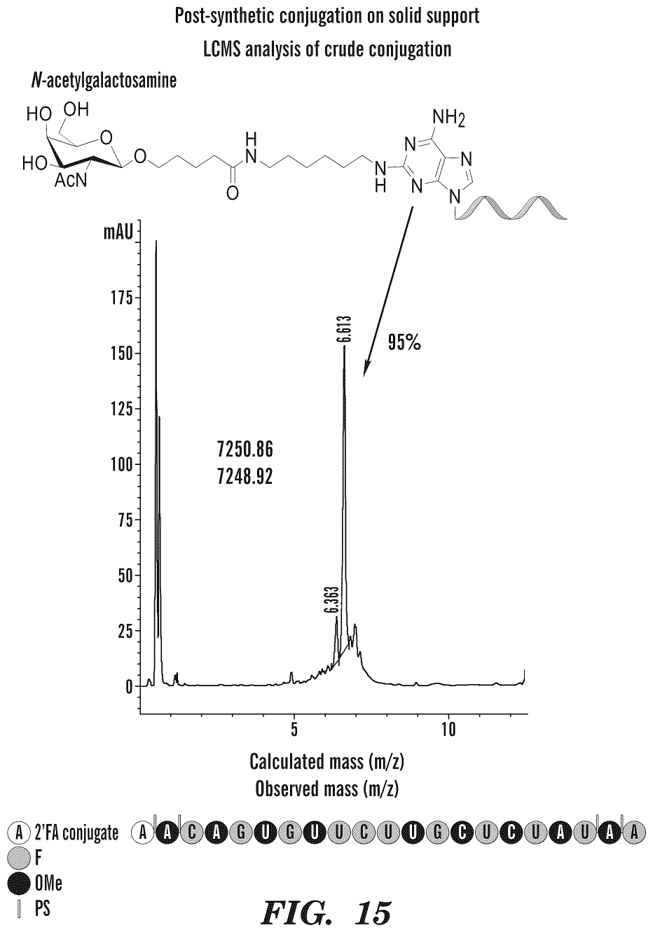

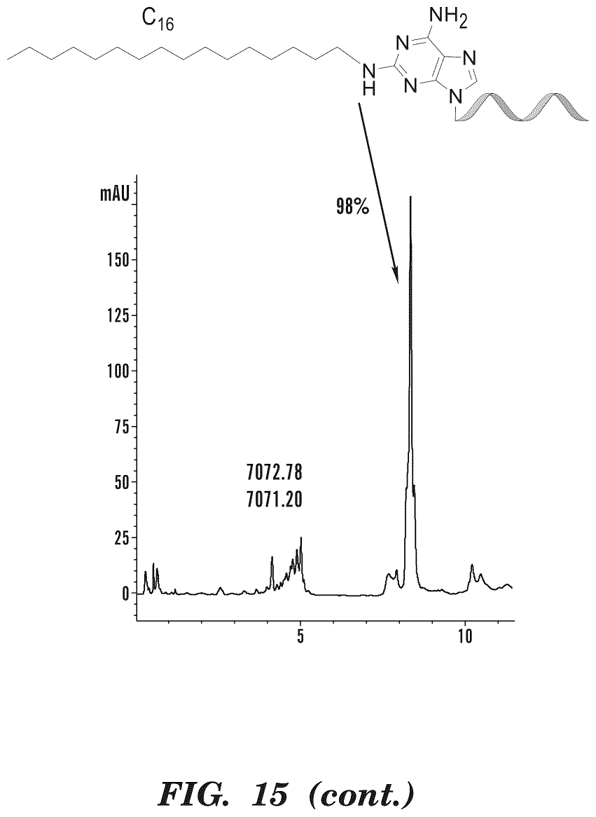

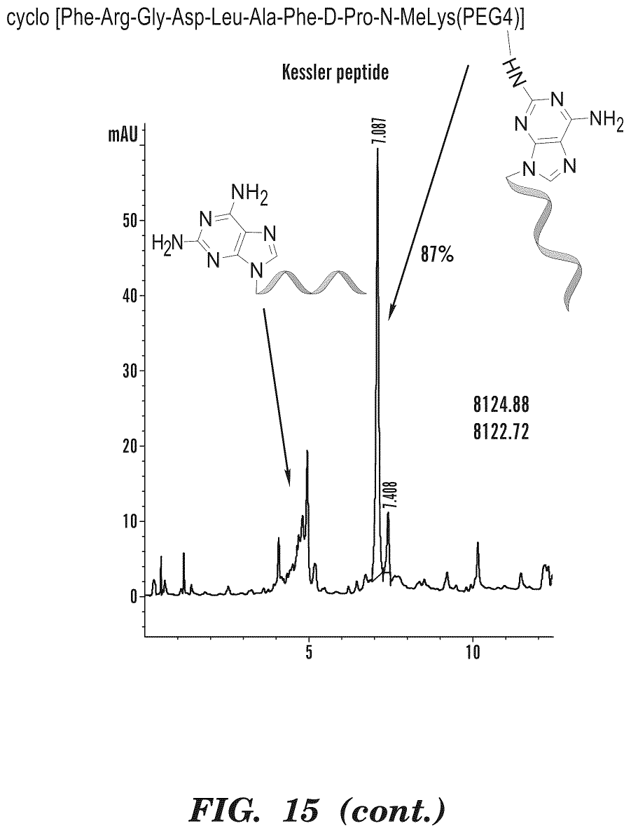

In a vial, 3 mg CPG, 100 μL of 0.2M ethanolic solution of amine and 10 μL DIPEA were combined, shaked and heated at 90° C. for 16h. Oligonucleotide conjugate was then deprotected and cleaved from the solid support using mixture of 28-30% NH₄OH for 5 h at 60° C. according to the method shown in FIG. 13 and Scheme 3. Illustrated examples are shown in FIGS. 14 and 15.

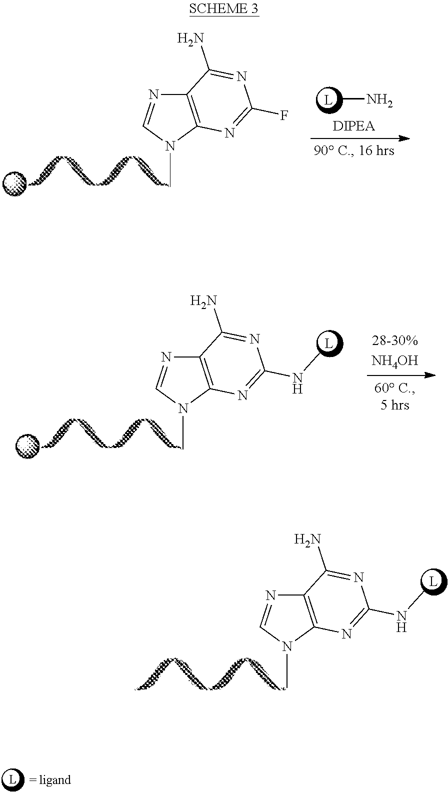

### Solution Phase Post-Synthesis Oligonucleotide Conjugation:

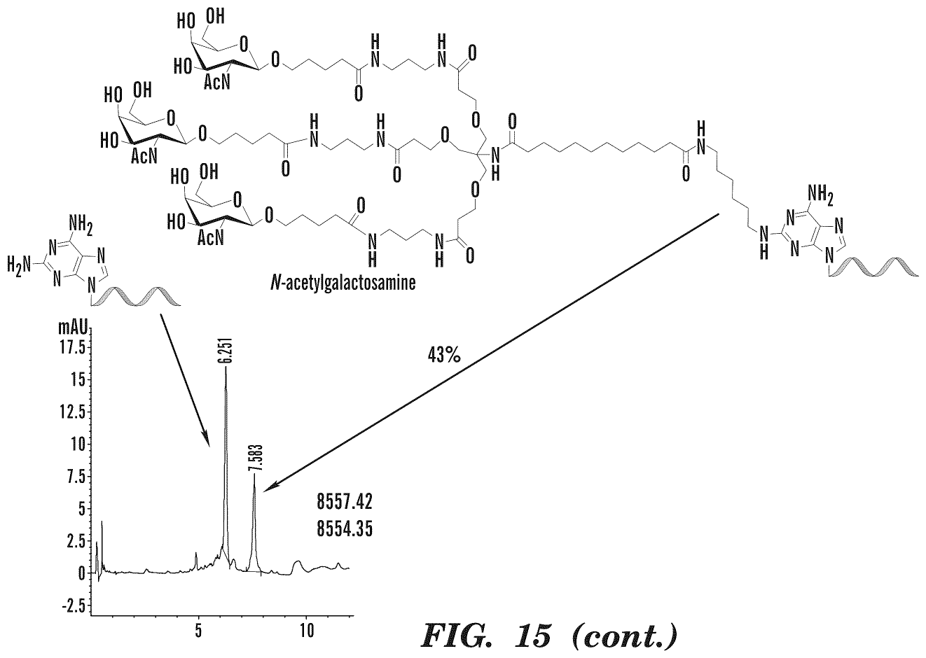

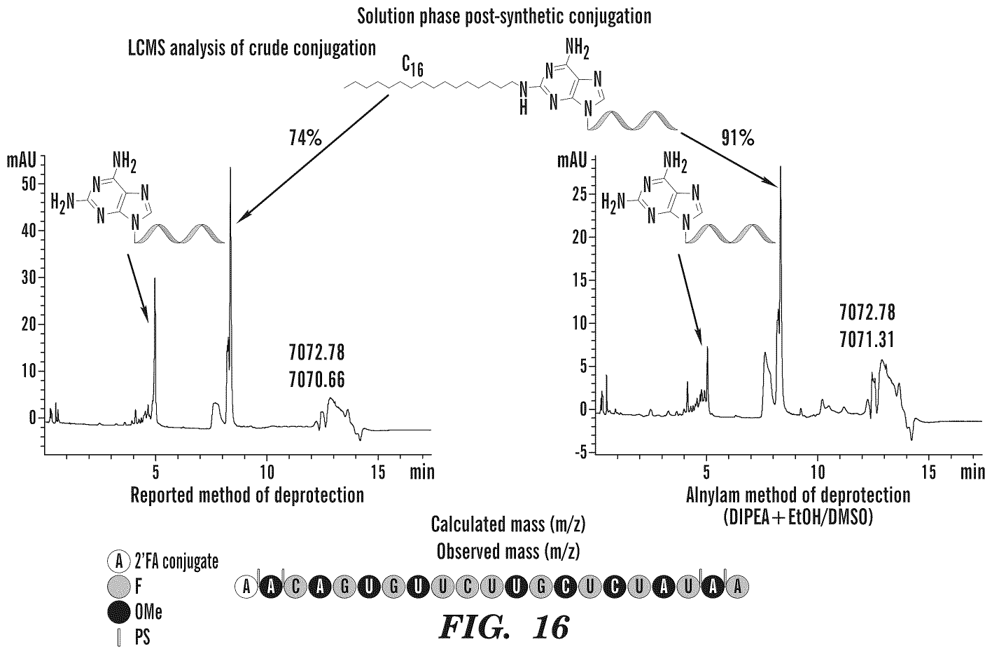

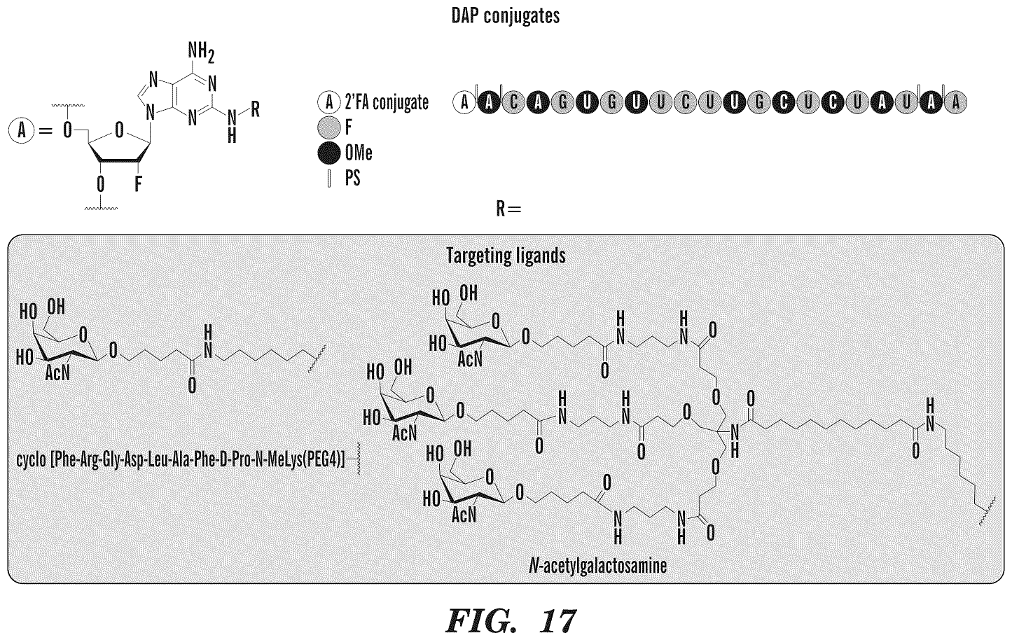

Reported method: In a vial, 3 mg CPG were treated with 1 mL mixture of 0.1M NaOH for 8 h at room temperature. The partially deprotected oligonucleotide was then filtrated from the solid support, purified by size exclusion chromatography and lyophized. 5 OD oligonucleotides, 34 L 8.25 μmol amino ligand were solubilized in 34 μL dry DMSO and heated at 75° C. for 24h according to the reported method by Decorte et al. [46] and shown in Scheme 4. Illustrated example is shown in FIG. 15. Illustrated example is shown in FIG. 16.

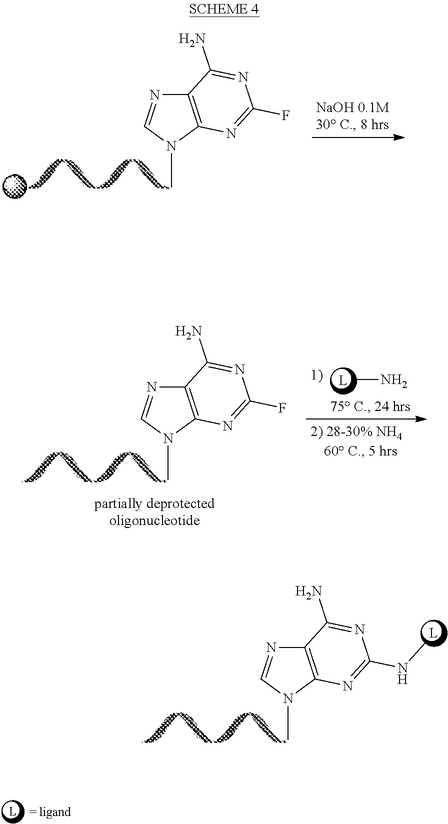

### Current Method:

In a vial, 26 mg CPG were treated with 500 L mixture of 28-30% NH₄OH for 2 h at room temperature. The partially deprotected oligonucleotide was then filtrated from the solid support, purified by size exclusion chromatography and lyophized. 5 OD oligonucleotides, 100 μL of 0.2M ethanolic solution of amine and 10 μL DIPEA were solubilized in 100 μL dry DMSO and heated at 90° C. for 16h according to the method shown in Scheme 5. Illustrated example is shown in FIG. 16.

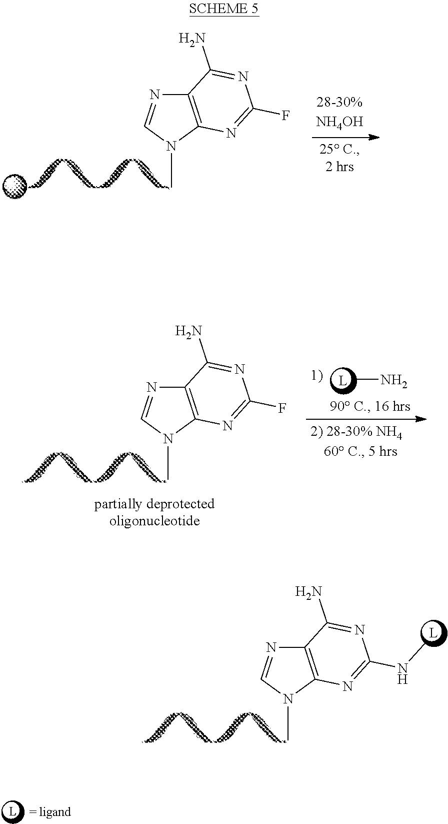

### Tm Measurement:

UV melting curves were recorded using a Cary 3500 UV-Visible Spectrophotometer Multicell Peltier. The concentration of oligonucleotide was 1 μM, and samples were prepared in PBS buffer (137 mM sodium chloride, 2.7 mM potassium chloride, 8 mM sodium phosphate dibasic, and 2 mM potassium phosphate monobasic, pH 7.4). Samples were annealed by heating to 85° C. and then slowly cooled to 10° C. Samples were then heated to 95° C. at a gradient of 1° C./min, and the change in UV absorbance at 260 nm was recorded. The melting temperature was calculated from the first derivative of the melting curve.

### Synthesis of Monomers

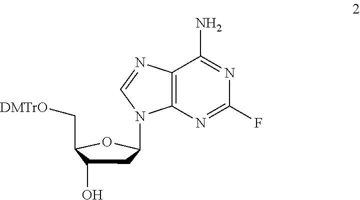

### Synthesis of 2:

To a stirred solution of (2R,5R)-5-(2,6-diaminopurin-9-yl)-2-(hydroxymethyl)tetrahydrofuran-3-ol (39.5 g, 148.35 mmol) in pyridine (300 mL) was added Hydrogen fluoride-pyridine (70% HF) (310.87 g, 2.20 mol, 272.69 mL, 70% purity) and tert-Butyl nitrite (34.00 g, 296.71 mmol, 39.21 mL, 90% purity) at 20° C. The reaction mixture was stirred for 40 h. The pH of the reaction mixture was adjusted to 7˜8 by adding NaHCO3 solution. The reaction mixture was extracted with ethyl acetate (5×1 μL). After evaporation of the solvent, the crude product was obtained as yellow solid and purified by recrystallization from methanol (500 mL) at 60° C. to give (2R,5R)-5-(6-amino-2-fluoro-purin-9-yl)-2-(hydroxymethyl)tetrahydrofuran-3-ol (22.7 g, 84.31 mmol, 56.83% yield) as yellow solid. 1H NMR (400 MHz, DMSO-d6) δ ppm: 8.31 (s, 1H), 7.82 (br s, 2H), 6.23 (t, J=6.8 Hz, 1H), 5.29 (d, J=4.0 Hz, 1H), 4.94 (t, J=5.6 Hz, 1H), 4.34-4.43 (m, 1H), 3.81-3.89 (m, 1H), 3.45-3.65 (m, 2H), 2.60-2.71 (m, 1H), 2.20-2.30 (m, 1H); ¹⁹F NMR δ ppm: −52.07 ppm

To a stirred solution of (2R,5R)-5-(6-amino-2-fluoro-purin-9-yl)-2-(hydroxymethyl) tetrahydrofuran-3-ol (22 g, 81.71 mmol) in pyridine (150 mL) was added 4,4′-Dimethoxytrityl chloride (29.07 g, 85.80 mmol) at 20° C., and the solution was stirred for 19 h. The reaction was quenched with methanol (20 mL). The solvent was evaporated under reduced pressure, and the residue was dissolved in ethyl acetate (200 mL) and washed with sat. aq. NaHCO₃ (2×100 mL), dried over Na₂SO₄, concentrated. The crude product was purified by column chromatography on silica gel eluting with 0˜5% methanol in ethyl acetate to give (2R,5R)-5-(6-amino-2-fluoro-purin-9-yl)-2-[[bis(4-methoxyphenyl)-phenyl-methoxy]methyl]tetrahydrofuran-3-ol (22.5 g, 39.36 mmol, 48.17% yield) as slightly yellow solid (95.95% pure). ¹H NMR (400 MHz, DMSO-d₆) δ ppm: 8.22 (s, 1H), 7.82 (br s, 2H), 7.31 (d, J=7.2 Hz, 2H), 7.13-7.25 (m, 7H), 6.79 (dd, J=12.4, 8.8 Hz, 4H), 6.26 (t, J=6.4 Hz, 1H), 5.36 (d, J=4.8 Hz, 1H), 4.36-4.52 (m, 1H), 3.93-3.98 (m, 1H), 3.71 (d, J=1.6 Hz, 6H), 3.06-3.24 (m, 2H), 2.75-2.85 (m, 1H), 2.25-2.38 (m, 1H); 19F NMR (376 MHz) δ ppm: −51.89; ¹³C NMR (101 MHz, DMSO-d₆) δ ppm: 159.51, 158.98, 157.73, 157.50, 150.39, 150.19, 144.89, 139.83, 135.56, 129.63, 127.68, 126.57, 117.69, 113.02, 85.83, 85.39, 83.39, 70.44, 63.98, 54.98; HRMS calculated for C31H30FN5O5[M+H]+ m/z=572.22, found 572.23.

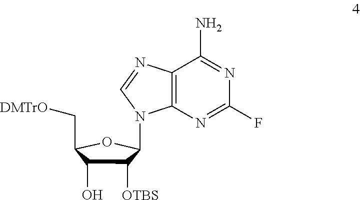

### Synthesis of 4:

4,4′-Dimethoxytrityl chloride (65.34 g, 192.83 mmol) was added to a solution of (2R,5R)-2-(6-amino-2-fluoro-purin-9-yl)-5-(hydroxymethyl)tetrahydrofuran-3,4-diol (50 g, 175.30 mmol) in pyridine (500 mL), then the reaction was stirred at 15° C. for 15 hrs. LCMS (RT of product was 0.840 min) showed the reaction was complete. The reaction mixture was added H₂O (500 mL), and extracted with EtOAc (500 mL×3), the combined organic layers were washed with brine (500 mL), then dried over Na₂SO₄, filtered and concentrated under reduced pressure to give a residue. The residue was purified by prep-HPLC (column: Phenomenex Titank C18 Bulk 250×100 mm 10u; mobile phase: [water(10 mM NH₄HCO₃)-ACN]; B %: 50%-70%, 20 min) to afford 5′-DMT-2-F-ribo-purine (85.5 g, 145.5 mmol, 83.0% yield) as a yellow solid. ¹H NMR DMSO-d₆, 400 MHz 6 8.24 (s, 1H), 7.84-7.91 (m, 2H), 7.17-7.35 (m, 9H), 6.79-6.83 (m, 4H), 5.83 (d, J=4.4 Hz, 1H), 5.57 (d, J=5.6 Hz, 1H), 5.23 (d, J=6 Hz, 1H), 4.60-4.64 (m, 1H), 4.25-4.30 (m, 1H), 3.99-4.06 (m, 2H), 3.72 (s, 6H), 3.16-3.24 (m, 2H) ppm.

AgNO₃ (33.8 g, 199.1 mmol, 1.21 eq) was added to a solution of 5′-DMT-2-F-ribo-purine (97.0 g, 165.0 mmol, 1.00 eq) and py (52.2 g, 660.9 mmol, 53.3 mL, 4.00 eq) in THF (776 mL), then the reaction was stirred at 15° C. for 20 min, then TBSCl (44.7 g, 297.1 mmol, 36.4 mL, 1.80 eq) was added to above reaction, then the reaction was stirred at 15° C. for 16 hrs under N2. TLC (Pet Ether/EtOAc=1/1, R_{f} of product=0.4) showed the reaction was complete. Also, the LCMS (ET43590-14-P1A) showed the reaction was complete. The reaction mixture was filtered and then the reaction was extracted with EtOAc (200 mL×3) and H2O (200 mL), the combined organic layers were washed with brine (200 mL), dried over Na₂SO₄, filtered and concentrated under reduced pressure to give a residue. The residue was purified by column chromatography (SiO₂, DCM/acetone=100/1 to 10/1). (2R,5R)-5-(6-amino-2-fluoro-purin-9-yl)-2-[[bis(4-methoxyphenyl)-phenyl-methoxy]methyl]-4-[tert-butyl(dimethyl)silyl]oxy-tetrahydrofuran-3-ol (25.2 g, 35.90 mmol, 20.48% yield) was obtained as a yellow solid which was the 3′-O-TBDMS isomer (96% purity). ¹H NMR DMSO-d₆, 400 MHz δ 8.23 (s, 1H), 7.85-7.92 (m, 2H), 7.18-7.39 (m, 9H), 6.82-6.85 (m, 4H), 5.84 (d, J=4.8 Hz, 1H), 5.17 (d, J=6 Hz, 1H), 4.71-4.73 (m, 1H), 4.18-4.22 (m, 1H), 4.06-4.10 (m, 1H), 3.72 (s, 6H), 3.23-3.30 (m, 2H), 0.76 (s, 9H), −0.067 (d, J=35.2 Hz, 6H) ppm.

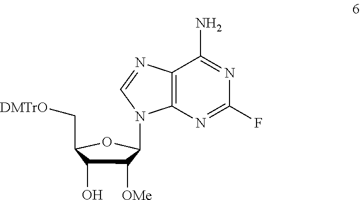

### Synthesis of 6:

To a 100 mL stainless steel Parr bomb was added 2′-O-Methylguanosine (10.0 g, 33.6 mmol, 1.00 eq), HMDS (27.1 g, 168.2 mmol, 35.2 mL, 5.00 eq) and toluene (10 mL), then TMSOTf (972 mg, 4.37 mmol, 0.13 eq) was added to above reaction. The reaction was stirred at 170° C. for 120 hrs. After cooling room temperature, the four reactions were dissolved with MeOH (200 mL) and concentrated under reduced pressure to give a mixture of multiple TMS-protected intermediates (50.0 g) without purification.

The intermediate from first step (30.0 g) was dissolved with MeOH (150 mL) and H₂O (48.7 mL), and stirred at 75° C. for 12 hrs. The reaction was concentrated under reduced pressure, the residue was purified by prep-HPLC (column: Phenomenex Titank C18 Bulk 250*100 mm 10 u; mobile phase: [water (10 mM NH₄HCO₃)-ACN]; B %: 1%-15%, 20 min) to give (2R,5R)-5-(2,6-diaminopurin-9-yl)-2-(hydroxymethyl)-4-methoxy-tetrahydrofuran-3-ol (10.8 g) as white solid. 1H NMR (DMSO-d₆, 400 MHz), δ ppm: 7.97 (s, 1H), 6.84 (s, 2H), 5.70-5.84 (m, 3H), 5.47-5.55 (m, 1H), 5.51 (d, J=4.0 Hz, 1H), 4.20-4.35 (m, 2H), 4.05-4.18 (m, 2H), 3.94 (s, 1H), 3.53-3.70 (m, 2H), 3.29 (s, 3H) ppm.

(2R,5R)-5-(2,6-diaminopurin-9-yl)-2-(hydroxymethyl)-4-methoxy-tetrahydrofuran-3-ol (9 g, 30.38 mmol) was added to a solution of Hydrogen fluoride-pyridine (70% HF) (34.41 g, 243.01 mmol, 30.18 mL, 70% purity) in pyridine (60 mL) at −20˜0° C., then tert-Butyl nitrite (10.96 g, 106.32 mmol, 12.65 mL) was added to above reaction at −10° C. 0° C. The reaction was stirred at 15° C. for 16 hrs. The reaction was added to cooled sat. aq. NaHCO₃ (100 mL), then the reaction was adjusted to pH 7˜8 with sat. aq. NaHCO₃, extracted with EtOAc (100 mL×15). The combined organic layers were dried over Na₂SO₄, filtered and concentrated under reduced pressure to give a brown solid. The solid was washed with EtOAc (18 mL) to give (2R,5R)-5-(6-amino-2-fluoro-purin-9-yl)-2-(hydroxymethyl)-4-methoxy-tetrahydrofuran-3-ol (8.4 g, 28.07 mmol, 92.41% yield) (1c) as yellow solid. ¹H NMR: (DMSO-d₆, 400 MHz) δ ppm: 8.34 (s, 2H), 7.88 (br, s, 2H), 5.90 (d, J=5.6 Hz, 1H), 4.25-4.37 (m, 2H), 3.94-3.97 (m, 1H), 3.62-3.69 (m, 1H), 3.50-3.57 (m, 1H), 3.32 (s, 3H); ¹⁹F NMR (DMSO-d₆, 376 MHz) δ ppm: −51.85.

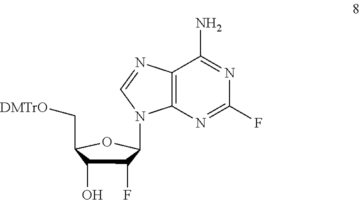

### Synthesis of 1d:

Prepared following the procedure mentioned in Manoharan, Muthiah; Rajeev, Kallanthottathil G. PCT Int. Appl. (2009), WO 2009091982 A1 20090723.

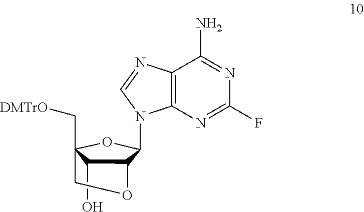

### Synthesis of 10:

N′-[9-[(4R,6R)-4-[[bis(4-methoxyphenyl)-phenyl-methoxy]methyl]-7-hydroxy-2,5-dioxabicyclo[2.2.1]heptan-6-yl]-6-oxo-1H-purin-2-yl]-N,N-dimethyl-formamidine (40 g, 61.28 mmol) was dissolved in aq. NaOH (4.00 M, 400 mL) and methanol (400 mL) at 20° C., stirred at 60° C. for 2 h. The reaction was concentrated to remove MeOH at 45° C., then neutralized by 1 N HCl to pH˜7, white solid was precipitated and collected by filtration, washed with water, azeotropy with ACN (500 mL×3) to removed water at 45° C. to give 2-amino-9-[(4R,6R)-4-[[bis(4-methoxyphenyl)-phenyl-methoxy]methyl]-7-hydroxy-2,5-dioxabicyclo[2.2.1]heptan-6-yl]-1H-purin-6-one (32 g, 53.55 mmol, 87.37% yield) as white solid. ¹H NMR (DMSO-d₆, 400 MHz) δ ppm: 7.67 (s, 1H), 7.57-6.76 (m, 15H), 5.76 (s, 2H), 4.33 (d, J=21.2 Hz, 2H), 4.05-3.61 (m, 8H), 3.55-3.20 (m, 2H) ppm.

2-amino-9-[(4R,6R)-4-[[bis(4-methoxyphenyl)-phenyl-methoxy]methyl]-7-hydroxy-2,5-dioxabicyclo[2.2.1]heptan-6-yl]-1H-purin-6-one (26.7 g, 44.68 mmol) was dissolved in dried Py (300 mL×3) (dried by KOH) and concentrated at 50° C., then dissolved in dry py (160 mL) and cooled to 0° C., Trifluoroacetic anhydride (65.69 g, 312.74 mmol, 44.08 mL) was added slowly at 0˜10° C. The reaction was stirred at 0˜10° C. for 45 min under N2. TLC (DCM/MeOH=10/1, R_{f} of ELN0132−364=0.05, R_{f} of intermediate=0.30) indicated ELN0132-364 was consumed completely. The light brown solution was divided evenly onto 6 parts and used directly for the next step immediately.

Liquid NH₃ (25 mL) was added slowly to one of the parts at −60° C., sealed and warmed to 10˜17° C. for 16 h. TLC (DCM/MeOH=5/1, R_{f}=0.40) indicated reaction was consumed completely. The reactions were combined and diluted with DCM (300 mL), adjusted to pH˜8 with sat. Aq. NaHCO₃ (700 mL), extracted with DCM (500 mL×7), the combined organic layers were concentrated. The residue was purified by column chromatography (SiO₂), DCM/MeOH (0.5% TEA)=0/1˜5/1) to give (4R,6R)-4-[[bis(4-methoxyphenyl)-phenyl-methoxy]methyl]-6-(2,6-diaminopurin-9-yl)-2,5-dioxabicyclo[2.2.1]heptan-7-ol (25 g, 41.90 mmol, 93.79% yield) (included THF) as brown solid. ¹H NMR (DMSO-d₆, 400 MHz) δ ppm: 7.81 (s, 1H), 7.42 (d, J=7.6 Hz, 2H), 7.36-7.21 (m, 7H), 7.10 (s, 2H), 6.90 (d, J=8.4 Hz, 4H), 6.19 (s, 2H), 5.80 (s, 1H), 5.74 (d, J=4.4 Hz, 1H), 4.38 (s, 1H), 4.33 (d, J=4.4 Hz, 1H), 3.98-3.85 (m, 2H), 3.74 (s, 6H), 3.49 (d, J=10.8 Hz, 1H), 3.31 (d, J=10.8 Hz, 1H) ppm.

References: Bioorganic & Medicinal Chemistry 2004, 12, 2385-2396; Tetrahedron Letters, 1994, 35, 2489-2492.

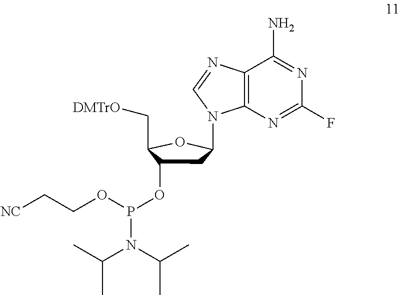

### 3-[[(2R,5R)-5-(6-amino-2-fluoro-purin-9-yl)-2-[[bis(4-methoxyphenyl)-phenyl-methoxy]methyl] tetrahydrofuran-3-yl]oxy-(diisopropylamino)phosphanyl]oxypropanenitrile

Compound 11: To a clear solution of 2 (2.00 g, 3.43 mmol) in DCM (20 mL) at 22° C. was added N-Methyl imidazole (563.04 mg, 6.86 mmol, 546.64 μL) and diisopropylethylamine (2.22 g, 17.14 mmol, 2.99 mL). The reaction mixture was stirred for 5 minutes at rt and 2-cyanoethyl-N,N-diisopropylchlorophosphoramidite (1.62 g, 6.86 mmol, 1.53 mL) was added slowly into it. Reaction was kept for stirring at 22° C. and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 40-80% EtOAc in hexane) to afford 11 (1.87 g, 71% yield) as white foam. ¹H NMR (500 MHz, CD₃CN) δ 8.00 (d, J=1.3 Hz, 1H), 7.36 (ddt, J=7.3, 6.1, 1.5 Hz, 3H), 7.23-7.14 (m, 6H), 6.83-6.72 (m, 6H), 6.43 (s, 3H), 6.27 (dt, J=6.9, 5.7 Hz, 2H), 4.91-4.70 (m, 1H), 4.17 (dq, J=15.4, 4.5 Hz, 2H), 3.85-3.69 (m, 11H), 3.68-3.52 (m, 3H), 3.36-3.23 (m, 3H), 3.00-2.90 (m, 1H), 2.67-2.49 (m, 5H), 1.20-1.13 (m, 15H), 1.07 (d, J=6.9 Hz, 5H) ppm. ¹³C NMR (101 MHz, CD₃CN) δ 160.93, 159.62, 159.58, 158.89, 158.68, 158.47, 151.95, 151.92, 151.75, 151.72, 146.06, 146.04, 141.17, 141.14, 141.12, 136.85, 136.80, 136.78, 136.74, 131.00, 130.98, 130.92, 130.90, 128.97, 128.91, 128.72, 127.76, 127.73, 119.56, 119.38, 119.31, 119.30, 119.27, 119.26, 113.94, 113.91, 87.00, 86.42, 86.38, 86.20, 86.14, 85.27, 85.19, 74.27, 74.10, 73.77, 73.61, 64.49, 64.35, 60.95, 59.65, 59.55, 59.46, 59.35, 55.85, 55.84, 44.09, 44.07, 43.97, 43.94, 39.12, 39.09, 38.94, 38.90, 24.95, 24.93, 24.91, 24.89, 24.88, 24.86, 24.84, 24.82, 21.15, 21.09, 21.02, 21.00, 20.93 ppm. 31p NMR (202 MHz, CD₃CN) δ 146.60, 146.55, 146.42, 146.40 ppm. ¹⁹F NMR (565 MHz, CD₃CN) δ −52.23, −52.39 ppm. HRMS calc. for C₄₀H₄₈FN₇O₆P [M+H]⁺ 772.3388, found 772.3391.

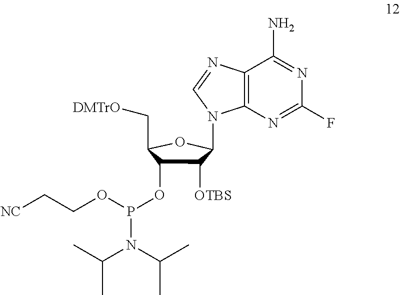

### 3-[[(2R,5R)-5-(6-amino-2-fluoro-purin-9-yl)-2-[[bis(4-methoxyphenyl)-phenyl-methoxy]methyl]-4-[tert-butyl(dimethyl)silyl]oxy-tetrahydrofuran-3-yl]oxy-(diisopropylamino)phosphanyl] oxypropanenitrile Compound 12

To a clear solution of 4 in DCM (20 mL) at 22° C. was added N-methyl imidazole (343.21 mg, 4.18 mmol, 333.21 μL) and diisopropylethylamine (1.80 g, 13.93 mmol, 2.43 mL)The reaction mixture was stirred for 5 minutes at rt and 2-cyanoethyl-N,N-diisopropylchlorophosphoramidite (1.32 g, 5.57 mmol, 1.24 mL) was added slowly into it. Reaction was kept for stirring at 22° C. and TLC was checked after 0.5 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 20-50% EtOAc in hexane) to afford 12 (2.25 g, 89% yield) as yellowish white foam. ¹H NMR (600 MHz, CD₃CN) δ 8.01 (dd, J=11.3, 2.0 Hz, 1H), 7.48-7.42 (m, 2H), 7.40-7.10 (m, 8H), 6.83 (ddd, J=9.0, 6.8, 2.2 Hz, 4H), 6.44 (s, 2H), 5.85 (dt, J=5.7, 2.8 Hz, 1H), 4.93 (dt, J=10.6, 5.1 Hz, 1H), 4.48-4.34 (m, 2H), 4.29 (q, J=3.8 Hz, 1H), 3.92-3.80 (m, 1H), 3.77-3.73 (m, 6H), 3.71-3.56 (m, 3H), 3.49-3.40 (m, 1H), 3.33 (dt, J=14.8, 7.4 Hz, 1H), 2.66 (q, J=6.2 Hz, 1H), 2.42 (td, J=6.1, 2.2 Hz, 1H), 1.29-1.10 (m, 13H), 1.05 (dd, J=6.9, 2.3 Hz, 3H), 0.77 (d, J=2.4 Hz, 9H), −0.01 (dd, J=8.1, 2.2 Hz, 3H), -0.15 (dd, J=11.0, 2.2 Hz, 3H) ppm. ¹³C NMR (151 MHz, CD₃CN) δ 160.66, 160.61, 159.63, 159.60, 159.30, 159.24, 158.67, 158.53, 152.20, 152.14, 152.06, 152.01, 146.04, 145.97, 141.16, 141.14, 140.89, 140.87, 136.75, 136.64, 136.55, 136.53, 131.09, 131.05, 131.02, 131.00, 128.97, 128.90, 128.80, 127.82, 119.48, 119.27, 113.99, 113.97, 89.35, 89.25, 87.25, 87.21, 84.65, 84.37, 84.34, 75.55, 75.54, 75.46, 75.43, 73.95, 73.88, 73.52, 73.43, 64.32, 64.29, 60.94, 59.82, 59.71, 58.96, 58.82, 55.84, 55.83, 44.12, 44.03, 43.75, 43.67, 25.99, 25.97, 25.17, 25.12, 25.01, 24.97, 24.93, 24.89, 24.88, 24.85, 21.14, 21.11, 21.07, 20.90, 20.85, 18.54, −4.37, −4.38, −4.41, −4.92 ppm. ¹⁹F NMR (565 MHz, CD₃CN) δ −52.61, −52.71 ppm. HRMS calc. for C₄₆H₆₂FN₇O₇PSi [M+H]⁺ 902.4202 found 902.4198.

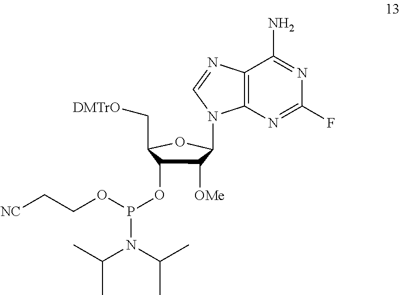

### 3-[[5-(6-amino-2-fluoro-purin-9-yl)-2-[[bis(4-methoxyphenyl)-phenyl-methoxy]methyl]-4-methoxy-tetrahydrofuran-3-yl]oxy-(diisopropylamino)phosphanyl]oxypropanenitrile Compound 13

To a clear solution of 6 (2.00 g, 3.32 mmol) in DCM (12 mL) at 22° C. was added N-Methyl imidazole (551.37 mg, 6.65 mmol, 535.31 μL) and diisopropylethylamine (2.16 g, 16.62 mmol, 2.91 mL). The reaction mixture was stirred for 5 minutes at rt and 2-cyanoethyl-N,N-diisopropylchlorophosphoramidite (1.18 g, 4.99 mmol, 1.11 mL) was added slowly into it. Reaction was kept for stirring at 22° C. and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 40-80% EtOAc in hexane) to afford 13 (2.36 g, 85% yield) as white foam. ¹H NMR (600 MHz, CD₃CN) δ 8.01 (d, 1H), 7.43-7.37 (m, 2H), 7.32-7.18 (m, 7H), 6.81 (dt, J=13.4, 8.4 Hz, 4H), 6.37 (s, 2H), 5.95 (t, J=4.5 Hz, 1H), 4.72-4.64 (m, 1H), 4.53 (q, J=5.1 Hz, 1H), 4.30-4.21 (m, 1H), 3.93-3.79 (m, 1H), 3.79-3.69 (m, 7H), 3.70-3.55 (m, 2H), 3.43 (d, 3H), 3.40-3.26 (m, 2H′), 2.67 (dt, 1H), 2.49 (t, J=6.0 Hz, 1H), 1.17 (t, 9H), 1.06 (d, J=6.8 Hz, 3H)¹³C NMR (151 MHz, CD₃CN) 160.21, 159.24, 158.85, 158.28, 158.15, 151.59, 151.46, 145.57, 140.85, 140.62, 136.39, 136.31, 136.27, 130.67, 130.63, 130.61, 130.58, 128.63, 128.56, 128.37, 128.35, 127.43, 127.39, 119.18, 118.93, 113.57, 113.55, 87.66, 87.36, 86.75, 86.73, 83.84, 83.82, 83.71, 83.67, 82.41, 82.39, 82.16, 82.13, 71.60, 71.51, 71.48, 71.37, 63.67, 63.32, 59.49, 59.37, 59.00, 58.87, 58.70, 58.68, 58.42, 58.41, 55.46, 55.44, 43.64, 24.47, 20.56. ¹⁹F NMR (565 MHz, CD₃CN) δ −52.56. ³¹P NMR (243 MHz, CD₃CN) δ 149.99, 149.71. HRMS calc. for C₄₁H₅₀FN₇O₇P [M+H]⁺ 802.3493, found 802.3503.

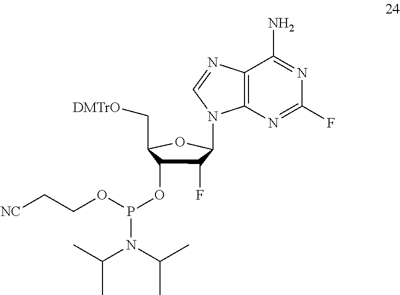

### 3-[[(2R,5R)-5-(6-amino-2-fluoro-purin-9-yl)-2-[[bis(4-methoxyphenyl)-phenyl-methoxy]methyl]-4-fluoro-tetrahydrofuran-3-yl]oxy-(diisopropylamino)phosphanyl]oxypropanenitrile Compound 24

Commercially available amidite showed little impurity in ³¹P NMR. To improve the amidite quality, 24 (1.1 g, 1.39 mmol) was dissolved in tert-butylmethylether (MTBE) (30 mL) and washed with 50% DMF in water (2×30 mL) and organic layer was separated. MTBE layer was then washed with brine (3×20 mL) and dried over anhydrous Na₂SO₄, filtered and the solvent from the filtrate was completely evaporated under high vacuum pump to afford yellowish white foam (1.0 g). ¹H NMR (600 MHz, CD₃CN) δ 8.08 (d, J=2.6 Hz, 1H), 7.41-7.34 (m, 2H), 7.28-7.17 (m, 7H), 6.84-6.75 (m, 4H), 6.67-6.52 (m, OH), 6.27-6.17 (m, 1H), 5.87-5.59 (m, 1H), 5.27-5.00 (m, 1H), 4.27 (ddd, J=8.3, 4.3, 2.0 Hz, 1H), 3.98-3.78 (m, 1H), 3.77-3.72 (m, 6H), 3.69-3.55 (m, 2H), 3.54-3.44 (m, 1H), 3.27 (ddd, J=11.1, 9.3, 4.5 Hz, 1H), 2.73-2.65 (m, 1H), 2.52 (t, J=6.0 Hz, 1H), 1.23-1.14 (m, 11H), 1.06 (d, J=6.8 Hz, 3H) ppm. ¹³C NMR (151 MHz, CD₃CN) δ 160.62, 160.58, 159.53, 159.50, 159.49, 159.25, 159.21, 158.70, 158.68, 158.56, 158.55, 151.42, 151.30, 151.28, 145.86, 145.80, 141.52, 141.50, 136.69, 136.64, 136.54, 136.49, 130.93, 130.91, 130.88, 130.85, 129.85, 129.15, 128.91, 128.83, 128.70, 128.69, 127.75, 127.71, 119.50, 119.26, 119.11, 119.08, 119.06, 113.87, 113.84, 94.44, 93.84, 93.82, 93.21, 92.59, 92.58, 88.78, 88.74, 88.54, 88.51, 86.89, 86.83, 82.26, 82.23, 82.18, 82.13, 71.16, 70.47, 62.88, 62.35, 59.94, 59.80, 59.67, 59.53, 55.80, 55.77, 55.29, 49.45, 44.07, 43.99, 27.19, 24.98, 24.95, 24.92, 24.90, 24.88, 24.83, 24.81, 24.76, 20.98, 20.93, 20.88, 20.84 ppm. 31p NMR (202 MHz, CD₃CN) δ 148.66, 148.63, 148.60 ppm. ¹⁹F NMR (565 MHz, CD₃CN) δ −52.22 (d, J=91.2 Hz), −196.69-−208.14 (m) ppm. HRMS calc. for C₄₀H₄₇F₂N₇O₆P [M+H]⁺ 790.3294, found 790.3307.

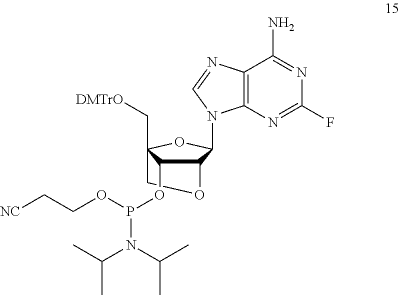

### 3-[[6-(6-amino-2-fluoro-purin-9-yl)-4-[[bis(4-methoxyphenyl)-phenyl-methoxy]methyl]-2,5-dioxabicyclo[2.2.1]heptan-7-yl]oxy-(diisopropylamino)phosphanyl]oxypropanenitrile Compound 15

To a clear solution of 10 (2.00 g, 3.34 mmol) in DCM (12 mL) at 22° C. was added N-Methyl imidazole (553.22 mg, 6.67 mmol, 537.11 μL) and diisopropylethylamine (2.17 g, 16.68 mmol, 2.92 mL). The reaction mixture was stirred for 5 minutes at rt and 2-cyanoethyl-N,N-diisopropylchlorophosphoramidite (1.18 g, 4.99 mmol, 1.11 mL) was added slowly into it. Reaction was kept for stirring at 22° C. and TLC was checked after 1 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 40-80% EtOAc in hexane) to afford 15 (2.36 g, 85% yield) as white foam. ¹H NMR (600 MHz, DMSO-d₆) δ 8.22-7.97 (m, 1H), 7.99-7.67 (m, 2H), 7.34 (t, J=8.6 Hz, 2H), 7.28-7.05 (m, 7H), 6.82 (td, J=9.4, 5.3 Hz, 4H), 5.94 (d, 1H), 4.70-4.60 (m, 1H), 4.60-4.51 (m, 1H), 4.00-3.86 (m, 1H), 3.86-3.71 (m, 1H), 3.67 (d, 6H), 3.64-3.57 (m, 1H), 3.50-3.22 (m, 6H), 2.60 (q, 1H), 2.49 (t, 1H), 0.99 (t, 6H), 0.86 (d, J=6.7 Hz, 4H), 0.78 (d, J=6.7 Hz, 3H). ¹³C NMR (151 MHz, DMSO-d₆) δ 158.65, 158.34, 158.22, 158.07, 150.20, 145.07, 138.73, 138.41, 135.75, 135.67, 135.50 (d, J=9.0 Hz), 130.25, 130.18, 130.12, 128.34, 128.12, 128.04, 127.28, 119.20, 119.03, 118.13, 113.70, 113.68, 87.45, 86.29, 86.05, 86.03, 78.25, 72.67, 72.53, 59.78, 59.35, 59.07, 58.95, 58.82, 58.70, 55.52, 55.49, 43.16, 43.08, 24.70, 24.65, 24.60, 24.52, 24.47, 24.37, 24.32, 20.17, 20.15, 20.13. ¹⁹F NMR (565 MHz, DMSO-d₆) δ −51.69. ³¹P NMR (243 MHz, DMSO-d₆) δ 148.15, 148.00. HRMS calc. for C₄₁H₄₈FN₇O₇P [M+H]⁺ 800.3337, found 800.3331.

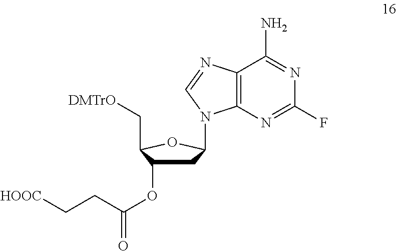

### 4-[(2R,5R)-5-(6-amino-2-fluoro-purin-9-yl)-2-[[bis(4-methoxyphenyl)-phenyl-methoxy] methyl] tetrahydrofuran-3-yl] oxy-4-oxo-butanoic acid Compound 16

To a clear solution of 2 (0.24 g, 419.88 μmol) in DCM (7 mL) and pyridine (3.5 mL) was added 4-(dimethylamino)pyridine (153.89 mg, 1.26 mmol) and succinic anhydride (126.05 mg, 1.26 mmol) sequentially. The resulting mixture was stirred for 2 hrs at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% MeOH in DCM) for afford 16 (0.201 g, 71% yield) as yellowish white solid. ¹H NMR (600 MHz, DMSO-d₆) δ 12.26 (s, 1H), 8.25 (d, J=2.0 Hz, 1H), 7.96-7.69 (m, 2H), 7.35-7.31 (m, 2H), 7.25-7.14 (m, 7H), 6.80 (ddd, J=16.7, 9.0, 2.2 Hz, 4H), 6.32-6.24 (m, 1H), 5.35 (dt, J=6.5, 2.7 Hz, 1H), 4.16 (dq, J=6.1, 2.8 Hz, 1H), 3.72 (d, J=2.3 Hz, 6H), 3.19 (dt, J=9.9, 3.3 Hz, 1H), 3.13-3.05 (m, 1H), 2.59-2.49 (m, 6H) ppm. ¹³C NMR (151 MHz, DMSO-d₆) δ 173.39, 171.75, 159.14, 158.05, 158.02, 157.99, 157.97, 157.79, 157.72, 157.58, 150.44, 150.35, 150.30, 144.88, 144.75, 139.90, 139.88, 135.58, 135.52, 135.43, 135.39, 129.66, 129.59, 127.70, 127.67, 127.65, 127.62, 126.63, 126.57, 117.73, 117.70, 113.07, 113.04, 113.01, 85.61, 83.69, 83.56, 74.67, 63.92, 54.98, 54.97, 54.91, 35.35, 28.81, 28.65 ppm. ¹⁹F NMR (565 MHz, DMSO-d₆) δ −51.72 ppm. HRMS calc. for C₃₅H₃₅FN₅O₈ [M+H]⁺ 672.2470, found 672.2481.

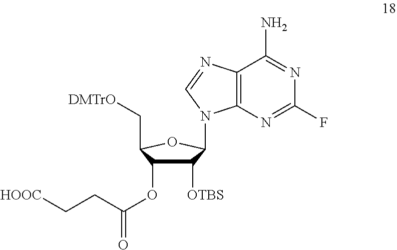

### 4-[(2R,5R)-5-(6-amino-2-fluoro-purin-9-yl)-2-[[bis(4-methoxyphenyl)-phenyl-methoxy] methyl]-4-[tert-butyl(dimethyl)silyl]oxy-tetrahydrofuran-3-yl]oxy-4-oxo-butanoic acid Compound 18

To a clear solution of 4 (0.7 g, 997.35 μmol) in DCM (8 mL) and pyridine (2 mL) was added 4-(dimethylamino)pyridine (365.54 mg, 2.99 mmol) and succinic anhydride (299.42 mg, 2.99 mmol) sequentially. The resulting mixture was stirred for 2 hrs at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% Methanol in DCM) for afford compound 18 (0.74 g, 93% yield) as yellowish white solid. ¹H NMR (600 MHz, DMSO-d₆) δ 12.30 (s, 1H), 8.57 (dd, J=4.4, 1.7 Hz, 1H), 8.29 (d, J=1.4 Hz, 1H), 8.01 (s, 1H), 7.90 (s, 1H), 7.41-7.34 (m, 3H), 7.29-7.19 (m, 7H), 6.87-6.81 (m, 4H), 5.84 (d, J=6.9 Hz, 1H), 5.31-5.27 (m, 1H), 5.13 (t, J=6.2 Hz, 1H), 4.21 (d, J=5.3 Hz, 1H), 3.72 (d, J=1.7 Hz, 7H), 3.39-3.34 (m, 2H), 3.30 (dd, J=10.6, 4.0 Hz, 1H), 2.64 (dt, J=16.9, 6.7 Hz, 1H), 2.60-2.50 (m, 3H), 0.66 (s, 9H), −0.10 (s, 3H), −0.30 (s, 3H) ppm. ¹³C NMR (151 MHz, DMSO-d₆) δ 173.24, 171.30, 159.35, 158.15, 157.99, 157.83, 157.69, 150.78, 150.65, 149.65, 144.80, 140.00, 136.18, 135.29, 129.79, 129.76, 128.96, 127.85, 127.69, 127.66, 127.46, 126.79, 123.98, 123.95, 117.69, 117.66, 113.20, 112.79, 87.04, 85.84, 81.59, 72.38, 63.39, 55.03, 28.72, 28.56, 25.21, 17.45, −5.37, −5.79 ppm. ¹⁹F NMR (565 MHz, DMSO-d₆) δ −51.55 ppm. HRMS calc. for C₄₁H₄₉FN₅O₉Si [M+H]⁺ 802.3284, found 802.3292.

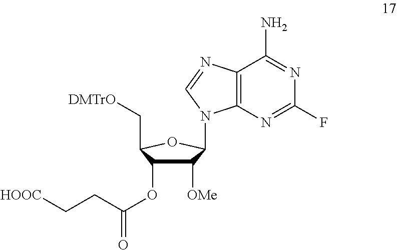

### 4-[5-(6-amino-2-fluoro-purin-9-yl)-2-[[bis(4-methoxyphenyl)-phenyl-methoxy]methyl]-4-methoxy-tetrahydrofuran-3-yl]oxy-4-oxo-butanoic acid Compound 17

To a clear solution of 6 (3 g, 4.99 mmol) in pyridine (15 mL) and was added 4-(dimethylamino)pyridine (1.23 g, 9.97 mmol) and succinic anhydride (1.01 g, 9.97 mmol) sequentially. The resulting mixture was stirred for 3 hrs at 22° C. Pyrimidine was evaporated under vacuum, then residue was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-20% MeOH in DCM) for afford pyridinium salt of 17 (2.75 g, 78% yield) as yellowish white solid. ¹H NMR (600 MHz, CD₃CN) δ 8.60-8.52 (m, 1H), 7.98 (s, 1H), 7.80-7.69 (m, 1H), 7.42-7.38 (m, 2H), 7.38-7.32 (m, 1H), 7.31-7.17 (m, 7H), 6.84-6.77 (m, 4H), 6.51 (s, 2H), 5.92 (d, J=5.9 Hz, 1H), 5.47 (t, J=4.5 Hz, 1H), 4.70 (t, J=5.6 Hz, 1H), 4.27-4.22 (m, 1H), 3.74 (s, 6H), 3.42-3.33 (m, 2H), 3.30 (s, 3H), 2.73-2.52 (m, 4H)¹³C NMR (151 MHz, CD₃CN) δ 174.01, 172.19, 160.29, 159.28, 159.25, 158.93, 158.30, 158.16, 151.75, 151.62, 149.98, 145.47, 140.64, 140.63, 136.87, 136.28, 136.22, 130.61, 130.54, 128.56, 128.39, 127.45, 124.48, 118.68, 118.65, 113.61, 113.60, 87.08, 86.92, 82.70, 81.10, 71.71, 63.81, 58.87, 55.45, 29.33, 28.90. ¹⁹F NMR (565 MHz, CD₃CN) δ −52.46. HRMS calc. for C₃₆H₃₇FN₅O₉ [M+H]⁺ 702.2575, found 702.2563.

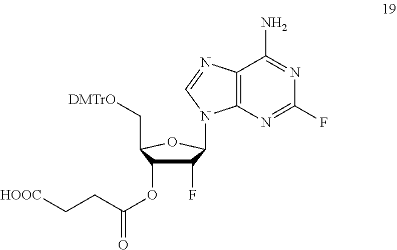

### 4-[5-(6-amino-2-fluoro-purin-9-yl)-2-[[bis(4-methoxyphenyl)-phenyl-methoxy]methyl]-4-fluoro-tetrahydrofuran-3-yl]oxy-4-oxo-butanoic acid Compound 19

To a clear solution of 8 (0.608 g, 1.03 mmol) in DCM (15 mL) and was added 4-(dimethylamino)pyridine (381.58 mg, 3.09 mmol) and succinic anhydride (208.37 mg, 2.06 mmol) sequentially. The resulting mixture was stirred for 3 hrs at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-20% MeOH in DCM) for afford 19 (0.546 g, 77% yield) as yellowish white solid. ¹H NMR (400 MHz, DMSO-d₆) δ 12.26 (s, 1H), 8.30 (s, 1H), 7.95 (s, 2H), 7.40-7.05 (m, 9H), 6.85-6.75 (m, 4H), 6.45-6.09 (m, 1H), 6.00-5.80 (m, 1H), 5.69 (ddd, J=16.9, 7.3, 5.0 Hz, 1H), 4.28 (dt, J=7.7, 4.0 Hz, 1H), 3.72 (s, 6H), 3.29 (d, J=4.1 Hz, 2H), 2.61 (dd, J=8.3, 6.0 Hz, 2H), 2.54-2.46 (m, 2H). ¹³C NMR (101 MHz, DMSO-d₆) δ 173.10, 171.22, 159.54, 158.03, 157.84, 157.63, 157.50, 150.13, 149.93, 144.60, 140.31, 135.30, 135.24, 129.61, 129.56, 127.71, 127.56, 126.63, 117.69, 117.65, 113.06 (d, J=2.0 Hz), 91.47, 89.59, 86.51, 86.17, 85.61, 79.60, 70.07, 69.92, 62.04, 54.96, 54.88, 28.49, 28.36. ¹⁹F NMR (471 MHz, DMSO-d₆) δ −51.43, −201.18-−208.79 (m). HRMS calc. for C₃₅H₃₄F₂N₅O₈ [M+H]⁺ 690.2375, found 690.2372

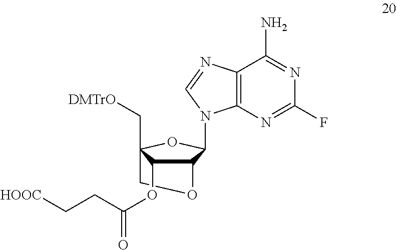

### 4-[5-(6-amino-2-fluoro-purin-9-yl)-2-[[bis(4-methoxyphenyl)-phenyl-methoxy]methyl]-4-methoxy-tetrahydrofuran-3-yl]oxy-4-oxo-butanoic acid Compound 20

To a clear solution of 10 (3 g, 4.99 mmol) in pyridine (15 mL) and was added 4-(dimethylamino)pyridine (1.23 g, 9.97 mmol) and succinic anhydride (1.01 g, 9.97 mmol) sequentially. The resulting mixture was stirred for 3 hrs at 22° C. Pyrimidine was evaporated under vacuum, then residue was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-20% MeOH in DCM) for afford 20 (3.3 g, 95% yield) as yellowish white solid. ¹H NMR (600 MHz, CD₃CN) δ 8.07 (s, 1H), 7.46 (d, J=7.8 Hz, 2H), 7.38-7.25 (m, 6H), 6.90 (d, J=8.6 Hz, 1H), 5.97 (s, 1H), 5.42 (s, 1H), 4.74 (s, 1H), 4.07-3.92 (m, 2H), 3.78 (s, 6H), 3.50 (s, 2H), 2.64-2.48 (m, 4H). ¹³C NMR (151 MHz, CD₃CN) δ 174.03, 172.06, 160.39, 159.36, 159.03, 158.20, 158.07, 151.01, 150.88, 150.02, 145.38, 138.59, 138.57, 136.82, 136.04, 135.98, 130.60, 130.57, 128.53, 128.46, 127.53, 124.46, 113.73, 87.26, 86.86, 86.52, 78.40, 73.02, 72.80, 59.20, 55.49, 29.29, 28.71. ¹⁹F NMR (565 MHz, CD₃CN) δ −52.58. HRMS calc. for C₃₆H₃₅FN₅O₉ [M+H]⁺ 700.2419, found 700.2421.

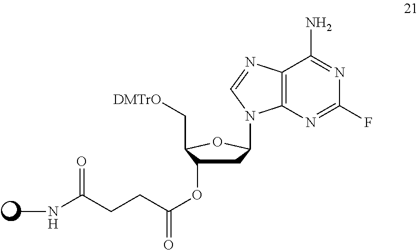

CPG 21: Added 11 (0.2 g, 297.77 μmol) and N-ethyl-N-isopropyl-propan-2-amine (153.93 mg, 1.19 mmol, 207.46 μL) into rb flask. Then added dry acetonitrile (50 mL). Stirred well to dissolve and then HBTU (118.57 mg, 312.65 μmol) to preactivated acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

### Checking the Loading:

Weighted out 38 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg).

Yield: 1.75 g, Loading: 96 μmol/g.

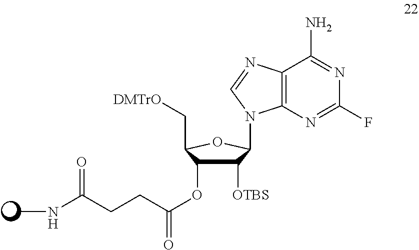

CPG 22: Added 4-[(2R,5R)-5-(6-amino-2-fluoro-purin-9-yl)-2-[[bis(4-methoxyphenyl)-phenyl-methoxy]methyl]-4-[tert-butyl(dimethyl)silyl]oxy-tetrahydrofuran-3-yl]oxy-4-oxo-butanoic acid (0.74 g, 922.77 μmol) and N-ethyl-N-isopropyl-propan-2-amine (477.04 mg, 3.69 mmol, 642.91 μL) into rb flask. Then added dry acetonitrile (50 mL). Stirred well to dissolve and then HBTU (367.45 mg, 968.91 μmol) to preactivated acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 mL total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

### Checking the Loading:

Weighted out 52 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 7.72 g, Loading: 89 umol/g

### General Procedure for CPG Preparation (for 23-25):

To a clear solution of the corresponding succinyl compound (323.43 μmol) in anhydrous acetonitrile (14 mL), N,N-diisopropylethylamine (168.04 mg, 1.29 mmol, 226.47 μL) was added followed by HBTU (122.66 mg, 323.43 μmol) then the activated mixture was stirred for 5 min. CPG 171 umol/g (2.4 g, 420.46 μmol) was added and the support was shacked overnight at 22° C.

Solid support was filtered and washed three times (3×50 mL) with acetonitrile, methanol, acetonitrile again and diethyl ether. Then the support was dried under vacuum and capped with a cap solution (CapA/CapB, v/v 1:1, 20 mL) under shaking overnight. The solid support was filtered and washed three times (3×50 mL) with acetonitrile, methanol, acetonitrile again and diethyl ether to give the corresponding CPG. The loading was calculated by titration of the detritylation solution.

From 18 (226.3 mg) to obtain 23 (2.4 g, 107.3 μmol/g)

From 19 (223.0 mg) to obtain 24 (2.4 g, 124 μmol/g)

From 20 (226.3 mg) to obtain 25 (2.4 g, 104 μmol/g)

### Additional Monomers

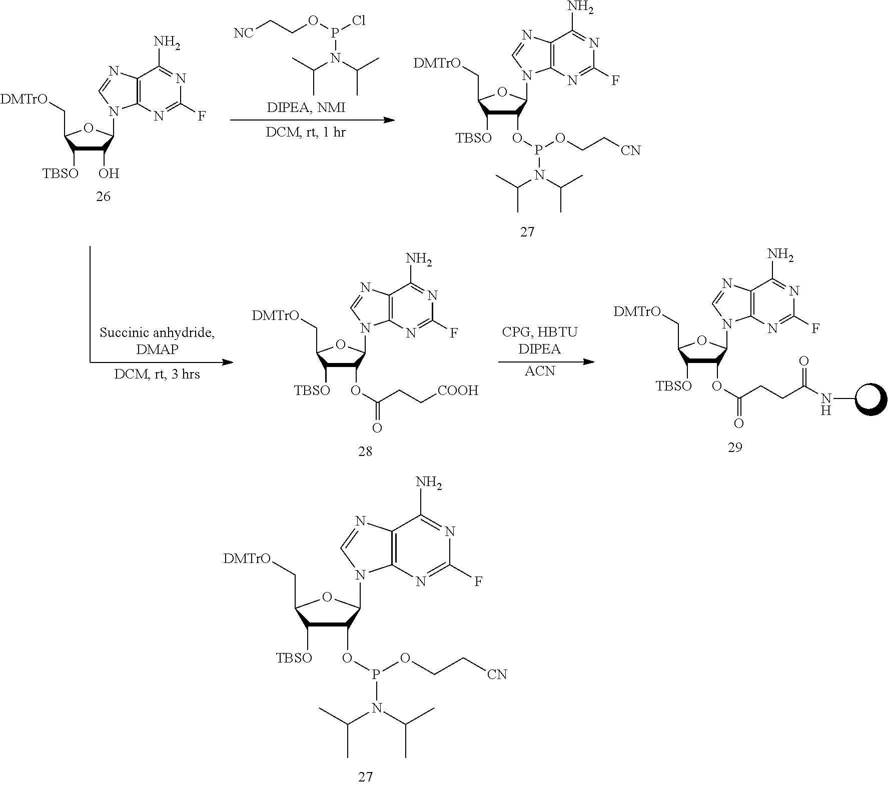

### 3-[[(2R,5R)-2-(6-amino-2-fluoro-purin-9-yl)-5-[[bis(4-methoxyphenyl)-phenyl-methoxy]methyl]-4-[tert-butyl(dimethyl)silyl]oxy-tetrahydrofuran-3-yl]oxy-(diisopropyl amino)phosphanyl] oxypropanenitrile 27

To a clear solution of 26 (0.95 g, 1.32 mmol) in DCM (20 mL) at 22° C. was added N-methyl imidazole (163.02 mg, 1.99 mmol, 158.27 μL) and DIPEA (855.42 mg, 6.62 mmol, 1.15 mL). The reaction mixture was stirred for 5 minutes at rt and 2-cyanoethyl-N,N-diisopropylchlorophosphoramidite (626.62 mg, 2.65 mmol, 591.15 μL) was added slowly into it. Reaction was kept for stirring at 22° C. and TLC was checked after 0.5 hr. Reaction mixture was diluted with DCM (20 mL) and washed with 10% NaHCO₃ solution (20×2 mL). Organic layer separated, dried over anhydrous Na₂SO₄, filtered and the filtrate was evaporated to dryness. The crude mass obtained was purified by flash column chromatography (gradient: 30-70% EtOAc in hexane) to afford 27 (0.76 g, 64% yield) as white solid. ¹H NMR (600 MHz, CD₃CN) δ 8.03 (dt, J=16.5, 1.7 Hz, 1H), 7.37-7.32 (m, 2H), 7.26-7.14 (m, 7H), 6.81-6.73 (m, 4H), 6.24 (s, 2H), 6.11-5.93 (m, 1H), 5.04-4.73 (m, 1H), 4.70-4.63 (m, 1H), 4.10 (dd, J=11.5, 6.8 Hz, 1H), 3.86-3.66 (m, 8H), 3.57 (dp, J=12.5, 6.5 Hz, 3H), 3.43 (td, J=9.0, 3.4 Hz, 1H), 3.16 (dd, J=10.8, 4.1 Hz, 1H), 2.61-2.45 (m, 2H), 1.12 (dd, J=9.5, 6.8 Hz, 11H), 0.94 (d, J=6.8 Hz, 2H), 0.85-0.75 (m, 9H), 0.13-−0.05 (m, 6H) ppm. ¹³C NMR (151 MHz, CD₃CN) δ 160.13, 159.11, 159.08, 159.05, 158.76, 158.05, 157.91, 151.43, 151.30, 145.38, 141.06, 136.22, 136.18, 136.11, 136.05, 130.59, 130.57, 130.53, 130.51, 128.52, 128.50, 128.37, 128.30, 128.28, 127.34, 127.29, 118.82, 118.80, 118.66, 113.54, 113.51, 113.49, 88.87, 86.76, 86.61, 84.24, 83.74, 76.17, 76.10, 75.42, 71.99, 71.44, 71.40, 63.31, 63.00, 60.60, 59.23, 59.10, 58.41, 58.27, 55.56, 55.55, 54.70, 43.73, 43.65, 43.47, 43.39, 26.04, 26.02, 24.91, 24.86, 24.73, 24.68, 24.49, 24.44, 21.09, 20.41, 20.36, 18.35, 18.33, 14.36, −4.15, −4.74, −4.87 ppm. ³¹P NMR (243 MHz, CD₃CN) δ 150.75, 149.67 ppm. ¹⁹F NMR (565 MHz, CD₃CN) δ −52.05, −52.17 ppm. HRMS calc. for C₄₆H₆₂FN₇O₇PSi [M+H]⁺ 902.4202 found 902.4189.

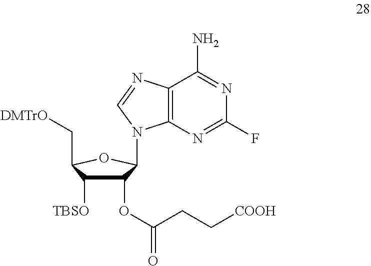

### 4-[(2R,5R)-2-(6-amino-2-fluoro-purin-9-yl)-5-[[bis(4-methoxyphenyl)-phenyl-methoxy]methyl]-4-[tert-butyl(dimethyl)silyl]oxy-tetrahydrofuran-3-yl]oxy-4-oxo-butanoic acid 28

To a clear solution of 26 (0.69 g, 983.11 μmol) in DCM (8 mL) and pyridine (2 mL) was added 4-(dimethylamino)pyridine (480.42 mg, 3.93 mmol) and succinic anhydride (295.14 mg, 2.95 mmol) sequentially. The resulting mixture was stirred for 2 hrs at 22° C. Reaction mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% Methanol in DCM) for afford 28 (0.54 g, 68% yield) as white foam. ¹H NMR (600 MHz, DMSO-d₆) δ 12.28 (s, 1H), 8.57 (tt, J=3.4, 1.5 Hz, 1H), 8.34 (d, J=1.5 Hz, 1H), 7.98 (s, 1H), 7.91 (s, 1H), 7.41-7.35 (m, 1H), 7.25 (d, J=8.0 Hz, 2H), 7.22-7.10 (m, 7H), 6.80-6.75 (m, 4H), 6.09 (t, J=1.9 Hz, 1H), 5.96 (dt, J=4.4, 1.8 Hz, 1H), 5.16-5.11 (m, 1H), 4.01 (dt, J=7.1, 3.4 Hz, 1H), 3.70 (d, J=1.4 Hz, 6H), 3.43-3.30 (m, 1H), 2.97 (dd, J=11.1, 4.0 Hz, 1H), 2.68-2.52 (m, 2H), 2.51-2.44 (m, 5H), 0.75 (d, J=1.4 Hz, 9H), 0.04 (d, J=1.4 Hz, 3H), −0.04 (d, J=1.4 Hz, 3H) ppm. ¹³C NMR (151 MHz, DMSO-d₆) δ 173.18, 171.36, 159.30, 158.04, 158.02, 157.94, 157.88, 157.74, 149.91, 149.77, 149.65, 144.68, 141.12, 141.10, 136.18, 135.52, 135.32, 129.63, 129.59, 127.67, 127.63, 126.62, 123.95, 117.69, 117.66, 113.04, 112.99, 86.59, 85.36, 82.18, 74.01, 69.28, 61.63, 55.01, 54.96, 28.70, 28.53, 25.53, 17.62, −5.07, −5.39 ppm. ¹⁹F NMR (565 MHz, DMSO-d₆) δ −51.66 ppm. HRMS calc. for C₄₁H₄₉FN₅O₉Si [M+H]⁺ 802.3284, found 802.3270.

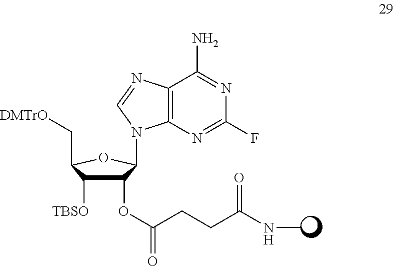

### CPG 29:

Added 4-[(2R,5R)-2-(6-amino-2-fluoro-purin-9-yl)-5-[[bis(4-methoxyphenyl)-phenyl-methoxy]methyl]-4-[tert-butyl(dimethyl)silyl]oxy-tetrahydrofuran-3-yl]oxy-4-oxo-butanoic acid (0.58 g, 723.26 μmol) and N-ethyl-N-isopropyl-propan-2-amine (373.89 mg, 2.89 mmol, 503.90 μL) into rb flask. Then added dry acetonitrile (50 mL). Stirred well to dissolve and then HBTU (288.00 mg, 759.42 μmol) to preactivated acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as described above. This time dried CPG in high vacuum overnight.

### Checking the Loading:

Weighted out 43 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 6.7 g, Loading: 84 μmol/g

### REFERENCES

1. Kirnos, M. D., et al., 2-Aminoadenine is an adenine substituting for a base in S-2L cyanophage DNA. Nature, 1977. 270(5635): p. 369-370.
2. Gaffney, B. L., L. A. Marky, and R. A. Jones, The influence of the purine 2-amino group on DNA conformation and stability-IL Synthesis and physical characterization of d[CGT(2-NH2)ACG], d[CGU(2—NH2)ACG], and d[CGT(2—NH2)AT(2—NH2)ACG]. Tetrahedron, 1984. 40(1): p. 3-13.
3. Gaffney, B. L., L. A. Marky, and R. A. Jones, The influence of the purine 2-amino group on DNA conformation and stability. Synthesis and conformational analysis of d[T(2-aminoA)] 3. Nucleic Acids Research, 1982. 10(14): p. 4351-4361.
4. Strobel, S. A., et al., The 2,6-Diaminopurine Riboside.cntdot.5-Methylisocytidine Wobble Base Pair: An Isoenergetic Substitution for the Study of G.cntdot. UPairs in RNA. Biochemistry, 1994. 33(46): p. 13824-13835.
5. Gryaznov, S. and R. G. Schultz, Stabilization of DNA:DNA and DNA:RNA duplexes by substitution of 2′-deoxyadenosine with 2′-deoxy-2-aminoadenosine. Tetrahedron Letters, 1994. 35(16): p. 2489-2492.
6. Kutyavin, I. V., et al., Oligonucleotides Containing 2-Aminoadenine and 2-Thiothymine Act as Selectively Binding Complementary Agents. Biochemistry, 1996. 35(34): p. 11170-11176.
7. Hughesman, C. B., R. F. B. Turner, and C. Haynes, Stability and Mismatch Discrimination of DNA duplexes containing 2,6-Diaminopurine and 2-Thiothymidine Locked Nucleic Acid bases. Nucleic Acids Symposium Series, 2008. 52(1): p. 245-246.
8. Kamiya, Y., et al., Incorporation of Pseudo-complementary Bases 2,6-Diaminopurine and 2-Thiouracil into Serinol Nucleic Acid (SNA) to Promote SNA RNA Hybridization. Chemistry—An Asian Journal, 2020. 15(8): p. 1266-1271.
9. Howard, F. B. and H. T. Miles, 2NH2A.cntdot.T helixes in the ribo- and deoxypolynucleotide series. Structural and energetic consequences of 2NH2A substitution. Biochemistry, 1984. 23(26): p. 6723-6732.
10. Kikuchi, K., Y. Taniyama, and R. Marumoto, Evaluation of the 2 NH2A-T Pair in Hybridization, I Synthesis of the DNA RNA Hybrid Oligomers Containing 2-Aminoadenosines. Zeitschrift für Naturforschung B, 1988. 43(5): p. 623-630.
11. Borah, B., et al., Poly(d2NH2A-dT): two-dimensional NMR shows a B to A conversion in high salt. Biochemistry, 1985. 24(25): p. 7456-7462.
12. Borah, B., et al., Conversions of poly(2-aminodeoxyadenylate-5-halodeoxyuridylate) from B to A forms in high salt. An NMR and circular dichroism study. Biochemistry, 1986. 25(23): p. 7464-7470.
13. Sproat, B. S., B. Beijer, and A. Iribarren, New synthetic routes to protected purine 2′-O-methylriboside-3′-O-phosphoramidites using a novel alkylation procedure. Nucleic Acids Research, 1990. 18(1): p. 41-49.
14. Hosono, K., et al., Cleavage effect of oligoribonucleotides substituted at the cleavage sites with modified pyrimidine- and purine-nucleosides. Biochimica et Biophysica Acta (BBA)—Gene Structure and Expression, 1997. 1354(3): p. 211-218.
15. Pasternak, A., et al., A chemical synthesis of LNA-2,6-diaminopurine riboside, and the influence of 2′-O-methyl-2,6-diaminopurine and LNA-2,6-diaminopurine ribosides on the thermodynamic properties of 2′-O-methyl RNA RNA heteroduplexes. Nucleic Acids Research, 2007. 35(12): p. 4055-4063.
16. Sproat, B. S., et al., New synthetic routes to synthons suitable for 2′-O-allyloligoribonucleotide assembly. Nucleic Acids Research, 1991. 19(4): p. 733-738.
17. Puj ari, S. S., P. Leonard, and F. Seela, Oligonucleotides with “Clickable” Sugar Residues: Synthesis, Duplex Stability, and Terminal versus Central Interstrand Cross-Linking of 2′-O-Propargylated 2-Aminoadenosine with a Bifunctional Azide. The Journal of Organic Chemistry, 2014. 79(10): p. 4423-4437.
18. Boudou, V., et al., Base pairing of anhydrohexitol nucleosides with 2,6-diaminopurine, 5-methylcytosine and uracil as base moiety. Nucleic Acids Research, 1999. 27(6): p. 1450-1456.
19. Boudou, V., et al., Oligonucleotides with 2,6-Diaminopurine Base Replacing for Adenine: Synthesis and Properties. Nucleosides and Nucleotides, 1999. 18(6-7): p. 1429-1431.
20. Wu, X., et al., 2,6-Diaminopurine in TNA: Effect on Duplex Stabilities and on the Efficiency of Template-Controlled Ligations1. Organic Letters, 2002. 4(8): p. 1283-1286.
21. Koshkin, A. A., Syntheses and Base-Pairing Properties of Locked Nucleic Acid Nucleotides Containing Hypoxanthine, 2,6-Diaminopurine, and 2-Aminopurine Nucleobases. The Journal of Organic Chemistry, 2004. 69(11): p. 3711-3718.
22. Rosenbohm, C., et al., LNA guanine and 2,6-diaminopurine. Synthesis, characterization and hybridization properties of LNA 2,6-diaminopurine containing oligonucleotides. Bioorganic & Medicinal Chemistry, 2004. 12(9): p. 2385-2396.
23. Langkjor, N., J. Wengel, and A. Pasternak, Watson-Crick hydrogen bonding ofunlocked nucleic acids. Bioorganic & Medicinal Chemistry Letters, 2015. 25(22): p. 5064-5066.
24. Hornum, M., et al., Base-Pairing Properties of Double-Headed Nucleotides. Chemistry—A European Journal, 2019. 25(30): p. 7387-7395.
25. Muraoka, M., et al., Synthesis and Properties of 2′4′- and 3′-5′-Linked Ribodinucleoside Monophosphates Containing 2-Aminoadenosine and Uridine. Nucleosides and Nucleotides, 1991. 10(6): p. 1317-1332.
26. Muraoka, M., S. Takahashi, and S. Uesugi, Interaction of (2′-5) and (3′-5) Linked 2-Aminoadenylyl-3-aminoadenosines with Polyuridylic Acid. Nucleosides and Nucleotides, 1995. 14(7): p. 1503-1518.
27. Muraoka, M., et al., Synthesis and Properties of Dinucleoside Monophosphates Containing 2-Aminoadenine 8,2′-S— and Uracil 6,2′-O-Cyclonucleosides. Nucleosides and Nucleotides, 1990. 9(2): p. 205-221.
28. Matray, T., et al., A Remarkable Stabilization of Complexes Formed by 2,6-Diaminopurine Oligonucleotide N3′→P5′ Phosphoramidates. Nucleosides, Nucleotides & Nucleic Acids, 2000. 19(10-12): p. 1553-1567.
29. Matray, T. J. and S. M. Gryaznov, Synthesis and properties of RNA analogs oligoribonucleotide N3′→P5′ phosphoramidates. Nucleic Acids Research, 1999. 27(20): p. 3976-3985.
30. Haaiima, G., et al., Increased DNA binding and sequence discrimination of PNA oligomers containing 2,6-diaminopurine. Nucleic Acids Res., 1997. 25(22): p. 4639-4643.
31. Kamiya, Y., et al., Introduction of 2,6-Diaminopurines into Serinol Nucleic Acid Improves Anti-miRNA Performance. ChemBioChem, 2017. 18(19): p. 1917-1922.
32. Reza, F., et al., Synthesis of 6-substituted 2′-deoxyguanosine derivatives using trifluoroacetic anhydride in pyridine. Tetrahedron Letters, 1990. 31(3): p. 319-321.
33. Gao, H., et al., 6-O-(Pentafluorophenyl)-2′-deoxyguanosine: a versatile synthon for nucleoside and oligonucleotide synthesis. The Journal of Organic Chemistry, 1992. 57(25): p. 6954-6959.
34. Porcher, S. and S. Pitsch, Synthesis of 2′-O-[(Triisopropylsilyl)oxy]methyl (=tom)-Protected Ribonucleoside Phosphoramidites Containing Various Nucleobase Analogues. Helvetica Chimica Acta, 2005. 88(10): p. 2683-2704.
35. Ross, B., R. Springer, and V. Ravikumar, An Efficient and Scalable Synthesis of 2,6-Diaminopurine Riboside. Nucleosides, Nucleotides & Nucleic Acids, 2008. 27(1): p. 67-69.
36. Liu, C., et al., Synthesis and antiviral evaluation of base-modified deoxythreosyl nucleoside phosphonates. Organic & Biomolecular Chemistry, 2017. 15(26): p. 5513-5528.
37. Vorlickovi, M., et al., Conformation of the synthetic DNA poly(amino 2 dA-dT) duplex in high-salt and aqueous alcohol solutions. Nucleic Acids Research, 1988. 16(1): p. 279-289.
38. Christensen, L. F., et al., Synthesis and biological activity of selected 2,6-disubstituted(2-deoxy-.alpha.-and-.beta.-D-erythro-pentofuranosyl)purines. Journal of Medicinal Chemistry, 1972. 15(7): p. 735-739.
39. Wright, G. E., et al., Convenient synthesis of 2-halo-2′-deoxyadenosines. The Journal of Organic Chemistry, 1987. 52(20): p. 4617-4618.
40. Brown, T. and A. G. Craig, The Incorporation of 2,6-Diaminopurine Into Oligodeoxyribonucleotides by the Phosphoramidite Method. Nucleosides and Nucleotides, 1989. 8(5-6): p. 1051-1051.
41. Arico, J. W., A. K. Calhoun, and L. W. McLaughlin, Preparation of the 2′-Deoxynucleosides of 2,6-Diaminopurine and Isoguanine by Direct Glycosylation. The Journal of Organic Chemistry, 2010. 75(5): p. 1360-1365.
42. Barabino, S. M. L., B. S. Sproat, and A. I. Lamond, Antisense probes targeted to an internal domain in U2 snRNP specifically inhibit the second step of pre-mRNA splicing. Nucleic Acids Research, 1992. 20(17): p. 4457-4464.
43. Canol, A., M. F. Goodman, and R. Eritja, Synthesis of Oligodeoxyribonucleotides Containing 2,6-Diaminopurine. Nucleosides and Nucleotides, 1994. 13(1-3): p. 501-509.
44. Luyten, I., et al., Protection of 2,6-Diaminopurine 2′-Deoxyriboside. Nucleosides and Nucleotides, 1997. 16(7-9): p. 1649-1652.
45. Srivastava, S. C. and N. P. Srivastava, N-fmoc nucleosides andphosphoramidites useful in the synthesis of oligonucleotides. 2010, ChemGenes Corporation, USA. p. 52 pp.
46. DeCorte, B. L., et al., Improved Strategies for Postoligomerization Synthesis of Oligodeoxynucleotides Bearing Structurally Defined Adducts at the N2 Position of Deoxyguanosine. Chemical Research in Toxicology, 1996. 9(3): p. 630-637.

### Example 2: ¹⁵N Labeling, 2-F a Retention and Iso-G Conversion in Oligonucleotides Derived from 2-F a Building Block Containing Oligonucleotides

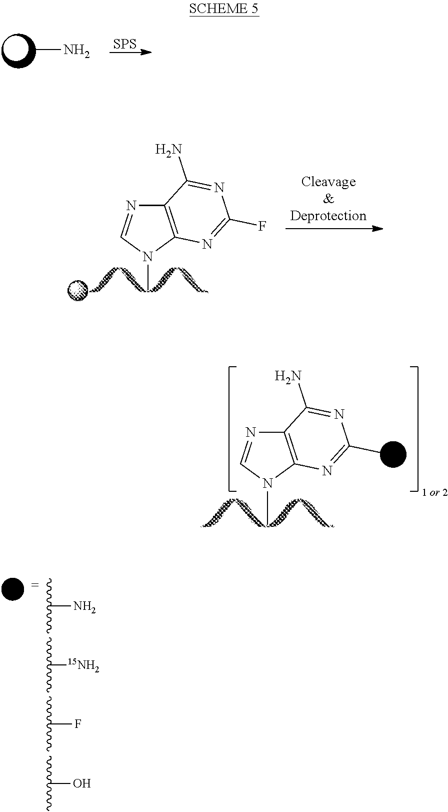

### Oligonucleotide Synthesis:

Oligonucleotides were synthesized on an ABI-394 DNA/RNA synthesizer using modified synthesis cycles based on those provided with the instrument. A solution of 0.25 M 5-(S-ethylthio)-1H-tetrazole in acetonitrile was used as the activator. The phosphoramidite solutions were 0.15 M in anhydrous acetonitrile. The oxidizing reagent was 0.02 M 12 in THF/pyridine/H₂O. The detritylation reagent was 3% dichloroacetic acid (DCA) in DCM. After completion of the automated synthesis, the solid support was dried with argon.

The oligonucleotides were cleaved from the solid-support as follows:

### For DAP:

The oligonucleotide was then manually released from support and deprotected using a mixture of 30% NH₄OH for 24 h at 65° C.

### For 2-¹⁵-N-DAP:

The oligonucleotide was then manually released from support and deprotected using a mixture of 16N ¹⁵NH₄OH for 48 h at 65° C.

### For 2-Fluoro-Deoxy-Adenosine:

The oligonucleotide was then manually released from support and deprotected using a mixture of 30% NH₄OH for 18 h at room temperature.

### For Deoxy-Iso-Guanosine

Starting from the 2-fluoro-deoxy-adenosine, the lyophilized oligonucleotides were dissolved in a mixture of 0.5M NaOH (4 mL) for 4 h at 65° C.

Solvent was collected by filtration and the support was rinsed with deionized water (6 mL). Crude oligonucleotides were purified by anion exchange glass column (10×300 mm) packed with TSKGel super Q-5PW and using 20 mM sodium phosphate, 15% ACN as buffer A and 20 mM sodium phosphate 15% ACN, 1M NaBr as buffer B. Purification gradient started from 18% to 30% B in 240 min at 60° C. All single strands were purified to >90% HPLC (260 nm) purity and then desalted by size exclusion chromatography using a custom packed with Sephadex G25 (GE Healthcare), eluted with sterile nuclease-free water.

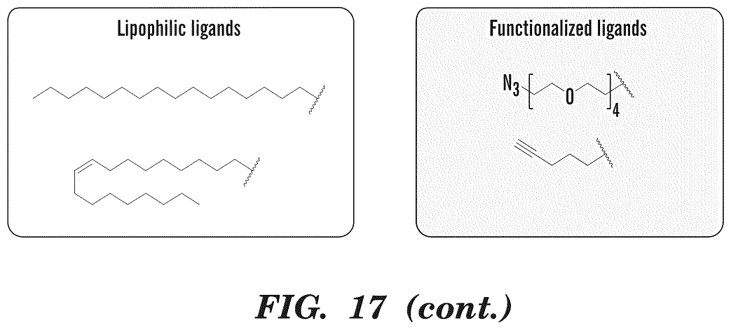

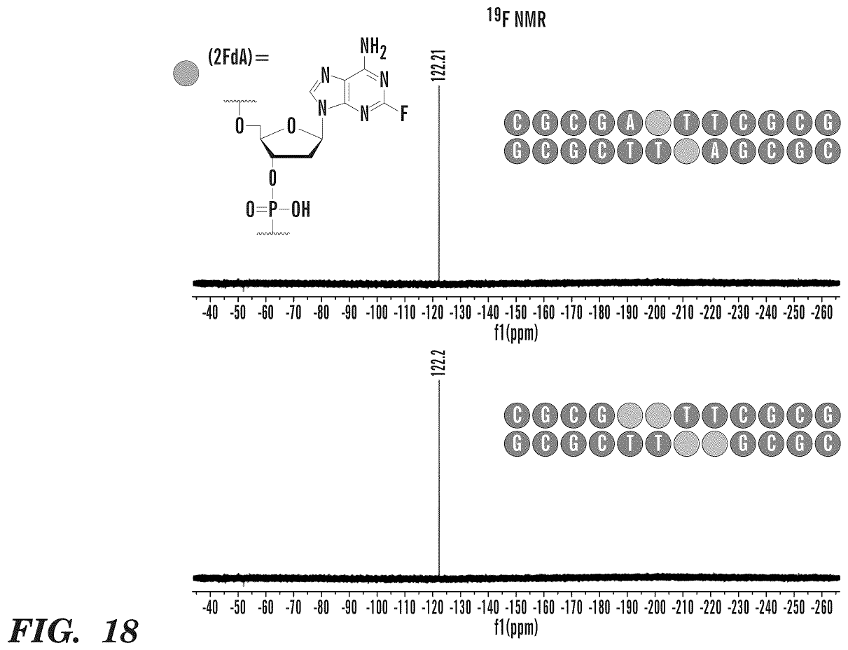

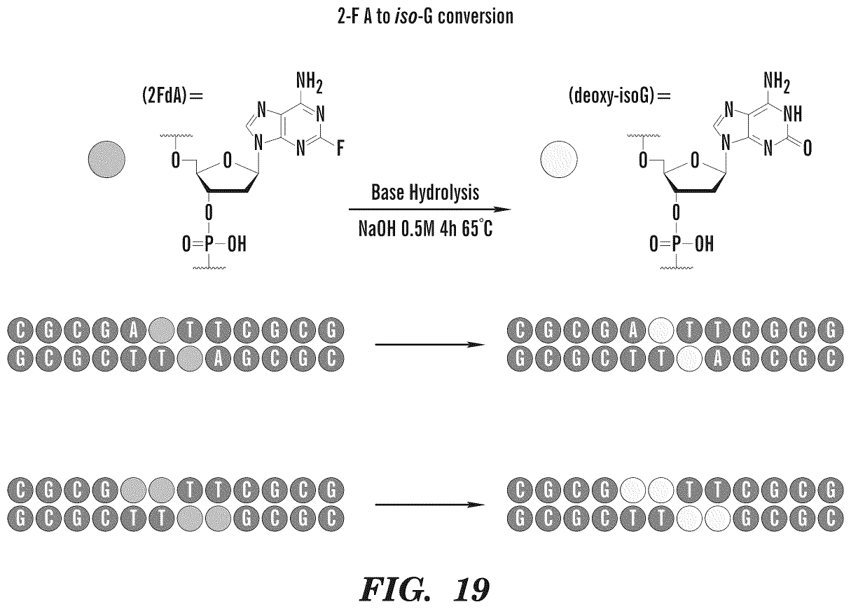

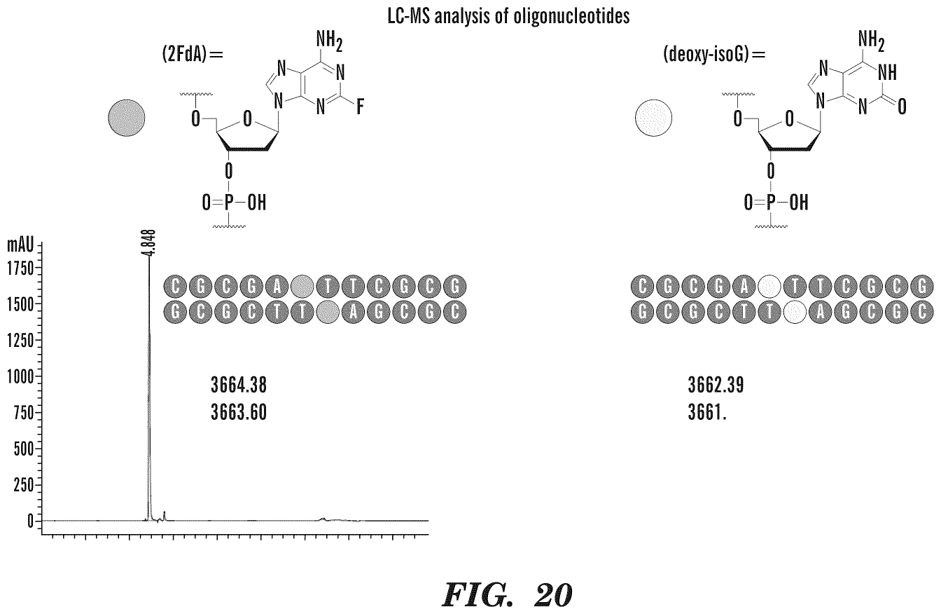

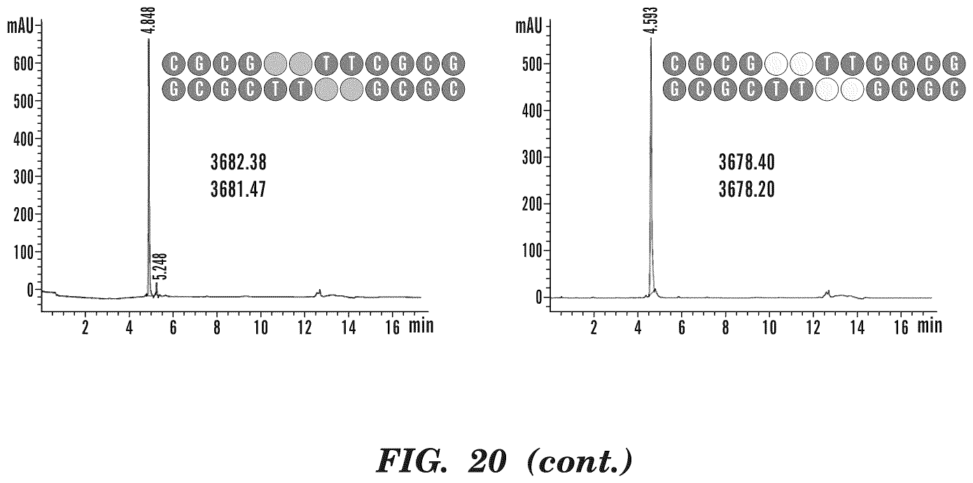

Oligonucleotide concentrations were calculated based on absorbance at 260 nm and the following extinction coefficients: A/2,6-diaminoA, 13.86 M⁻¹ cm⁻¹; T/U, 7.92 M⁻¹ cm⁻¹; C, 6.57 M⁻¹ cm⁻¹; and G, 10.53 M⁻¹ cm⁻¹. The purity and identity of oligonucleotides were verified by analytical anion exchange chromatography and mass spectrometry. Results are shown in FIGS. 18-20.

TABLE 10
Sequences and mass spectroscopy characterization of oligonucleotides
made for Dickerson-Dodecamer NMR study

| | | Mass (m/z) | |
|---|---|---|---|
| Strand | Sequence 5′-3′ | Calc. | Obs. |
| ON1 | dCdGdCdGdAdAdTdTdCdGdCdG | 3646.39 | 3645.58 |
| | (SEQ ID NO: 254) | | |
| ON2 | dCdGdCdGddAdTdTdCdGdCdG | 3661.41 | 3660.62 |
| | (SEQ ID NO: 255) | | |
| ON3 | dCdGdCdGdAddTdTdCdGdCdG | 3661.41 | 3660.89 |
| | (SEQ ID NO: 256) | | |
| ON4 | dCdGdCdGdddTdTdCdGdCdG | 3676.42 | 3675.61 |
| | (SEQ ID NO: 257) | | |
| ON5 | dCdGdCdG¹⁵ddAdTdTdCdGdCdG | 3662.41 | 3661.71 |
| | (SEQ ID NO: 258) | | |
| ON6 | dCdGdCdGdA¹⁵ddTdTdCdGdCdG | 3662.41 | 3661.70 |
| | (SEQ ID NO: 259) | | |
| ON7 | dCdGdCdG¹⁵d¹⁵ddTdTdCdGdCdG | 3677.42 | 3677.73 |
| | (SEQ ID NO: 260) | | |
| ON8 | dCdGdCdGdA(2FdA)dTdTdCdGdCdG | 3664.38 | 3663.41 |
| | (SEQ ID NO: 261) | | |
| ON9 | dCdGdCdG(2FdA)(2FdA)dTdTdCdGdCdG | 3682.38 | 3681.47 |
| | (SEQ ID NO: 262) | | |
| ON10 | dCdGdCdGdA(isodG)dTdTdCdGdCdG | 3662.39 | 3662.44 |
| | (SEQ ID NO: 263) | | |
| ON11 | dCdGdCdG(isodG)(isodG)dTdTdCdGdCdG | 3680.39 | 3677.83 |
| | (SEQ ID NO: 264) | | |

Deoxy-adenosine (dA), deoxy-Cytidine (dC), deoxy-Guanosine (dG), deoxy-Thymidine (dT), deoxy-2,6-diaminopurine (d), deoxy-2-¹⁵N,6-diaminopurine (15d), 2-fluoro-deoxy-adenosine ((2FdA)) and deoxy-isoguanosine (isodG)

TABLE 11
Abbreviations used in sequences

| | Nucleotide Name* |
|---|---|
| A | 2′-deoxy-2′-fluoroadenosine-3′-phosphate |
| C | 2′-deoxy-2′-fluorocytidine-3′-phosphate |
| G | 2′-deoxy-2′-fluoroguanosine-3′-phosphate |
| U | 2′-deoxy-2′-fluorouridine-3′-phosphate |
| a | 2′-O-methyladenosine-3′-phosphate |
| c | 2′-O-methylcytidine-3′-phosphate |
| g | 2′-O-methylguanosine-3′-phosphate |
| u | 2′-O-methyluridine-3′-phosphate |
| d | 2′-deoxy-2-aminoadenosine-3′-phosphate (deoxy-2,6-diaminopurine) |
| R | 2′-aminoadenosine-3′-phosphate (ribo-2,6-diaminopurine) |
| X | 2′-deoxy-2′-fluoro-2-aminoadenosine-3′-phosphate (2′-F-2,6-diaminopurine) |
| Y | 2-amino-2′-O,4′-C-methylene adenosine-3′-phosphate (LNA-2,6-diaminopurine) |
| Z | 2′-O-methyl-2-aminoadenosine-3′-phosphate (2′OMe-2,6-diaminopurine) |
| (L) | tri-N-acetylgalactoamine (tri-GalNAc) ligand |
| | 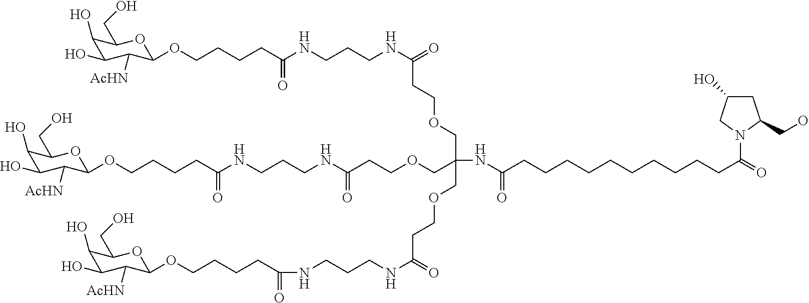 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| dA | 2′-deoxyadenosine-3′-phosphate (Deoxy-Adenosine) |
| dC | 2′-deoxycytidine-3′-phosphate (Deoxy-Cytidine) |
| dG | 2′-deoxyguanosine-3′-phosphate (Deoxy-Guanosine) |
| dT | 2′-deoxyuridine-3′-phosphate (Deoxy-Thymidine) |
| d¹⁵ | 2′-deoxy-2-(¹⁵N) aminoadenosine-3′-phosphate (deoxy-2,6-diaminopurine 2-¹⁵N labelling) |
| X₂₀₁ | 2-(N-(6-(O-cholesterol)oxcarbonylaminohexyl)amino)-2′-deoxy-2′-fluoro-adenosine 3′-phosphate |
| | 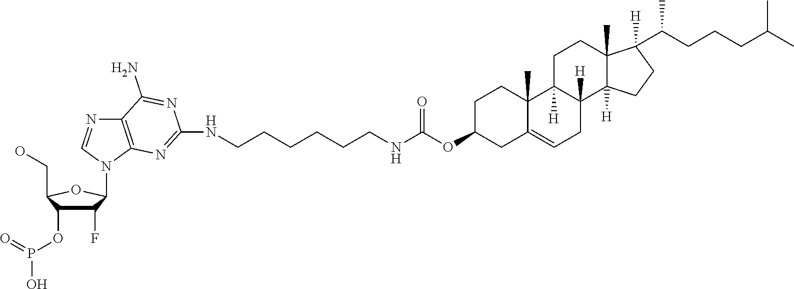 |
| | (2′F-2-cholesterol-adenosine) |
| X₂₀₄ | See FIG. 7A, 2′F-2-N-acetylgalactosamine-adenosine 3′-phosphate |
| X₂₀₅ | See FIG. 7A, 2′F-2-tri-N-acetylgalactosamine 3′-phosphate |
| X₂₀₆ | See FIG. 7A, 2′F-2-cRGD Kessler peptide 3′-phosphate |
| X₂₀₉ | 2-(N-hexadecylamino)-2′-deoxy-2′-fluoro-adenosine-3′-phosphate (2′F-2-Cl6-adenosine); see also, FIG. 7B |
| X₂₁₁ | 2-(N-nonylamino)-2′-deoxy-2′-fluoro-adenosine-3′-phosphate (2′F-2-C9-adenosine); see also, FIG. 7B |
| X₂₁₂ | 2-(N-hexylamino)-2′-deoxy-2′-fluoro-adenosine-3′-phosphate (2′F-2-C6-adenosine); see also, FIG. 7B |
| Z₂₀₄ | See, FIG. 8, 2′OMe-2-N-acetylgalactosamine-adenosine 3′-phosphate |
| Z₂₀₉ | 2-(N-hexyldecylamino)-2′-O-methyladenosine-3′-phosphate (2′OMe-2-C16-adenosine); see also, FIG. 8 |
| Z₂₁₁ | 2-(N-nonylamino)-2′-O-methyladenosine-3′-phosphate (2′OMe-2-C9-adenosine); see also, FIG. 8 |
| Z₂₁₂ | 2-(N-hexylamino)-2′-O-methyladenosine-3′-phosphate (2′OMe-2-C6-adenosine); see also, FIG. 8 |
| “•” | Phosphorothioate linkages; Indicates modification of 3′-phosphate for 3′-phosphorothioate on the |
| symbol | preceding nucleotide |

*3′-terminal nucleotides are 3′-OH unless conjugated to (L) or otherwise indicated

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. A method for preparing an oligonucleotide comprising a nucleoside of Formula (I): 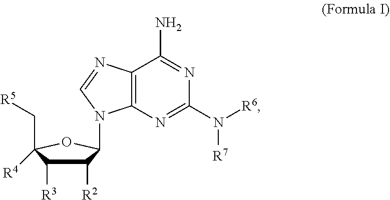
the method comprising reacting an oligonucleotide comprising a nucleoside of Formula (II): 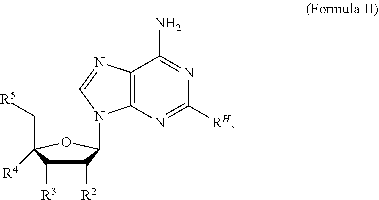
with an amine of formula HNR⁶R⁷,
wherein:
R^{H} is halogen (e.g., chloro or fluoro);
R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R³ is a bond to an internucleotide linkage to a subsequent nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆ alkenyl or optionally substituted C₂-C₆ alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵ represents a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R¹)(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is 0 or S;
a and b are each independently 1-10;
each R⁶ and R⁷ is independently hydrogen or -L-R^{L}, provided that at least one of R⁶ and R⁷ is not H;
or R⁶ and R⁷taken together with the nitrogen atom to which they are attached form a 3-10 membered heterocyclyl or a 3-10 membered heteroaryl group, the heterocyclyl or heteroaryl comprising one -L-R^{L} group,
wherein
L is absent or a linker;
each R^{L} is a ligand, (e.g., selected independently from the group consisting of carbohydrates, peptides, lipids, therapeutic agents, diagnostic agents, detectable labels, antibodies or fragments thereof, vitamins, optionally substituted C₁₋₃₀ alkyl, optionally substituted C₁₋₃₀ alkenyl, or optionally substituted C₁₋₃₀ alkynyl);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl,
provided that,
(i) no more than one of R² and R³ is a bond to an internucleotide linkage to a subsequent nucleotide; and
(ii) when both of R² and R³ are not a bond, then R⁵ is a bond to an internucleotide linkage to a preceding nucleotide.

2. The method of claim 1, wherein R^{H} is fluoro.

3.-4. (canceled)

5. The method of claim 1, wherein at least one R^{L} is selected independently from the group consisting of carbohydrates, lipids, vitamins, peptides, proteins, lipoproteins, peptidomimetics, polyamines, nucleosides and nucleotides, oligonucleotides, detectable labels, diagnostic agents, fluorescent dyes, polyethylene glycols (PEGs), antibodies, antibody fragments.

6.-7. (canceled)

8. The method of claim 1, wherein R² is hydrogen,
hydroxyl, protected hydroxyl, halogen, optionally substituted C₁₋₆ alkoxy (e.g., methyl) or alkoxyalkyl (e.g. 2-methoxyethyl); or R⁴ and R² taken together are 4′-C(R¹⁰R¹¹)ᵥ—Y-2′ or 4′-Y—C(R¹⁰R¹¹)ᵥ-2′.

9.-10. (canceled)

11. The method of claim 1, wherein R⁴ is H.

12. The method of claim 1, wherein R³ is a bond to an internucleotide linkage to a subsequent nucleotide, hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

13-14. (canceled)

15. The method of claim 1, wherein R³ is hydroxyl.

16. The method of claim 1, wherein R⁵ is a bond to an internucleotide linkage to a preceding nucleotide, hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates and phosphate mimics.

17. (canceled)

18. The method of claim 1, wherein R⁵ is hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, or gamma-thiotriphosphate.

19. (canceled)

20. The method of claim 1, wherein:
(i) the oligonucleotide comprises at least one ribonucleotide,
(ii) the oligonucleotide comprises at least one 2′-deoxyribonucleotide;
(iii) the oligonucleotide comprises at least one nucleotide with a modified or non-natural nucleobase;
(iv) the oligonucleotide comprises at least one nucleotide with a modified ribose sugar;
(v) the oligonucleotide comprises at least one nucleotide comprising a group other than H or OH at the 2′-position of the ribose sugar;
(vi) the oligonucleotide comprises at least one nucleotide with a 2′-F ribose;
(vii) the oligonucleotide comprises at least one nucleotide with a 2′-OMe ribose;
(viii) the oligonucleotide comprises at least one nucleotide comprising a moiety other than a ribose sugar;
(ix) the oligonucleotide comprises at least one modified internucleotide linkage;
(x) the oligonucleotide comprises at least 2 consecutive independently selected monomers of the Formula (I) and/or (II):
(xi) the oligonucleotide is attached to a solid support; and/or
(xii) the oligonucleotide comprises at least one hydroxyl, phosphate or amino protecting group.

21.-33. (canceled)

34. A compound of Formula (III): 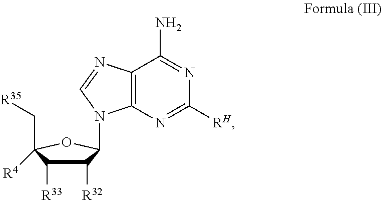
wherein:
R^{H} is halogen;
R³² is hydrogen, hydroxy, halogen protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support;
R³³ is hydrogen, hydroxy, halogen protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, or a linker covalently bonded (e.g., —C(O)CH₂CH₂C(O)—) to a solid support, and optionally, only one of R³² and R³³ is a phosphate group, a reactive phosphorous group, a solid support or a linker to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R³² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P-0-(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

35. The compound of claim 34, wherein R^{H} is fluoro.

36. The compound of claim 34, wherein R³² is halogen; or R⁴ and R³² taken together are 4′-C(R¹⁰R¹¹)ᵥ—Y-2′ or 4′-Y—C(R¹⁰R¹¹)ᵥ-2′.

37.-38. (canceled)

39. The compound of claim 34, wherein R⁴ is H.

40. The compound of claim 34, wherein R³³ is hydroxy, protected hydroxy, a phosphate group, a solid support, or a linker covalently bonded (e.g., —C(O)CH₂CH₂C(O)—) to a solid support.

41. (canceled)

42. The compound of claim 34, wherein R³⁵ is hydroxy, protected hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphates and phosphate mimics.

43. (canceled)

44. The compound of claim 34, wherein:
(i) R³³ is a reactive phosphorous group and R³⁵ is a protected hydroxyl; or
(ii) R³² is a reactive phosphorous group and R³⁵ is a protected hydroxyl; or
(ii) R³⁵ is a vinylphosphonate (VP) group, cyclopropylphosphonate, or a phosphate mimic, and R³³ is a reactive phosphorous group; or
(iv) R³⁵ is a vinylphosphonate (VP) group, cyclopropylphosphonate, or a phosphate mimic, and R³² is a reactive phosphorous group.

45.-48. (canceled)

49. The compound of claim 34, wherein R³⁵ is a triphosphate group and R³³ is allyloxy, azidomethoxy, or aminooxy.

50.-51. (canceled)

52. A method for preparing an oligonucleotide comprising a nucleoside of Formula (X): 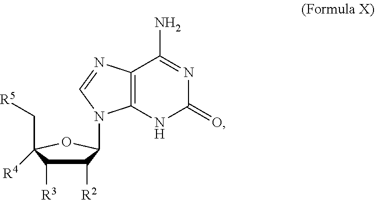
the method comprising reacting an oligonucleotide comprising a nucleoside of Formula (XI): 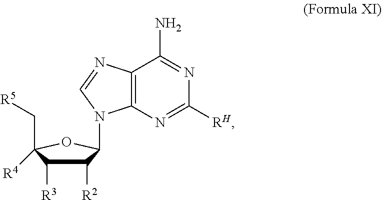
with an alkali hydroxide or alkali earth hydroxide (e.g., NaOH);
wherein:
R^{H} is halogen (e.g., chloro or fluoro);
R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R³ is a bond to an internucleotide linkage to a subsequent nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R^{a13})C(O)—, or —C(O)N(R^{a13})—;
R¹⁰ and R¹¹ and R^{a13} independently are H, optionally substituted C₁-C₆alkyl, optionally substituted C₂-C₆ alkenyl or optionally substituted C₂-C₆ alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵ represents a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is 0 or S;
a and b are each independently 1-10;
each R⁶ and R⁷ is independently hydrogen or -L-R^{L}, provided that at least one of R⁶ and R⁷ is not H;
or R⁶ and R⁷taken together with the nitrogen atom to which they are attached form a 3-10 membered heterocyclyl or a 3-10 membered heteroaryl group, the heterocyclyl or heteroaryl comprising one -L-R^{L} group,
wherein
L is absent or a linker;
each R^{L} is a ligand, (e.g., selected independently from the group consisting of carbohydrates, peptides, lipids, therapeutic agents, diagnostic agents, detectable labels, antibodies or fragments thereof, vitamins, optionally substituted C₁₋₃₀ alkyl, optionally substituted C₁₋₃₀ alkenyl, or optionally substituted C₁₋₃₀ alkynyl);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl,
provided that,
(i) no more than one of R² and R³ is a bond to an internucleotide linkage to a subsequent nucleotide; and
(ii) when both of R² and R³ are not a bond, then R⁵ is a bond to an internucleotide linkage to a preceding nucleotide.

53.-87. (canceled)
